SEQUENCE LISTING (1212 sequences)

SEQ ID NO: 1 - DNA, 20 bp, synthetic construct
  source 1..20: mol_type = other DNA
taatcggcagttgtccacac

SEQ ID NO: 2 - DNA, 20 bp, synthetic construct
  source 1..20: mol_type = other DNA
gcgctggcaagacgtgtcga

SEQ ID NO: 3 - DNA, 20 bp, synthetic construct
  source 1..20: mol_type = other DNA
ggcatcgtgtactaccacgg

SEQ ID NO: 4 - DNA, 20 bp, synthetic construct
  source 1..20: mol_type = other DNA
cagctggtgactaacgcaca

SEQ ID NO: 5 - DNA, 20 bp, synthetic construct
  source 1..20: mol_type = other DNA
ccacgttttgcaactaacga

SEQ ID NO: 6 - DNA, 20 bp, synthetic construct
  source 1..20: mol_type = other DNA
gcacaaattgtcgtcctgac

SEQ ID NO: 7 - DNA, 20 bp, synthetic construct
  source 1..20: mol_type = other DNA
cgcatgacctcgaccatctg

SEQ ID NO: 8 - DNA, 20 bp, synthetic construct
  source 1..20: mol_type = other DNA
accctcgtgtgcctcttcgt

SEQ ID NO: 9 - DNA, 20 bp, synthetic construct
  source 1..20: mol_type = other DNA
tgccagatagcaccgtccaa

SEQ ID NO: 10 - DNA, 20 bp, synthetic construct
  source 1..20: mol_type = other DNA
ggcgggccacatacaccgac

SEQ ID NO: 11 - DNA, 20 bp, synthetic construct
  source 1..20: mol_type = other DNA
actcgacttcgaagacccat

SEQ ID NO: 12 - DNA, 20 bp, synthetic construct
  source 1..20: mol_type = other DNA
ctggtaagtgtagtagacga

SEQ ID NO: 13 - DNA, 20 bp, synthetic construct
  source 1..20: mol_type = other DNA
acctggtctcaacgccatcc

SEQ ID NO: 14 - DNA, 20 bp, synthetic construct
  source 1..20: mol_type = other DNA
tcgtgtgggagcacgacatc

SEQ ID NO: 15 - DNA, 20 bp, synthetic construct
  source 1..20: mol_type = other DNA
catgtggcagaccgactgat

SEQ ID NO: 16 - DNA, 20 bp, synthetic construct
  source 1..20: mol_type = other DNA
cgtgcaaaaagacgacggcc

SEQ ID NO: 17 - DNA, 20 bp, synthetic construct
  source 1..20: mol_type = other DNA
atacatccgcttccgacacc

SEQ ID NO: 18 - DNA, 20 bp, synthetic construct
  source 1..20: mol_type = other DNA
ttggacgaagtagtagaccc

SEQ ID NO: 19 - DNA, 20 bp, synthetic construct
  source 1..20: mol_type = other DNA
gattgtcagttgagtactgc

SEQ ID NO: 20 - DNA, 20 bp, synthetic construct
  source 1..20: mol_type = other DNA
gcctggacgacattggccat

SEQ ID NO: 21 - DNA, 20 bp, synthetic construct
  source 1..20: mol_type = other DNA
agggacgtgtgtatcactac

SEQ ID NO: 22 - DNA, 20 bp, synthetic construct
  source 1..20: mol_type = other DNA
tcgacacgccggatgccaga

SEQ ID NO: 23 - DNA, 20 bp, synthetic construct
  source 1..20: mol_type = other DNA
aagctgctctactcatcgac

SEQ ID NO: 24 - DNA, 20 bp, synthetic construct
  source 1..20: mol_type = other DNA
tcaagctttaccccaccata

SEQ ID NO: 25 - DNA, 20 bp, synthetic construct
  source 1..20: mol_type = other DNA
gccgccgagacgatgaccac

SEQ ID NO: 26 - DNA, 20 bp, synthetic construct
  source 1..20: mol_type = other DNA
ggataggtcgcggttgacaa

SEQ ID NO: 27 - DNA, 20 bp, synthetic construct
  source 1..20: mol_type = other DNA
gcatctgacccaagaaacta

SEQ ID NO: 28 - DNA, 20 bp, synthetic construct
  source 1..20: mol_type = other DNA
ttgcacgtgagctcgcccat

SEQ ID NO: 29 - DNA, 20 bp, synthetic construct
  source 1..20: mol_type = other DNA
gcaataggcactctccacgg

SEQ ID NO: 30 - DNA, 20 bp, synthetic construct
  source 1..20: mol_type = other DNA
gagcgtcccggctgtaccaa

SEQ ID NO: 31 - DNA, 20 bp, synthetic construct
  source 1..20: mol_type = other DNA
gtcaggatgaccgaatacgt

SEQ ID NO: 32 - DNA, 20 bp, synthetic construct
  source 1..20: mol_type = other DNA
tttccggctagcacgtacca

SEQ ID NO: 33 - DNA, 20 bp, synthetic construct
  source 1..20: mol_type = other DNA
atgaagcgcccacacgaaat

SEQ ID NO: 34 - DNA, 20 bp, synthetic construct
  source 1..20: mol_type = other DNA
aagaagcgttcgtattcggt

SEQ ID NO: 35 - DNA, 20 bp, synthetic construct
  source 1..20: mol_type = other DNA
ggcttgttacacgtactcta

SEQ ID NO: 36 - DNA, 20 bp, synthetic construct
  source 1..20: mol_type = other DNA
aatacaatggactccaccgc

SEQ ID NO: 37 - DNA, 20 bp, synthetic construct
  source 1..20: mol_type = other DNA
gtctctatgtgaacggatct

SEQ ID NO: 38 - DNA, 20 bp, synthetic construct
  source 1..20: mol_type = other DNA
tgggacgtcccacaatggat

SEQ ID NO: 39 - DNA, 20 bp, synthetic construct
  source 1..20: mol_type = other DNA
gtgctttgatccaccgacac

SEQ ID NO: 40 - DNA, 20 bp, synthetic construct
  source 1..20: mol_type = other DNA
gagggctcggtcataagtac

SEQ ID NO: 41 - DNA, 20 bp, synthetic construct
  source 1..20: mol_type = other DNA
tgtaggagcactgtcgaccc

SEQ ID NO: 42 - DNA, 20 bp, synthetic construct
  source 1..20: mol_type = other DNA
actggtgttgaaccgtgtta

SEQ ID NO: 43 - DNA, 20 bp, synthetic construct
  source 1..20: mol_type = other DNA
cacctcatatgggtcgtccg

SEQ ID NO: 44 - DNA, 20 bp, synthetic construct
  source 1..20: mol_type = other DNA
tacgagtcaaactccccttc

SEQ ID NO: 45 - DNA, 20 bp, synthetic construct
  source 1..20: mol_type = other DNA
ccacgtagttggcgacttcc

SEQ ID NO: 46 - DNA, 20 bp, synthetic construct
  source 1..20: mol_type = other DNA
gccagtatcagtacgtgtaa

SEQ ID NO: 47 - DNA, 20 bp, synthetic construct
  source 1..20: mol_type = other DNA
ctcggactggacccaccacg

SEQ ID NO: 48 - DNA, 20 bp, synthetic construct
  source 1..20: mol_type = other DNA
gacgctaagcacgatggtgt

SEQ ID NO: 49 - DNA, 20 bp, synthetic construct
  source 1..20: mol_type = other DNA
taaccgaacatgtgctccac

SEQ ID NO: 50 - DNA, 20 bp, synthetic construct
  source 1..20: mol_type = other DNA
tcaaggttttgagtcggttc

SEQ ID NO: 51 - DNA, 20 bp, synthetic construct
  source 1..20: mol_type = other DNA
accggatcaacgccacggtg

SEQ ID NO: 52 - DNA, 20 bp, synthetic construct
  source 1..20: mol_type = other DNA
ctacggacgcgcatcaagag

SEQ ID NO: 53 - DNA, 20 bp, synthetic construct
  source 1..20: mol_type = other DNA
tattaaagtatcggtacgat

SEQ ID NO: 54 - DNA, 20 bp, synthetic construct
  source 1..20: mol_type = other DNA
tttgagtccgaccaccaatc

SEQ ID NO: 55 - DNA, 20 bp, synthetic construct
  source 1..20: mol_type = other DNA
ctacgaggagcatttgcact

SEQ ID NO: 56 - DNA, 20 bp, synthetic construct
  source 1..20: mol_type = other DNA
cttgcaggacctgaagcaac

SEQ ID NO: 57 - DNA, 20 bp, synthetic construct
  source 1..20: mol_type = other DNA
cctgatagcctatacgttca

SEQ ID NO: 58 - DNA, 20 bp, synthetic construct
  source 1..20: mol_type = other DNA
agcccaagggaagtcaccgc

SEQ ID NO: 59 - DNA, 20 bp, synthetic construct
  source 1..20: mol_type = other DNA
gcggcctcaacgacgagacc

SEQ ID NO: 60 - DNA, 20 bp, synthetic construct
  source 1..20: mol_type = other DNA
caacgtgttcgtgacttcgc

SEQ ID NO: 61 - DNA, 20 bp, synthetic construct
  source 1..20: mol_type = other DNA
gaactcctcgatctcgtcgt

SEQ ID NO: 62 - DNA, 20 bp, synthetic construct
  source 1..20: mol_type = other DNA
ataagagctgctcatcgcat

SEQ ID NO: 63 - DNA, 20 bp, synthetic construct
  source 1..20: mol_type = other DNA
aaggcgatgatgagcaccgt

SEQ ID NO: 64 - DNA, 20 bp, synthetic construct
  source 1..20: mol_type = other DNA
tggtgcaccgctatctgacg

SEQ ID NO: 65 - DNA, 20 bp, synthetic construct
  source 1..20: mol_type = other DNA
tggaatattgtgcttgactc

SEQ ID NO: 66 - DNA, 20 bp, synthetic construct
  source 1..20: mol_type = other DNA
tggtggtgctggagataccg

SEQ ID NO: 67 - DNA, 20 bp, synthetic construct
  source 1..20: mol_type = other DNA
attcccatgttgaaccccga

SEQ ID NO: 68 - DNA, 20 bp, synthetic construct
  source 1..20: mol_type = other DNA
gatcgacgtgtaccactacg

SEQ ID NO: 69 - DNA, 20 bp, synthetic construct
  source 1..20: mol_type = other DNA
gtagcaccacatcaacggca

SEQ ID NO: 70 - DNA, 20 bp, synthetic construct
  source 1..20: mol_type = other DNA
catcgaccggaagcgcacgg

SEQ ID NO: 71 - DNA, 20 bp, synthetic construct
  source 1..20: mol_type = other DNA
gtaccaatgagtgcaaagcg

SEQ ID NO: 72 - DNA, 20 bp, synthetic construct
  source 1..20: mol_type = other DNA
aaggataacatcgttaccac

SEQ ID NO: 73 - DNA, 20 bp, synthetic construct
  source 1..20: mol_type = other DNA
cggatcttcttaaacacgtt

SEQ ID NO: 74 - DNA, 20 bp, synthetic construct
  source 1..20: mol_type = other DNA
ggccccgctgaacgacacca

SEQ ID NO: 75 - DNA, 20 bp, synthetic construct
  source 1..20: mol_type = other DNA
tgcggaaatgagatccttat

SEQ ID NO: 76 - DNA, 20 bp, synthetic construct
  source 1..20: mol_type = other DNA
ccaaggttgccatcggaacc

SEQ ID NO: 77 - DNA, 20 bp, synthetic construct
  source 1..20: mol_type = other DNA
tcctgattgatggctacccg

SEQ ID NO: 78 - DNA, 20 bp, synthetic construct
  source 1..20: mol_type = other DNA
gagtggccgttcctaccacg

SEQ ID NO: 79 - DNA, 20 bp, synthetic construct
  source 1..20: mol_type = other DNA
attctgcacaatctgtttgc

SEQ ID NO: 80 - DNA, 20 bp, synthetic construct
  source 1..20: mol_type = other DNA
agaagcgggactatttctac

SEQ ID NO: 81 - DNA, 20 bp, synthetic construct
  source 1..20: mol_type = other DNA
acgccaatggcaactacact

SEQ ID NO: 82 - DNA, 20 bp, synthetic construct
  source 1..20: mol_type = other DNA
aagaatatagtcgttatcag

SEQ ID NO: 83 - DNA, 20 bp, synthetic construct
  source 1..20: mol_type = other DNA
gaacgttgcttttccaccga

SEQ ID NO: 84 - DNA, 20 bp, synthetic construct
  source 1..20: mol_type = other DNA
ggacacccccattgattact

SEQ ID NO: 85 - DNA, 20 bp, synthetic construct
  source 1..20: mol_type = other DNA
acggagctgacttcgccaag

SEQ ID NO: 86 - DNA, 20 bp, synthetic construct
  source 1..20: mol_type = other DNA
tcgtttataaccactacgag

SEQ ID NO: 87 - DNA, 20 bp, synthetic construct
  source 1..20: mol_type = other DNA
ttccatggacgttacgcccc

SEQ ID NO: 88 - DNA, 20 bp, synthetic construct
  source 1..20: mol_type = other DNA
gtggcactcactctctgttc

SEQ ID NO: 89 - DNA, 20 bp, synthetic construct
  source 1..20: mol_type = other DNA
acatccaggtctgcatcccc

SEQ ID NO: 90 - DNA, 20 bp, synthetic construct
  source 1..20: mol_type = other DNA
tgtccccgcacggagcccac

SEQ ID NO: 91 - DNA, 20 bp, synthetic construct
  source 1..20: mol_type = other DNA
acggagaccccgaagtttac

SEQ ID NO: 92 - DNA, 20 bp, synthetic construct
  source 1..20: mol_type = other DNA
ccgctacgaatacgatcact

SEQ ID NO: 93 - DNA, 20 bp, synthetic construct
  source 1..20: mol_type = other DNA
gcaaatgagtacggcttgtt

SEQ ID NO: 94 - DNA, 20 bp, synthetic construct
  source 1..20: mol_type = other DNA
ggattcatacgacgtgactg

SEQ ID NO: 95 - DNA, 20 bp, synthetic construct
  source 1..20: mol_type = other DNA
cgtcgagcccatacaggaac

SEQ ID NO: 96 - DNA, 20 bp, synthetic construct
  source 1..20: mol_type = other DNA
gataaccctaacctacaccg

SEQ ID NO: 97 - DNA, 20 bp, synthetic construct
  source 1..20: mol_type = other DNA
acaatggtgtcgcgtacatg

SEQ ID NO: 98 - DNA, 20 bp, synthetic construct
  source 1..20: mol_type = other DNA
gagtggatatggcctcgacc

SEQ ID NO: 99 - DNA, 20 bp, synthetic construct
  source 1..20: mol_type = other DNA
gcaccaccaaatcatccccg

SEQ ID NO: 100 - DNA, 20 bp, synthetic construct
  source 1..20: mol_type = other DNA
acgattacacctgtcgcctg

SEQ ID NO: 101 - DNA, 20 bp, synthetic construct
  source 1..20: mol_type = other DNA
atctttacccaagagactcg

SEQ ID NO: 102 - DNA, 20 bp, synthetic construct
  source 1..20: mol_type = other DNA
gattaagtgctggaacggcg

SEQ ID NO: 103 - DNA, 20 bp, synthetic construct
  source 1..20: mol_type = other DNA
acattgtgagccgggtcaac

SEQ ID NO: 104 - DNA, 20 bp, synthetic construct
  source 1..20: mol_type = other DNA
acaagacggaccggaaccac

SEQ ID NO: 105 - DNA, 20 bp, synthetic construct
  source 1..20: mol_type = other DNA
cccattcggtcttgcacatc

SEQ ID NO: 106 - DNA, 20 bp, synthetic construct
  source 1..20: mol_type = other DNA
ggtattctcacgggatcccg

SEQ ID NO: 107 - DNA, 20 bp, synthetic construct
  source 1..20: mol_type = other DNA
cttcgacacaatgccaacgt

SEQ ID NO: 108 - DNA, 20 bp, synthetic construct
  source 1..20: mol_type = other DNA
tagactggatgctgctcgac

SEQ ID NO: 109 - DNA, 20 bp, synthetic construct
  source 1..20: mol_type = other DNA
ccattcgagtcaagcttggt

SEQ ID NO: 110 - DNA, 20 bp, synthetic construct
  source 1..20: mol_type = other DNA
caaagtttccaaacgacccc

SEQ ID NO: 111 - DNA, 20 bp, synthetic construct
  source 1..20: mol_type = other DNA
ggccactcacgtgaacacta

SEQ ID NO: 112 - DNA, 20 bp, synthetic construct
  source 1..20: mol_type = other DNA
tcggaaggcatatatcgtca

SEQ ID NO: 113 - DNA, 20 bp, synthetic construct
  source 1..20: mol_type = other DNA
ccacgttgaggctgctcaac

SEQ ID NO: 114 - DNA, 20 bp, synthetic construct
  source 1..20: mol_type = other DNA
agaagactgcactacgatcg

SEQ ID NO: 115 - DNA, 20 bp, synthetic construct
  source 1..20: mol_type = other DNA
gctatacggttcgggccaag

SEQ ID NO: 116 - DNA, 20 bp, synthetic construct
  source 1..20: mol_type = other DNA
ggaccggtttttcagatcat

SEQ ID NO: 117 - DNA, 20 bp, synthetic construct
  source 1..20: mol_type = other DNA
gggggccaacgtttacaccc

SEQ ID NO: 118 - DNA, 20 bp, synthetic construct
  source 1..20: mol_type = other DNA
agtgagtactctcctagtac

SEQ ID NO: 119 - DNA, 20 bp, synthetic construct
  source 1..20: mol_type = other DNA
agagattgtgcatcgttacg

SEQ ID NO: 120 - DNA, 20 bp, synthetic construct
  source 1..20: mol_type = other DNA
aagcgcttgcacgaattagt

SEQ ID NO: 121 - DNA, 20 bp, synthetic construct
  source 1..20: mol_type = other DNA
acaaccgttcgaaggatggt

SEQ ID NO: 122 - DNA, 20 bp, synthetic construct
  source 1..20: mol_type = other DNA
tttccatctagtcctcaagc

SEQ ID NO: 123 - DNA, 20 bp, synthetic construct
  source 1..20: mol_type = other DNA
tctttgcacacggttggatc

SEQ ID NO: 124 - DNA, 20 bp, synthetic construct
  source 1..20: mol_type = other DNA
ttgggacaacgttgtccagc

SEQ ID NO: 125 - DNA, 20 bp, synthetic construct
  source 1..20: mol_type = other DNA
accaggtgacattgtaccgc

SEQ ID NO: 126 - DNA, 20 bp, synthetic construct
  source 1..20: mol_type = other DNA
cggtcaagatggaccagcac

SEQ ID NO: 127 - DNA, 20 bp, synthetic construct
  source 1..20: mol_type = other DNA
tccaaggcactggagacgtc

SEQ ID NO: 128 - DNA, 20 bp, synthetic construct
  source 1..20: mol_type = other DNA
gcaacaacaaggagtacccg

SEQ ID NO: 129 - DNA, 20 bp, synthetic construct
  source 1..20: mol_type = other DNA
gaaccattgccacccgtctc

SEQ ID NO: 130 - DNA, 20 bp, synthetic construct
  source 1..20: mol_type = other DNA
acgagcccaagcccgcaact

SEQ ID NO: 131 - DNA, 20 bp, synthetic construct
  source 1..20: mol_type = other DNA
tgtaaaagtgaacaggtcga

SEQ ID NO: 132 - DNA, 20 bp, synthetic construct
  source 1..20: mol_type = other DNA
gcggaattgacaagttccga

SEQ ID NO: 133 - DNA, 20 bp, synthetic construct
  source 1..20: mol_type = other DNA
ctgggacgcaacctctctcg

SEQ ID NO: 134 - DNA, 20 bp, synthetic construct
  source 1..20: mol_type = other DNA
ggactatttatgactacgtg

SEQ ID NO: 135 - DNA, 20 bp, synthetic construct
  source 1..20: mol_type = other DNA
aggacaagattcgacccctc

SEQ ID NO: 136 - DNA, 20 bp, synthetic construct
  source 1..20: mol_type = other DNA
cccccaacttcagtttgtac

SEQ ID NO: 137 - DNA, 20 bp, synthetic construct
  source 1..20: mol_type = other DNA
gtccaccacgagcggaagta

SEQ ID NO: 138 - DNA, 20 bp, synthetic construct
  source 1..20: mol_type = other DNA
catcgtgtacccacccgagt

SEQ ID NO: 139 - DNA, 20 bp, synthetic construct
  source 1..20: mol_type = other DNA
tcaagacttggcatactcgc

SEQ ID NO: 140 - DNA, 20 bp, synthetic construct
  source 1..20: mol_type = other DNA
aaggtgttcggacggtcaat

SEQ ID NO: 141 - DNA, 20 bp, synthetic construct
  source 1..20: mol_type = other DNA
tcggatttccacacgacgta

SEQ ID NO: 142 - DNA, 20 bp, synthetic construct
  source 1..20: mol_type = other DNA
gatggcttggatcatcgaca

SEQ ID NO: 143 - DNA, 20 bp, synthetic construct
  source 1..20: mol_type = other DNA
tgccaattggataccgctgt

SEQ ID NO: 144 - DNA, 20 bp, synthetic construct
  source 1..20: mol_type = other DNA
gttctcgtcaaggacggcgt

SEQ ID NO: 145 - DNA, 20 bp, synthetic construct
  source 1..20: mol_type = other DNA
catggcaactaactctgatt

SEQ ID NO: 146 - DNA, 20 bp, synthetic construct
  source 1..20: mol_type = other DNA
tggtcgacacgacgttatcg

SEQ ID NO: 147 - DNA, 20 bp, synthetic construct
  source 1..20: mol_type = other DNA
tgccacgatgtcagtaagat

SEQ ID NO: 148 - DNA, 20 bp, synthetic construct
  source 1..20: mol_type = other DNA
aactactttgagagcaccga

SEQ ID NO: 149 - DNA, 20 bp, synthetic construct
  source 1..20: mol_type = other DNA
tgccactattatctagccca

SEQ ID NO: 150 - DNA, 20 bp, synthetic construct
  source 1..20: mol_type = other DNA
ctaccccacgacgttcgtta

SEQ ID NO: 151 - DNA, 20 bp, synthetic construct
  source 1..20: mol_type = other DNA
ttacctgctggagtcaacgg

SEQ ID NO: 152 - DNA, 20 bp, synthetic construct
  source 1..20: mol_type = other DNA
actactgagtagccctgacc

SEQ ID NO: 153 - DNA, 20 bp, synthetic construct
  source 1..20: mol_type = other DNA
cacacggatgtcgtcatcga

SEQ ID NO: 154 - DNA, 20 bp, synthetic construct
  source 1..20: mol_type = other DNA
ttgaacttgcctctccggac

SEQ ID NO: 155 - DNA, 20 bp, synthetic construct
  source 1..20: mol_type = other DNA
ctcacgcggctggaaaccac

SEQ ID NO: 156 - DNA, 20 bp, synthetic construct
  source 1..20: mol_type = other DNA
agtacaatttgcaccaccgg

SEQ ID NO: 157 - DNA, 20 bp, synthetic construct
  source 1..20: mol_type = other DNA
ctaaggattccacggcctct

SEQ ID NO: 158 - DNA, 20 bp, synthetic construct
  source 1..20: mol_type = other DNA
tgatcacggcgtatcgcaac

SEQ ID NO: 159 - DNA, 20 bp, synthetic construct
  source 1..20: mol_type = other DNA
atggctttacctcgtctcac

SEQ ID NO: 160 - DNA, 20 bp, synthetic construct
  source 1..20: mol_type = other DNA
gcgagtcaagtgcgtcaacg

SEQ ID NO: 161 - DNA, 20 bp, synthetic construct
  source 1..20: mol_type = other DNA
agaaccggagcaaaattcgc

SEQ ID NO: 162 - DNA, 20 bp, synthetic construct
  source 1..20: mol_type = other DNA
tgcctacgaccggcaccttt

SEQ ID NO: 163 - DNA, 20 bp, synthetic construct
  source 1..20: mol_type = other DNA
cgtgtggtactgttatcacg

SEQ ID NO: 164 - DNA, 20 bp, synthetic construct
  source 1..20: mol_type = other DNA
tgggatggctcgtagactat

SEQ ID NO: 165 - DNA, 20 bp, synthetic construct
  source 1..20: mol_type = other DNA
cggcttttaaccacccaacc

SEQ ID NO: 166 - DNA, 20 bp, synthetic construct
  source 1..20: mol_type = other DNA
gagacccacgcgttctttgt

SEQ ID NO: 167 - DNA, 20 bp, synthetic construct
  source 1..20: mol_type = other DNA
gacggtggtgcccaaatcgg

SEQ ID NO: 168 - DNA, 20 bp, synthetic construct
  source 1..20: mol_type = other DNA
ttcggcgtcaacgagagtac

SEQ ID NO: 169 - DNA, 20 bp, synthetic construct
  source 1..20: mol_type = other DNA
ttgcacagatctgggagtat

SEQ ID NO: 170 - DNA, 20 bp, synthetic construct
  source 1..20: mol_type = other DNA
gccaagctggatcttgatgc

SEQ ID NO: 171 - DNA, 20 bp, synthetic construct
  source 1..20: mol_type = other DNA
gtactacaccacggttgagc

SEQ ID NO: 172 - DNA, 20 bp, synthetic construct
  source 1..20: mol_type = other DNA
gaaacaggcgattacggagc

SEQ ID NO: 173 - DNA, 20 bp, synthetic construct
  source 1..20: mol_type = other DNA
tgtttggataggggtacacg

SEQ ID NO: 174 - DNA, 20 bp, synthetic construct
  source 1..20: mol_type = other DNA
ttcagtgctgcaactgccac

SEQ ID NO: 175 - DNA, 20 bp, synthetic construct
  source 1..20: mol_type = other DNA
gccaacaaccgtgcctacaa

SEQ ID NO: 176 - DNA, 20 bp, synthetic construct
  source 1..20: mol_type = other DNA
taatcaggttgtcaaaccgc

SEQ ID NO: 177 - DNA, 20 bp, synthetic construct
  source 1..20: mol_type = other DNA
gttcggcagcaacgttgagt

SEQ ID NO: 178 - DNA, 20 bp, synthetic construct
  source 1..20: mol_type = other DNA
tgcgaagcccataacgccaa

SEQ ID NO: 179 - DNA, 20 bp, synthetic construct
  source 1..20: mol_type = other DNA
actctaacacgttggggacg

SEQ ID NO: 180 - DNA, 20 bp, synthetic construct
  source 1..20: mol_type = other DNA
cccacggcgcaagaggtagc

SEQ ID NO: 181 - DNA, 20 bp, synthetic construct
  source 1..20: mol_type = other DNA
accaatgggcgcttacgaag

SEQ ID NO: 182 - DNA, 20 bp, synthetic construct
  source 1..20: mol_type = other DNA
ggaactctgagtcatagcgt

SEQ ID NO: 183 - DNA, 20 bp, synthetic construct
  source 1..20: mol_type = other DNA
ccaaaagcaccgagacttcg

SEQ ID NO: 184 - DNA, 20 bp, synthetic construct
  source 1..20: mol_type = other DNA
ggtttgggggacacacgggt

SEQ ID NO: 185 - DNA, 20 bp, synthetic construct
  source 1..20: mol_type = other DNA
accggagcatctgacaaacc

SEQ ID NO: 186 - DNA, 20 bp, synthetic construct
  source 1..20: mol_type = other DNA
gccaccaataatcgcaagag

SEQ ID NO: 187 - DNA, 20 bp, synthetic construct
  source 1..20: mol_type = other DNA
ggtgaatcaccagttccccc

SEQ ID NO: 188 - DNA, 20 bp, synthetic construct
  source 1..20: mol_type = other DNA
ctgcggaaccccactttcca

SEQ ID NO: 189 - DNA, 20 bp, synthetic construct
  source 1..20: mol_type = other DNA
taccgcccaaggagcattaa

SEQ ID NO: 190 - DNA, 20 bp, synthetic construct
  source 1..20: mol_type = other DNA
gtcaactatgtgcggtacaa

SEQ ID NO: 191 - DNA, 20 bp, synthetic construct
  source 1..20: mol_type = other DNA
tcaccgccacgtttgagatc

SEQ ID NO: 192 - DNA, 20 bp, synthetic construct
  source 1..20: mol_type = other DNA
gaatggcctgcaacgttgac

SEQ ID NO: 193 - DNA, 20 bp, synthetic construct
  source 1..20: mol_type = other DNA
gtgtgcctagaggaaatcgt

SEQ ID NO: 194 - DNA, 20 bp, synthetic construct
  source 1..20: mol_type = other DNA
atgttatcgacaagcctatt

SEQ ID NO: 195 - DNA, 20 bp, synthetic construct
  source 1..20: mol_type = other DNA
cgaccccggggaacaccctc

SEQ ID NO: 196 - DNA, 20 bp, synthetic construct
  source 1..20: mol_type = other DNA
caacgaggcagccgacacgt

SEQ ID NO: 197 - DNA, 20 bp, synthetic construct
  source 1..20: mol_type = other DNA
gatccaccaaagcttctgtc

SEQ ID NO: 198 - DNA, 20 bp, synthetic construct
  source 1..20: mol_type = other DNA
gtgtgtctaacaatacaact

SEQ ID NO: 199 - DNA, 20 bp, synthetic construct
  source 1..20: mol_type = other DNA
acacgaagccaatcaggttc

SEQ ID NO: 200 - DNA, 20 bp, synthetic construct
  source 1..20: mol_type = other DNA
agagagcaagctcccgggtt

SEQ ID NO: 201 - DNA, 20 bp, synthetic construct
  source 1..20: mol_type = other DNA
cttcctcaacgacgcggaca

SEQ ID NO: 202 - DNA, 20 bp, synthetic construct
  source 1..20: mol_type = other DNA
tggtgaagagcgtccaccgg

SEQ ID NO: 203 - DNA, 20 bp, synthetic construct
  source 1..20: mol_type = other DNA
cgtccacgaagaacccacta

SEQ ID NO: 204 - DNA, 20 bp, synthetic construct
  source 1..20: mol_type = other DNA
ggtgttccgaatgggaccac

SEQ ID NO: 205 - DNA, 20 bp, synthetic construct
  source 1..20: mol_type = other DNA
ggtgggaccgaagttacctc

SEQ ID NO: 206 - DNA, 20 bp, synthetic construct
  source 1..20: mol_type = other DNA
gtacgatgacttcccccacg

SEQ ID NO: 207 - DNA, 20 bp, synthetic construct
  source 1..20: mol_type = other DNA
gaacttctgtactacaacgc

SEQ ID NO: 208 - DNA, 20 bp, synthetic construct
  source 1..20: mol_type = other DNA
ccggaggttacccactgtga

SEQ ID NO: 209 - DNA, 20 bp, synthetic construct
  source 1..20: mol_type = other DNA
ttctcccttgaacgtggtac

SEQ ID NO: 210 - DNA, 20 bp, synthetic construct
  source 1..20: mol_type = other DNA
gcaaccaagaggaaacggcg

SEQ ID NO: 211 - DNA, 20 bp, synthetic construct
  source 1..20: mol_type = other DNA
caggacgtccgccacatact

SEQ ID NO: 212 - DNA, 20 bp, synthetic construct
  source 1..20: mol_type = other DNA
tcaacgccagatcttgtcgt

SEQ ID NO: 213 - DNA, 20 bp, synthetic construct
  source 1..20: mol_type = other DNA
aagctttttgtcatcagctg

SEQ ID NO: 214 - DNA, 20 bp, synthetic construct
  source 1..20: mol_type = other DNA
gagggaacttctgatggtac

SEQ ID NO: 215 - DNA, 20 bp, synthetic construct
  source 1..20: mol_type = other DNA
taatcttcacgtcgaagtga

SEQ ID NO: 216 - DNA, 20 bp, synthetic construct
  source 1..20: mol_type = other DNA
gtagtctaccaccatgccac

SEQ ID NO: 217 - DNA, 20 bp, synthetic construct
  source 1..20: mol_type = other DNA
aactgaaaccggtactgatt

SEQ ID NO: 218 - DNA, 20 bp, synthetic construct
  source 1..20: mol_type = other DNA
tcggagtcgctgcaaagtcg

SEQ ID NO: 219 - DNA, 20 bp, synthetic construct
  source 1..20: mol_type = other DNA
gggggacctttggcacacgc

SEQ ID NO: 220 - DNA, 20 bp, synthetic construct
  source 1..20: mol_type = other DNA
accatcacggccagacgcgt

SEQ ID NO: 221 - DNA, 20 bp, synthetic construct
  source 1..20: mol_type = other DNA
atgcttaccgggggaccaaa

SEQ ID NO: 222 - DNA, 20 bp, synthetic construct
  source 1..20: mol_type = other DNA
ctgggccacaaaagggatac

SEQ ID NO: 223 - DNA, 20 bp, synthetic construct
  source 1..20: mol_type = other DNA
gtcctgggtgctgatgtcat

SEQ ID NO: 224 - DNA, 20 bp, synthetic construct
  source 1..20: mol_type = other DNA
agacgctggacagcatacga

SEQ ID NO: 225 - DNA, 20 bp, synthetic construct
  source 1..20: mol_type = other DNA
tcggtgctgaagtcctcgtt

SEQ ID NO: 226 - DNA, 20 bp, synthetic construct
  source 1..20: mol_type = other DNA
ggggtacggctgaagactcg

SEQ ID NO: 227 - DNA, 20 bp, synthetic construct
  source 1..20: mol_type = other DNA
ctggtttcatggttcctccg

SEQ ID NO: 228 - DNA, 20 bp, synthetic construct
  source 1..20: mol_type = other DNA
acggaatcccacagctggta

SEQ ID NO: 229 - DNA, 20 bp, synthetic construct
  source 1..20: mol_type = other DNA
atcctctgtccagaatgagc

SEQ ID NO: 230 - DNA, 20 bp, synthetic construct
  source 1..20: mol_type = other DNA
cgcaccccccaacatctacg

SEQ ID NO: 231 - DNA, 20 bp, synthetic construct
  source 1..20: mol_type = other DNA
aaggatgcacgctccccacc

SEQ ID NO: 232 - DNA, 20 bp, synthetic construct
  source 1..20: mol_type = other DNA
ccgagtccacatgttagccc

SEQ ID NO: 233 - DNA, 20 bp, synthetic construct
  source 1..20: mol_type = other DNA
gcgggccaacttcactctgc

SEQ ID NO: 234 - DNA, 20 bp, synthetic construct
  source 1..20: mol_type = other DNA
gcccaccaaacccccgacga

SEQ ID NO: 235 - DNA, 20 bp, synthetic construct
  source 1..20: mol_type = other DNA
gtccccacaaagttcagggc

SEQ ID NO: 236 - DNA, 20 bp, synthetic construct
  source 1..20: mol_type = other DNA
gctaccggagcacagtgcac

SEQ ID NO: 237 - DNA, 20 bp, synthetic construct
  source 1..20: mol_type = other DNA
gggggcctacaccttccaac

SEQ ID NO: 238 - DNA, 20 bp, synthetic construct
  source 1..20: mol_type = other DNA
aacccaaggagttaatccta

SEQ ID NO: 239 - DNA, 20 bp, synthetic construct
  source 1..20: mol_type = other DNA
ttattatggactggtgctta

SEQ ID NO: 240 - DNA, 20 bp, synthetic construct
  source 1..20: mol_type = other DNA
ctcagcaaggacgaacgcca

SEQ ID NO: 241 - DNA, 20 bp, synthetic construct
  source 1..20: mol_type = other DNA
tgtgctggtaggatttgtgc

SEQ ID NO: 242 - DNA, 20 bp, synthetic construct
  source 1..20: mol_type = other DNA
caggtctttgatcaactcga

SEQ ID NO: 243 - DNA, 20 bp, synthetic construct
  source 1..20: mol_type = other DNA
cactagaacgccacccaaag

SEQ ID NO: 244 - DNA, 20 bp, synthetic construct
  source 1..20: mol_type = other DNA
tctatcgtccacacggagga

SEQ ID NO: 245 - DNA, 20 bp, synthetic construct
  source 1..20: mol_type = other DNA
caccctggacatagcacgtc

SEQ ID NO: 246 - DNA, 20 bp, synthetic construct
  source 1..20: mol_type = other DNA
gttcaccagctccgtgtcga

SEQ ID NO: 247 - DNA, 20 bp, synthetic construct
  source 1..20: mol_type = other DNA
gccaccacacagccgacgaa

SEQ ID NO: 248 - DNA, 20 bp, synthetic construct
  source 1..20: mol_type = other DNA
acctctgggaccttggcggt

SEQ ID NO: 249 - DNA, 20 bp, synthetic construct
  source 1..20: mol_type = other DNA
cgacgcacactgcattaatg

SEQ ID NO: 250 - DNA, 20 bp, synthetic construct
  source 1..20: mol_type = other DNA
tggatgatcacttaatacga

SEQ ID NO: 251 - DNA, 20 bp, synthetic construct
  source 1..20: mol_type = other DNA
gggttgaccccaaagctaac

SEQ ID NO: 252 - DNA, 20 bp, synthetic construct
  source 1..20: mol_type = other DNA
cggcattgaactcggtcagt

SEQ ID NO: 253 - DNA, 20 bp, synthetic construct
  source 1..20: mol_type = other DNA
ccttgtacagatgcaaacgg

SEQ ID NO: 254 - DNA, 20 bp, synthetic construct
  source 1..20: mol_type = other DNA
aagaatgccacgcttgtcgc

SEQ ID NO: 255 - DNA, 20 bp, synthetic construct
  source 1..20: mol_type = other DNA
gtcaaaggacgccctgaatc

SEQ ID NO: 256 - DNA, 86 bp, synthetic construct
  source 1..86: mol_type = other DNA
gtttttgggatgcccccaacaaataatcggcagttgtccagaattccactggcacaaacccctaagaggcgcccatttctgcaaaa

SEQ ID NO: 257 - DNA, 86 bp, synthetic construct
  source 1..86: mol_type = other DNA
gtggagcgcgtggcgcgcgagcggcgctggcaagacgtgtgaattccgatggcgcgcatgcgcacgttgcacgcggcgctgggcgg

SEQ ID NO: 258 - DNA, 86 bp, synthetic construct
  source 1..86: mol_type = other DNA
catccacctgcaccctgaagcctggcatcgtgtactaccagaattccggtggcgggggcgtcatggggagtttgagtaagaaccat

SEQ ID NO: 259 - DNA, 86 bp, synthetic construct
  source 1..86: mol_type = other DNA
ggtctcctgagaagacggaggagcagctggtgactaacgcgaattcacagggtacgccagcagccatagctgcgctcgcgagccgg

SEQ ID NO: 260 - DNA, 86 bp, synthetic construct
  source 1..86: mol_type = other DNA
tgctggttgtgcagaaggtttatccacgttttgcaactaagaattccgaaggtttcagaaccctccagaagtctgttaaacatctg

SEQ ID NO: 261 - DNA, 86 bp, synthetic construct
  source 1..86: mol_type = other DNA
taatggctcttattgtaaaatctgcacaaattgtcgtcctgaattcgactggttttgtgatggtcttcaccctctttctcctctat

SEQ ID NO: 262 - DNA, 86 bp, synthetic construct
  source 1..86: mol_type = other DNA
accaaatggcccttttcattcagcgcatgacctcgaccatgaattcctgtggtttcctgttgggatttttcaggggttggaaactg

SEQ ID NO: 263 - DNA, 86 bp, synthetic construct
  source 1..86: mol_type = other DNA
tgcgggcctcgtggctggtcatcaccctcgtgtgcctcttgaattccgtgggcatctatgccatggtgaagctgctgctcttctca

SEQ ID NO: 264 - DNA, 86 bp, synthetic construct
  source 1..86: mol_type = other DNA
cccaccagcttcagcagtccaagtgccagatagcaccgtcgaattccaaaggccccatacaaggccctccacagccgtgccccctc

SEQ ID NO: 265 - DNA, 86 bp, synthetic construct
  source 1..86: mol_type = other DNA
gcagcgggccagccttccgtccaggcgggccacatacaccgaattcgacaggaaggtgcggctcaccaaggcggccgagtgcaggg

SEQ ID NO: 266 - DNA, 86 bp, synthetic construct
  source 1..86: mol_type = other DNA
tggttgtttctgggctattcctaactcgacttcgaagaccgaattccattggtaagatgacaataactgagcaaaagtatgaagga

SEQ ID NO: 267 - DNA, 86 bp, synthetic construct
  source 1..86: mol_type = other DNA
gggaggcttcacctttggaagcactggtaagtgtagtagagaattccgagggtgaacggggagatgatgagcttgctggccatgct

SEQ ID NO: 268 - DNA, 86 bp, synthetic construct
  source 1..86: mol_type = other DNA
tgtgtttacagtgggatcatgaaacctggtctcaacgccagaattctcctgggacccacaggtggaggcaaatcttcgtgagtata

SEQ ID NO: 269 - DNA, 86 bp, synthetic construct
  source 1..86: mol_type = other DNA
cccgccagagagcagggctgtcctcgtgtgggagcacgacgaattcatcaggcccagtgccgtcaggatctctgtcacctggcccc

SEQ ID NO: 270 - DNA, 86 bp, synthetic construct
  source 1..86: mol_type = other DNA
tcatggcagagctgagtctgagccatgtggcagaccgactgaattcgattggcaactacagcttggggggcatttccacgggtgag

SEQ ID NO: 271 - DNA, 86 bp, synthetic construct
  source 1..86: mol_type = other DNA
gttagttttgctgccgaagagcccgtgcaaaaagacgacggaattcgccgggagggctgcctccccgtccagaagcctgtaggaaa

SEQ ID NO: 272 - DNA, 86 bp, synthetic construct
  source 1..86: mol_type = other DNA
tccgacctcaaggaggcggtcacatacatccgcttccgacgaattcacccggcggcgccgctgttcgcggtgagcgaaggctcggg

SEQ ID NO: 273 - DNA, 86 bp, synthetic construct
  source 1..86: mol_type = other DNA
ctatgcttttgacctattgggttttggacgaagtagtagagaattccccaggtttgacagtgatgcagaagaagtggagaatcagt

SEQ ID NO: 274 - DNA, 86 bp, synthetic construct
  source 1..86: mol_type = other DNA
ttctttgagccgttgtacaaattgattgtcagttgagtacgaattctgcaggcctgtgggattcaaacccaaatggcttgatgata

SEQ ID NO: 275 - DNA, 86 bp, synthetic construct
  source 1..86: mol_type = other DNA
caggagacccctcaactttggctgcctggacgacattggcgaattccatgggatcaaggtagagagaggggccctcctctttccct

SEQ ID NO: 276 - DNA, 86 bp, synthetic construct
  source 1..86: mol_type = other DNA
gctgtccatctcgctcaggtacgagggacgtgtgtatcacgaattctacaggatcaataccactgcagatggcaaggtaagactcc

SEQ ID NO: 277 - DNA, 86 bp, synthetic construct
  source 1..86: mol_type = other DNA
ctgtcttcagttctggctgcttgtcgacacgccggatgccgaattcagatggaggaacctgttgacaaattcccaagagggaaatg

SEQ ID NO: 278 - DNA, 86 bp, synthetic construct
  source 1..86: mol_type = other DNA
ttaacaccaagtataataacagaaagctgctctactcatcgaattcgactggaacattccttatataaacctcaaaaagggtaaat

SEQ ID NO: 279 - DNA, 86 bp, synthetic construct
  source 1..86: mol_type = other DNA
gcccgtcttttacaatcaaatcttcaagctttaccccaccgaattcatatggtgttgatcctctgttcattacatatggaacattg

SEQ ID NO: 280 - DNA, 86 bp, synthetic construct
  source 1..86: mol_type = other DNA
gccactcacctatgatggtccgcgccgccgagacgatgacgaattccacaggatctgagcctgcattcatcttgcttctcctgccg

SEQ ID NO: 281 - DNA, 86 bp, synthetic construct
  source 1..86: mol_type = other DNA
acagagatgtcaatcccacggatggataggtcgcggttgagaattccaaaggcagcttcacaatcactcagttggggaactggatc

SEQ ID NO: 282 - DNA, 86 bp, synthetic construct
  source 1..86: mol_type = other DNA
ccaaaggactgcaagttcatcaagcatctgacccaagaaagaattcctaagggttgcatggaagtagacggatccaattcctagaa

SEQ ID NO: 283 - DNA, 86 bp, synthetic construct
  source 1..86: mol_type = other DNA
tggagaagatgaggtgtgtgactttgcacgtgagctcgccgaattccatgggccactggttgtgctgcacgagactgaccacccag

SEQ ID NO: 284 - DNA, 86 bp, synthetic construct
  source 1..86: mol_type = other DNA
aagtagaagaagagcataccaatgcaataggcactctccagaattccggcggtttgacagccacgttagtagataacatatcaaca

SEQ ID NO: 285 - DNA, 86 bp, synthetic construct
  source 1..86: mol_type = other DNA
ggcctcatctcctgctgtccttcgagcgtcccggctgtacgaattccaatggagcctgaagagttcggcgcagttcctggggtctc

SEQ ID NO: 286 - DNA, 86 bp, synthetic construct
  source 1..86: mol_type = other DNA
acctggagccctggcttatgggagtcaggatgaccgaatagaattccgtcggggtgagtctgccttgagacagggaagtggttgaa

SEQ ID NO: 287 - DNA, 86 bp, synthetic construct
  source 1..86: mol_type = other DNA
agacattgccaaggtggtcatggtttccggctagcacgtagaattcccagggcactttgcgaagggagcggtcagagaatacgtcc

SEQ ID NO: 288 - DNA, 86 bp, synthetic construct
  source 1..86: mol_type = other DNA
agtggggcaggcagcccggcccaatgaagcgcccacacgagaattcaatgggaggggtgatctccacgagggcaatgtcatttccc

SEQ ID NO: 289 - DNA, 86 bp, synthetic construct
  source 1..86: mol_type = other DNA
tccaggttggagtcagcagtgcgaagaagcgttcgtattcgaattcggtaggagagagagggctgcctggagtggaggcctgagtc

SEQ ID NO: 290 - DNA, 86 bp, synthetic construct
  source 1..86: mol_type = other DNA
gtacagtggatcatctccgaattggcttgttacacgtactgaattcctatggtagctgtacctctgtatgacaccttgggaccaga

SEQ ID NO: 291 - DNA, 86 bp, synthetic construct
  source 1..86: mol_type = other DNA
ttgtcttaaggtcaggacactccaatacaatggactccacgaattccgctggggccacctcctcactggccacaatgcacttggcc

SEQ ID NO: 292 - DNA, 86 bp, synthetic construct
  source 1..86: mol_type = other DNA
tagtttgcaggcttctcttcagagtctctatgtgaacggagaattctcttggattccagctgcagtcttctcatcactacaggtga

SEQ ID NO: 293 - DNA, 86 bp, synthetic construct
  source 1..86: mol_type = other DNA
cataattttcctcacctgatgtctgggacgtcccacaatggaattcgatgggaaaacagccctgggagcatcgtccccagcaaagc

SEQ ID NO: 294 - DNA, 86 bp, synthetic construct
  source 1..86: mol_type = other DNA
ctggaaggatggcctcggcttctgtgctttgatccaccgagaattccaccggcccgagctgattgactacgggaagctgcggaagg

SEQ ID NO: 295 - DNA, 86 bp, synthetic construct
  source 1..86: mol_type = other DNA
ctcgaatttccctaggattcaaagagggctcggtcataaggaattctacaggctgttggctgggttttactccaagctcccgctca

SEQ ID NO: 296 - DNA, 86 bp, synthetic construct
  source 1..86: mol_type = other DNA
cacatttggtctctttttcttcctgtaggagcactgtcgagaattcccctggcatgcccatactgtcagcatcgcagctatggaca

SEQ ID NO: 297 - DNA, 86 bp, synthetic construct
  source 1..86: mol_type = other DNA
gggaaaaagacagaaccaatcaaactggtgttgaaccgtggaattcttatggtgacaaagataaacggcggcattgttttgctacc

SEQ ID NO: 298 - DNA, 86 bp, synthetic construct
  source 1..86: mol_type = other DNA
tggttcagctctcgggctcttgtcacctcatatgggtcgtgaattcccggggtgggcctggaactgtggagacgggaatgggggac

SEQ ID NO: 299 - DNA, 86 bp, synthetic construct
  source 1..86: mol_type = other DNA
gcttctcaggaatggtgacttcatacgagtcaaactccccgaattcttcagggtgaaaaattacatcttcgccaagagacttaagg

SEQ ID NO: 300 - DNA, 86 bp, synthetic construct
  source 1..86: mol_type = other DNA
ctccccgccgcccccaacctggtccacgtagttggcgactgaattctccaggagacgctgtttggtgtggttcaagtttcggtgcc

SEQ ID NO: 301 - DNA, 86 bp, synthetic construct
  source 1..86: mol_type = other DNA
tagtacacgaatatgattcagctgccagtatcagtacgtggaattctaatggtctaaggtaatgcagaatatctttctttttcctt

SEQ ID NO: 302 - DNA, 86 bp, synthetic construct
  source 1..86: mol_type = other DNA
caaaaaatgcccactggcatctactcggactggacccaccgaattcacgtggtattctggcaggcccttgataaaatgctctgaaa

SEQ ID NO: 303 - DNA, 86 bp, synthetic construct
  source 1..86: mol_type = other DNA
cctcccggtgttgcagacaggcagacgctaagcacgatgggaattctgtgggaggaggaggtgaggacgctggcaatgatggcgtc

SEQ ID NO: 304 - DNA, 86 bp, synthetic construct
  source 1..86: mol_type = other DNA
cacctgcgacccttgaccacaagtaaccgaacatgtgctcgaattccactgggaccactcttccacaccagattcacctgcgtgca

SEQ ID NO: 305 - DNA, 86 bp, synthetic construct
  source 1..86: mol_type = other DNA
cttaaaaaagcatatgaaagaattcaaggttttgagtcgggaattcttcaggtcacccaatttcggtaagtaacacaatggtctta

SEQ ID NO: 306 - DNA, 86 bp, synthetic construct
  source 1..86: mol_type = other DNA
ggacatggtgtccttcgaggaggaccggatcaacgccacggaattcgtgtggaagctccagcccacagccggcctccaggacctgc

SEQ ID NO: 307 - DNA, 86 bp, synthetic construct
  source 1..86: mol_type = other DNA
cgcgcaacatcgtcaagtatgacctacggacgcgcatcaagaattcgagcggggagacggtcatcaataccgccaactaccatgac

SEQ ID NO: 308 - DNA, 86 bp, synthetic construct
  source 1..86: mol_type = other DNA
aaatcttctaaagaagcatattctattaaagtatcggtacgaattcgatagggagtccagggcatgaaataaattttatctgcagc

SEQ ID NO: 309 - DNA, 86 bp, synthetic construct
  source 1..86: mol_type = other DNA
gagtataccagagtgaacatttgtttgagtccgaccaccagaattcatctggggcgtggtgcaaagaccctctgcaggcatctgac

SEQ ID NO: 310 - DNA, 86 bp, synthetic construct
  source 1..86: mol_type = other DNA
cttctgcactagattaagatgttctacgaggagcatttgcgaattcacttggacgatgagatccgctacatcctggatggcagtgg

SEQ ID NO: 311 - DNA, 86 bp, synthetic construct
  source 1..86: mol_type = other DNA
cccgaagccatggcaagcaagggcttgcaggacctgaagcgaattcaacaggtggaggggaccgcccaggaagccggtgaggatag

SEQ ID NO: 312 - DNA, 86 bp, synthetic construct
  source 1..86: mol_type = other DNA
tgtgtgcccaatcatggcagtgacctgatagcctatacgtgaattctcatggtcttcgatgacatgctgtaccaaagcttcatagg

SEQ ID NO: 313 - DNA, 86 bp, synthetic construct
  source 1..86: mol_type = other DNA
gaaataacagctcctgttcattcagcccaagggaagtcacgaattccgcgggaaagacctgctaacaaaaacaacaacaggtatct

SEQ ID NO: 314 - DNA, 86 bp, synthetic construct
  source 1..86: mol_type = other DNA
cgacggcgccgcctacccgcagtgcggcctcaacgacgaggaattcacctggtacatcctgtcctcctgcatcggctccttcttcg

SEQ ID NO: 315 - DNA, 86 bp, synthetic construct
  source 1..86: mol_type = other DNA
actccgagactccagaccatgaccaacgtgttcgtgacttgaattccgctggccgcagccgacctggtgatgggactcctggtggt

SEQ ID NO: 316 - DNA, 86 bp, synthetic construct
  source 1..86: mol_type = other DNA
agctctgacaggaagccctcgaggaactcctcgatctcgtgaattccgtcggtcagcaccgtctgcgggcgccctccagggcacag

SEQ ID NO: 317 - DNA, 86 bp, synthetic construct
  source 1..86: mol_type = other DNA
tcctcctcttcatcatacgactcataagagctgctcatcggaattccatcggaatcataatctgtaaaaaataaacatacactggt

SEQ ID NO: 318 - DNA, 86 bp, synthetic construct
  source 1..86: mol_type = other DNA
ctcttctgatggtgtagagcaagaaggcgatgatgagcacgaattccgtaggcagcatgctcagcagaaaagagctggggacacac

SEQ ID NO: 319 - DNA, 86 bp, synthetic construct
  source 1..86: mol_type = other DNA
cctgtctagcgggaagtcagccctggtgcaccgctatctggaattcacggggacctatgtccaggaggagtcccctgaaggtgagc

SEQ ID NO: 320 - DNA, 86 bp, synthetic construct
  source 1..86: mol_type = other DNA
caccttctgtttgagagttcctctggaatattgtgcttgagaattcctctggattggcagaaaggttaaactccaaagctatatgt

SEQ ID NO: 321 - DNA, 86 bp, synthetic construct
  source 1..86: mol_type = other DNA
ggggggcgggttgagcttggggctggtggtgctggagatagaattcccggggctgaagcatatggagtcacccagcccggtgtcca

SEQ ID NO: 322 - DNA, 86 bp, synthetic construct
  source 1..86: mol_type = other DNA
gtcgcctcttcgtctttaagctgattcccatgttgaacccgaattccgatggtgtggtccggggacactaccggtaagtggcttcc

SEQ ID NO: 323 - DNA, 86 bp, synthetic construct
  source 1..86: mol_type = other DNA
tactttgaggtggacatccctaagatcgacgtgtaccactgaattcacgaggtggacatcaagccggataagtgtccccgtagagt

SEQ ID NO: 324 - DNA, 86 bp, synthetic construct
  source 1..86: mol_type = other DNA
ccacttcatggagggcagatgtcgtagcaccacatcaacggaattcgcatggacagggttagtgctgattggcttgtctaattcca

SEQ ID NO: 325 - DNA, 86 bp, synthetic construct
  source 1..86: mol_type = other DNA
tgctggctggcaggagtcatcttcatcgaccggaagcgcagaattccgggggatgccatcagtgtcatgtctgaggtcgcccagac

SEQ ID NO: 326 - DNA, 86 bp, synthetic construct
  source 1..86: mol_type = other DNA
gggccggccccgggaggctctgagtaccaatgagtgcaaagaattcgcgcggagagccgcgtcggcggccacggcagcgcccacgg

SEQ ID NO: 327 - DNA, 86 bp, synthetic construct
  source 1..86: mol_type = other DNA
ttaaaaagcaaaacagtgaggtgaaggataacatcgttacgaattccactggacactgttggcttcattgtctgaatatatctgag

SEQ ID NO: 328 - DNA, 86 bp, synthetic construct
  source 1..86: mol_type = other DNA
tcacgctctcttccacaatgcctcggatcttcttaaacacgaattcgtttggatacttcttataaacccagtgggttaagtctccc

SEQ ID NO: 329 - DNA, 86 bp, synthetic construct
  source 1..86: mol_type = other DNA
gtgtgccaagactgccccctgctggccccgctgaacgacagaattcccagggtggtgcacgccgcgaaagctgccctggccgcctt

SEQ ID NO: 330 - DNA, 86 bp, synthetic construct
  source 1..86: mol_type = other DNA
agaacgctgaactccttcaatcctgcggaaatgagatcctgaattctattggccttaagaagagccatctgcctgggtagagaaaa

SEQ ID NO: 331 - DNA, 86 bp, synthetic construct
  source 1..86: mol_type = other DNA
ctctgttgccaggtactttattaccaaggttgccatcggagaattcaccaggaatgacattactcactatcagaattgagaaaatt

SEQ ID NO: 332 - DNA, 86 bp, synthetic construct
  source 1..86: mol_type = other DNA
caaagtcaatacttccaaaggcttcctgattgatggctacgaattcccgcgggaggtgcagcaaggagaagagtttgagcgacggg

SEQ ID NO: 333 - DNA, 86 bp, synthetic construct
  source 1..86: mol_type = other DNA
tctttcaggctgattcaccccaagagtggccgttcctaccgaattcacgaggagttcaaccctccaaaagagcccatgaaagatga

SEQ ID NO: 334 - DNA, 86 bp, synthetic construct
  source 1..86: mol_type = other DNA
tgctgcacagcttgcaggattgcattctgcacaatctgttgaattctgctggcattctgcagcttcacctcttcaggctcatttcc

SEQ ID NO: 335 - DNA, 86 bp, synthetic construct
  source 1..86: mol_type = other DNA
tttcctgacaattcttttagtggagaagcgggactatttcgaattctactggctactgtaagtgtagaggaagattcttgggtttc

SEQ ID NO: 336 - DNA, 86 bp, synthetic construct
  source 1..86: mol_type = other DNA
gcgcattctgcaaggtctgcaggacgccaatggcaactacgaattcactaggcagacggacattctgcaagctttctctgaagaga

SEQ ID NO: 337 - DNA, 86 bp, synthetic construct
  source 1..86: mol_type = other DNA
gcaaatttttcagaacataaaacaagaatatagtcgttatgaattccagaggtggagacatttagaagttgttcttaatcagagtg

SEQ ID NO: 338 - DNA, 86 bp, synthetic construct
  source 1..86: mol_type = other DNA
gaacctacctcccaggcatccaagaacgttgcttttccacgaattccgatggcattacctttatcatctttggggaaaaggtcatc

SEQ ID NO: 339 - DNA, 86 bp, synthetic construct
  source 1..86: mol_type = other DNA
cttgagcagctccgtggtctcagggacacccccattgattgaattcactgggtacagatccttccatgcaaggagagaggggaggc

SEQ ID NO: 340 - DNA, 86 bp, synthetic construct
  source 1..86: mol_type = other DNA
gcgctgtgcccagtacaagaaggacggagctgacttcgccgaattcaagtggcgttgtgtgctgaagattggggaacacaccccct

SEQ ID NO: 341 - DNA, 86 bp, synthetic construct
  source 1..86: mol_type = other DNA
gctgatgattgttcatcaacttttcgtttataaccactacgaattcgagtggctttcctttttccagagtctccaaacagcttcct

SEQ ID NO: 342 - DNA, 86 bp, synthetic construct
  source 1..86: mol_type = other DNA
ctgcgaatatgggtagtgcttcgttccatggacgttacgcgaattccccgggagtctctcagtatcttggtagtggctgggtccgg

SEQ ID NO: 343 - DNA, 86 bp, synthetic construct
  source 1..86: mol_type = other DNA
gcaccggctcttcatgaagctgggtggcactcactctctggaattcttcagggcctggtaggcctcccctcctcagctgccttctc

SEQ ID NO: 344 - DNA, 86 bp, synthetic construct
  source 1..86: mol_type = other DNA
catccaccagaagcatttccagcacatccaggtctgcatcgaattcccctggctggagggccgaggactacccccgcttctaggtg

SEQ ID NO: 345 - DNA, 86 bp, synthetic construct
  source 1..86: mol_type = other DNA
actcttcctgctggcgctggctttgtccccgcacggagccgaattccacgggaggccccgggggcgcaggggagcgcgcgtcacgg

SEQ ID NO: 346 - DNA, 86 bp, synthetic construct
  source 1..86: mol_type = other DNA
tgcaggtataaagaagcgactaaacggagaccccgaagttgaattctactggagaaactgcgttgggtgaccgtaggctaccatta

SEQ ID NO: 347 - DNA, 86 bp, synthetic construct
  source 1..86: mol_type = other DNA
ctggagcccgagctgcgccgccgccgctacgaatacgatcgaattcactgggacgcggtgagaccggcagcgccgggggcgaggga

SEQ ID NO: 348 - DNA, 86 bp, synthetic construct
  source 1..86: mol_type = other DNA
attcttctgtagttctgtatcttgcaaatgagtacggcttgaattcgttaggtggcattaagagagcatccaccagtccacatttc

SEQ ID NO: 349 - DNA, 86 bp, synthetic construct
  source 1..86: mol_type = other DNA
attgcctgttctccttcccaggtggattcatacgacgtgagaattcctgtggacgaggaactgggcgagatccagctggtcagaat

SEQ ID NO: 350 - DNA, 86 bp, synthetic construct
  source 1..86: mol_type = other DNA
gacctgcagaagttttccagagacgtcgagcccatacagggaattcaaccggtccaccaagaacacaatagccgccgcagccgcac

SEQ ID NO: 351 - DNA, 86 bp, synthetic construct
  source 1..86: mol_type = other DNA
agtgactatgaactttgcccagagataaccctaacctacagaattcccgaggagttttcagatgatgacctggagtatctggaatg

SEQ ID NO: 352 - DNA, 86 bp, synthetic construct
  source 1..86: mol_type = other DNA
cttccatagtggagcagtgacagacaatggtgtcgcgtacgaattcatgtggggagagaattctgctggccagtgtgcagtagcca

SEQ ID NO: 353 - DNA, 86 bp, synthetic construct
  source 1..86: mol_type = other DNA
ggcaccacgctggtggagtgcaagagtggatatggcctcggaattcacctggagaccgagctcaagatgctgcgcgtgattgagcg

SEQ ID NO: 354 - DNA, 86 bp, synthetic construct
  source 1..86: mol_type = other DNA
tacgagcccatgggtggatggctgcaccaccaaatcatccgaattcccgtggtgtcccaacagcaccccctgactcacaccctgca

SEQ ID NO: 355 - DNA, 86 bp, synthetic construct
  source 1..86: mol_type = other DNA
tagctcagtgatggattttaagaacgattacacctgtcgcgaattcctgtggtctgactccaggcactcgcgtcaggtacttctgc

SEQ ID NO: 356 - DNA, 86 bp, synthetic construct
  source 1..86: mol_type = other DNA
tgttgtgggaataacaatgattgatctttacccaagagacgaattctcgtggaattttgtctttggacaggcctctttgacagatg

SEQ ID NO: 357 - DNA, 86 bp, synthetic construct
  source 1..86: mol_type = other DNA
ggctctgctgccatgaaggtgaagattaagtgctggaacggaattcgcgtggccacttggctctgggtggccaacgatgagaactg

SEQ ID NO: 358 - DNA, 86 bp, synthetic construct
  source 1..86: mol_type = other DNA
gacactcacggtgcaggcggctgacattgtgagccgggtcgaattcaactggctgggccatctcgggcagcctctttcttcgggca

SEQ ID NO: 359 - DNA, 86 bp, synthetic construct
  source 1..86: mol_type = other DNA
tactggtgtcactggtgctacccacaagacggaccggaacgaattccacaggcaccagtgggggtggcggcaggacctggggtgac

SEQ ID NO: 360 - DNA, 86 bp, synthetic construct
  source 1..86: mol_type = other DNA
cagccctcattgtgggagtacagcccattcggtcttgcacgaattcatcagggtgagcaccaaggtcaaggagaaagctgaccata

SEQ ID NO: 361 - DNA, 86 bp, synthetic construct
  source 1..86: mol_type = other DNA
gccctcaatctggaatctcaaatggtattctcacgggatcgaattcccgaggctgaagaaatagaagctgaatatgctgcattaga

SEQ ID NO: 362 - DNA, 86 bp, synthetic construct
  source 1..86: mol_type = other DNA
acctggagtctgtgagagtgctccttcgacacaatgccaagaattccgtgggcaaagagaaccgccagggctgggcaggtactgca

SEQ ID NO: 363 - DNA, 86 bp, synthetic construct
  source 1..86: mol_type = other DNA
aatgtaggactcaaactgacaagtagactggatgctgctcgaattcgacaggcttcctttgcagggtactaagacctggaaaaaac

SEQ ID NO: 364 - DNA, 86 bp, synthetic construct
  source 1..86: mol_type = other DNA
gaggactgacttacggggactggccattcgagtcaagcttgaattcggtgggtcgggcagatttcctggaggccagggcagccacg

SEQ ID NO: 365 - DNA, 86 bp, synthetic construct
  source 1..86: mol_type = other DNA
acatccagttctaccagtgctttcaaagtttccaaacgacgaattccccaggctgcagcacaatgtaagagtgtgtaccctaacaa

SEQ ID NO: 366 - DNA, 86 bp, synthetic construct
  source 1..86: mol_type = other DNA
gtgcggagactcctttctgaaaaggccactcacgtgaacagaattcctagggatgaagatgagtatacccctcttcatcgagcagc

SEQ ID NO: 367 - DNA, 86 bp, synthetic construct
  source 1..86: mol_type = other DNA
cgaaaaataagtttagatgaccttcggaaggcatatatcggaattctcaaggatgttcagcagtacattcttcatcgtttagatca

SEQ ID NO: 368 - DNA, 86 bp, synthetic construct
  source 1..86: mol_type = other DNA
agcactgcgcccgctcctgtcagccacgttgaggctgctcgaattcaacaggggtgccagagcctcagcacacttggtggcccggt

SEQ ID NO: 369 - DNA, 86 bp, synthetic construct
  source 1..86: mol_type = other DNA
aaactctatgaagccatcatgagagaagactgcactacgagaattctcgaggtactcctgagaaatcaccctgtcaaccagcccat

SEQ ID NO: 370 - DNA, 86 bp, synthetic construct
  source 1..86: mol_type = other DNA
ggtgacacacaaaacttttcaccgctatacggttcgggccgaattcaagcggggcacagcccaggggcttcgggatgcccgaggtg

SEQ ID NO: 371 - DNA, 86 bp, synthetic construct
  source 1..86: mol_type = other DNA
cagttgagtgtaccaaaggctgaggaccggtttttcagatgaattccattgggtttcagaggcaatttgatgtgcattatctcagc

SEQ ID NO: 372 - DNA, 86 bp, synthetic construct
  source 1..86: mol_type = other DNA
taggctcaaaaggctcggaaactgggggccaacgtttacagaattcccctgggtgtggctgattataatctggatcaggtaattcc

SEQ ID NO: 373 - DNA, 86 bp, synthetic construct
  source 1..86: mol_type = other DNA
gcagtcccaaatcacagctgtccagtgagtactctcctaggaattctactggatacaaaggaagaggccacggcccacgatcttct

SEQ ID NO: 374 - DNA, 86 bp, synthetic construct
  source 1..86: mol_type = other DNA
caggacaatgagctcttgacgctagagattgtgcatcgttgaattcacgtggagctgctggacaaatattttggaaatgtaagtgt

SEQ ID NO: 375 - DNA, 86 bp, synthetic construct
  source 1..86: mol_type = other DNA
tctaatacttgacagaactctcaaagcgcttgcacgaattgaattcagttggtttgggtcctaaaaatagtgcaaaaatattcacc

SEQ ID NO: 376 - DNA, 86 bp, synthetic construct
  source 1..86: mol_type = other DNA
ctgccgtacctgtgatggtgttgacaaccgttcgaaggatgaattcggtaggaggctttatgagttctttaagaatgttcgactcg

SEQ ID NO: 377 - DNA, 86 bp, synthetic construct
  source 1..86: mol_type = other DNA
caacagcagattgttcgagagactttccatctagtcctcagaattcagcgggatgacaacatctgtaacttcttggagggtggaag

SEQ ID NO: 378 - DNA, 86 bp, synthetic construct
  source 1..86: mol_type = other DNA
gggcttctgatgaaactgcagaatctttgcacacggttgggaattcatcaggatgagagtttttcccagaggcttccacttaatat

SEQ ID NO: 379 - DNA, 86 bp, synthetic construct
  source 1..86: mol_type = other DNA
ggggagctgggtgaggcactgtcttgggacaacgttgtccgaattcagctggactcctcatcgctcggtgccaggttctggatccg

SEQ ID NO: 380 - DNA, 86 bp, synthetic construct
  source 1..86: mol_type = other DNA
tgcacaggggctggaggacacataccaggtgacattgtacgaattccgcagggctgggtcgaaggctggcaggatggtgttgaaga

SEQ ID NO: 381 - DNA, 86 bp, synthetic construct
  source 1..86: mol_type = other DNA
ggagcgcctggacaatggcacctcggtcaagatggaccaggaattccacaggggctttctacacaaccaggtgaccgacccagcca

SEQ ID NO: 382 - DNA, 86 bp, synthetic construct
  source 1..86: mol_type = other DNA
tgtttccaaactccttcagcttatccaaggcactggagacgaattcgtctggggtcccctgggctggggctgggcctgccaggaag

SEQ ID NO: 383 - DNA, 86 bp, synthetic construct
  source 1..86: mol_type = other DNA
cagaggccaggagcgccaggagggcaacaacaaggagtacgaattcccggggctgcatggcacctctgttcctgcaaggaagtgtc

SEQ ID NO: 384 - DNA, 86 bp, synthetic construct
  source 1..86: mol_type = other DNA
cccagcccagcacaccctcaccagaaccattgccacccgtgaattcctctggtcctgttcaccactgtctccagcagctccttcat

SEQ ID NO: 385 - DNA, 86 bp, synthetic construct
  source 1..86: mol_type = other DNA
gaactagtaaacaaattgcgggcacgagcccaagcccgcagaattcacttggaccaaagcggcaccaatgtagcaaaggtgatgaa

SEQ ID NO: 386 - DNA, 86 bp, synthetic construct
  source 1..86: mol_type = other DNA
caatggagattcattttcaggtatgtaaaagtgaacaggtgaattccgaaggtttgaatatttatctgggggtcctatccaatcat

SEQ ID NO: 387 - DNA, 86 bp, synthetic construct
  source 1..86: mol_type = other DNA
tgactacggcatgttgctgccctgcggaattgacaagttcgaattccgaggggtagagtttgtgtgttgcccactggctgaagaaa

SEQ ID NO: 388 - DNA, 86 bp, synthetic construct
  source 1..86: mol_type = other DNA
ggcggccacggcggctccaggctctgggacgcaacctctcgaattctcggggtggcactccagggccgactgcggctgtgaaggtt

SEQ ID NO: 389 - DNA, 86 bp, synthetic construct
  source 1..86: mol_type = other DNA
ggaccgcgagcgccgaggggaccggactatttatgactacgaattcgtgcggggaaattacctggatccccggtcttgcaaagtct

SEQ ID NO: 390 - DNA, 86 bp, synthetic construct
  source 1..86: mol_type = other DNA
tacagtgtgggatgtgggtggccaggacaagattcgacccgaattcctctggagacactacttccagaacacccaaggtatgctca

SEQ ID NO: 391 - DNA, 86 bp, synthetic construct
  source 1..86: mol_type = other DNA
ggttgggatggtggtgacaatctcccccaacttcagtttggaattctacaggattgtggtcttgccagccgcatccaagccaactg

SEQ ID NO: 392 - DNA, 86 bp, synthetic construct
  source 1..86: mol_type = other DNA
ttgggcagcgcctagaggaaacagtccaccacgagcggaagaattcgtatggcccccgcctggcgcccctgctggtggagcagtgt

SEQ ID NO: 393 - DNA, 86 bp, synthetic construct
  source 1..86: mol_type = other DNA
tttggcttcactctgcgccacttcatcgtgtacccacccggaattcagtcggccgtgcactgcagcctgaaggtatgcccggctcg

SEQ ID NO: 394 - DNA, 86 bp, synthetic construct
  source 1..86: mol_type = other DNA
gctgaggaagcaaggaggctttgtcaagacttggcatactgaattccgctggtttgtgctcaagggggatcagctctattatttca

SEQ ID NO: 395 - DNA, 86 bp, synthetic construct
  source 1..86: mol_type = other DNA
tcaaaatcagaaaccacttatccaaggtgttcggacggtcgaattcaataggcagagctacagaaagagaaaaaagaaaagataca

SEQ ID NO: 396 - DNA, 86 bp, synthetic construct
  source 1..86: mol_type = other DNA
gacgcgcagcgctggtccctacctcggatttccacacgacgaattcgtaggggtgcccgcgactgtcgggtggggactggaacttc

SEQ ID NO: 397 - DNA, 86 bp, synthetic construct
  source 1..86: mol_type = other DNA
tgcatttcttcttgtttggaagtgatggcttggatcatcggaattcacatggcattgctcagctcttcctaaaaaataagaattgc

SEQ ID NO: 398 - DNA, 86 bp, synthetic construct
  source 1..86: mol_type = other DNA
ggccccgggagtccgaccctggatgccaattggataccgcgaattctgtgggcctggagggccataggaacctcctgggtacggac

SEQ ID NO: 399 - DNA, 86 bp, synthetic construct
  source 1..86: mol_type = other DNA
gctgctgggagcagcactgctcagttctcgtcaaggacgggaattccgtgggcgtgggtgagtctgccacaaaacttataaaaagc

SEQ ID NO: 400 - DNA, 86 bp, synthetic construct
  source 1..86: mol_type = other DNA
tggttacgttttcttacctccaacatggcaactaactctggaattcatttggaaatgccaattcggtctcggtcacaactgtctac

SEQ ID NO: 401 - DNA, 86 bp, synthetic construct
  source 1..86: mol_type = other DNA
aaccccagcacagtacccacctgtggtcgacacgacgttagaattctcggggctttcactgcagagtttggacaacagactggttt

SEQ ID NO: 402 - DNA, 86 bp, synthetic construct
  source 1..86: mol_type = other DNA
tcccggatggtgatctttctctgtgccacgatgtcagtaagaattcgattggtgcccacctataggaaaaaccacgatgcaaaaag

SEQ ID NO: 403 - DNA, 86 bp, synthetic construct
  source 1..86: mol_type = other DNA
agtccctgcggtcctactggcggaactactttgagagcacgaattccgatggcctcatctgggtagtggacagcgcagaccgccag

SEQ ID NO: 404 - DNA, 86 bp, synthetic construct
  source 1..86: mol_type = other DNA
aagtttgttaatgatcgtcgttttgccactattatctagcgaattcccaaggcacaaaacatgaacctccttcttcttcaggccaa

SEQ ID NO: 405 - DNA, 86 bp, synthetic construct
  source 1..86: mol_type = other DNA
gtccttcgccggatgaagaagcgctaccccacgacgttcggaattcttatggtggtcatgttggcgagctatttccttatctccat

SEQ ID NO: 406 - DNA, 86 bp, synthetic construct
  source 1..86: mol_type = other DNA
gctgaacaacttcagaaactcctttacctgctggagtcaagaattccggaggatcctgtaattattgaaagagctttgattacttt

SEQ ID NO: 407 - DNA, 86 bp, synthetic construct
  source 1..86: mol_type = other DNA
gggggattagagccactcatcagactactgagtagccctggaattcacccggatgtaaagaagaactctatggaatgcatttacaa

SEQ ID NO: 408 - DNA, 86 bp, synthetic construct
  source 1..86: mol_type = other DNA
tggttgtgggcatggccaaagggcacacggatgtcgtcatgaattccgaaggtcatgacacgcagggcccagcaggcttccctgac

SEQ ID NO: 409 - DNA, 86 bp, synthetic construct
  source 1..86: mol_type = other DNA
tatcatcaattttataaggaaagttgaacttgcctctccggaattcgacaggccatgttgtagctggagccctggtgctcttactt

SEQ ID NO: 410 - DNA, 86 bp, synthetic construct
  source 1..86: mol_type = other DNA
tagctgttggtcttgtccctggactcacgcggctggaaacgaattccacaggagcaatgcactggttctcctctctcaacacttta

SEQ ID NO: 411 - DNA, 86 bp, synthetic construct
  source 1..86: mol_type = other DNA
tatgtgcggctggttgtccggaaagtacaatttgcaccacgaattccggaggcaggccctggcccctcagcccagaccatccgccg

SEQ ID NO: 412 - DNA, 86 bp, synthetic construct
  source 1..86: mol_type = other DNA
gttctgggacaggtggctgttgcctaaggattccacggccgaattctctcggtactgctcttcattctccacatggcctgtggagg

SEQ ID NO: 413 - DNA, 86 bp, synthetic construct
  source 1..86: mol_type = other DNA
tcggcaataagagtgattgcttttgatcacggcgtatcgcgaattcaacaggaaaccattctgaatgtcgatgttggcattgtggt

SEQ ID NO: 414 - DNA, 86 bp, synthetic construct
  source 1..86: mol_type = other DNA
atggatcactgattgaataatagatggctttacctcgtctgaattccacaggagccttgcgttccttttcaaatgtcaccaagcaa

SEQ ID NO: 415 - DNA, 86 bp, synthetic construct
  source 1..86: mol_type = other DNA
aagcgcaacgagcgcgagaggcagcgagtcaagtgcgtcagaattcacgagggctacgctcgcctccgcggccacctccccggcgc

SEQ ID NO: 416 - DNA, 86 bp, synthetic construct
  source 1..86: mol_type = other DNA
tcatgggatacacaatattgttcagaaccggagcaaaattgaattccgcagggtgctctggttggtggtggttctgggctcagtct

SEQ ID NO: 417 - DNA, 86 bp, synthetic construct
  source 1..86: mol_type = other DNA
atggagaagttcaacgcgtccattgcctacgaccggcaccgaattctttgggaggtggatgttcaaggcagcaaagcctacagcag

SEQ ID NO: 418 - DNA, 86 bp, synthetic construct
  source 1..86: mol_type = other DNA
gtggcctgcctcctggtcctcttcgtgtggtactgttatcgaattcacgtgggcagggagcagccccggccctacgtctccgtcaa

SEQ ID NO: 419 - DNA, 86 bp, synthetic construct
  source 1..86: mol_type = other DNA
ggccctgctcccagctagtgggctgggatggctcgtagacgaattctatgggaaactccccccggcccctgcccccctggctccct

SEQ ID NO: 420 - DNA, 86 bp, synthetic construct
  source 1..86: mol_type = other DNA
tggagatcttgctaaagctggtccggcttttaaccacccagaattcacctggggagacgtaggggcagcgcccagcgagatgaagc

SEQ ID NO: 421 - DNA, 86 bp, synthetic construct
  source 1..86: mol_type = other DNA
ctaagcaacaaattttctttactgagacccacgcgttcttgaattctgttggaatgttctctataagaaagacagcatataaattc

SEQ ID NO: 422 - DNA, 86 bp, synthetic construct
  source 1..86: mol_type = other DNA
gaagcagaagaagaggctcgggtgacggtggtgcccaaatgaattccggaggaggaggaagaagaggaggagaaagaagaggagga

SEQ ID NO: 423 - DNA, 86 bp, synthetic construct
  source 1..86: mol_type = other DNA
cggtggaggaggtgctgggccacttcggcgtcaacgagaggaattctacggggctgagcctggaacaggtcaagaagcttaaggag

SEQ ID NO: 424 - DNA, 86 bp, synthetic construct
  source 1..86: mol_type = other DNA
cttcagtgcatctctcactgcttttgcacagatctgggaggaattctatcggatgtagctgggagaaaatgagagaaggtatatgg

SEQ ID NO: 425 - DNA, 86 bp, synthetic construct
  source 1..86: mol_type = other DNA
ctggaggtcaaactgggggagctgccaagctggatcttgagaattctgcgggacttcagtcctagtggcattttcggagcgtttca

SEQ ID NO: 426 - DNA, 86 bp, synthetic construct
  source 1..86: mol_type = other DNA
aagcctcgattggccgcattttggtactacaccacggttggaattcagctggttcctcccacccctgctgagatccctagagctat

SEQ ID NO: 427 - DNA, 86 bp, synthetic construct
  source 1..86: mol_type = other DNA
tgaactgagaacagagttttcttgaaacaggcgattacgggaattcagctggcgcaaggtgactgaaaggtcttcaaacactgagt

SEQ ID NO: 428 - DNA, 86 bp, synthetic construct
  source 1..86: mol_type = other DNA
gatctccctccgctcctcggctgtgtttggataggggtacgaattcacgtggcagtggtagctggaagacaagagttgaacacagt

SEQ ID NO: 429 - DNA, 86 bp, synthetic construct
  source 1..86: mol_type = other DNA
ttctcctgggtctccttctccacttcagtgctgcaactgcgaattccacgggatcccaatgcctggagatggggagtgatgtcacg

SEQ ID NO: 430 - DNA, 86 bp, synthetic construct
  source 1..86: mol_type = other DNA
cagcattcacctggaaggtccaggccaacaaccgtgcctagaattccaacgggcagttcaaggagaaggtgatcctgtgctggcaa

SEQ ID NO: 431 - DNA, 86 bp, synthetic construct
  source 1..86: mol_type = other DNA
tacttggtggtgtgtaacgactgtaatcaggttgtcaaacgaattccgcaggcatttcaatcacattatggtaagtgcttaaccat

SEQ ID NO: 432 - DNA, 86 bp, synthetic construct
  source 1..86: mol_type = other DNA
cttcgggatttttacctggaccagttcggcagcaacgttggaattcagtcggaggcagagaggcagctcttgaagggctcgaacca

SEQ ID NO: 433 - DNA, 86 bp, synthetic construct
  source 1..86: mol_type = other DNA
tgaacaagacatcctctttctcctgcgaagcccataacgcgaattccaagggggtcaccacatcccgcacagccaccatcacaggt

SEQ ID NO: 434 - DNA, 86 bp, synthetic construct
  source 1..86: mol_type = other DNA
ggctgggtcccagccatccaggaactctaacacgttgggggaattcacgtggacaaagacatcgtcatctccctttagcatgaaat

SEQ ID NO: 435 - DNA, 86 bp, synthetic construct
  source 1..86: mol_type = other DNA
cctgcgggggctttgcgggggcgcccacggcgcaagaggtgaattcagccggaggccgggcgcgtcccgggtgctcgcgtacagga

SEQ ID NO: 436 - DNA, 86 bp, synthetic construct
  source 1..86: mol_type = other DNA
tgcaggccttgggcttctcaggaaccaatgggcgcttacggaattcaagtgggtaattcttgcggcgccctgtgaggtgacctggg

SEQ ID NO: 437 - DNA, 86 bp, synthetic construct
  source 1..86: mol_type = other DNA
ctgcaggtgctgcaacatggtctggaactctgagtcataggaattccgtcggttgatgtcgtccccgatgatggcgagctgccgtc

SEQ ID NO: 438 - DNA, 86 bp, synthetic construct
  source 1..86: mol_type = other DNA
agcctgatcaagatgacaacctcccaaaagcaccgagactgaattctcgtggcagagcccatgggggagaagccagtggggagcct

SEQ ID NO: 439 - DNA, 86 bp, synthetic construct
  source 1..86: mol_type = other DNA
ctcctgtgggcaagagaataggtggtttgggggacacacggaattcggttggaggcccgtgcatatcccaggtgagaaatggcacc

SEQ ID NO: 440 - DNA, 86 bp, synthetic construct
  source 1..86: mol_type = other DNA
agaaagtaggcatagtaagactcaccggagcatctgacaagaattcaccaggagaagtttcaaaacttgggaaaaggatgggtttc

SEQ ID NO: 441 - DNA, 86 bp, synthetic construct
  source 1..86: mol_type = other DNA
ttgaaaccttgcagagcacaattgccaccaataatcgcaagaattcgaggggaagaaatgtctcccaatgtccccagcacaattgc

SEQ ID NO: 442 - DNA, 86 bp, synthetic construct
  source 1..86: mol_type = other DNA
gcaagccttcccagaaacatgccggtgaatcaccagttccgaattcccctggcctcatccatggaccttctgagcagcaggtcccc

SEQ ID NO: 443 - DNA, 86 bp, synthetic construct
  source 1..86: mol_type = other DNA
cgctggactgggggatccggcggctgcggaaccccactttgaattcccacggcacgccgcttagacctggacgccatgttgccgct

SEQ ID NO: 444 - DNA, 86 bp, synthetic construct
  source 1..86: mol_type = other DNA
ttcctagcttcataaagagatgttaccgcccaaggagcatgaattctaaaggatccactttgcagaatggctctggtgaacaatga

SEQ ID NO: 445 - DNA, 86 bp, synthetic construct
  source 1..86: mol_type = other DNA
cagccatgactgacaatactaatgtcaactatgtgcggtagaattccaagggtgattactacctctgcactgagaccaactttatg

SEQ ID NO: 446 - DNA, 86 bp, synthetic construct
  source 1..86: mol_type = other DNA
ccggctgaaggaaatcaactgtttcaccgccacgtttgaggaattcatctgggtggagggccccttgggcgtcatcaataacttca

SEQ ID NO: 447 - DNA, 86 bp, synthetic construct
  source 1..86: mol_type = other DNA
cctcatggtcttgagttcttgtagaatggcctgcaacgttgaattcgactggcagttacaagtacagcagtttgatccagctgatg

SEQ ID NO: 448 - DNA, 86 bp, synthetic construct
  source 1..86: mol_type = other DNA
ccctttggatgctggtgaatactgtgtgcctagaggaaatgaattccgtaggcggttccgcgttaggcagcccatcctgcagtata

SEQ ID NO: 449 - DNA, 86 bp, synthetic construct
  source 1..86: mol_type = other DNA
ctccatctccatctatcatatcaatgttatcgacaagcctgaattcattgggcacatcctctccaaaacccggggcaggtggagcc

SEQ ID NO: 450 - DNA, 86 bp, synthetic construct
  source 1..86: mol_type = other DNA
ctggcggccggggagcggccgtccgaccccggggaacaccgaattcctcaggccgagcctgggtctctggccgagggggccggacc

SEQ ID NO: 451 - DNA, 86 bp, synthetic construct
  source 1..86: mol_type = other DNA
aggctgtgggccagttctgactgcaacgaggcagccgacagaattccgtcggcgtcctggaggcgtgactcctcctccagctcgga

SEQ ID NO: 452 - DNA, 86 bp, synthetic construct
  source 1..86: mol_type = other DNA
aggtccataccccacattgaggtgatccaccaaagcttctgaattcgtcaggcaggtcgctgcagtgatagcctctcgcctcctct

SEQ ID NO: 453 - DNA, 86 bp, synthetic construct
  source 1..86: mol_type = other DNA
aaattttgaaatctcttgttccagtgtgtctaacaatacagaattcacttggttctgtctgaaacaaaaacacacagccaagacaa

SEQ ID NO: 454 - DNA, 86 bp, synthetic construct
  source 1..86: mol_type = other DNA
caaagacattgtgagccacctcgacacgaagccaatcagggaattcttcaggaacgctgaggaagggtgtggattccgcaagtccc

SEQ ID NO: 455 - DNA, 86 bp, synthetic construct
  source 1..86: mol_type = other DNA
aggctctgggcaaacaggtcagcagagagcaagctcccgggaattcgttgggtcaccggctccccatcctctggttggaacacatc

SEQ ID NO: 456 - DNA, 86 bp, synthetic construct
  source 1..86: mol_type = other DNA
ctgaccagcgcgcaggacagcgccttcctcaacgacgcgggaattcacatggtcatgagctttgtgaacctgggtaaggatttggg

SEQ ID NO: 457 - DNA, 86 bp, synthetic construct
  source 1..86: mol_type = other DNA
ggagcaggagaagctctccggtgtggtgaagagcgtccacgaattccggcggctccgcaagaagtaccgagaaggtaaacagcgcg

SEQ ID NO: 458 - DNA, 86 bp, synthetic construct
  source 1..86: mol_type = other DNA
ctgctggcaacacaggaacttcccgtccacgaagaacccagaattcctatggtacttgaccagcaggtgggggttaccccttatct

SEQ ID NO: 459 - DNA, 86 bp, synthetic construct
  source 1..86: mol_type = other DNA
ggagaagatggacatcactggagggtgttccgaatgggacgaattccacgggagcagcgcgtagacatgactgtcattgagcccta

SEQ ID NO: 460 - DNA, 86 bp, synthetic construct
  source 1..86: mol_type = other DNA
tgaaattacttacaggccaaggcggtgggaccgaagttacgaattcctctggagtatgaaaataagcaacaccattatggtaagta

SEQ ID NO: 461 - DNA, 86 bp, synthetic construct
  source 1..86: mol_type = other DNA
gtgggcaacgtgcccttggagtggtacgatgacttcccccgaattcacgtgggctacgacctggatggcaggcgcatctacaagcc

SEQ ID NO: 462 - DNA, 86 bp, synthetic construct
  source 1..86: mol_type = other DNA
gtcataattaacacacatcagtggaacttctgtactacaagaattccgctggtgaaatttataaccacatgtttgacagcggaaac

SEQ ID NO: 463 - DNA, 86 bp, synthetic construct
  source 1..86: mol_type = other DNA
cgtcaagcagtaaaaacacccccccggaggttacccactggaattctgatggttcgctctcctatagattctgcctccccaggagg

SEQ ID NO: 464 - DNA, 86 bp, synthetic construct
  source 1..86: mol_type = other DNA
tctgtatttcctaagacatctatttctcccttgaacgtgggaattctacagggagcttcagtcaactccagttcacaaactgcggc

SEQ ID NO: 465 - DNA, 86 bp, synthetic construct
  source 1..86: mol_type = other DNA
gcgggcggcgaggcaagatggcggcaaccaagaggaaacggaattcgcgtggaggctttgcagttcaggcgaagaagccaaaaaga

SEQ ID NO: 466 - DNA, 86 bp, synthetic construct
  source 1..86: mol_type = other DNA
actgctctttgattcttccccggcaggacgtccgccacatgaattcacttggtgatggcagcactctgtaactgcagcctctcaca

SEQ ID NO: 467 - DNA, 86 bp, synthetic construct
  source 1..86: mol_type = other DNA
aaccttgcgtttattttcaatgctcaacgccagatcttgtgaattccgttggcattctacttcagcccagttgagagctgcctcaa

SEQ ID NO: 468 - DNA, 86 bp, synthetic construct
  source 1..86: mol_type = other DNA
gccagtttttttagagaactctgaagctttttgtcatcaggaattcctgtggctgttctatgtaccaccttcttctttctgcgagc

SEQ ID NO: 469 - DNA, 86 bp, synthetic construct
  source 1..86: mol_type = other DNA
ctggctgtcttttccaggccgaggagggaacttctgatgggaattctacaggatacaccctccccagagctgcctctcatgatcac

SEQ ID NO: 470 - DNA, 86 bp, synthetic construct
  source 1..86: mol_type = other DNA
tatccttccacagcactgggttctaatcttcacgtcgaaggaattctgaagggttatgaggatggagggatccatctggagtgcag

SEQ ID NO: 471 - DNA, 86 bp, synthetic construct
  source 1..86: mol_type = other DNA
ttctttgctttggcactgttagggtagtctaccaccatgcgaattccaccggagaagcctgcctttgtggcctgggttgtgatcag

SEQ ID NO: 472 - DNA, 86 bp, synthetic construct
  source 1..86: mol_type = other DNA
caagactgtattattcaaggcttaactgaaaccggtactggaattcatttggaagcagtagctaagtttcttgatgcttctggagc

SEQ ID NO: 473 - DNA, 86 bp, synthetic construct
  source 1..86: mol_type = other DNA
agcaatggagactcaggctaccatcggagtcgctgcaaaggaattctcggggacaagtgatgaagcggccgaataacattttaaag

SEQ ID NO: 474 - DNA, 86 bp, synthetic construct
  source 1..86: mol_type = other DNA
tcactgcttttcctcctccttgagggggacctttggcacagaattccgctgggaggccatgaaagccaaggccaccgagctgcggg

SEQ ID NO: 475 - DNA, 86 bp, synthetic construct
  source 1..86: mol_type = other DNA
cccgcagagggtttgcatccatcaccatcacggccagacggaattccgtgggccccccagcccgcgccctggtgtgggggactgct

SEQ ID NO: 476 - DNA, 86 bp, synthetic construct
  source 1..86: mol_type = other DNA
agaactggaaaaacagatggaagatgcttaccgggggaccgaattcaaaaggaaaatgctacccagcagttcaaggtgaagttgca

SEQ ID NO: 477 - DNA, 86 bp, synthetic construct
  source 1..86: mol_type = other DNA
ggcttcacttcacaggtaggaggctgggccacaaaagggagaattctacaggaaggaatgctggtgcttacatcctgctccacttc

SEQ ID NO: 478 - DNA, 86 bp, synthetic construct
  source 1..86: mol_type = other DNA
gtcagaatagggacacttacccagtcctgggtgctgatgtgaattccatgggtgtcatggagcagttgctcatccctaaagtggac

SEQ ID NO: 479 - DNA, 86 bp, synthetic construct
  source 1..86: mol_type = other DNA
tgggagcaaacaagggaacgtgcagacgctggacagcatagaattccgatggatgcctgcaactcctgtacctgcccccgaatgcc

SEQ ID NO: 480 - DNA, 86 bp, synthetic construct
  source 1..86: mol_type = other DNA
cagaggagttaaacagggagtactcggtgctgaagtcctcgaattcgttgggcgagtccatgaaagctccccccatcatgggcagc

SEQ ID NO: 481 - DNA, 86 bp, synthetic construct
  source 1..86: mol_type = other DNA
tgatggaactcgagctgggggccggggtacggctgaagacgaattctcggggcgatggtcccttcctgggtgagcaatgcaaccct

SEQ ID NO: 482 - DNA, 86 bp, synthetic construct
  source 1..86: mol_type = other DNA
tgcccttagccttgcttcctggcctggtttcatggttcctgaattcccggggactagagagggaaatgcccaggtcctcttgggcc

SEQ ID NO: 483 - DNA, 86 bp, synthetic construct
  source 1..86: mol_type = other DNA
cccagacgaccgagctgacctgcacggaatcccacagctggaattcgtagggcaagtagtccgggctgacgctatcagggaagccc

SEQ ID NO: 484 - DNA, 86 bp, synthetic construct
  source 1..86: mol_type = other DNA
tttctcgagggagaaaaaggggaatcctctgtccagaatggaattcagcaggaaggagagccaagcctacagtcacccagcttaga

SEQ ID NO: 485 - DNA, 86 bp, synthetic construct
  source 1..86: mol_type = other DNA
caaggacgccccggccaccctgacgcaccccccaacatctgaattcacgaggggggcctggggtccccgcagccgcagtgccccag

SEQ ID NO: 486 - DNA, 86 bp, synthetic construct
  source 1..86: mol_type = other DNA
gcagtcactgatgtcccttttcaaaggatgcacgctccccgaattcaccgggcgccggaggtgttttgcagccgctcttccagagg

SEQ ID NO: 487 - DNA, 86 bp, synthetic construct
  source 1..86: mol_type = other DNA
ctcacccccgacggcttcttcttccgagtccacatgttaggaattcccctggactcctccagctgcaataagccgtgtccagagtt

SEQ ID NO: 488 - DNA, 86 bp, synthetic construct
  source 1..86: mol_type = other DNA
ctgccagagccagtgtctgagctgcgggccaacttcactcgaattctgcaggacagaggggcaggccccagggtggagatgatctg

SEQ ID NO: 489 - DNA, 86 bp, synthetic construct
  source 1..86: mol_type = other DNA
ccaacggcgagtcccggtgggccgcccaccaaacccccgagaattccgaaggccatggcccctgtgaccagggcacccttcccaga

SEQ ID NO: 490 - DNA, 86 bp, synthetic construct
  source 1..86: mol_type = other DNA
ccacgggggagatcccaagctcagtccccacaaagttcaggaattcggccggtcggaggcaggggcaggtccgggtccaaaggtaa

SEQ ID NO: 491 - DNA, 86 bp, synthetic construct
  source 1..86: mol_type = other DNA
cggccctgactacgacttcgcgcgctaccggagcacagtggaattccacggggtgacccaggccttcgccgccgcctcgcgggagg

SEQ ID NO: 492 - DNA, 86 bp, synthetic construct
  source 1..86: mol_type = other DNA
tgcctcctgcacagcgccctgctgggggcctacaccttccgaattcaacaggccttgccctcttgcccctgctgctcccaggcagg

SEQ ID NO: 493 - DNA, 86 bp, synthetic construct
  source 1..86: mol_type = other DNA
ttttctcttgtagtttatttggcaacccaaggagttaatcgaattcctaaggaacatccagtaaaacaggaattggtaagatttta

SEQ ID NO: 494 - DNA, 86 bp, synthetic construct
  source 1..86: mol_type = other DNA
atctttgtgtttcaactggctttttattatggactggtgcgaattcttaaggctctgcctaaatgaataaaaagaaaagaatacaa

SEQ ID NO: 495 - DNA, 86 bp, synthetic construct
  source 1..86: mol_type = other DNA
aggaggtccagaggagaccatcactcagcaaggacgaacggaattcccaaggacagtaactgagtccagctcatcccaccctcctg

SEQ ID NO: 496 - DNA, 86 bp, synthetic construct
  source 1..86: mol_type = other DNA
ggcactggggctcgggcatggcctgtgctggtaggatttggaattctgctgggggctgtggtcctctcgctcctcattgcacttgc

SEQ ID NO: 497 - DNA, 86 bp, synthetic construct
  source 1..86: mol_type = other DNA
gcctggcgcacatcgtaaccgaccaggtctttgatcaactgaattccgatggctgcggaagggcagaggtgctggagagaggcccc

SEQ ID NO: 498 - DNA, 86 bp, synthetic construct
  source 1..86: mol_type = other DNA
caaaaggattatgtgattcttgccactagaacgccacccagaattcaagaggagcaaagtgagaacctcaaacatcccaaagctaa

SEQ ID NO: 499 - DNA, 86 bp, synthetic construct
  source 1..86: mol_type = other DNA
ctgtccgtggtgctggatgtggctctatcgtccacacggagaattcggaaggagggtgggagggtggtgcagaacccacccgcccc

SEQ ID NO: 500 - DNA, 86 bp, synthetic construct
  source 1..86: mol_type = other DNA
ataatagctctggcaaggagaagcaccctggacatagcacgaattcgtctgggtctggcagggagctggacttttgatgtaggtca

SEQ ID NO: 501 - DNA, 86 bp, synthetic construct
  source 1..86: mol_type = other DNA
gccaggtcgaaggcgccgtccaggttcaccagctccgtgtgaattccgaagggcaccgcctggaagtggtcggagctgtgcaggcc

SEQ ID NO: 502 - DNA, 86 bp, synthetic construct
  source 1..86: mol_type = other DNA
tgagaccctcaactgctcctcctgccaccacacagccgacgaattcgaatggaactggcttgatgcgtgctccaggaagactatgg

SEQ ID NO: 503 - DNA, 86 bp, synthetic construct
  source 1..86: mol_type = other DNA
agtcttggctggactcactgcccacctctgggaccttggcgaattcggtggggcgggaaggaggacctcaaaggctcagcgagtcc

SEQ ID NO: 504 - DNA, 86 bp, synthetic construct
  source 1..86: mol_type = other DNA
ctgaacaggatcgttcagctgcacgacgcacactgcattagaattcatgtggcctgtaattaaatagaagggcatcgtgttggcgt

SEQ ID NO: 505 - DNA, 86 bp, synthetic construct
  source 1..86: mol_type = other DNA
tgctatacatatggattcaaaagtggatgatcacttaatagaattccgagggactgaaaaaagcaggttggaaccagcgactcagt

SEQ ID NO: 506 - DNA, 86 bp, synthetic construct
  source 1..86: mol_type = other DNA
tagatactgtagagaaatctgtggggttgaccccaaagctgaattcaacaggtagagctaaggaatccttagggatgctgctgcag

SEQ ID NO: 507 - DNA, 86 bp, synthetic construct
  source 1..86: mol_type = other DNA
catgagtccagggggcacgtaggcggcattgaactcggtcgaattcagtagggtgcccgcgctgcgggaggccatggtgggtgcgc

SEQ ID NO: 508 - DNA, 86 bp, synthetic construct
  source 1..86: mol_type = other DNA
aaggtaaagagacaaagaaagtgccttgtacagatgcaaagaattccggaggtgtagactgtgcagctgccaaagtggtgacaagc

SEQ ID NO: 509 - DNA, 86 bp, synthetic construct
  source 1..86: mol_type = other DNA
tggctgttggggtctactcagccaagaatgccacgcttgtgaattccgccggccgcttcatcgaggctcggctggggaagccgtcc

SEQ ID NO: 510 - DNA, 86 bp, synthetic construct
  source 1..86: mol_type = other DNA
gtgtccctgcatctgcaggccatgtcaaaggacgccctgagaattcatctggcgcagatgcaggagcagacgctgcagttggagca

SEQ ID NO: 511 - DNA, 57 bp, synthetic construct
  source 1..57: mol_type = other DNA
acactctttccctacacgacgctcttccgatctcaccttcagtaacctttttcatct

SEQ ID NO: 512 - DNA, 51 bp, synthetic construct
  source 1..51: mol_type = other DNA
acactctttccctacacgacgctcttccgatctaaaagtgccgctgaagtg

SEQ ID NO: 513 - DNA, 54 bp, synthetic construct
  source 1..54: mol_type = other DNA
acactctttccctacacgacgctcttccgatctgctcagaatctgttctatgcc

SEQ ID NO: 514 - DNA, 52 bp, synthetic construct
  source 1..52: mol_type = other DNA
acactctttccctacacgacgctcttccgatctcgcctatctactcacgttg

SEQ ID NO: 515 - DNA, 55 bp, synthetic construct
  source 1..55: mol_type = other DNA
acactctttccctacacgacgctcttccgatctacagcagatgtttaacagactt

SEQ ID NO: 516 - DNA, 56 bp, synthetic construct
  source 1..56: mol_type = other DNA
acactctttccctacacgacgctcttccgatctattaaccaactcaccaaagacag

SEQ ID NO: 517 - DNA, 56 bp, synthetic construct
  source 1..56: mol_type = other DNA
acactctttccctacacgacgctcttccgatctagagggctgacagaaataataac

SEQ ID NO: 518 - DNA, 52 bp, synthetic construct
  source 1..52: mol_type = other DNA
acactctttccctacacgacgctcttccgatctgccgagtgaaatgtacgtc

SEQ ID NO: 519 - DNA, 50 bp, synthetic construct
  source 1..50: mol_type = other DNA
acactctttccctacacgacgctcttccgatctcacagactgcagccaac

SEQ ID NO: 520 - DNA, 50 bp, synthetic construct
  source 1..50: mol_type = other DNA
acactctttccctacacgacgctcttccgatctcaaagctggcatgaacc

SEQ ID NO: 521 - DNA, 56 bp, synthetic construct
  source 1..56: mol_type = other DNA
acactctttccctacacgacgctcttccgatctcccagaactttgtgtatctttct

SEQ ID NO: 522 - DNA, 54 bp, synthetic construct
  source 1..54: mol_type = other DNA
acactctttccctacacgacgctcttccgatcttaagcttctcttggaccttga

SEQ ID NO: 523 - DNA, 57 bp, synthetic construct
  source 1..57: mol_type = other DNA
acactctttccctacacgacgctcttccgatctcacattaaaagtgcacagaaaacg

SEQ ID NO: 524 - DNA, 51 bp, synthetic construct
  source 1..51: mol_type = other DNA
acactctttccctacacgacgctcttccgatctagactccgaagctgacct

SEQ ID NO: 525 - DNA, 53 bp, synthetic construct
  source 1..53: mol_type = other DNA
acactctttccctacacgacgctcttccgatctgctagtaacagttctgggtg

SEQ ID NO: 526 - DNA, 51 bp, synthetic construct
  source 1..51: mol_type = other DNA
acactctttccctacacgacgctcttccgatctcaagatcttggcgatgga

SEQ ID NO: 527 - DNA, 49 bp, synthetic construct
  source 1..49: mol_type = other DNA
acactctttccctacacgacgctcttccgatctactcgcccaggtagga

SEQ ID NO: 528 - DNA, 57 bp, synthetic construct
  source 1..57: mol_type = other DNA
acactctttccctacacgacgctcttccgatctggcactgaattttggagatctttg

SEQ ID NO: 529 - DNA, 53 bp, synthetic construct
  source 1..53: mol_type = other DNA
acactctttccctacacgacgctcttccgatcttattaactctgggctgctgt

SEQ ID NO: 530 - DNA, 50 bp, synthetic construct
  source 1..50: mol_type = other DNA
acactctttccctacacgacgctcttccgatctaaggtcatcgccccaga

SEQ ID NO: 531 - DNA, 53 bp, synthetic construct
  source 1..53: mol_type = other DNA
acactctttccctacacgacgctcttccgatcttgaccagggagtcttacctt

SEQ ID NO: 532 - DNA, 52 bp, synthetic construct
  source 1..52: mol_type = other DNA
acactctttccctacacgacgctcttccgatctccaccaaccttgtttctgt

SEQ ID NO: 533 - DNA, 53 bp, synthetic construct
  source 1..53: mol_type = other DNA
acactctttccctacacgacgctcttccgatctcagtgactggccaacattta

SEQ ID NO: 534 - DNA, 61 bp, synthetic construct
  source 1..61: mol_type = other DNA
acactctttccctacacgacgctcttccgatctgggtttgcaaaatatttgtattaacatt

SEQ ID NO: 535 - DNA, 51 bp, synthetic construct
  source 1..51: mol_type = other DNA
acactctttccctacacgacgctcttccgatctcggcaggagaagcaagat

SEQ ID NO: 536 - DNA, 53 bp, synthetic construct
  source 1..53: mol_type = other DNA
acactctttccctacacgacgctcttccgatctaagtgcctcttccttctgag

SEQ ID NO: 537 - DNA, 59 bp, synthetic construct
  source 1..59: mol_type = other DNA
acactctttccctacacgacgctcttccgatctgttgtaattgattctaggaattggat

SEQ ID NO: 538 - DNA, 53 bp, synthetic construct
  source 1..53: mol_type = other DNA
acactctttccctacacgacgctcttccgatctctgctacatcttgaacctgg

SEQ ID NO: 539 - DNA, 57 bp, synthetic construct
  source 1..57: mol_type = other DNA
acactctttccctacacgacgctcttccgatctcttagattactcttgtctctgctg

SEQ ID NO: 540 - DNA, 53 bp, synthetic construct
  source 1..53: mol_type = other DNA
acactctttccctacacgacgctcttccgatctccccattcagttgttctcag

SEQ ID NO: 541 - DNA, 52 bp, synthetic construct
  source 1..52: mol_type = other DNA
acactctttccctacacgacgctcttccgatctcattcaaccacttccctgt

SEQ ID NO: 542 - DNA, 53 bp, synthetic construct
  source 1..53: mol_type = other DNA
acactctttccctacacgacgctcttccgatcttagagtatgcaatctgggca

SEQ ID NO: 543 - DNA, 53 bp, synthetic construct
  source 1..53: mol_type = other DNA
acactctttccctacacgacgctcttccgatctacagagggaaatgacattgc

SEQ ID NO: 544 - DNA, 51 bp, synthetic construct
  source 1..51: mol_type = other DNA
acactctttccctacacgacgctcttccgatctcaggtagtctctgccttc

SEQ ID NO: 545 - DNA, 55 bp, synthetic construct
  source 1..55: mol_type = other DNA
acactctttccctacacgacgctcttccgatctcctccagtccttacttgaactt

SEQ ID NO: 546 - DNA, 53 bp, synthetic construct
  source 1..53: mol_type = other DNA
acactctttccctacacgacgctcttccgatctctgacagcaaaagacatcct

SEQ ID NO: 547 - DNA, 53 bp, synthetic construct
  source 1..53: mol_type = other DNA
acactctttccctacacgacgctcttccgatctcctcagactttcttcccttc

SEQ ID NO: 548 - DNA, 53 bp, synthetic construct
  source 1..53: mol_type = other DNA
acactctttccctacacgacgctcttccgatctcagaggaacctaatctgtgt

SEQ ID NO: 549 - DNA, 51 bp, synthetic construct
  source 1..51: mol_type = other DNA
acactctttccctacacgacgctcttccgatctataccttccgcagcttcc

SEQ ID NO: 550 - DNA, 54 bp, synthetic construct
  source 1..54: mol_type = other DNA
acactctttccctacacgacgctcttccgatctcaccgcatgattagacaggta

SEQ ID NO: 551 - DNA, 54 bp, synthetic construct
  source 1..54: mol_type = other DNA
acactctttccctacacgacgctcttccgatctcagcatttaccagattgcact

SEQ ID NO: 552 - DNA, 60 bp, synthetic construct
  source 1..60: mol_type = other DNA
acactctttccctacacgacgctcttccgatcttaggtgctatacttggtagatcagaaa

SEQ ID NO: 553 - DNA, 51 bp, synthetic construct
  source 1..51: mol_type = other DNA
acactctttccctacacgacgctcttccgatctgcaggtcaaggacaaagt

SEQ ID NO: 554 - DNA, 55 bp, synthetic construct
  source 1..55: mol_type = other DNA
acactctttccctacacgacgctcttccgatctcttttaactgcagtaggtagga

SEQ ID NO: 555 - DNA, 50 bp, synthetic construct
  source 1..50: mol_type = other DNA
acactctttccctacacgacgctcttccgatcttcttccccaaaccccac

SEQ ID NO: 556 - DNA, 57 bp, synthetic construct
  source 1..57: mol_type = other DNA
acactctttccctacacgacgctcttccgatcttcattgtttttaccaaggatccat

SEQ ID NO: 557 - DNA, 56 bp, synthetic construct
  source 1..56: mol_type = other DNA
acactctttccctacacgacgctcttccgatctgaaattctgtagtacacccagtc

SEQ ID NO: 558 - DNA, 53 bp, synthetic construct
  source 1..53: mol_type = other DNA
acactctttccctacacgacgctcttccgatctgcccgtaggtatcgttcttc

SEQ ID NO: 559 - DNA, 57 bp, synthetic construct
  source 1..57: mol_type = other DNA
acactctttccctacacgacgctcttccgatctcatgtttatttgttgtcttgcacg

SEQ ID NO: 560 - DNA, 56 bp, synthetic construct
  source 1..56: mol_type = other DNA
acactctttccctacacgacgctcttccgatctgcccttctaagaccattgtgtta

SEQ ID NO: 561 - DNA, 51 bp, synthetic construct
  source 1..51: mol_type = other DNA
acactctttccctacacgacgctcttccgatcttggagtcgaaactgacct

SEQ ID NO: 562 - DNA, 52 bp, synthetic construct
  source 1..52: mol_type = other DNA
acactctttccctacacgacgctcttccgatctgaggtgtcatggtagttgg

SEQ ID NO: 563 - DNA, 54 bp, synthetic construct
  source 1..54: mol_type = other DNA
acactctttccctacacgacgctcttccgatctgaccttgaaagcaattgtgga

SEQ ID NO: 564 - DNA, 56 bp, synthetic construct
  source 1..56: mol_type = other DNA
acactctttccctacacgacgctcttccgatctccttccaacagtttttctttgtc

SEQ ID NO: 565 - DNA, 51 bp, synthetic construct
  source 1..51: mol_type = other DNA
acactctttccctacacgacgctcttccgatctgaagtacccactgccatc

SEQ ID NO: 566 - DNA, 50 bp, synthetic construct
  source 1..50: mol_type = other DNA
acactctttccctacacgacgctcttccgatctagctgctcttgacgact

SEQ ID NO: 567 - DNA, 54 bp, synthetic construct
  source 1..54: mol_type = other DNA
acactctttccctacacgacgctcttccgatctgcaattagcatcaagggtttg

SEQ ID NO: 568 - DNA, 56 bp, synthetic construct
  source 1..56: mol_type = other DNA
acactctttccctacacgacgctcttccgatctcttttactcctcccatgttcttt

SEQ ID NO: 569 - DNA, 49 bp, synthetic construct
  source 1..49: mol_type = other DNA
acactctttccctacacgacgctcttccgatcttgtggctcatctcggc

SEQ ID NO: 570 - DNA, 50 bp, synthetic construct
  source 1..50: mol_type = other DNA
acactctttccctacacgacgctcttccgatctaccaccaggagtcccat

SEQ ID NO: 571 - DNA, 49 bp, synthetic construct
  source 1..49: mol_type = other DNA
acactctttccctacacgacgctcttccgatctctcagctgcctcctgg

SEQ ID NO: 572 - DNA, 53 bp, synthetic construct
  source 1..53: mol_type = other DNA
acactctttccctacacgacgctcttccgatctgttttcttccccttcccatc

SEQ ID NO: 573 - DNA, 51 bp, synthetic construct
  source 1..51: mol_type = other DNA
acactctttccctacacgacgctcttccgatctccttggcagtggtttctc

SEQ ID NO: 574 - DNA, 49 bp, synthetic construct
  source 1..49: mol_type = other DNA
acactctttccctacacgacgctcttccgatcttctgccctctgaccca

SEQ ID NO: 575 - DNA, 58 bp, synthetic construct
  source 1..58: mol_type = other DNA
acactctttccctacacgacgctcttccgatctctccctctatacatatagctttgga

SEQ ID NO: 576 - DNA, 51 bp, synthetic construct
  source 1..51: mol_type = other DNA
acactctttccctacacgacgctcttccgatctttcaaagtgccacgtttg

SEQ ID NO: 577 - DNA, 53 bp, synthetic construct
  source 1..53: mol_type = other DNA
acactctttccctacacgacgctcttccgatctatggctttctggacttcatc

SEQ ID NO: 578 - DNA, 53 bp, synthetic construct
  source 1..53: mol_type = other DNA
acactctttccctacacgacgctcttccgatctgaaaccaatcaagctcctgg

SEQ ID NO: 579 - DNA, 53 bp, synthetic construct
  source 1..53: mol_type = other DNA
acactctttccctacacgacgctcttccgatctgccactggaattagacaagc

SEQ ID NO: 580 - DNA, 50 bp, synthetic construct
  source 1..50: mol_type = other DNA
acactctttccctacacgacgctcttccgatctccatttccccaggatga

SEQ ID NO: 581 - DNA, 51 bp, synthetic construct
  source 1..51: mol_type = other DNA
acactctttccctacacgacgctcttccgatctgttctctctggccatctg

SEQ ID NO: 582 - DNA, 56 bp, synthetic construct
  source 1..56: mol_type = other DNA
acactctttccctacacgacgctcttccgatctgcagaacccactaatacaaagga

SEQ ID NO: 583 - DNA, 56 bp, synthetic construct
  source 1..56: mol_type = other DNA
acactctttccctacacgacgctcttccgatctgtcttctggctgttctatagatc

SEQ ID NO: 584 - DNA, 51 bp, synthetic construct
  source 1..51: mol_type = other DNA
acactctttccctacacgacgctcttccgatcttggtttcctctctccgag

SEQ ID NO: 585 - DNA, 54 bp, synthetic construct
  source 1..54: mol_type = other DNA
acactctttccctacacgacgctcttccgatctctacccttttctctacccagg

SEQ ID NO: 586 - DNA, 56 bp, synthetic construct
  source 1..56: mol_type = other DNA
acactctttccctacacgacgctcttccgatctccagcatctttcaaaccaatttt

SEQ ID NO: 587 - DNA, 50 bp, synthetic construct
  source 1..50: mol_type = other DNA
acactctttccctacacgacgctcttccgatctttgggtcagtgccttac

SEQ ID NO: 588 - DNA, 58 bp, synthetic construct
  source 1..58: mol_type = other DNA
acactctttccctacacgacgctcttccgatcttcctgagtttacatacgtcatcttt

SEQ ID NO: 589 - DNA, 55 bp, synthetic construct
  source 1..55: mol_type = other DNA
acactctttccctacacgacgctcttccgatctgtcactgattctctcttctctg

SEQ ID NO: 590 - DNA, 62 bp, synthetic construct
  source 1..62: mol_type = other DNA
acactctttccctacacgacgctcttccgatctcacatatgtacacacaagaaaatcacata

SEQ ID NO: 591 - DNA, 57 bp, synthetic construct
  source 1..57: mol_type = other DNA
acactctttccctacacgacgctcttccgatctcttgaaacagagttctccctaaag

SEQ ID NO: 592 - DNA, 56 bp, synthetic construct
  source 1..56: mol_type = other DNA
acactctttccctacacgacgctcttccgatctaagatctttgtctcttcccactt

SEQ ID NO: 593 - DNA, 57 bp, synthetic construct
  source 1..57: mol_type = other DNA
acactctttccctacacgacgctcttccgatctagatgtggtatttagcaagagtca

SEQ ID NO: 594 - DNA, 52 bp, synthetic construct
  source 1..52: mol_type = other DNA
acactctttccctacacgacgctcttccgatcttccaggtcacagttcttgt

SEQ ID NO: 595 - DNA, 54 bp, synthetic construct
  source 1..54: mol_type = other DNA
acactctttccctacacgacgctcttccgatcttcttctcttagggttggatgg

SEQ ID NO: 596 - DNA, 54 bp, synthetic construct
  source 1..54: mol_type = other DNA
acactctttccctacacgacgctcttccgatcttatttgtaggtgcaggaagct

SEQ ID NO: 597 - DNA, 52 bp, synthetic construct
  source 1..52: mol_type = other DNA
acactctttccctacacgacgctcttccgatcttcgcctcctcgatacttac

SEQ ID NO: 598 - DNA, 49 bp, synthetic construct
  source 1..49: mol_type = other DNA
acactctttccctacacgacgctcttccgatcttgggttcccaggtctg

SEQ ID NO: 599 - DNA, 52 bp, synthetic construct
  source 1..52: mol_type = other DNA
acactctttccctacacgacgctcttccgatctagtattacagccgcctcat

SEQ ID NO: 600 - DNA, 50 bp, synthetic construct
  source 1..50: mol_type = other DNA
acactctttccctacacgacgctcttccgatctttgggctccttatccgt

SEQ ID NO: 601 - DNA, 55 bp, synthetic construct
  source 1..55: mol_type = other DNA
acactctttccctacacgacgctcttccgatctctgtcccagttataatggtagc

SEQ ID NO: 602 - DNA, 50 bp, synthetic construct
  source 1..50: mol_type = other DNA
acactctttccctacacgacgctcttccgatctagacgctgagccgagaa

SEQ ID NO: 603 - DNA, 56 bp, synthetic construct
  source 1..56: mol_type = other DNA
acactctttccctacacgacgctcttccgatctccactacttcttttccattgagg

SEQ ID NO: 604 - DNA, 52 bp, synthetic construct
  source 1..52: mol_type = other DNA
acactctttccctacacgacgctcttccgatctaacacctccagaacaaagg

SEQ ID NO: 605 - DNA, 50 bp, synthetic construct
  source 1..50: mol_type = other DNA
acactctttccctacacgacgctcttccgatctgatgttccacgctgttc

SEQ ID NO: 606 - DNA, 52 bp, synthetic construct
  source 1..52: mol_type = other DNA
acactctttccctacacgacgctcttccgatctcaggttcctttgccaaact

SEQ ID NO: 607 - DNA, 53 bp, synthetic construct
  source 1..53: mol_type = other DNA
acactctttccctacacgacgctcttccgatctcgccaactgtaaaatcctga

SEQ ID NO: 608 - DNA, 51 bp, synthetic construct
  source 1..51: mol_type = other DNA
acactctttccctacacgacgctcttccgatctgctccagtgcatgatgag

SEQ ID NO: 609 - DNA, 51 bp, synthetic construct
  source 1..51: mol_type = other DNA
acactctttccctacacgacgctcttccgatcttggtgatgagactgcagg

SEQ ID NO: 610 - DNA, 54 bp, synthetic construct
  source 1..54: mol_type = other DNA
acactctttccctacacgacgctcttccgatctaggaaccattgatgatgctgt

SEQ ID NO: 611 - DNA, 53 bp, synthetic construct
  source 1..53: mol_type = other DNA
acactctttccctacacgacgctcttccgatctcatgcaggtgaattacacga

SEQ ID NO: 612 - DNA, 53 bp, synthetic construct
  source 1..53: mol_type = other DNA
acactctttccctacacgacgctcttccgatctgttggtgcctaaacgttcta

SEQ ID NO: 613 - DNA, 51 bp, synthetic construct
  source 1..51: mol_type = other DNA
acactctttccctacacgacgctcttccgatctgtcccatcctagtttggc

SEQ ID NO: 614 - DNA, 54 bp, synthetic construct
  source 1..54: mol_type = other DNA
acactctttccctacacgacgctcttccgatcttaatgtcgacttacccacagg

SEQ ID NO: 615 - DNA, 54 bp, synthetic construct
  source 1..54: mol_type = other DNA
acactctttccctacacgacgctcttccgatctctttgcacttagcctcagttt

SEQ ID NO: 616 - DNA, 53 bp, synthetic construct
  source 1..53: mol_type = other DNA
acactctttccctacacgacgctcttccgatctcaacaccacaaagatttggc

SEQ ID NO: 617 - DNA, 52 bp, synthetic construct
  source 1..52: mol_type = other DNA
acactctttccctacacgacgctcttccgatctctcttctctcctgcccttt

SEQ ID NO: 618 - DNA, 53 bp, synthetic construct
  source 1..53: mol_type = other DNA
acactctttccctacacgacgctcttccgatctggtctgaaaatgctcttcca

SEQ ID NO: 619 - DNA, 52 bp, synthetic construct
  source 1..52: mol_type = other DNA
acactctttccctacacgacgctcttccgatctcttcaaaagggagccacat

SEQ ID NO: 620 - DNA, 56 bp, synthetic construct
  source 1..56: mol_type = other DNA
acactctttccctacacgacgctcttccgatctctgagaatatctagcagcaacat

SEQ ID NO: 621 - DNA, 54 bp, synthetic construct
  source 1..54: mol_type = other DNA
acactctttccctacacgacgctcttccgatctttcttctcagcttaccacagt

SEQ ID NO: 622 - DNA, 58 bp, synthetic construct
  source 1..58: mol_type = other DNA
acactctttccctacacgacgctcttccgatctcgcaaagcttcttcttgatctaaac

SEQ ID NO: 623 - DNA, 51 bp, synthetic construct
  source 1..51: mol_type = other DNA
acactctttccctacacgacgctcttccgatctcaagatgcccactatgca

SEQ ID NO: 624 - DNA, 51 bp, synthetic construct
  source 1..51: mol_type = other DNA
acactctttccctacacgacgctcttccgatctcatctgcagcacttcact

SEQ ID NO: 625 - DNA, 53 bp, synthetic construct
  source 1..53: mol_type = other DNA
acactctttccctacacgacgctcttccgatcttggtctccagtactgagtct

SEQ ID NO: 626 - DNA, 56 bp, synthetic construct
  source 1..56: mol_type = other DNA
acactctttccctacacgacgctcttccgatcttgtacgtatgctgagataatgca

SEQ ID NO: 627 - DNA, 53 bp, synthetic construct
  source 1..53: mol_type = other DNA
acactctttccctacacgacgctcttccgatctcacatgcatttcaggacact

SEQ ID NO: 628 - DNA, 52 bp, synthetic construct
  source 1..52: mol_type = other DNA
acactctttccctacacgacgctcttccgatctagagtccgttttgccagta

SEQ ID NO: 629 - DNA, 54 bp, synthetic construct
  source 1..54: mol_type = other DNA
acactctttccctacacgacgctcttccgatctgggactgtagctaatcctaac

SEQ ID NO: 630 - DNA, 59 bp, synthetic construct
  source 1..59: mol_type = other DNA
acactctttccctacacgacgctcttccgatctagcatcatgataggtacaataattgg

SEQ ID NO: 631 - DNA, 52 bp, synthetic construct
  source 1..52: mol_type = other DNA
acactctttccctacacgacgctcttccgatcttttccattggctaccgagt

SEQ ID NO: 632 - DNA, 53 bp, synthetic construct
  source 1..53: mol_type = other DNA
acactctttccctacacgacgctcttccgatctctgacagtttaccttccacc

SEQ ID NO: 633 - DNA, 57 bp, synthetic construct
  source 1..57: mol_type = other DNA
acactctttccctacacgacgctcttccgatctggccttaagttcattattctttcc

SEQ ID NO: 634 - DNA, 51 bp, synthetic construct
  source 1..51: mol_type = other DNA
acactctttccctacacgacgctcttccgatctaggctcacgttcctctct

SEQ ID NO: 635 - DNA, 53 bp, synthetic construct
  source 1..53: mol_type = other DNA
acactctttccctacacgacgctcttccgatcttttcttcaacaccatcctgc

SEQ ID NO: 636 - DNA, 52 bp, synthetic construct
  source 1..52: mol_type = other DNA
acactctttccctacacgacgctcttccgatctggcataagacctacctgtg

SEQ ID NO: 637 - DNA, 52 bp, synthetic construct
  source 1..52: mol_type = other DNA
acactctttccctacacgacgctcttccgatctattttgaacccctgcccat

SEQ ID NO: 638 - DNA, 51 bp, synthetic construct
  source 1..51: mol_type = other DNA
acactctttccctacacgacgctcttccgatctacaggacacttccttgca

SEQ ID NO: 639 - DNA, 52 bp, synthetic construct
  source 1..52: mol_type = other DNA
acactctttccctacacgacgctcttccgatcttaaagatgagtcgctggag

SEQ ID NO: 640 - DNA, 54 bp, synthetic construct
  source 1..54: mol_type = other DNA
acactctttccctacacgacgctcttccgatctactccttcatcacctttgcta

SEQ ID NO: 641 - DNA, 53 bp, synthetic construct
  source 1..53: mol_type = other DNA
acactctttccctacacgacgctcttccgatctaaaggtctcaagattctgcc

SEQ ID NO: 642 - DNA, 54 bp, synthetic construct
  source 1..54: mol_type = other DNA
acactctttccctacacgacgctcttccgatctcacattgtcactttcttcagc

SEQ ID NO: 643 - DNA, 51 bp, synthetic construct
  source 1..51: mol_type = other DNA
acactctttccctacacgacgctcttccgatctaacagcaaccttcacagc

SEQ ID NO: 644 - DNA, 53 bp, synthetic construct
  source 1..53: mol_type = other DNA
acactctttccctacacgacgctcttccgatcttccaatcccaggagactttg

SEQ ID NO: 645 - DNA, 52 bp, synthetic construct
  source 1..52: mol_type = other DNA
acactctttccctacacgacgctcttccgatctactctctgctcatacccaa

SEQ ID NO: 646 - DNA, 50 bp, synthetic construct
  source 1..50: mol_type = other DNA
acactctttccctacacgacgctcttccgatctatccctgtgcccctttc

SEQ ID NO: 647 - DNA, 53 bp, synthetic construct
  source 1..53: mol_type = other DNA
acactctttccctacacgacgctcttccgatctatgaagtccacacactgctc

SEQ ID NO: 648 - DNA, 52 bp, synthetic construct
  source 1..52: mol_type = other DNA
acactctttccctacacgacgctcttccgatcttgctgctgtacaaaagtcc

SEQ ID NO: 649 - DNA, 56 bp, synthetic construct
  source 1..56: mol_type = other DNA
acactctttccctacacgacgctcttccgatctgtttgctaactaggaaagtccat

SEQ ID NO: 650 - DNA, 52 bp, synthetic construct
  source 1..52: mol_type = other DNA
acactctttccctacacgacgctcttccgatctcagatcagggcattgggat

SEQ ID NO: 651 - DNA, 51 bp, synthetic construct
  source 1..51: mol_type = other DNA
acactctttccctacacgacgctcttccgatctcttagtgggtgccttgct

SEQ ID NO: 652 - DNA, 53 bp, synthetic construct
  source 1..53: mol_type = other DNA
acactctttccctacacgacgctcttccgatctccccatgtaccacgttaaaa

SEQ ID NO: 653 - DNA, 52 bp, synthetic construct
  source 1..52: mol_type = other DNA
acactctttccctacacgacgctcttccgatctgcagtaaccctcattctca

SEQ ID NO: 654 - DNA, 55 bp, synthetic construct
  source 1..55: mol_type = other DNA
acactctttccctacacgacgctcttccgatctaaggaaaacctactctctctgg

SEQ ID NO: 655 - DNA, 53 bp, synthetic construct
  source 1..53: mol_type = other DNA
acactctttccctacacgacgctcttccgatctaatgactgccccacatttta

SEQ ID NO: 656 - DNA, 53 bp, synthetic construct
  source 1..53: mol_type = other DNA
acactctttccctacacgacgctcttccgatcttgcacaggaaactaggacat

SEQ ID NO: 657 - DNA, 54 bp, synthetic construct
  source 1..54: mol_type = other DNA
acactctttccctacacgacgctcttccgatctgcccatataggattacaaccc

SEQ ID NO: 658 - DNA, 51 bp, synthetic construct
  source 1..51: mol_type = other DNA
acactctttccctacacgacgctcttccgatctctccttgcccagattcaa

SEQ ID NO: 659 - DNA, 59 bp, synthetic construct
  source 1..59: mol_type = other DNA
acactctttccctacacgacgctcttccgatctccaatattttccataacttaaggtgc

SEQ ID NO: 660 - DNA, 52 bp, synthetic construct
  source 1..52: mol_type = other DNA
acactctttccctacacgacgctcttccgatctggcagcccacaataaagac

SEQ ID NO: 661 - DNA, 59 bp, synthetic construct
  source 1..59: mol_type = other DNA
acactctttccctacacgacgctcttccgatctttcttatgcagaagacttaactgatg

SEQ ID NO: 662 - DNA, 55 bp, synthetic construct
  source 1..55: mol_type = other DNA
acactctttccctacacgacgctcttccgatctcaaacatgtctgcagagtacac

SEQ ID NO: 663 - DNA, 52 bp, synthetic construct
  source 1..52: mol_type = other DNA
acactctttccctacacgacgctcttccgatctcgcttgcctgaaacatgaa

SEQ ID NO: 664 - DNA, 53 bp, synthetic construct
  source 1..53: mol_type = other DNA
acactctttccctacacgacgctcttccgatctgggcaagtggaaaatccaag

SEQ ID NO: 665 - DNA, 53 bp, synthetic construct
  source 1..53: mol_type = other DNA
acactctttccctacacgacgctcttccgatctgcccataggtaaagtgttga

SEQ ID NO: 666 - DNA, 54 bp, synthetic construct
  source 1..54: mol_type = other DNA
acactctttccctacacgacgctcttccgatctctggctaatctcttggtctct

SEQ ID NO: 667 - DNA, 52 bp, synthetic construct
  source 1..52: mol_type = other DNA
acactctttccctacacgacgctcttccgatcttgaacacggccaagtttag

SEQ ID NO: 668 - DNA, 52 bp, synthetic construct
  source 1..52: mol_type = other DNA
acactctttccctacacgacgctcttccgatcttcgccattatccgagagag

SEQ ID NO: 669 - DNA, 56 bp, synthetic construct
  source 1..56: mol_type = other DNA
acactctttccctacacgacgctcttccgatctcaaatctacctttaagtcagcca

SEQ ID NO: 670 - DNA, 51 bp, synthetic construct
  source 1..51: mol_type = other DNA
acactctttccctacacgacgctcttccgatcttctccaccttgctgagtc

SEQ ID NO: 671 - DNA, 54 bp, synthetic construct
  source 1..54: mol_type = other DNA
acactctttccctacacgacgctcttccgatcttagatctgccatgtcacaagt

SEQ ID NO: 672 - DNA, 53 bp, synthetic construct
  source 1..53: mol_type = other DNA
acactctttccctacacgacgctcttccgatctaattgttcttgctctcctgg

SEQ ID NO: 673 - DNA, 49 bp, synthetic construct
  source 1..49: mol_type = other DNA
acactctttccctacacgacgctcttccgatctgctgtcatcactgtgg

SEQ ID NO: 674 - DNA, 51 bp, synthetic construct
  source 1..51: mol_type = other DNA
acactctttccctacacgacgctcttccgatcttgtgctacagccatgtca

SEQ ID NO: 675 - DNA, 51 bp, synthetic construct
  source 1..51: mol_type = other DNA
acactctttccctacacgacgctcttccgatctgctgtcatgcaagtgctt

SEQ ID NO: 676 - DNA, 55 bp, synthetic construct
  source 1..55: mol_type = other DNA
acactctttccctacacgacgctcttccgatctcaagtcaaaaacattcaagggc

SEQ ID NO: 677 - DNA, 52 bp, synthetic construct
  source 1..52: mol_type = other DNA
acactctttccctacacgacgctcttccgatctcactgttccaggatgatcc

SEQ ID NO: 678 - DNA, 50 bp, synthetic construct
  source 1..50: mol_type = other DNA
acactctttccctacacgacgctcttccgatctaagccatggagaacgcg

SEQ ID NO: 679 - DNA, 54 bp, synthetic construct
  source 1..54: mol_type = other DNA
acactctttccctacacgacgctcttccgatctccagaagtcttctcagcattt

SEQ ID NO: 680 - DNA, 53 bp, synthetic construct
  source 1..53: mol_type = other DNA
acactctttccctacacgacgctcttccgatctctgacctctttgaaacgctc

SEQ ID NO: 681 - DNA, 53 bp, synthetic construct
  source 1..53: mol_type = other DNA
acactctttccctacacgacgctcttccgatctctaccagtctgagcactact

SEQ ID NO: 682 - DNA, 59 bp, synthetic construct
  source 1..59: mol_type = other DNA
acactctttccctacacgacgctcttccgatcttcttcttacagagagtgtatatggta

SEQ ID NO: 683 - DNA, 54 bp, synthetic construct
  source 1..54: mol_type = other DNA
acactctttccctacacgacgctcttccgatctagttctagtgctgacagatgt

SEQ ID NO: 684 - DNA, 54 bp, synthetic construct
  source 1..54: mol_type = other DNA
acactctttccctacacgacgctcttccgatctgttctgataatccctccgtga

SEQ ID NO: 685 - DNA, 50 bp, synthetic construct
  source 1..50: mol_type = other DNA
acactctttccctacacgacgctcttccgatctccgcccaccttgtattt

SEQ ID NO: 686 - DNA, 52 bp, synthetic construct
  source 1..52: mol_type = other DNA
acactctttccctacacgacgctcttccgatctgtccagcccatgatgattt

SEQ ID NO: 687 - DNA, 52 bp, synthetic construct
  source 1..52: mol_type = other DNA
acactctttccctacacgacgctcttccgatcttttctcctcctgccctaat

SEQ ID NO: 688 - DNA, 52 bp, synthetic construct
  source 1..52: mol_type = other DNA
acactctttccctacacgacgctcttccgatctacccttccctcatatgact

SEQ ID NO: 689 - DNA, 52 bp, synthetic construct
  source 1..52: mol_type = other DNA
acactctttccctacacgacgctcttccgatctaggcccatttcatgctaaa

SEQ ID NO: 690 - DNA, 51 bp, synthetic construct
  source 1..51: mol_type = other DNA
acactctttccctacacgacgctcttccgatctaggagcaagatctggcag

SEQ ID NO: 691 - DNA, 52 bp, synthetic construct
  source 1..52: mol_type = other DNA
acactctttccctacacgacgctcttccgatctgagaaagtcccttcccatg

SEQ ID NO: 692 - DNA, 53 bp, synthetic construct
  source 1..53: mol_type = other DNA
acactctttccctacacgacgctcttccgatctgtggcaatcttggtgaagta

SEQ ID NO: 693 - DNA, 56 bp, synthetic construct
  source 1..56: mol_type = other DNA
acactctttccctacacgacgctcttccgatcttcttactatgagattgctcgtgg

SEQ ID NO: 694 - DNA, 52 bp, synthetic construct
  source 1..52: mol_type = other DNA
acactctttccctacacgacgctcttccgatctgcccttcataaccacctac

SEQ ID NO: 695 - DNA, 54 bp, synthetic construct
  source 1..54: mol_type = other DNA
acactctttccctacacgacgctcttccgatctcagctacccactttggatttt

SEQ ID NO: 696 - DNA, 56 bp, synthetic construct
  source 1..56: mol_type = other DNA
acactctttccctacacgacgctcttccgatctataccgtccaaaagagatcactt

SEQ ID NO: 697 - DNA, 58 bp, synthetic construct
  source 1..58: mol_type = other DNA
acactctttccctacacgacgctcttccgatcttgtatagacacatcttgataggcat

SEQ ID NO: 698 - DNA, 50 bp, synthetic construct
  source 1..50: mol_type = other DNA
acactctttccctacacgacgctcttccgatctgctactatgggctggtc

SEQ ID NO: 699 - DNA, 55 bp, synthetic construct
  source 1..55: mol_type = other DNA
acactctttccctacacgacgctcttccgatctagctaagttcaacgttctgttc

SEQ ID NO: 700 - DNA, 57 bp, synthetic construct
  source 1..57: mol_type = other DNA
acactctttccctacacgacgctcttccgatctaaataactctagggtttggtttca

SEQ ID NO: 701 - DNA, 51 bp, synthetic construct
  source 1..51: mol_type = other DNA
acactctttccctacacgacgctcttccgatctagaaccagctgcagtatg

SEQ ID NO: 702 - DNA, 56 bp, synthetic construct
  source 1..56: mol_type = other DNA
acactctttccctacacgacgctcttccgatctgatccttgtacctgcttgaattt

SEQ ID NO: 703 - DNA, 54 bp, synthetic construct
  source 1..54: mol_type = other DNA
acactctttccctacacgacgctcttccgatcttccaagcctatgcatcatatc

SEQ ID NO: 704 - DNA, 53 bp, synthetic construct
  source 1..53: mol_type = other DNA
acactctttccctacacgacgctcttccgatctccttagctctctcatctcct

SEQ ID NO: 705 - DNA, 52 bp, synthetic construct
  source 1..52: mol_type = other DNA
acactctttccctacacgacgctcttccgatctggagctagaactggcgtta

SEQ ID NO: 706 - DNA, 50 bp, synthetic construct
  source 1..50: mol_type = other DNA
acactctttccctacacgacgctcttccgatcttgcaaccctctcgatgg

SEQ ID NO: 707 - DNA, 58 bp, synthetic construct
  source 1..58: mol_type = other DNA
acactctttccctacacgacgctcttccgatctcaactagcagaatagtaatggatgg

SEQ ID NO: 708 - DNA, 61 bp, synthetic construct
  source 1..61: mol_type = other DNA
acactctttccctacacgacgctcttccgatctcactttaaatatgtagagtttgtcttgg

SEQ ID NO: 709 - DNA, 55 bp, synthetic construct
  source 1..55: mol_type = other DNA
acactctttccctacacgacgctcttccgatctcctacagtgttttcagactcca

SEQ ID NO: 710 - DNA, 52 bp, synthetic construct
  source 1..52: mol_type = other DNA
acactctttccctacacgacgctcttccgatctagagtctgggtagctttgt

SEQ ID NO: 711 - DNA, 50 bp, synthetic construct
  source 1..50: mol_type = other DNA
acactctttccctacacgacgctcttccgatcttcaaccgcaagagcctt

SEQ ID NO: 712 - DNA, 50 bp, synthetic construct
  source 1..50: mol_type = other DNA
acactctttccctacacgacgctcttccgatctttcctccctcactcagc

SEQ ID NO: 713 - DNA, 54 bp, synthetic construct
  source 1..54: mol_type = other DNA
acactctttccctacacgacgctcttccgatctcttgttttcttcctgtctgct

SEQ ID NO: 714 - DNA, 53 bp, synthetic construct
  source 1..53: mol_type = other DNA
acactctttccctacacgacgctcttccgatctgttgtatgtgggatgtgact

SEQ ID NO: 715 - DNA, 62 bp, synthetic construct
  source 1..62: mol_type = other DNA
acactctttccctacacgacgctcttccgatctggttgatgtgtgttattatttgtaattat

SEQ ID NO: 716 - DNA, 51 bp, synthetic construct
  source 1..51: mol_type = other DNA
acactctttccctacacgacgctcttccgatctaactggtccagctcatcc

SEQ ID NO: 717 - DNA, 58 bp, synthetic construct
  source 1..58: mol_type = other DNA
acactctttccctacacgacgctcttccgatctcctcacagacttttagacatcgtag

SEQ ID NO: 718 - DNA, 54 bp, synthetic construct
  source 1..54: mol_type = other DNA
acactctttccctacacgacgctcttccgatctctcttccatagtggttggagt

SEQ ID NO: 719 - DNA, 58 bp, synthetic construct
  source 1..58: mol_type = other DNA
acactctttccctacacgacgctcttccgatctcgcaacagaaaaagtatttaagcag

SEQ ID NO: 720 - DNA, 49 bp, synthetic construct
  source 1..49: mol_type = other DNA
acactctttccctacacgacgctcttccgatctgagccgccgaaccata

SEQ ID NO: 721 - DNA, 53 bp, synthetic construct
  source 1..53: mol_type = other DNA
acactctttccctacacgacgctcttccgatctgaacaagattgtggaccagt

SEQ ID NO: 722 - DNA, 57 bp, synthetic construct
  source 1..57: mol_type = other DNA
acactctttccctacacgacgctcttccgatctgaaactctgaatgccaaagaaatt

SEQ ID NO: 723 - DNA, 57 bp, synthetic construct
  source 1..57: mol_type = other DNA
acactctttccctacacgacgctcttccgatcttgtttggttatttttcagggtaca

SEQ ID NO: 724 - DNA, 54 bp, synthetic construct
  source 1..54: mol_type = other DNA
acactctttccctacacgacgctcttccgatctagtagcagcatctgtgatcat

SEQ ID NO: 725 - DNA, 56 bp, synthetic construct
  source 1..56: mol_type = other DNA
acactctttccctacacgacgctcttccgatctcaggagctatccagaatttaggc

SEQ ID NO: 726 - DNA, 51 bp, synthetic construct
  source 1..51: mol_type = other DNA
acactctttccctacacgacgctcttccgatctgctgcctttctttcctca

SEQ ID NO: 727 - DNA, 54 bp, synthetic construct
  source 1..54: mol_type = other DNA
acactctttccctacacgacgctcttccgatctaggtttgaccctactcagttt

SEQ ID NO: 728 - DNA, 53 bp, synthetic construct
  source 1..53: mol_type = other DNA
acactctttccctacacgacgctcttccgatctgtcttctaagttctggccaa

SEQ ID NO: 729 - DNA, 52 bp, synthetic construct
  source 1..52: mol_type = other DNA
acactctttccctacacgacgctcttccgatcttgaattcccgagcttctcg

SEQ ID NO: 730 - DNA, 50 bp, synthetic construct
  source 1..50: mol_type = other DNA
acactctttccctacacgacgctcttccgatctaacagtgtgtcctcagc

SEQ ID NO: 731 - DNA, 60 bp, synthetic construct
  source 1..60: mol_type = other DNA
acactctttccctacacgacgctcttccgatctaggaatcagatatgtggaaaataagag

SEQ ID NO: 732 - DNA, 52 bp, synthetic construct
  source 1..52: mol_type = other DNA
acactctttccctacacgacgctcttccgatctttccaggagaagtggagca

SEQ ID NO: 733 - DNA, 54 bp, synthetic construct
  source 1..54: mol_type = other DNA
acactctttccctacacgacgctcttccgatctcagaatgactcttctctgtgt

SEQ ID NO: 734 - DNA, 57 bp, synthetic construct
  source 1..57: mol_type = other DNA
acactctttccctacacgacgctcttccgatcttgtttcagtagagatggcatattt

SEQ ID NO: 735 - DNA, 52 bp, synthetic construct
  source 1..52: mol_type = other DNA
acactctttccctacacgacgctcttccgatctcatggccctggataattct

SEQ ID NO: 736 - DNA, 49 bp, synthetic construct
  source 1..49: mol_type = other DNA
acactctttccctacacgacgctcttccgatctggttgcattgctcacc

SEQ ID NO: 737 - DNA, 52 bp, synthetic construct
  source 1..52: mol_type = other DNA
acactctttccctacacgacgctcttccgatctgggacttcagttagtgaca

SEQ ID NO: 738 - DNA, 49 bp, synthetic construct
  source 1..49: mol_type = other DNA
acactctttccctacacgacgctcttccgatctgcactgtcctctgccc

SEQ ID NO: 739 - DNA, 52 bp, synthetic construct
  source 1..52: mol_type = other DNA
acactctttccctacacgacgctcttccgatcttcacagccaacattcagag

SEQ ID NO: 740 - DNA, 51 bp, synthetic construct
  source 1..51: mol_type = other DNA
acactctttccctacacgacgctcttccgatctaagttcttgggcttgctt

SEQ ID NO: 741 - DNA, 49 bp, synthetic construct
  source 1..49: mol_type = other DNA
acactctttccctacacgacgctcttccgatcttgattcccagccagtg

SEQ ID NO: 742 - DNA, 54 bp, synthetic construct
  source 1..54: mol_type = other DNA
acactctttccctacacgacgctcttccgatctcaggtttaaactctggacacg

SEQ ID NO: 743 - DNA, 51 bp, synthetic construct
  source 1..51: mol_type = other DNA
acactctttccctacacgacgctcttccgatctctaccttctcccaccctg

SEQ ID NO: 744 - DNA, 52 bp, synthetic construct
  source 1..52: mol_type = other DNA
acactctttccctacacgacgctcttccgatcttgtgagacacctgcactta

SEQ ID NO: 745 - DNA, 52 bp, synthetic construct
  source 1..52: mol_type = other DNA
acactctttccctacacgacgctcttccgatctcaaccacccaacttctctc

SEQ ID NO: 746 - DNA, 49 bp, synthetic construct
  source 1..49: mol_type = other DNA
acactctttccctacacgacgctcttccgatcttcagggtccccacatg

SEQ ID NO: 747 - DNA, 50 bp, synthetic construct
  source 1..50: mol_type = other DNA
acactctttccctacacgacgctcttccgatcttgtgcctgacttcccag

SEQ ID NO: 748 - DNA, 61 bp, synthetic construct
  source 1..61: mol_type = other DNA
acactctttccctacacgacgctcttccgatctgctcactttcataatttcaactcgaatt

SEQ ID NO: 749 - DNA, 59 bp, synthetic construct
  source 1..59: mol_type = other DNA
acactctttccctacacgacgctcttccgatctgaggtcctaagttacttgatgtgtta

SEQ ID NO: 750 - DNA, 54 bp, synthetic construct
  source 1..54: mol_type = other DNA
acactctttccctacacgacgctcttccgatctcttctggcaatgtggatattc

SEQ ID NO: 751 - DNA, 51 bp, synthetic construct
  source 1..51: mol_type = other DNA
acactctttccctacacgacgctcttccgatcttttggcagcaagtgcaat

SEQ ID NO: 752 - DNA, 53 bp, synthetic construct
  source 1..53: mol_type = other DNA
acactctttccctacacgacgctcttccgatctctgcacagtgctgatcagta

SEQ ID NO: 753 - DNA, 59 bp, synthetic construct
  source 1..59: mol_type = other DNA
acactctttccctacacgacgctcttccgatctgctttttaatttgttgttgaagtgtt

SEQ ID NO: 754 - DNA, 54 bp, synthetic construct
  source 1..54: mol_type = other DNA
acactctttccctacacgacgctcttccgatctcaaagctgactgcaaacaatt

SEQ ID NO: 755 - DNA, 54 bp, synthetic construct
  source 1..54: mol_type = other DNA
acactctttccctacacgacgctcttccgatctcaacctatgtaaaatgcccaa

SEQ ID NO: 756 - DNA, 50 bp, synthetic construct
  source 1..50: mol_type = other DNA
acactctttccctacacgacgctcttccgatctcaggtgtgcacgttgag

SEQ ID NO: 757 - DNA, 53 bp, synthetic construct
  source 1..53: mol_type = other DNA
acactctttccctacacgacgctcttccgatctggtaccccatagtcttcctg

SEQ ID NO: 758 - DNA, 53 bp, synthetic construct
  source 1..53: mol_type = other DNA
acactctttccctacacgacgctcttccgatctccaccaacccaaatcctttc

SEQ ID NO: 759 - DNA, 53 bp, synthetic construct
  source 1..53: mol_type = other DNA
acactctttccctacacgacgctcttccgatctaaggtgaattcctcttccca

SEQ ID NO: 760 - DNA, 55 bp, synthetic construct
  source 1..55: mol_type = other DNA
acactctttccctacacgacgctcttccgatctttcacatttgttcagctatcct

SEQ ID NO: 761 - DNA, 56 bp, synthetic construct
  source 1..56: mol_type = other DNA
acactctttccctacacgacgctcttccgatctcagaatcttcagaaatggcacaa

SEQ ID NO: 762 - DNA, 49 bp, synthetic construct
  source 1..49: mol_type = other DNA
acactctttccctacacgacgctcttccgatctaggctcgctgtactcg

SEQ ID NO: 763 - DNA, 54 bp, synthetic construct
  source 1..54: mol_type = other DNA
acactctttccctacacgacgctcttccgatctcaccttaaaatcagggccatt

SEQ ID NO: 764 - DNA, 49 bp, synthetic construct
  source 1..49: mol_type = other DNA
acactctttccctacacgacgctcttccgatctcagtgcggtgtctctg

SEQ ID NO: 765 - DNA, 52 bp, synthetic construct
  source 1..52: mol_type = other DNA
acactctttccctacacgacgctcttccgatcttatcctaacacctgccctc

SEQ ID NO: 766 - DNA, 57 bp, synthetic construct
  source 1..57: mol_type = other DNA
gtgactggagttcagacgtgtgctcttccgatcttcaacactatgaacccaaacatc

SEQ ID NO: 767 - DNA, 54 bp, synthetic construct
  source 1..54: mol_type = other DNA
gtgactggagttcagacgtgtgctcttccgatcttgtactacgtgttcaccgag

SEQ ID NO: 768 - DNA, 57 bp, synthetic construct
  source 1..57: mol_type = other DNA
gtgactggagttcagacgtgtgctcttccgatctggtctgagaaaatggttcttact

SEQ ID NO: 769 - DNA, 51 bp, synthetic construct
  source 1..51: mol_type = other DNA
gtgactggagttcagacgtgtgctcttccgatctggtcattccgagaacgc

SEQ ID NO: 770 - DNA, 55 bp, synthetic construct
  source 1..55: mol_type = other DNA
gtgactggagttcagacgtgtgctcttccgatctctgcaaaccttgaaacagaat

SEQ ID NO: 771 - DNA, 56 bp, synthetic construct
  source 1..56: mol_type = other DNA
gtgactggagttcagacgtgtgctcttccgatctgtttgatgtatgctggcttcat

SEQ ID NO: 772 - DNA, 57 bp, synthetic construct
  source 1..57: mol_type = other DNA
gtgactggagttcagacgtgtgctcttccgatctaaaatcaatgatgccatagctga

SEQ ID NO: 773 - DNA, 52 bp, synthetic construct
  source 1..52: mol_type = other DNA
gtgactggagttcagacgtgtgctcttccgatctcattggtgtggccaagaa

SEQ ID NO: 774 - DNA, 52 bp, synthetic construct
  source 1..52: mol_type = other DNA
gtgactggagttcagacgtgtgctcttccgatctgaatcccaacatggtccc

SEQ ID NO: 775 - DNA, 50 bp, synthetic construct
  source 1..50: mol_type = other DNA
gtgactggagttcagacgtgtgctcttccgatctcgatgaggagccactg

SEQ ID NO: 776 - DNA, 57 bp, synthetic construct
  source 1..57: mol_type = other DNA
gtgactggagttcagacgtgtgctcttccgatctgtctttacctgtttgtgatgagc

SEQ ID NO: 777 - DNA, 54 bp, synthetic construct
  source 1..54: mol_type = other DNA
gtgactggagttcagacgtgtgctcttccgatcttccagctactctgtgtcatt

SEQ ID NO: 778 - DNA, 59 bp, synthetic construct
  source 1..59: mol_type = other DNA
gtgactggagttcagacgtgtgctcttccgatctcagtgcctaccctaaattaatagaa

SEQ ID NO: 779 - DNA, 52 bp, synthetic construct
  source 1..52: mol_type = other DNA
gtgactggagttcagacgtgtgctcttccgatctttgttgaccagctccagg

SEQ ID NO: 780 - DNA, 55 bp, synthetic construct
  source 1..55: mol_type = other DNA
gtgactggagttcagacgtgtgctcttccgatctcccatgaattgaaatagcagc

SEQ ID NO: 781 - DNA, 53 bp, synthetic construct
  source 1..53: mol_type = other DNA
gtgactggagttcagacgtgtgctcttccgatcttcagactctgacctcgatc

SEQ ID NO: 782 - DNA, 53 bp, synthetic construct
  source 1..53: mol_type = other DNA
gtgactggagttcagacgtgtgctcttccgatctgctactacccggtcatctt

SEQ ID NO: 783 - DNA, 58 bp, synthetic construct
  source 1..58: mol_type = other DNA
gtgactggagttcagacgtgtgctcttccgatctcacatctccactcttcaatggatt

SEQ ID NO: 784 - DNA, 55 bp, synthetic construct
  source 1..55: mol_type = other DNA
gtgactggagttcagacgtgtgctcttccgatctgacataggagcagagctgaag

SEQ ID NO: 785 - DNA, 50 bp, synthetic construct
  source 1..50: mol_type = other DNA
gtgactggagttcagacgtgtgctcttccgatctgggaaagaggagggcc

SEQ ID NO: 786 - DNA, 55 bp, synthetic construct
  source 1..55: mol_type = other DNA
gtgactggagttcagacgtgtgctcttccgatctcagcagtctaatcaatggcag

SEQ ID NO: 787 - DNA, 54 bp, synthetic construct
  source 1..54: mol_type = other DNA
gtgactggagttcagacgtgtgctcttccgatctccctgcaacatgggaaataa

SEQ ID NO: 788 - DNA, 55 bp, synthetic construct
  source 1..55: mol_type = other DNA
gtgactggagttcagacgtgtgctcttccgatctctgcttcacaaaaacttgcag

SEQ ID NO: 789 - DNA, 61 bp, synthetic construct
  source 1..61: mol_type = other DNA
gtgactggagttcagacgtgtgctcttccgatctcagtattggacattagatagcatttat

SEQ ID NO: 790 - DNA, 53 bp, synthetic construct
  source 1..53: mol_type = other DNA
gtgactggagttcagacgtgtgctcttccgatctcatacgcccctctcctaca

SEQ ID NO: 791 - DNA, 55 bp, synthetic construct
  source 1..55: mol_type = other DNA
gtgactggagttcagacgtgtgctcttccgatcttaggcattgtagtcctggaag

SEQ ID NO: 792 - DNA, 59 bp, synthetic construct
  source 1..59: mol_type = other DNA
gtgactggagttcagacgtgtgctcttccgatctcattccgaaagatctttggtagata

SEQ ID NO: 793 - DNA, 54 bp, synthetic construct
  source 1..54: mol_type = other DNA
gtgactggagttcagacgtgtgctcttccgatctttggtgaagtaggtgatgga

SEQ ID NO: 794 - DNA, 58 bp, synthetic construct
  source 1..58: mol_type = other DNA
gtgactggagttcagacgtgtgctcttccgatctgcagagccattgttgatatgttat

SEQ ID NO: 795 - DNA, 55 bp, synthetic construct
  source 1..55: mol_type = other DNA
gtgactggagttcagacgtgtgctcttccgatctaaccctaatgatctgaccaac

SEQ ID NO: 796 - DNA, 54 bp, synthetic construct
  source 1..54: mol_type = other DNA
gtgactggagttcagacgtgtgctcttccgatctgtaatgagagatgggctcac

SEQ ID NO: 797 - DNA, 54 bp, synthetic construct
  source 1..54: mol_type = other DNA
gtgactggagttcagacgtgtgctcttccgatctctacaggagacctttgagga

SEQ ID NO: 798 - DNA, 54 bp, synthetic construct
  source 1..54: mol_type = other DNA
gtgactggagttcagacgtgtgctcttccgatctttctatatatccccagccgg

SEQ ID NO: 799 - DNA, 51 bp, synthetic construct
  source 1..51: mol_type = other DNA
gtgactggagttcagacgtgtgctcttccgatctcaggattcgactcaggc

SEQ ID NO: 800 - DNA, 55 bp, synthetic construct
  source 1..55: mol_type = other DNA
gtgactggagttcagacgtgtgctcttccgatcttatgtacgatggcttctggtc

SEQ ID NO: 801 - DNA, 52 bp, synthetic construct
  source 1..52: mol_type = other DNA
gtgactggagttcagacgtgtgctcttccgatctacccattccagctttgtg

SEQ ID NO: 802 - DNA, 54 bp, synthetic construct
  source 1..54: mol_type = other DNA
gtgactggagttcagacgtgtgctcttccgatctgtagccccaaataccaatga

SEQ ID NO: 803 - DNA, 54 bp, synthetic construct
  source 1..54: mol_type = other DNA
gtgactggagttcagacgtgtgctcttccgatctctccagctatgtgtgaagaa

SEQ ID NO: 804 - DNA, 53 bp, synthetic construct
  source 1..53: mol_type = other DNA
gtgactggagttcagacgtgtgctcttccgatctagcctcatttaccagcctg

SEQ ID NO: 805 - DNA, 54 bp, synthetic construct
  source 1..54: mol_type = other DNA
gtgactggagttcagacgtgtgctcttccgatcttatgaggccctacatttgca

SEQ ID NO: 806 - DNA, 56 bp, synthetic construct
  source 1..56: mol_type = other DNA
gtgactggagttcagacgtgtgctcttccgatctgctggccatcattattactgtg

SEQ ID NO: 807 - DNA, 60 bp, synthetic construct
  source 1..60: mol_type = other DNA
gtgactggagttcagacgtgtgctcttccgatctcagaaatattcttccaggtagcaaaa

SEQ ID NO: 808 - DNA, 53 bp, synthetic construct
  source 1..53: mol_type = other DNA
gtgactggagttcagacgtgtgctcttccgatctccccttaaaccactgaaga

SEQ ID NO: 809 - DNA, 56 bp, synthetic construct
  source 1..56: mol_type = other DNA
gtgactggagttcagacgtgtgctcttccgatctgacaatgctccttaagtctctt

SEQ ID NO: 810 - DNA, 52 bp, synthetic construct
  source 1..52: mol_type = other DNA
gtgactggagttcagacgtgtgctcttccgatcttgccttcccagttcttga

SEQ ID NO: 811 - DNA, 57 bp, synthetic construct
  source 1..57: mol_type = other DNA
gtgactggagttcagacgtgtgctcttccgatcttaaatcataggctacagctgaaa

SEQ ID NO: 812 - DNA, 57 bp, synthetic construct
  source 1..57: mol_type = other DNA
gtgactggagttcagacgtgtgctcttccgatctggctttgctttactaccaatctc

SEQ ID NO: 813 - DNA, 55 bp, synthetic construct
  source 1..55: mol_type = other DNA
gtgactggagttcagacgtgtgctcttccgatcttgggatgtggaaagtcattct

SEQ ID NO: 814 - DNA, 57 bp, synthetic construct
  source 1..57: mol_type = other DNA
gtgactggagttcagacgtgtgctcttccgatctcgtaaggtgatacacaagttctg

SEQ ID NO: 815 - DNA, 58 bp, synthetic construct
  source 1..58: mol_type = other DNA
gtgactggagttcagacgtgtgctcttccgatctgctcaaatgtctttctgaagtacg

SEQ ID NO: 816 - DNA, 51 bp, synthetic construct
  source 1..51: mol_type = other DNA
gtgactggagttcagacgtgtgctcttccgatctgagtggatgtgcaggtc

SEQ ID NO: 817 - DNA, 52 bp, synthetic construct
  source 1..52: mol_type = other DNA
gtgactggagttcagacgtgtgctcttccgatcttgccgtcttctacaacaa

SEQ ID NO: 818 - DNA, 55 bp, synthetic construct
  source 1..55: mol_type = other DNA
gtgactggagttcagacgtgtgctcttccgatctccatcatacaccacaaatcca

SEQ ID NO: 819 - DNA, 58 bp, synthetic construct
  source 1..58: mol_type = other DNA
gtgactggagttcagacgtgtgctcttccgatctgatgaatactcagtcagggtatca

SEQ ID NO: 820 - DNA, 53 bp, synthetic construct
  source 1..53: mol_type = other DNA
gtgactggagttcagacgtgtgctcttccgatctgtgacaggtctctggtaga

SEQ ID NO: 821 - DNA, 50 bp, synthetic construct
  source 1..50: mol_type = other DNA
gtgactggagttcagacgtgtgctcttccgatcttaggtcgcgcgctatc

SEQ ID NO: 822 - DNA, 56 bp, synthetic construct
  source 1..56: mol_type = other DNA
gtgactggagttcagacgtgtgctcttccgatctccttttcatgccaaagtcctat

SEQ ID NO: 823 - DNA, 57 bp, synthetic construct
  source 1..57: mol_type = other DNA
gtgactggagttcagacgtgtgctcttccgatctgtcataagacatcttaggagcca

SEQ ID NO: 824 - DNA, 50 bp, synthetic construct
  source 1..50: mol_type = other DNA
gtgactggagttcagacgtgtgctcttccgatctatgatgaggcagggcg

SEQ ID NO: 825 - DNA, 52 bp, synthetic construct
  source 1..52: mol_type = other DNA
gtgactggagttcagacgtgtgctcttccgatctaacctgctggtcatcgtg

SEQ ID NO: 826 - DNA, 51 bp, synthetic construct
  source 1..51: mol_type = other DNA
gtgactggagttcagacgtgtgctcttccgatctgggctcaggttctagct

SEQ ID NO: 827 - DNA, 55 bp, synthetic construct
  source 1..55: mol_type = other DNA
gtgactggagttcagacgtgtgctcttccgatctggtccctttctcattcagtta

SEQ ID NO: 828 - DNA, 52 bp, synthetic construct
  source 1..52: mol_type = other DNA
gtgactggagttcagacgtgtgctcttccgatctagtcctaacccgtgttgc

SEQ ID NO: 829 - DNA, 50 bp, synthetic construct
  source 1..50: mol_type = other DNA
gtgactggagttcagacgtgtgctcttccgatctggcctgtgacgctcac

SEQ ID NO: 830 - DNA, 58 bp, synthetic construct
  source 1..58: mol_type = other DNA
gtgactggagttcagacgtgtgctcttccgatctctcaaagaagcctaaacaaagtac

SEQ ID NO: 831 - DNA, 50 bp, synthetic construct
  source 1..50: mol_type = other DNA
gtgactggagttcagacgtgtgctcttccgatctctagccacatcagcct

SEQ ID NO: 832 - DNA, 55 bp, synthetic construct
  source 1..55: mol_type = other DNA
gtgactggagttcagacgtgtgctcttccgatcttattagggtgcaagaggactg

SEQ ID NO: 833 - DNA, 53 bp, synthetic construct
  source 1..53: mol_type = other DNA
gtgactggagttcagacgtgtgctcttccgatcttgtgcatcacttaccggtt

SEQ ID NO: 834 - DNA, 55 bp, synthetic construct
  source 1..55: mol_type = other DNA
gtgactggagttcagacgtgtgctcttccgatctaaaggtttaaccaagggaagc

SEQ ID NO: 835 - DNA, 50 bp, synthetic construct
  source 1..50: mol_type = other DNA
gtgactggagttcagacgtgtgctcttccgatctgagctactgtgggctg

SEQ ID NO: 836 - DNA, 53 bp, synthetic construct
  source 1..53: mol_type = other DNA
gtgactggagttcagacgtgtgctcttccgatctgctactcaccgcttctttg

SEQ ID NO: 837 - DNA, 58 bp, synthetic construct
  source 1..58: mol_type = other DNA
gtgactggagttcagacgtgtgctcttccgatctcacagacaagccctcagatatatt

SEQ ID NO: 838 - DNA, 56 bp, synthetic construct
  source 1..56: mol_type = other DNA
gtgactggagttcagacgtgtgctcttccgatcttccagaatctgttacctgtgaa

SEQ ID NO: 839 - DNA, 53 bp, synthetic construct
  source 1..53: mol_type = other DNA
gtgactggagttcagacgtgtgctcttccgatcttccagctgaaaattggagc

SEQ ID NO: 840 - DNA, 55 bp, synthetic construct
  source 1..55: mol_type = other DNA
gtgactggagttcagacgtgtgctcttccgatctaaatggaaagcttggactcac

SEQ ID NO: 841 - DNA, 58 bp, synthetic construct
  source 1..58: mol_type = other DNA
gtgactggagttcagacgtgtgctcttccgatctactgtgaaatgatatggagctttt

SEQ ID NO: 842 - DNA, 52 bp, synthetic construct
  source 1..52: mol_type = other DNA
gtgactggagttcagacgtgtgctcttccgatctgagacagtgttggacatg

SEQ ID NO: 843 - DNA, 59 bp, synthetic construct
  source 1..59: mol_type = other DNA
gtgactggagttcagacgtgtgctcttccgatctcgtactgtcagttaacctaactcat

SEQ ID NO: 844 - DNA, 56 bp, synthetic construct
  source 1..56: mol_type = other DNA
gtgactggagttcagacgtgtgctcttccgatcttaggtgagaaacctggaaatga

SEQ ID NO: 845 - DNA, 63 bp, synthetic construct
  source 1..63: mol_type = other DNA
gtgactggagttcagacgtgtgctcttccgatctaataatagaactaacagcactcagaatca

SEQ ID NO: 846 - DNA, 58 bp, synthetic construct
  source 1..58: mol_type = other DNA
gtgactggagttcagacgtgtgctcttccgatctgcttaacttcaaggaaaacactca

SEQ ID NO: 847 - DNA, 58 bp, synthetic construct
  source 1..58: mol_type = other DNA
gtgactggagttcagacgtgtgctcttccgatctcacaagcttcactctgattaagaa

SEQ ID NO: 848 - DNA, 59 bp, synthetic construct
  source 1..59: mol_type = other DNA
gtgactggagttcagacgtgtgctcttccgatctcatggataactgaagatttctctcc

SEQ ID NO: 849 - DNA, 52 bp, synthetic construct
  source 1..52: mol_type = other DNA
gtgactggagttcagacgtgtgctcttccgatcttggtctttgctgagcctc

SEQ ID NO: 850 - DNA, 56 bp, synthetic construct
  source 1..56: mol_type = other DNA
gtgactggagttcagacgtgtgctcttccgatctaacattggcattttccatgatg

SEQ ID NO: 851 - DNA, 54 bp, synthetic construct
  source 1..54: mol_type = other DNA
gtgactggagttcagacgtgtgctcttccgatcttgaccctctttgtgtagctg

SEQ ID NO: 852 - DNA, 52 bp, synthetic construct
  source 1..52: mol_type = other DNA
gtgactggagttcagacgtgtgctcttccgatcttgtgcgttctcgttctag

SEQ ID NO: 853 - DNA, 50 bp, synthetic construct
  source 1..50: mol_type = other DNA
gtgactggagttcagacgtgtgctcttccgatctacctctggacctgctg

SEQ ID NO: 854 - DNA, 53 bp, synthetic construct
  source 1..53: mol_type = other DNA
gtgactggagttcagacgtgtgctcttccgatcttcagaaaaggtacaccccg

SEQ ID NO: 855 - DNA, 50 bp, synthetic construct
  source 1..50: mol_type = other DNA
gtgactggagttcagacgtgtgctcttccgatctcttgcgaacatgcggc

SEQ ID NO: 856 - DNA, 56 bp, synthetic construct
  source 1..56: mol_type = other DNA
gtgactggagttcagacgtgtgctcttccgatctcctcttttgtcaccaatctttg

SEQ ID NO: 857 - DNA, 51 bp, synthetic construct
  source 1..51: mol_type = other DNA
gtgactggagttcagacgtgtgctcttccgatctcgccccatctgatgctc

SEQ ID NO: 858 - DNA, 55 bp, synthetic construct
  source 1..55: mol_type = other DNA
gtgactggagttcagacgtgtgctcttccgatcttcccggttttagagaaatgtg

SEQ ID NO: 859 - DNA, 54 bp, synthetic construct
  source 1..54: mol_type = other DNA
gtgactggagttcagacgtgtgctcttccgatctaggtaccagtcgtcattcag

SEQ ID NO: 860 - DNA, 51 bp, synthetic construct
  source 1..51: mol_type = other DNA
gtgactggagttcagacgtgtgctcttccgatctttgtggagacctttggc

SEQ ID NO: 861 - DNA, 54 bp, synthetic construct
  source 1..54: mol_type = other DNA
gtgactggagttcagacgtgtgctcttccgatctagctttccagtgaagactct

SEQ ID NO: 862 - DNA, 55 bp, synthetic construct
  source 1..55: mol_type = other DNA
gtgactggagttcagacgtgtgctcttccgatcttagccccattctagaaaatgc

SEQ ID NO: 863 - DNA, 52 bp, synthetic construct
  source 1..52: mol_type = other DNA
gtgactggagttcagacgtgtgctcttccgatctgcagtaggtagccgagat

SEQ ID NO: 864 - DNA, 54 bp, synthetic construct
  source 1..54: mol_type = other DNA
gtgactggagttcagacgtgtgctcttccgatctagccaatggtaaacctgcat

SEQ ID NO: 865 - DNA, 55 bp, synthetic construct
  source 1..55: mol_type = other DNA
gtgactggagttcagacgtgtgctcttccgatctgctggtcttttggtatcgtag

SEQ ID NO: 866 - DNA, 55 bp, synthetic construct
  source 1..55: mol_type = other DNA
gtgactggagttcagacgtgtgctcttccgatctaataccatctgtcaaagaggc

SEQ ID NO: 867 - DNA, 53 bp, synthetic construct
  source 1..53: mol_type = other DNA
gtgactggagttcagacgtgtgctcttccgatctcagatgccacagttctcat

SEQ ID NO: 868 - DNA, 52 bp, synthetic construct
  source 1..52: mol_type = other DNA
gtgactggagttcagacgtgtgctcttccgatcttggacctgacaaggagag

SEQ ID NO: 869 - DNA, 57 bp, synthetic construct
  source 1..57: mol_type = other DNA
gtgactggagttcagacgtgtgctcttccgatcttgcaattacacctgactttctcc

SEQ ID NO: 870 - DNA, 54 bp, synthetic construct
  source 1..54: mol_type = other DNA
gtgactggagttcagacgtgtgctcttccgatcttccattgacaattcatggcc

SEQ ID NO: 871 - DNA, 55 bp, synthetic construct
  source 1..55: mol_type = other DNA
gtgactggagttcagacgtgtgctcttccgatctaaactgacctctcgtttgtct

SEQ ID NO: 872 - DNA, 53 bp, synthetic construct
  source 1..53: mol_type = other DNA
gtgactggagttcagacgtgtgctcttccgatctttgtcctctgcagtacctg

SEQ ID NO: 873 - DNA, 55 bp, synthetic construct
  source 1..55: mol_type = other DNA
gtgactggagttcagacgtgtgctcttccgatctttctgctgaggtggtaaatgg

SEQ ID NO: 874 - DNA, 52 bp, synthetic construct
  source 1..52: mol_type = other DNA
gtgactggagttcagacgtgtgctcttccgatctaaccgccatgatcagaag

SEQ ID NO: 875 - DNA, 56 bp, synthetic construct
  source 1..56: mol_type = other DNA
gtgactggagttcagacgtgtgctcttccgatctgtctgtttgatctcaccatctt

SEQ ID NO: 876 - DNA, 57 bp, synthetic construct
  source 1..57: mol_type = other DNA
gtgactggagttcagacgtgtgctcttccgatctgctcctgaacaatatctaagtgt

SEQ ID NO: 877 - DNA, 60 bp, synthetic construct
  source 1..60: mol_type = other DNA
gtgactggagttcagacgtgtgctcttccgatctgttgacattctctttgaagatatggt

SEQ ID NO: 878 - DNA, 53 bp, synthetic construct
  source 1..53: mol_type = other DNA
gtgactggagttcagacgtgtgctcttccgatctttcagcagatgtgaatgcc

SEQ ID NO: 879 - DNA, 52 bp, synthetic construct
  source 1..52: mol_type = other DNA
gtgactggagttcagacgtgtgctcttccgatctcagaatggtgatgggctg

SEQ ID NO: 880 - DNA, 53 bp, synthetic construct
  source 1..53: mol_type = other DNA
gtgactggagttcagacgtgtgctcttccgatctcttcattgtagcgcctcag

SEQ ID NO: 881 - DNA, 57 bp, synthetic construct
  source 1..57: mol_type = other DNA
gtgactggagttcagacgtgtgctcttccgatctctacactgaggactttggtaaac

SEQ ID NO: 882 - DNA, 52 bp, synthetic construct
  source 1..52: mol_type = other DNA
gtgactggagttcagacgtgtgctcttccgatctggttggctcgaaagtgac

SEQ ID NO: 883 - DNA, 52 bp, synthetic construct
  source 1..52: mol_type = other DNA
gtgactggagttcagacgtgtgctcttccgatctagcctccacctattgtga

SEQ ID NO: 884 - DNA, 54 bp, synthetic construct
  source 1..54: mol_type = other DNA
gtgactggagttcagacgtgtgctcttccgatctttcgtgggaaaaactgtctc

SEQ ID NO: 885 - DNA, 61 bp, synthetic construct
  source 1..61: mol_type = other DNA
gtgactggagttcagacgtgtgctcttccgatcttgctattgtcatattacaccctttaag

SEQ ID NO: 886 - DNA, 54 bp, synthetic construct
  source 1..54: mol_type = other DNA
gtgactggagttcagacgtgtgctcttccgatctctgtaggatggccactatct

SEQ ID NO: 887 - DNA, 55 bp, synthetic construct
  source 1..55: mol_type = other DNA
gtgactggagttcagacgtgtgctcttccgatctggatgtagtttttcaggcttg

SEQ ID NO: 888 - DNA, 58 bp, synthetic construct
  source 1..58: mol_type = other DNA
gtgactggagttcagacgtgtgctcttccgatcttagcctcttcaatattaagtggaa

SEQ ID NO: 889 - DNA, 51 bp, synthetic construct
  source 1..51: mol_type = other DNA
gtgactggagttcagacgtgtgctcttccgatctggccaaaaccgactgtg

SEQ ID NO: 890 - DNA, 55 bp, synthetic construct
  source 1..55: mol_type = other DNA
gtgactggagttcagacgtgtgctcttccgatctcaggttcttggtcttgctaag

SEQ ID NO: 891 - DNA, 53 bp, synthetic construct
  source 1..53: mol_type = other DNA
gtgactggagttcagacgtgtgctcttccgatcttgacaaacactgcaggaaa

SEQ ID NO: 892 - DNA, 52 bp, synthetic construct
  source 1..52: mol_type = other DNA
gtgactggagttcagacgtgtgctcttccgatctagccttgtcctccagtgt

SEQ ID NO: 893 - DNA, 51 bp, synthetic construct
  source 1..51: mol_type = other DNA
gtgactggagttcagacgtgtgctcttccgatctccaccaagtgcttacgg

SEQ ID NO: 894 - DNA, 52 bp, synthetic construct
  source 1..52: mol_type = other DNA
gtgactggagttcagacgtgtgctcttccgatctctttcctcctccctgaga

SEQ ID NO: 895 - DNA, 56 bp, synthetic construct
  source 1..56: mol_type = other DNA
gtgactggagttcagacgtgtgctcttccgatctgattacctgcagtgtggtagaa

SEQ ID NO: 896 - DNA, 55 bp, synthetic construct
  source 1..55: mol_type = other DNA
gtgactggagttcagacgtgtgctcttccgatctgtttgcccagaactgttgatt

SEQ ID NO: 897 - DNA, 56 bp, synthetic construct
  source 1..56: mol_type = other DNA
gtgactggagttcagacgtgtgctcttccgatctccttacactttgtagacatgca

SEQ ID NO: 898 - DNA, 52 bp, synthetic construct
  source 1..52: mol_type = other DNA
gtgactggagttcagacgtgtgctcttccgatctagctgagtcatcctcgtc

SEQ ID NO: 899 - DNA, 53 bp, synthetic construct
  source 1..53: mol_type = other DNA
gtgactggagttcagacgtgtgctcttccgatctacgtgtagtcagcttcctc

SEQ ID NO: 900 - DNA, 54 bp, synthetic construct
  source 1..54: mol_type = other DNA
gtgactggagttcagacgtgtgctcttccgatctcaaagggttcaatgtggaga

SEQ ID NO: 901 - DNA, 51 bp, synthetic construct
  source 1..51: mol_type = other DNA
gtgactggagttcagacgtgtgctcttccgatctcgggctcacctatggtt

SEQ ID NO: 902 - DNA, 52 bp, synthetic construct
  source 1..52: mol_type = other DNA
gtgactggagttcagacgtgtgctcttccgatctagggtgtgactagctcct

SEQ ID NO: 903 - DNA, 52 bp, synthetic construct
  source 1..52: mol_type = other DNA
gtgactggagttcagacgtgtgctcttccgatctccctcatgaaagcttccc

SEQ ID NO: 904 - DNA, 58 bp, synthetic construct
  source 1..58: mol_type = other DNA
gtgactggagttcagacgtgtgctcttccgatctggcccttaatgctttacattttct

SEQ ID NO: 905 - DNA, 52 bp, synthetic construct
  source 1..52: mol_type = other DNA
gtgactggagttcagacgtgtgctcttccgatctcctctgctgtcctctcaa

SEQ ID NO: 906 - DNA, 51 bp, synthetic construct
  source 1..51: mol_type = other DNA
gtgactggagttcagacgtgtgctcttccgatctaagttccagtccccacc

SEQ ID NO: 907 - DNA, 55 bp, synthetic construct
  source 1..55: mol_type = other DNA
gtgactggagttcagacgtgtgctcttccgatctctgttgaagctgctcgatttt

SEQ ID NO: 908 - DNA, 51 bp, synthetic construct
  source 1..51: mol_type = other DNA
gtgactggagttcagacgtgtgctcttccgatctgagggtactgcattccg

SEQ ID NO: 909 - DNA, 58 bp, synthetic construct
  source 1..58: mol_type = other DNA
gtgactggagttcagacgtgtgctcttccgatctgagtcaaagataaacacttcatgc

SEQ ID NO: 910 - DNA, 54 bp, synthetic construct
  source 1..54: mol_type = other DNA
gtgactggagttcagacgtgtgctcttccgatctctctaggccatactggagat

SEQ ID NO: 911 - DNA, 53 bp, synthetic construct
  source 1..53: mol_type = other DNA
gtgactggagttcagacgtgtgctcttccgatctgtttgcataggcctcacag

SEQ ID NO: 912 - DNA, 54 bp, synthetic construct
  source 1..54: mol_type = other DNA
gtgactggagttcagacgtgtgctcttccgatctctcttcaggagttcacaacg

SEQ ID NO: 913 - DNA, 52 bp, synthetic construct
  source 1..52: mol_type = other DNA
gtgactggagttcagacgtgtgctcttccgatctcatctgctgagaaggcag

SEQ ID NO: 914 - DNA, 62 bp, synthetic construct
  source 1..62: mol_type = other DNA
gtgactggagttcagacgtgtgctcttccgatctgcatctaacaaagatacttacatttgaa

SEQ ID NO: 915 - DNA, 54 bp, synthetic construct
  source 1..54: mol_type = other DNA
gtgactggagttcagacgtgtgctcttccgatctcacaaagaccatgactcctc

SEQ ID NO: 916 - DNA, 60 bp, synthetic construct
  source 1..60: mol_type = other DNA
gtgactggagttcagacgtgtgctcttccgatcttgcattgttacccaaagtaatcaaag

SEQ ID NO: 917 - DNA, 57 bp, synthetic construct
  source 1..57: mol_type = other DNA
gtgactggagttcagacgtgtgctcttccgatctcttacctgcaccaagttgtaaat

SEQ ID NO: 918 - DNA, 54 bp, synthetic construct
  source 1..54: mol_type = other DNA
gtgactggagttcagacgtgtgctcttccgatctcctttgttctcctgcacaat

SEQ ID NO: 919 - DNA, 54 bp, synthetic construct
  source 1..54: mol_type = other DNA
gtgactggagttcagacgtgtgctcttccgatctttgttacccagagtgaccaa

SEQ ID NO: 920 - DNA, 54 bp, synthetic construct
  source 1..54: mol_type = other DNA
gtgactggagttcagacgtgtgctcttccgatctgtggaaactctgtcatgtgt

SEQ ID NO: 921 - DNA, 55 bp, synthetic construct
  source 1..55: mol_type = other DNA
gtgactggagttcagacgtgtgctcttccgatctatcagtgaagaaagggcatca

SEQ ID NO: 922 - DNA, 52 bp, synthetic construct
  source 1..52: mol_type = other DNA
gtgactggagttcagacgtgtgctcttccgatctagctaactccaagctccc

SEQ ID NO: 923 - DNA, 54 bp, synthetic construct
  source 1..54: mol_type = other DNA
gtgactggagttcagacgtgtgctcttccgatctcctctctgaaggaacattcg

SEQ ID NO: 924 - DNA, 56 bp, synthetic construct
  source 1..56: mol_type = other DNA
gtgactggagttcagacgtgtgctcttccgatctgtttcaggtttcttgctgtatg

SEQ ID NO: 925 - DNA, 52 bp, synthetic construct
  source 1..52: mol_type = other DNA
gtgactggagttcagacgtgtgctcttccgatctaataccccttcgagccag

SEQ ID NO: 926 - DNA, 56 bp, synthetic construct
  source 1..56: mol_type = other DNA
gtgactggagttcagacgtgtgctcttccgatctagcgaaagaagtttgaccatga

SEQ ID NO: 927 - DNA, 54 bp, synthetic construct
  source 1..54: mol_type = other DNA
gtgactggagttcagacgtgtgctcttccgatcttctaggccatggagtatctg

SEQ ID NO: 928 - DNA, 50 bp, synthetic construct
  source 1..50: mol_type = other DNA
gtgactggagttcagacgtgtgctcttccgatctcgtagccattctgcag

SEQ ID NO: 929 - DNA, 52 bp, synthetic construct
  source 1..52: mol_type = other DNA
gtgactggagttcagacgtgtgctcttccgatctaaggacctcatagggagc

SEQ ID NO: 930 - DNA, 51 bp, synthetic construct
  source 1..51: mol_type = other DNA
gtgactggagttcagacgtgtgctcttccgatctcacagaggtggatgctg

SEQ ID NO: 931 - DNA, 55 bp, synthetic construct
  source 1..55: mol_type = other DNA
gtgactggagttcagacgtgtgctcttccgatctgtgtttctaatggtgcatcct

SEQ ID NO: 932 - DNA, 54 bp, synthetic construct
  source 1..54: mol_type = other DNA
gtgactggagttcagacgtgtgctcttccgatctctctggatcccacaggtatt

SEQ ID NO: 933 - DNA, 51 bp, synthetic construct
  source 1..51: mol_type = other DNA
gtgactggagttcagacgtgtgctcttccgatctcacctaccgttggagcc

SEQ ID NO: 934 - DNA, 53 bp, synthetic construct
  source 1..53: mol_type = other DNA
gtgactggagttcagacgtgtgctcttccgatctcacagtaacagctgtctgg

SEQ ID NO: 935 - DNA, 54 bp, synthetic construct
  source 1..54: mol_type = other DNA
gtgactggagttcagacgtgtgctcttccgatctccgtttcagtaccagtgaag

SEQ ID NO: 936 - DNA, 54 bp, synthetic construct
  source 1..54: mol_type = other DNA
gtgactggagttcagacgtgtgctcttccgatcttgctgtgacttacttgaagc

SEQ ID NO: 937 - DNA, 59 bp, synthetic construct
  source 1..59: mol_type = other DNA
gtgactggagttcagacgtgtgctcttccgatctcttacttcattgttcctactcagag

SEQ ID NO: 938 - DNA, 55 bp, synthetic construct
  source 1..55: mol_type = other DNA
gtgactggagttcagacgtgtgctcttccgatctcacttccagcttactcacagt

SEQ ID NO: 939 - DNA, 54 bp, synthetic construct
  source 1..54: mol_type = other DNA
gtgactggagttcagacgtgtgctcttccgatctggttgtccaagacttcatcc

SEQ ID NO: 940 - DNA, 50 bp, synthetic construct
  source 1..50: mol_type = other DNA
gtgactggagttcagacgtgtgctcttccgatctggtgtttgtcctgggc

SEQ ID NO: 941 - DNA, 54 bp, synthetic construct
  source 1..54: mol_type = other DNA
gtgactggagttcagacgtgtgctcttccgatctacattatgtcccatgcatgc

SEQ ID NO: 942 - DNA, 54 bp, synthetic construct
  source 1..54: mol_type = other DNA
gtgactggagttcagacgtgtgctcttccgatctgtgagttattggttcgagcc

SEQ ID NO: 943 - DNA, 52 bp, synthetic construct
  source 1..52: mol_type = other DNA
gtgactggagttcagacgtgtgctcttccgatctctcccaactctgtcacct

SEQ ID NO: 944 - DNA, 54 bp, synthetic construct
  source 1..54: mol_type = other DNA
gtgactggagttcagacgtgtgctcttccgatcttggctctggacatgacatat

SEQ ID NO: 945 - DNA, 50 bp, synthetic construct
  source 1..50: mol_type = other DNA
gtgactggagttcagacgtgtgctcttccgatctgtgcgctctggtcctt

SEQ ID NO: 946 - DNA, 52 bp, synthetic construct
  source 1..52: mol_type = other DNA
gtgactggagttcagacgtgtgctcttccgatctattctccaggcgagtcag

SEQ ID NO: 947 - DNA, 52 bp, synthetic construct
  source 1..52: mol_type = other DNA
gtgactggagttcagacgtgtgctcttccgatcttggttactggctcacctg

SEQ ID NO: 948 - DNA, 56 bp, synthetic construct
  source 1..56: mol_type = other DNA
gtgactggagttcagacgtgtgctcttccgatctacaccttgtcaaaacccctttc

SEQ ID NO: 949 - DNA, 54 bp, synthetic construct
  source 1..54: mol_type = other DNA
gtgactggagttcagacgtgtgctcttccgatctatgacatggttgctgaatgg

SEQ ID NO: 950 - DNA, 55 bp, synthetic construct
  source 1..55: mol_type = other DNA
gtgactggagttcagacgtgtgctcttccgatctgtcctcagttgggaactattt

SEQ ID NO: 951 - DNA, 56 bp, synthetic construct
  source 1..56: mol_type = other DNA
gtgactggagttcagacgtgtgctcttccgatctcttttcttaggtagcagatggg

SEQ ID NO: 952 - DNA, 58 bp, synthetic construct
  source 1..58: mol_type = other DNA
gtgactggagttcagacgtgtgctcttccgatctgaattatggctgcaggaaaatttg

SEQ ID NO: 953 - DNA, 51 bp, synthetic construct
  source 1..51: mol_type = other DNA
gtgactggagttcagacgtgtgctcttccgatcttttcctcttcctcgtcg

SEQ ID NO: 954 - DNA, 54 bp, synthetic construct
  source 1..54: mol_type = other DNA
gtgactggagttcagacgtgtgctcttccgatcttagcctgcatttgctttctc

SEQ ID NO: 955 - DNA, 59 bp, synthetic construct
  source 1..59: mol_type = other DNA
gtgactggagttcagacgtgtgctcttccgatctctttattcataaagttggtctcagt

SEQ ID NO: 956 - DNA, 53 bp, synthetic construct
  source 1..53: mol_type = other DNA
gtgactggagttcagacgtgtgctcttccgatctggcccaacctgaagttatt

SEQ ID NO: 957 - DNA, 57 bp, synthetic construct
  source 1..57: mol_type = other DNA
gtgactggagttcagacgtgtgctcttccgatctgtgaattcatcagctggatcaaa

SEQ ID NO: 958 - DNA, 54 bp, synthetic construct
  source 1..54: mol_type = other DNA
gtgactggagttcagacgtgtgctcttccgatctgcagtaaacagtctcagcat

SEQ ID NO: 959 - DNA, 53 bp, synthetic construct
  source 1..53: mol_type = other DNA
gtgactggagttcagacgtgtgctcttccgatctcaaagttgtggagaaggcc

SEQ ID NO: 960 - DNA, 52 bp, synthetic construct
  source 1..52: mol_type = other DNA
gtgactggagttcagacgtgtgctcttccgatctgctcatgtcctggttcct

SEQ ID NO: 961 - DNA, 50 bp, synthetic construct
  source 1..50: mol_type = other DNA
gtgactggagttcagacgtgtgctcttccgatcttccgagctggaggagg

SEQ ID NO: 962 - DNA, 59 bp, synthetic construct
  source 1..59: mol_type = other DNA
gtgactggagttcagacgtgtgctcttccgatctgaggaaactgatgttgataagaggt

SEQ ID NO: 963 - DNA, 63 bp, synthetic construct
  source 1..63: mol_type = other DNA
gtgactggagttcagacgtgtgctcttccgatctccaaagcattaatatccaacatagaatga

SEQ ID NO: 964 - DNA, 57 bp, synthetic construct
  source 1..57: mol_type = other DNA
gtgactggagttcagacgtgtgctcttccgatctctgggctttccatgaattatgaa

SEQ ID NO: 965 - DNA, 53 bp, synthetic construct
  source 1..53: mol_type = other DNA
gtgactggagttcagacgtgtgctcttccgatctaggatgatgtgttccaacc

SEQ ID NO: 966 - DNA, 54 bp, synthetic construct
  source 1..54: mol_type = other DNA
gtgactggagttcagacgtgtgctcttccgatctcgttaccccaaatccttacc

SEQ ID NO: 967 - DNA, 51 bp, synthetic construct
  source 1..51: mol_type = other DNA
gtgactggagttcagacgtgtgctcttccgatctgccgaaatactgctcgt

SEQ ID NO: 968 - DNA, 55 bp, synthetic construct
  source 1..55: mol_type = other DNA
gtgactggagttcagacgtgtgctcttccgatctcacattacatgcttcccagag

SEQ ID NO: 969 - DNA, 54 bp, synthetic construct
  source 1..54: mol_type = other DNA
gtgactggagttcagacgtgtgctcttccgatctatgctagccattacctccat

SEQ ID NO: 970 - DNA, 62 bp, synthetic construct
  source 1..62: mol_type = other DNA
gtgactggagttcagacgtgtgctcttccgatctctaacaacttcaactggatatccttata

SEQ ID NO: 971 - DNA, 53 bp, synthetic construct
  source 1..53: mol_type = other DNA
gtgactggagttcagacgtgtgctcttccgatcttaggatcagatgccgacat

SEQ ID NO: 972 - DNA, 58 bp, synthetic construct
  source 1..58: mol_type = other DNA
gtgactggagttcagacgtgtgctcttccgatcttcaccttagcattttgtgactttt

SEQ ID NO: 973 - DNA, 58 bp, synthetic construct
  source 1..58: mol_type = other DNA
gtgactggagttcagacgtgtgctcttccgatctctgctttagtaatgcaacatacct

SEQ ID NO: 974 - DNA, 59 bp, synthetic construct
  source 1..59: mol_type = other DNA
gtgactggagttcagacgtgtgctcttccgatctttgctagacgctgaagactaatttt

SEQ ID NO: 975 - DNA, 52 bp, synthetic construct
  source 1..52: mol_type = other DNA
gtgactggagttcagacgtgtgctcttccgatctcggctccgcatctatttc

SEQ ID NO: 976 - DNA, 55 bp, synthetic construct
  source 1..55: mol_type = other DNA
gtgactggagttcagacgtgtgctcttccgatcttccctaagtctaaggccttat

SEQ ID NO: 977 - DNA, 57 bp, synthetic construct
  source 1..57: mol_type = other DNA
gtgactggagttcagacgtgtgctcttccgatctcaagtaaagtgcctttcctagaa

SEQ ID NO: 978 - DNA, 59 bp, synthetic construct
  source 1..59: mol_type = other DNA
gtgactggagttcagacgtgtgctcttccgatctgcaaagtgatcatacctcttcaata

SEQ ID NO: 979 - DNA, 54 bp, synthetic construct
  source 1..54: mol_type = other DNA
gtgactggagttcagacgtgtgctcttccgatctaggttgactgcagacactaa

SEQ ID NO: 980 - DNA, 57 bp, synthetic construct
  source 1..57: mol_type = other DNA
gtgactggagttcagacgtgtgctcttccgatctagatgctgctacttcatataggc

SEQ ID NO: 981 - DNA, 52 bp, synthetic construct
  source 1..52: mol_type = other DNA
gtgactggagttcagacgtgtgctcttccgatctgctatgccactaccctcc

SEQ ID NO: 982 - DNA, 55 bp, synthetic construct
  source 1..55: mol_type = other DNA
gtgactggagttcagacgtgtgctcttccgatctcgacggtaatcaagttttgct

SEQ ID NO: 983 - DNA, 53 bp, synthetic construct
  source 1..53: mol_type = other DNA
gtgactggagttcagacgtgtgctcttccgatctgaatatcccgtccagtgtc

SEQ ID NO: 984 - DNA, 54 bp, synthetic construct
  source 1..54: mol_type = other DNA
gtgactggagttcagacgtgtgctcttccgatctgatcgcagggctcattatgt

SEQ ID NO: 985 - DNA, 52 bp, synthetic construct
  source 1..52: mol_type = other DNA
gtgactggagttcagacgtgtgctcttccgatctgattaggaatcccggcac

SEQ ID NO: 986 - DNA, 61 bp, synthetic construct
  source 1..61: mol_type = other DNA
gtgactggagttcagacgtgtgctcttccgatctgaattattttagttctcagagctgcaa

SEQ ID NO: 987 - DNA, 54 bp, synthetic construct
  source 1..54: mol_type = other DNA
gtgactggagttcagacgtgtgctcttccgatctctgtggagtacctcttccgt

SEQ ID NO: 988 - DNA, 54 bp, synthetic construct
  source 1..54: mol_type = other DNA
gtgactggagttcagacgtgtgctcttccgatctccatctcccttgaacattgt

SEQ ID NO: 989 - DNA, 57 bp, synthetic construct
  source 1..57: mol_type = other DNA
gtgactggagttcagacgtgtgctcttccgatctcctgacagttctaataaggtacc

SEQ ID NO: 990 - DNA, 52 bp, synthetic construct
  source 1..52: mol_type = other DNA
gtgactggagttcagacgtgtgctcttccgatctaatccatagcaagacggc

SEQ ID NO: 991 - DNA, 50 bp, synthetic construct
  source 1..50: mol_type = other DNA
gtgactggagttcagacgtgtgctcttccgatctacacagtggcgttctg

SEQ ID NO: 992 - DNA, 53 bp, synthetic construct
  source 1..53: mol_type = other DNA
gtgactggagttcagacgtgtgctcttccgatctgaaactgagaaccctgcta

SEQ ID NO: 993 - DNA, 51 bp, synthetic construct
  source 1..51: mol_type = other DNA
gtgactggagttcagacgtgtgctcttccgatctcgtgttcctgcctcagg

SEQ ID NO: 994 - DNA, 52 bp, synthetic construct
  source 1..52: mol_type = other DNA
gtgactggagttcagacgtgtgctcttccgatctgctctaagctgggtgact

SEQ ID NO: 995 - DNA, 52 bp, synthetic construct
  source 1..52: mol_type = other DNA
gtgactggagttcagacgtgtgctcttccgatctctgtctcctctgcagatg

SEQ ID NO: 996 - DNA, 51 bp, synthetic construct
  source 1..51: mol_type = other DNA
gtgactggagttcagacgtgtgctcttccgatctcgtttctccagggtaga

SEQ ID NO: 997 - DNA, 54 bp, synthetic construct
  source 1..54: mol_type = other DNA
gtgactggagttcagacgtgtgctcttccgatctgggtaatgctcttctccaaa

SEQ ID NO: 998 - DNA, 52 bp, synthetic construct
  source 1..52: mol_type = other DNA
gtgactggagttcagacgtgtgctcttccgatctcttcccgatcaggctgtt

SEQ ID NO: 999 - DNA, 53 bp, synthetic construct
  source 1..53: mol_type = other DNA
gtgactggagttcagacgtgtgctcttccgatctatcatgaagctgctgtgct

SEQ ID NO: 1000 - DNA, 52 bp, synthetic construct
  source 1..52: mol_type = other DNA
gtgactggagttcagacgtgtgctcttccgatctgacttacctttggacccg

SEQ ID NO: 1001 - DNA, 50 bp, synthetic construct
  source 1..50: mol_type = other DNA
gtgactggagttcagacgtgtgctcttccgatctagctctgcttccaccg

SEQ ID NO: 1002 - DNA, 51 bp, synthetic construct
  source 1..51: mol_type = other DNA
gtgactggagttcagacgtgtgctcttccgatctatacgcctggacaccct

SEQ ID NO: 1003 - DNA, 63 bp, synthetic construct
  source 1..63: mol_type = other DNA
gtgactggagttcagacgtgtgctcttccgatctcattcaccattttattccatgaaattttt

SEQ ID NO: 1004 - DNA, 59 bp, synthetic construct
  source 1..59: mol_type = other DNA
gtgactggagttcagacgtgtgctcttccgatctaggagaaaagaatgtcttcacacaa

SEQ ID NO: 1005 - DNA, 54 bp, synthetic construct
  source 1..54: mol_type = other DNA
gtgactggagttcagacgtgtgctcttccgatcttcaggaagtcattgctttcc

SEQ ID NO: 1006 - DNA, 52 bp, synthetic construct
  source 1..52: mol_type = other DNA
gtgactggagttcagacgtgtgctcttccgatctgtccctttctctgcagca

SEQ ID NO: 1007 - DNA, 53 bp, synthetic construct
  source 1..53: mol_type = other DNA
gtgactggagttcagacgtgtgctcttccgatctgagatctccttgaccgacg

SEQ ID NO: 1008 - DNA, 61 bp, synthetic construct
  source 1..61: mol_type = other DNA
gtgactggagttcagacgtgtgctcttccgatctcattcttcatccaagttatccaactta

SEQ ID NO: 1009 - DNA, 56 bp, synthetic construct
  source 1..56: mol_type = other DNA
gtgactggagttcagacgtgtgctcttccgatcttctcttccctttagcttctcac

SEQ ID NO: 1010 - DNA, 54 bp, synthetic construct
  source 1..54: mol_type = other DNA
gtgactggagttcagacgtgtgctcttccgatctcttgcaggagcttgaacata

SEQ ID NO: 1011 - DNA, 50 bp, synthetic construct
  source 1..50: mol_type = other DNA
gtgactggagttcagacgtgtgctcttccgatctctacgccgccttctcc

SEQ ID NO: 1012 - DNA, 55 bp, synthetic construct
  source 1..55: mol_type = other DNA
gtgactggagttcagacgtgtgctcttccgatcttgtacttggtaccacagcatt

SEQ ID NO: 1013 - DNA, 55 bp, synthetic construct
  source 1..55: mol_type = other DNA
gtgactggagttcagacgtgtgctcttccgatctcttgcgagtctcaggtactaa

SEQ ID NO: 1014 - DNA, 55 bp, synthetic construct
  source 1..55: mol_type = other DNA
gtgactggagttcagacgtgtgctcttccgatctaaaataaacgccaacacgatg

SEQ ID NO: 1015 - DNA, 55 bp, synthetic construct
  source 1..55: mol_type = other DNA
gtgactggagttcagacgtgtgctcttccgatctgaaataactgagtcgctggtt

SEQ ID NO: 1016 - DNA, 56 bp, synthetic construct
  source 1..56: mol_type = other DNA
gtgactggagttcagacgtgtgctcttccgatctattttggtacctgaagatctgg

SEQ ID NO: 1017 - DNA, 50 bp, synthetic construct
  source 1..50: mol_type = other DNA
gtgactggagttcagacgtgtgctcttccgatctgaggagggcgctagtg

SEQ ID NO: 1018 - DNA, 54 bp, synthetic construct
  source 1..54: mol_type = other DNA
gtgactggagttcagacgtgtgctcttccgatctattgcttgtcaccactttgg

SEQ ID NO: 1019 - DNA, 50 bp, synthetic construct
  source 1..50: mol_type = other DNA
gtgactggagttcagacgtgtgctcttccgatctcgctgctacctggatg

SEQ ID NO: 1020 - DNA, 51 bp, synthetic construct
  source 1..51: mol_type = other DNA
gtgactggagttcagacgtgtgctcttccgatctaatctccacgcccgaac

SEQ ID NO: 1021 - DNA, 20 bp, synthetic construct
  source 1..20: mol_type = other DNA
cgcatgacctcgaccatctg

SEQ ID NO: 1022 - DNA, 20 bp, synthetic construct
  source 1..20: mol_type = other DNA
tgccagatagcaccgtccaa

SEQ ID NO: 1023 - DNA, 20 bp, synthetic construct
  source 1..20: mol_type = other DNA
tcgtgtgggagcacgacatc

SEQ ID NO: 1024 - DNA, 20 bp, synthetic construct
  source 1..20: mol_type = other DNA
gcctggacgacattggccat

SEQ ID NO: 1025 - DNA, 20 bp, synthetic construct
  source 1..20: mol_type = other DNA
gtcaggatgaccgaatacgt

SEQ ID NO: 1026 - DNA, 20 bp, synthetic construct
  source 1..20: mol_type = other DNA
tttccggctagcacgtacca

SEQ ID NO: 1027 - DNA, 20 bp, synthetic construct
  source 1..20: mol_type = other DNA
atgaagcgcccacacgaaat

SEQ ID NO: 1028 - DNA, 20 bp, synthetic construct
  source 1..20: mol_type = other DNA
aagaagcgttcgtattcggt

SEQ ID NO: 1029 - DNA, 20 bp, synthetic construct
  source 1..20: mol_type = other DNA
ggcttgttacacgtactcta

SEQ ID NO: 1030 - DNA, 20 bp, synthetic construct
  source 1..20: mol_type = other DNA
ataagagctgctcatcgcat

SEQ ID NO: 1031 - DNA, 20 bp, synthetic construct
  source 1..20: mol_type = other DNA
gatcgacgtgtaccactacg

SEQ ID NO: 1032 - DNA, 20 bp, synthetic construct
  source 1..20: mol_type = other DNA
ggccccgctgaacgacacca

SEQ ID NO: 1033 - DNA, 20 bp, synthetic construct
  source 1..20: mol_type = other DNA
acggagctgacttcgccaag

SEQ ID NO: 1034 - DNA, 20 bp, synthetic construct
  source 1..20: mol_type = other DNA
gcaaatgagtacggcttgtt

SEQ ID NO: 1035 - DNA, 20 bp, synthetic construct
  source 1..20: mol_type = other DNA
gagtggatatggcctcgacc

SEQ ID NO: 1036 - DNA, 20 bp, synthetic construct
  source 1..20: mol_type = other DNA
acattgtgagccgggtcaac

SEQ ID NO: 1037 - DNA, 20 bp, synthetic construct
  source 1..20: mol_type = other DNA
cttcgacacaatgccaacgt

SEQ ID NO: 1038 - DNA, 20 bp, synthetic construct
  source 1..20: mol_type = other DNA
ccattcgagtcaagcttggt

SEQ ID NO: 1039 - DNA, 20 bp, synthetic construct
  source 1..20: mol_type = other DNA
ggccactcacgtgaacacta

SEQ ID NO: 1040 - DNA, 20 bp, synthetic construct
  source 1..20: mol_type = other DNA
agagattgtgcatcgttacg

SEQ ID NO: 1041 - DNA, 20 bp, synthetic construct
  source 1..20: mol_type = other DNA
gcaacaacaaggagtacccg

SEQ ID NO: 1042 - DNA, 20 bp, synthetic construct
  source 1..20: mol_type = other DNA
gaaccattgccacccgtctc

SEQ ID NO: 1043 - DNA, 20 bp, synthetic construct
  source 1..20: mol_type = other DNA
tgtaaaagtgaacaggtcga

SEQ ID NO: 1044 - DNA, 20 bp, synthetic construct
  source 1..20: mol_type = other DNA
gttctcgtcaaggacggcgt

SEQ ID NO: 1045 - DNA, 20 bp, synthetic construct
  source 1..20: mol_type = other DNA
catggcaactaactctgatt

SEQ ID NO: 1046 - DNA, 20 bp, synthetic construct
  source 1..20: mol_type = other DNA
ctcacgcggctggaaaccac

SEQ ID NO: 1047 - DNA, 20 bp, synthetic construct
  source 1..20: mol_type = other DNA
ttgcacagatctgggagtat

SEQ ID NO: 1048 - DNA, 20 bp, synthetic construct
  source 1..20: mol_type = other DNA
gccaacaaccgtgcctacaa

SEQ ID NO: 1049 - DNA, 20 bp, synthetic construct
  source 1..20: mol_type = other DNA
gttcggcagcaacgttgagt

SEQ ID NO: 1050 - DNA, 20 bp, synthetic construct
  source 1..20: mol_type = other DNA
actctaacacgttggggacg

SEQ ID NO: 1051 - DNA, 20 bp, synthetic construct
  source 1..20: mol_type = other DNA
gccaccaataatcgcaagag

SEQ ID NO: 1052 - DNA, 20 bp, synthetic construct
  source 1..20: mol_type = other DNA
caacgaggcagccgacacgt

SEQ ID NO: 1053 - DNA, 20 bp, synthetic construct
  source 1..20: mol_type = other DNA
gatccaccaaagcttctgtc

SEQ ID NO: 1054 - DNA, 20 bp, synthetic construct
  source 1..20: mol_type = other DNA
gtgtgtctaacaatacaact

SEQ ID NO: 1055 - DNA, 20 bp, synthetic construct
  source 1..20: mol_type = other DNA
acacgaagccaatcaggttc

SEQ ID NO: 1056 - DNA, 20 bp, synthetic construct
  source 1..20: mol_type = other DNA
tggtgaagagcgtccaccgg

SEQ ID NO: 1057 - DNA, 20 bp, synthetic construct
  source 1..20: mol_type = other DNA
ggtgttccgaatgggaccac

SEQ ID NO: 1058 - DNA, 20 bp, synthetic construct
  source 1..20: mol_type = other DNA
gtacgatgacttcccccacg

SEQ ID NO: 1059 - DNA, 20 bp, synthetic construct
  source 1..20: mol_type = other DNA
tcaacgccagatcttgtcgt

SEQ ID NO: 1060 - DNA, 20 bp, synthetic construct
  source 1..20: mol_type = other DNA
gtagtctaccaccatgccac

SEQ ID NO: 1061 - DNA, 20 bp, synthetic construct
  source 1..20: mol_type = other DNA
ctgggccacaaaagggatac

SEQ ID NO: 1062 - DNA, 20 bp, synthetic construct
  source 1..20: mol_type = other DNA
ccgagtccacatgttagccc

SEQ ID NO: 1063 - DNA, 20 bp, synthetic construct
  source 1..20: mol_type = other DNA
gcccaccaaacccccgacga

SEQ ID NO: 1064 - DNA, 20 bp, synthetic construct
  source 1..20: mol_type = other DNA
gtccccacaaagttcagggc

SEQ ID NO: 1065 - DNA, 20 bp, synthetic construct
  source 1..20: mol_type = other DNA
ctcagcaaggacgaacgcca

SEQ ID NO: 1066 - DNA, 20 bp, synthetic construct
  source 1..20: mol_type = other DNA
cactagaacgccacccaaag

SEQ ID NO: 1067 - DNA, 20 bp, synthetic construct
  source 1..20: mol_type = other DNA
gttcaccagctccgtgtcga

SEQ ID NO: 1068 - DNA, 20 bp, synthetic construct
  source 1..20: mol_type = other DNA
gggttgaccccaaagctaac

SEQ ID NO: 1069 - DNA, 86 bp, synthetic construct; identical to SEQ ID NO: 262
  source 1..86: mol_type = other DNA

SEQ ID NO: 1070 - DNA, 86 bp, synthetic construct; identical to SEQ ID NO: 264
  source 1..86: mol_type = other DNA

SEQ ID NO: 1071 - DNA, 86 bp, synthetic construct; identical to SEQ ID NO: 269
  source 1..86: mol_type = other DNA

SEQ ID NO: 1072 - DNA, 86 bp, synthetic construct; identical to SEQ ID NO: 275
  source 1..86: mol_type = other DNA

SEQ ID NO: 1073 - DNA, 86 bp, synthetic construct; identical to SEQ ID NO: 286
  source 1..86: mol_type = other DNA

SEQ ID NO: 1074 - DNA, 86 bp, synthetic construct; identical to SEQ ID NO: 287
  source 1..86: mol_type = other DNA

SEQ ID NO: 1075 - DNA, 86 bp, synthetic construct; identical to SEQ ID NO: 289
  source 1..86: mol_type = other DNA

SEQ ID NO: 1076 - DNA, 86 bp, synthetic construct; identical to SEQ ID NO: 288
  source 1..86: mol_type = other DNA

SEQ ID NO: 1077 - DNA, 86 bp, synthetic construct; identical to SEQ ID NO: 290
  source 1..86: mol_type = other DNA

SEQ ID NO: 1078 - DNA, 86 bp, synthetic construct; identical to SEQ ID NO: 317
  source 1..86: mol_type = other DNA

SEQ ID NO: 1079 - DNA, 86 bp, synthetic construct; identical to SEQ ID NO: 323
  source 1..86: mol_type = other DNA

SEQ ID NO: 1080 - DNA, 86 bp, synthetic construct; identical to SEQ ID NO: 329
  source 1..86: mol_type = other DNA

SEQ ID NO: 1081 - DNA, 86 bp, synthetic construct; identical to SEQ ID NO: 340
  source 1..86: mol_type = other DNA

SEQ ID NO: 1082 - DNA, 86 bp, synthetic construct; identical to SEQ ID NO: 348
  source 1..86: mol_type = other DNA

SEQ ID NO: 1083 - DNA, 86 bp, synthetic construct; identical to SEQ ID NO: 353
  source 1..86: mol_type = other DNA

SEQ ID NO: 1084 - DNA, 86 bp, synthetic construct; identical to SEQ ID NO: 358
  source 1..86: mol_type = other DNA

SEQ ID NO: 1085 - DNA, 86 bp, synthetic construct; identical to SEQ ID NO: 362
  source 1..86: mol_type = other DNA

SEQ ID NO: 1086 - DNA, 86 bp, synthetic construct; identical to SEQ ID NO: 364
  source 1..86: mol_type = other DNA

SEQ ID NO: 1087 - DNA, 86 bp, synthetic construct; identical to SEQ ID NO: 366
  source 1..86: mol_type = other DNA

SEQ ID NO: 1088 - DNA, 86 bp, synthetic construct; identical to SEQ ID NO: 374
  source 1..86: mol_type = other DNA

SEQ ID NO: 1089 - DNA, 86 bp, synthetic construct; identical to SEQ ID NO: 383
  source 1..86: mol_type = other DNA

SEQ ID NO: 1090 - DNA, 86 bp, synthetic construct; identical to SEQ ID NO: 384
  source 1..86: mol_type = other DNA

SEQ ID NO: 1091 - DNA, 86 bp, synthetic construct; identical to SEQ ID NO: 386
  source 1..86: mol_type = other DNA

SEQ ID NO: 1092 - DNA, 86 bp, synthetic construct; identical to SEQ ID NO: 399
  source 1..86: mol_type = other DNA

SEQ ID NO: 1093 - DNA, 86 bp, synthetic construct; identical to SEQ ID NO: 400
  source 1..86: mol_type = other DNA

SEQ ID NO: 1094 - DNA, 86 bp, synthetic construct; identical to SEQ ID NO: 410
  source 1..86: mol_type = other DNA

SEQ ID NO: 1095 - DNA, 86 bp, synthetic construct; identical to SEQ ID NO: 424
  source 1..86: mol_type = other DNA

SEQ ID NO: 1096 - DNA, 86 bp, synthetic construct; identical to SEQ ID NO: 430
  source 1..86: mol_type = other DNA

SEQ ID NO: 1097 - DNA, 86 bp, synthetic construct; identical to SEQ ID NO: 432
  source 1..86: mol_type = other DNA

SEQ ID NO: 1098 - DNA, 86 bp, synthetic construct; identical to SEQ ID NO: 434
  source 1..86: mol_type = other DNA

SEQ ID NO: 1099 - DNA, 86 bp, synthetic construct; identical to SEQ ID NO: 441
  source 1..86: mol_type = other DNA

SEQ ID NO: 1100 - DNA, 86 bp, synthetic construct; identical to SEQ ID NO: 451
  source 1..86: mol_type = other DNA

SEQ ID NO: 1101 - DNA, 86 bp, synthetic construct; identical to SEQ ID NO: 452
  source 1..86: mol_type = other DNA

SEQ ID NO: 1102 - DNA, 86 bp, synthetic construct; identical to SEQ ID NO: 453
  source 1..86: mol_type = other DNA

SEQ ID NO: 1103 - DNA, 86 bp, synthetic construct; identical to SEQ ID NO: 454
  source 1..86: mol_type = other DNA

SEQ ID NO: 1104 - DNA, 86 bp, synthetic construct; identical to SEQ ID NO: 457
  source 1..86: mol_type = other DNA

SEQ ID NO: 1105 - DNA, 86 bp, synthetic construct; identical to SEQ ID NO: 459
  source 1..86: mol_type = other DNA

SEQ ID NO: 1106 - DNA, 86 bp, synthetic construct; identical to SEQ ID NO: 461
  source 1..86: mol_type = other DNA

SEQ ID NO: 1107 - DNA, 86 bp, synthetic construct; identical to SEQ ID NO: 467
  source 1..86: mol_type = other DNA

SEQ ID NO: 1108 - DNA, 86 bp, synthetic construct; identical to SEQ ID NO: 471
  source 1..86: mol_type = other DNA

SEQ ID NO: 1109 - DNA, 86 bp, synthetic construct; identical to SEQ ID NO: 477
  source 1..86: mol_type = other DNA

SEQ ID NO: 1110 - DNA, 86 bp, synthetic construct; identical to SEQ ID NO: 487
  source 1..86: mol_type = other DNA

SEQ ID NO: 1111 - DNA, 86 bp, synthetic construct; identical to SEQ ID NO: 489
  source 1..86: mol_type = other DNA

SEQ ID NO: 1112 - DNA, 86 bp, synthetic construct; identical to SEQ ID NO: 490
  source 1..86: mol_type = other DNA

SEQ ID NO: 1113 - DNA, 86 bp, synthetic construct; identical to SEQ ID NO: 495
  source 1..86: mol_type = other DNA

SEQ ID NO: 1114 - DNA, 86 bp, synthetic construct; identical to SEQ ID NO: 498
  source 1..86: mol_type = other DNA

SEQ ID NO: 1115 - DNA, 86 bp, synthetic construct; identical to SEQ ID NO: 501
  source 1..86: mol_type = other DNA

SEQ ID NO: 1116 - DNA, 86 bp, synthetic construct; identical to SEQ ID NO: 506
  source 1..86: mol_type = other DNA

SEQ ID NO: 1117 - DNA, 56 bp, synthetic construct; identical to SEQ ID NO: 517
  source 1..56: mol_type = other DNA

SEQ ID NO: 1118 - DNA, 50 bp, synthetic construct; identical to SEQ ID NO: 519
  source 1..50: mol_type = other DNA

SEQ ID NO: 1119 - DNA, 51 bp, synthetic construct; identical to SEQ ID NO: 524
  source 1..51: mol_type = other DNA

SEQ ID NO: 1120 - DNA, 50 bp, synthetic construct; identical to SEQ ID NO: 530
  source 1..50: mol_type = other DNA

SEQ ID NO: 1121 - DNA, 52 bp, synthetic construct; identical to SEQ ID NO: 541
  source 1..52: mol_type = other DNA

SEQ ID NO: 1122 - DNA, 53 bp, synthetic construct; identical to SEQ ID NO: 542
  source 1..53: mol_type = other DNA

SEQ ID NO: 1123 - DNA, 51 bp, synthetic construct; identical to SEQ ID NO: 544
  source 1..51: mol_type = other DNA

SEQ ID NO: 1124 - DNA, 53 bp, synthetic construct; identical to SEQ ID NO: 543
  source 1..53: mol_type = other DNA

SEQ ID NO: 1125 - DNA, 55 bp, synthetic construct; identical to SEQ ID NO: 545
  source 1..55: mol_type = other DNA

SEQ ID NO: 1126 - DNA, 53 bp, synthetic construct; identical to SEQ ID NO: 572
  source 1..53: mol_type = other DNA

SEQ ID NO: 1127 - DNA, 53 bp, synthetic construct; identical to SEQ ID NO: 578
  source 1..53: mol_type = other DNA

SEQ ID NO: 1128 - DNA, 51 bp, synthetic construct; identical to SEQ ID NO: 584
  source 1..51: mol_type = other DNA

SEQ ID NO: 1129 - DNA, 54 bp, synthetic construct; identical to SEQ ID NO: 595
  source 1..54: mol_type = other DNA

SEQ ID NO: 1130 - DNA, 56 bp, synthetic construct; identical to SEQ ID NO: 603
  source 1..56: mol_type = other DNA

SEQ ID NO: 1131 - DNA, 51 bp, synthetic construct; identical to SEQ ID NO: 608
  source 1..51: mol_type = other DNA

SEQ ID NO: 1132 - DNA, 51 bp, synthetic construct; identical to SEQ ID NO: 613
  source 1..51: mol_type = other DNA

SEQ ID NO: 1133 - DNA, 52 bp, synthetic construct; identical to SEQ ID NO: 617
  source 1..52: mol_type = other DNA

SEQ ID NO: 1134 - DNA, 52 bp, synthetic construct; identical to SEQ ID NO: 619
  source 1..52: mol_type = other DNA

SEQ ID NO: 1135 - DNA, 54 bp, synthetic construct; identical to SEQ ID NO: 621
  source 1..54: mol_type = other DNA

SEQ ID NO: 1136 - DNA, 54 bp, synthetic construct; identical to SEQ ID NO: 629
  source 1..54: mol_type = other DNA

SEQ ID NO: 1137 - DNA, 51 bp, synthetic construct; identical to SEQ ID NO: 638
  source 1..51: mol_type = other DNA

SEQ ID NO: 1138 - DNA, 52 bp, synthetic construct; identical to SEQ ID NO: 639
  source 1..52: mol_type = other DNA

SEQ ID NO: 1139 - DNA, 53 bp, synthetic construct; identical to SEQ ID NO: 641
  source 1..53: mol_type = other DNA

SEQ ID NO: 1140 - DNA, 55 bp, synthetic construct; identical to SEQ ID NO: 654
  source 1..55: mol_type = other DNA

SEQ ID NO: 1141 - DNA, 53 bp, synthetic construct; identical to SEQ ID NO: 655
  source 1..53: mol_type = other DNA

SEQ ID NO: 1142 - DNA, 53 bp, synthetic construct; identical to SEQ ID NO: 665
  source 1..53: mol_type = other DNA

SEQ ID NO: 1143 - DNA, 54 bp, synthetic construct; identical to SEQ ID NO: 679
  source 1..54: mol_type = other DNA

SEQ ID NO: 1144 - DNA, 50 bp, synthetic construct; identical to SEQ ID NO: 685
  source 1..50: mol_type = other DNA

SEQ ID NO: 1145 - DNA, 52 bp, synthetic construct; identical to SEQ ID NO: 687
  source 1..52: mol_type = other DNA

SEQ ID NO: 1146 - DNA, 52 bp, synthetic construct; identical to SEQ ID NO: 689
  source 1..52: mol_type = other DNA

SEQ ID NO: 1147 - DNA, 56 bp, synthetic construct; identical to SEQ ID NO: 696
  source 1..56: mol_type = other DNA

SEQ ID NO: 1148 - DNA, 50 bp, synthetic construct; identical to SEQ ID NO: 706
  source 1..50: mol_type = other DNA

SEQ ID NO: 1149 - DNA, 58 bp, synthetic construct; identical to SEQ ID NO: 707
  source 1..58: mol_type = other DNA

SEQ ID NO: 1150 - DNA, 61 bp, synthetic construct; identical to SEQ ID NO: 708
  source 1..61: mol_type = other DNA

SEQ ID NO: 1151 - DNA, 55 bp, synthetic construct; identical to SEQ ID NO: 709
  source 1..55: mol_type = other DNA

SEQ ID NO: 1152 - DNA, 50 bp, synthetic construct; identical to SEQ ID NO: 712
  source 1..50: mol_type = other DNA

SEQ ID NO: 1153 - DNA, 53 bp, synthetic construct; identical to SEQ ID NO: 714
  source 1..53: mol_type = other DNA

SEQ ID NO: 1154 - DNA, 51 bp, synthetic construct; identical to SEQ ID NO: 716
  source 1..51: mol_type = other DNA

SEQ ID NO: 1155 - DNA, 57 bp, synthetic construct; identical to SEQ ID NO: 722
  source 1..57: mol_type = other DNA

SEQ ID NO: 1156 - DNA, 51 bp, synthetic construct; identical to SEQ ID NO: 726
  source 1..51: mol_type = other DNA

SEQ ID NO: 1157 - DNA, 52 bp, synthetic construct; identical to SEQ ID NO: 732
  source 1..52: mol_type = other DNA

SEQ ID NO: 1158 - DNA, 54 bp, synthetic construct; identical to SEQ ID NO: 742
  source 1..54: mol_type = other DNA

SEQ ID NO: 1159 - DNA, 52 bp, synthetic construct; identical to SEQ ID NO: 744
  source 1..52: mol_type = other DNA

SEQ ID NO: 1160 - DNA, 52 bp, synthetic construct; identical to SEQ ID NO: 745
  source 1..52: mol_type = other DNA

SEQ ID NO: 1161 - DNA, 54 bp, synthetic construct; identical to SEQ ID NO: 750
  source 1..54: mol_type = other DNA

SEQ ID NO: 1162 - DNA, 59 bp, synthetic construct; identical to SEQ ID NO: 753
  source 1..59: mol_type = other DNA

SEQ ID NO: 1163 - DNA, 50 bp, synthetic construct; identical to SEQ ID NO: 756
  source 1..50: mol_type = other DNA

SEQ ID NO: 1164 - DNA, 56 bp, synthetic construct; identical to SEQ ID NO: 761
  source 1..56: mol_type = other DNA

SEQ ID NO: 1165 - DNA, 57 bp, synthetic construct; identical to SEQ ID NO: 772
  source 1..57: mol_type = other DNA

SEQ ID NO: 1166 - DNA, 52 bp, synthetic construct; identical to SEQ ID NO: 774
  source 1..52: mol_type = other DNA

SEQ ID NO: 1167 - DNA, 52 bp, synthetic construct; identical to SEQ ID NO: 779
  source 1..52: mol_type = other DNA

SEQ ID NO: 1168 - DNA, 50 bp, synthetic construct; identical to SEQ ID NO: 785
  source 1..50: mol_type = other DNA

SEQ ID NO: 1169 - DNA, 54 bp, synthetic construct; identical to SEQ ID NO: 796
  source 1..54: mol_type = other DNA

SEQ ID NO: 1170 - DNA, 54 bp, synthetic construct; identical to SEQ ID NO: 797
  source 1..54: mol_type = other DNA

SEQ ID NO: 1171 - DNA, 51 bp, synthetic construct; identical to SEQ ID NO: 799
  source 1..51: mol_type = other DNA

SEQ ID NO: 1172 - DNA, 54 bp, synthetic construct; identical to SEQ ID NO: 798
  source 1..54: mol_type = other DNA

SEQ ID NO: 1173 - DNA, 55 bp, synthetic construct; identical to SEQ ID NO: 800
  source 1..55: mol_type = other DNA

SEQ ID NO: 1174 - DNA, 55 bp, synthetic construct; identical to SEQ ID NO: 827
  source 1..55: mol_type = other DNA

SEQ ID NO: 1175 - DNA, 53 bp, synthetic construct; identical to SEQ ID NO: 833
  source 1..53: mol_type = other DNA

SEQ ID NO: 1176 - DNA, 53 bp, synthetic construct; identical to SEQ ID NO: 839
  source 1..53: mol_type = other DNA

SEQ ID NO: 1177 - DNA, 56 bp, synthetic construct; identical to SEQ ID NO: 850
  source 1..56: mol_type = other DNA

SEQ ID NO: 1178 - DNA, 55 bp, synthetic construct; identical to SEQ ID NO: 858
  source 1..55: mol_type = other DNA

SEQ ID NO: 1179 - DNA, 52 bp, synthetic construct; identical to SEQ ID NO: 863
  source 1..52: mol_type = other DNA

SEQ ID NO: 1180 - DNA, 52 bp, synthetic construct; identical to SEQ ID NO: 868
  source 1..52: mol_type = other DNA

SEQ ID NO: 1181 - DNA, 53 bp, synthetic construct; identical to SEQ ID NO: 872
  source 1..53: mol_type = other DNA

SEQ ID NO: 1182 - DNA, 52 bp, synthetic construct; identical to SEQ ID NO: 874
  source 1..52: mol_type = other DNA

SEQ ID NO: 1183 - DNA, 57 bp, synthetic construct; identical to SEQ ID NO: 876
  source 1..57: mol_type = other DNA

SEQ ID NO: 1184 - DNA, 54 bp, synthetic construct; identical to SEQ ID NO: 884
  source 1..54: mol_type = other DNA

SEQ ID NO: 1185 - DNA, 51 bp, synthetic construct; identical to SEQ ID NO: 893
  source 1..51: mol_type = other DNA

SEQ ID NO: 1186 - DNA, 52 bp, synthetic construct; identical to SEQ ID NO: 894
  source 1..52: mol_type = other DNA

SEQ ID NO: 1187 - DNA, 55 bp, synthetic construct; identical to SEQ ID NO: 896
  source 1..55: mol_type = other DNA

SEQ ID NO: 1188 - DNA, 58 bp, synthetic construct; identical to SEQ ID NO: 909
  source 1..58: mol_type = other DNA

SEQ ID NO: 1189 - DNA, 54 bp, synthetic construct; identical to SEQ ID NO: 910
  source 1..54: mol_type = other DNA

SEQ ID NO: 1190 - DNA, 54 bp, synthetic construct; identical to SEQ ID NO: 920
  source 1..54: mol_type = other DNA

SEQ ID NO: 1191 - DNA, 53 bp, synthetic construct; identical to SEQ ID NO: 934
  source 1..53: mol_type = other DNA

SEQ ID NO: 1192 - DNA, 50 bp, synthetic construct; identical to SEQ ID NO: 940
  source 1..50: mol_type = other DNA

SEQ ID NO: 1193 - DNA, 54 bp, synthetic construct; identical to SEQ ID NO: 942
  source 1..54: mol_type = other DNA

SEQ ID NO: 1194 - DNA, 54 bp, synthetic construct; identical to SEQ ID NO: 944
  source 1..54: mol_type = other DNA

SEQ ID NO: 1195 - DNA, 56 bp, synthetic construct; identical to SEQ ID NO: 951
  source 1..56: mol_type = other DNA

SEQ ID NO: 1196 - DNA, 50 bp, synthetic construct; identical to SEQ ID NO: 961
  source 1..50: mol_type = other DNA

SEQ ID NO: 1197 - DNA, 59 bp, synthetic construct; identical to SEQ ID NO: 962
  source 1..59: mol_type = other DNA

SEQ ID NO: 1198 - DNA, 63 bp, synthetic construct; identical to SEQ ID NO: 963
  source 1..63: mol_type = other DNA

SEQ ID NO: 1199 - DNA, 57 bp, synthetic construct; identical to SEQ ID NO: 964
  source 1..57: mol_type = other DNA

SEQ ID NO: 1200 - DNA, 51 bp, synthetic construct; identical to SEQ ID NO: 967
  source 1..51: mol_type = other DNA

SEQ ID NO: 1201 - DNA, 54 bp, synthetic construct; identical to SEQ ID NO: 969
  source 1..54: mol_type = other DNA

SEQ ID NO: 1202 - DNA, 53 bp, synthetic construct; identical to SEQ ID NO: 971
  source 1..53: mol_type = other DNA

SEQ ID NO: 1203 - DNA, 57 bp, synthetic construct; identical to SEQ ID NO: 977
  source 1..57: mol_type = other DNA

SEQ ID NO: 1204 - DNA, 52 bp, synthetic construct; identical to SEQ ID NO: 981
  source 1..52: mol_type = other DNA

SEQ ID NO: 1205 - DNA, 54 bp, synthetic construct; identical to SEQ ID NO: 987
  source 1..54: mol_type = other DNA

SEQ ID NO: 1206 - DNA, 54 bp, synthetic construct; identical to SEQ ID NO: 997
  source 1..54: mol_type = other DNA

SEQ ID NO: 1207 - DNA, 53 bp, synthetic construct; identical to SEQ ID NO: 999
  source 1..53: mol_type = other DNA

SEQ ID NO: 1208 - DNA, 52 bp, synthetic construct; identical to SEQ ID NO: 1000
  source 1..52: mol_type = other DNA

SEQ ID NO: 1209 - DNA, 54 bp, synthetic construct; identical to SEQ ID NO: 1005
  source 1..54: mol_type = other DNA

SEQ ID NO: 1210 - DNA, 61 bp, synthetic construct; identical to SEQ ID NO: 1008
  source 1..61: mol_type = other DNA

SEQ ID NO: 1211 - DNA, 50 bp, synthetic construct; identical to SEQ ID NO: 1011
  source 1..50: mol_type = other DNA

SEQ ID NO: 1212 - DNA, 56 bp, synthetic construct; identical to SEQ ID NO: 1016
  source 1..56: mol_type = other DNA

(19) United States
(12) Patent Application Publication
(10) Pub. No.: US 2024/0312559 A1
(43) Pub. Date: Sep. 19, 2024

(54) PREDICTION MODEL FOR gRNA HDR POTENTIAL BASED ON INDEL PROFILES

(71) Applicant: INTEGRATED DNA TECHNOLOGIES, INC., Coralville, IA (US)

(72) Inventors: Jessica Woodley, North Liberty, IA (US); Gavin Kurgan, Timnath, CO (US); Karthik Murugan, Coralville, IA (US); Rolf Turk, Iowa City, IA (US); Garrett Rettig, Coralville, IA (US); Bernice Thommandru, Coralville, IA (US)

(21) Appl. No.: 18/606,255

(22) Filed: Mar. 15, 2024

Related U.S. Application Data
(60) Provisional application No. 63/490,977, filed on Mar. 17, 2023.

(51) Int. Cl. G16B 20/00; G16B 30/10; G16B 40/00

(52) U.S. Cl. CPC G16B 20/00; G16B 30/10; G16B 40/00

(57) ABSTRACT

Described herein is a method and application for predicting gRNA homology directed repair (HDR) potential based on indel profiles from HDR empirical data or in silico predictions. The application uses machine learning to predict preferred gRNAs and editing sites for HDR in vitro applications.

### CROSS REFERENCE TO RELATED APPLICATIONS

This application claims priority to U.S. Provisional Patent Application No. 63/490,977, filed on Mar. 17, 2023, which is incorporated by reference in its entirety.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “013670-0019-US02_sequence_listing_xml_7-MAR-2024.xml,” was created on Mar. 7, 2024, contains 1212 sequences, has a file size of 1.05 Mbytes, and is incorporated by reference in its entirety into the specification.

### BACKGROUND

The CRISPR-Cas9 system has been widely utilized to perform site-specific genome editing in eukaryotic cells. A sequence specific guide RNA is required to recruit Cas9 protein to the target site, and the Cas9 endonuclease cleaves both strands of the target DNA creating a double stranded break (DSB). This DSB is corrected by the cell's innate DNA damage repair pathways. The main pathways of DSB repair are the error prone non-homologous end joining (NHEJ) pathway, the alternative microhomology-mediated end joining (MMEJ) pathway, and the homology directed repair (HDR) pathway. The dominant, rapid NHEJ pathway results in either a correct repair that restores the Cas9 target site (and thus allows re-cutting by the Cas9) or a small insertion or deletion (indel) event in the target DNA. The MMEJ pathway, which relies on short microhomologous sequences at the break sites, typically results in larger deletion events. NHEJ and MMEJ repair events together create a unique indel profile that is consistent for a given Cas9 guide RNA (gRNA) and cell type. In contrast, the HDR pathway relies on a homologous DNA template (typically a sister chromatid in natural settings) to precisely repair the DSB. The HDR pathway has been frequently utilized in combination with CRISPR Cas9 to generate a specific desired mutation in the target DNA. To do so, an artificial repair template is provided for HDR which is either single or double stranded DNA and contains the target mutated DNA sequence with regions of homology to either side of the DSB. However, the limited frequency of repair via the HDR pathway poses a challenge to achieving high HDR rates for this CRISPR application.

HDR outcomes may be improved by the selection of gRNAs with a greater potential for HDR, namely gRNAs with a higher frequency of MMEJ-based edits (i.e., large deletions) in their indel profile.

What is needed are methods for predicting HDR outcomes and ranking HDR potential for gRNAs.

### SUMMARY

One embodiment described herein is a method for predicting the homology-directed repair (HDR) potential of one or more Cas guide RNAs (gRNAs), the process comprising: (a) generating an empirical indel profile for one or more candidate gRNAs by: (i) performing one or more Cas enzyme editing experiments using one or more candidate gRNAs and obtaining edited genomic DNA; (ii) for each editing experiment, amplifying and sequencing the edited genomic DNA to generate sequenced edited genomic DNA; executing on a processor, for each editing experiment: (iii) receiving the sequenced edited genomic DNA; and (iv) analyzing the sequenced edited genomic DNA and outputting an empirical indel profile; (b) inputting the empirical indel profile from step (a) into an HDR predictive model and analyzing the indel profiles; and (c) outputting an HDR rate threshold, HDR score, or rank ordered listing of the candidate gRNAs indicating preferred candidate gRNAs for an HDR editing experiment and optimal editing sites.

Another embodiment described herein is a method for predicting the homology-directed repair (HDR) potential of one or more Cas guide RNAs (gRNAs), the process comprising: (a) generating an in silico indel profile for one or more candidate gRNAs by executing on a processor: (i) inputting a candidate gRNA sequence and editing locus; and (ii) receiving an in silico indel profile; (b) inputting the in silico indel profile from step (a) into an HDR predictive model and analyzing the indel profiles; and (c) outputting an HDR rate threshold, HDR score, or rank ordered listing of the candidate gRNAs indicating preferred candidate gRNAs for an HDR editing experiment and optimal editing sites.

Another embodiment described herein is a method for predicting the homology-directed repair (HDR) potential of one or more Cas guide RNAs (gRNAs), the process comprising: (a) generating an empirical indel profile for one or more candidate gRNAs by: (i) performing one or more Cas enzyme editing experiments using one or more candidate gRNAs and obtaining edited genomic DNA; (ii) for each editing experiment, amplifying and sequencing the edited genomic DNA to generate sequenced edited genomic DNA; executing on a processor, for each editing experiment: (iii) receiving the sequenced edited genomic DNA; and (iv) analyzing the sequenced edited genomic DNA and outputting an empirical indel profile; or (b) generating an in silico indel profile for one or more candidate gRNAs by executing on a processor: (i) inputting a candidate gRNA sequence and editing locus; and (ii) receiving an in silico indel profile; (c) inputting the empirical indel profile from step (a) or in silico indel profile from step (b) into an HDR predictive model and analyzing the indel profiles; and (d) outputting an HDR rate threshold, HDR score, or rank ordered listing of the candidate gRNAs indicating preferred candidate gRNAs for an HDR editing experiment and optimal editing sites.

In one aspect, step (a)(ii) comprises amplifying the genomic DNA using RNase H-dependent PCR (rhPCR) and performing next generation sequencing (NGS) to generate sequenced edited genomic DNA. In another aspect, the analyzing the sequenced edited genomic DNA in step (a)(iv) comprises merging the sequenced edited genomic DNA, binning the merged sequenced edited genomic DNA by alignment to the genome, and providing alignments of the edited genomic DNA and a characterization and quantitation of the empirical indel frequency. In another aspect, the analysis is performed using rhAmpSeq CRISPR Analysis System or CRISPAltRations. In another aspect, the empirical indel profile comprises one or more of allele frequency, templated insertion frequency, microhomology-mediated end joining (MMEJ) deletion frequency, entropy, insertion size frequency, GC insertion motif frequency, deletion size frequency, or combinations thereof. In another aspect, generating the in silico indel profile comprises predicting guide RNA efficacy and producing alignments and editing frequency, and mutational outcomes resulting from double stranded breaks. In another aspect, the input is a guide sequence, and the output is a set of alignments and predictions for on-target base editing efficacy. In another aspect, the generating the in silico indel profile is performed using FORECasT. In another aspect, the HDR predictive model in step comprises a gradient boosted regressor, ensemble method, lasso regression, Structural Equation Modeling (SEM), or traditional machine learning process that transforms the multi-dimensional indel profile into an HDR rate threshold, HDR score, or rank ordered output for the candidate gRNAs. In another aspect, the HDR predictive model is trained by executing on a processor: (i) creating a training set of data using the empirical indel profile or in silico indel profile; (ii) creating a test set of data using the empirical indel profile or in silico indel profile; and (iii) training and testing the HDR predictive model, wherein the HDR predictive model is trained using the training set of data, and wherein the HDR predictive model is tested using the testing set of data. In another aspect, the HDR predictive model is capable of accurately ranking candidate gRNAs for overall HDR potential with a Spearman correlation value of greater than 0.5. In another aspect, the HDR rates and preferred candidate gRNAs are specific for a particular cell type or cell line. In another aspect, the candidate gRNA sequences have a variable region from about 17 nucleotides to about 24 nucleotides in length. In another aspect, the candidate gRNA sequences have a variable region of about 20 nucleotides in length. In another aspect, the candidate gRNA sequences comprise one or more modifications on their 5′-termini, 3′-termini, or a combination thereof. In another aspect, the modification comprises a termini-blocking modification. In another aspect, the editing site or editing locus is Cas-enzyme specific and comprises from about 1 nucleotide to about 15 nucleotides. In another aspect, the Cas enzyme is Cas9 or Cas 12a. In another aspect, the genomic DNA is from a population of cells or subjects. In another aspect, the candidate gRNA sequences comprise sequences from one or more of SEQ ID NO: 1-255 or 1021-1068.

### DESCRIPTION OF THE DRAWINGS

FIG. 1 shows a block diagram illustrating an example system for predicting the homology-directed repair (HDR) potential of one or more Cas guide RNAs (gRNAs), in accordance with various aspects of the present disclosure.

FIG. 2 shows a flow chart illustrating an exemplary process for predicting the homology-directed repair (HDR) potential of one or more Cas guide RNAs (gRNAs), in accordance with various aspects of the present disclosure.

FIG. 3A-C show the correlation between HDR editing frequencies and indel profile attributes of the RNP only control samples in HAP1 cells. TopAF (FIG. 3A), Entropy (FIG. 3B), and Deletion 3+ (FIG. 3C). N=150 sites.

FIG. 4 shows the performance of a Gradient Booster Regression HDR prediction model on test data based on empirical indel profile data in HAP1 cells (n=150). A 75/25 train-test split was performed for all modeling. Data presented graphically here is a representative sample (Pearson R²=0.55). 100 bootstraps were conducted on unique train/test splits to determine more generalized metrics for this model. Pearson R²=0.45±0.13, Spearman correlation=0.67±0.09.

FIG. 5 shows the performance of the HAP1 HDR prediction model using indel profile and HDR data generated in Jurkat cells. No correlation between predicted and measured HDR was observed. N=188 sites after filtering.

FIG. 6A-B show the assessment of Jurkat-specific repair factors and their potential effect on the NHEJ repair profile. FIG. 6A shows a box plot illustrating higher expression (in transcripts per million; TPM) of the DNTT gene encoding terminal deoxynucleotidyl transferase is observed relative to other commonly used laboratory cell lines in public data deposited in the Genotype-Tissue Expression database (GTEx v8). FIG. 6B shows the investigation of the Jurkat Cas9 indel profile of the same loci in the original HAP1 dataset demonstrates that it is enriched for insertions 2+ bp and greater, activity which could be characteristic of a template-independent polymerase adding nucleotides during repair.

FIG. 7A-C show the correlation between HDR editing frequencies and indel profile attributes of the RNP only control samples in K562 cells: TopAF (FIG. 7A), Entropy (FIG. 7B), and Deletion 3+ (FIG. 7C). N=40 sites, filtered on >70% editing.

FIG. 8A-D show comparisons of editing outcomes for target sites in K562 and HAP1 cells. Attributes assessed were: perfect HDR editing (FIG. 8A), Entropy (RNP only indel profile) (FIG. 8B), TopAF (RNP only indel profile) (FIG. 8C), and Deletions of 3+ bp (RNP only indel profile) (FIG. 8D). N=40 sites, filtered on >70% editing.

FIG. 9A-C show the correlation between HDR editing frequencies and indel profile attributes of the RNP only control samples in iPSCs: TopAF (FIG. 9A), Entropy (FIG. 9B), and Deletion 3+ (FIG. 9C). N=40 sites, filtered on >70% editing.

FIG. 10A-D show comparisons of editing outcomes for target sites in iPSCs and HAP1 cells. Attributes assessed were: perfect HDR editing (FIG. 10A), Entropy (RNP only indel profile) (FIG. 10B), TopAF (RNP only indel profile) (FIG. 10C), and Deletions of 3+ bp (RNP only indel profile) (FIG. 10D). N=40 sites, filtered on >70% editing and sequencing read depth.

FIG. 11A-C show comparisons of editing outcomes for target sites in in K562 cells, iPSCs, and primary T cells. TopAF (FIG. 11), Entropy(FIG. 11B), and Deletion 3+ (FIG. 11C). N=40 sites, filtered on >70% editing.

FIG. 12A-D show comparisons of editing outcomes for target sites in in K562 cells, iPSCs, and primary T cells. Attributes assessed were: perfect HDR editing (FIG. 12A), Entropy (RNP only indel profile) (FIG. 12B), TopAF (RNP only indel profile) (FIG. 12C), and Deletions of 3+ bp (RNP only indel profile) (FIG. 12D). N=40 sites, filtered on >70% editing and sequencing read depth.

FIG. 13A-C show the performance of the HAP1 HDR prediction model using indel profile and HDR data generated in K562 cells (FIG. 13A; N=36 data points after filtering), iPSCs (FIG. 11B; N=76 data points after filtering), and primary T cells (FIG. 13C; N=45 data points after filtering).

FIG. 14A-C show the performance of the HAP1 HDR prediction model using 3+DelFreq and HDR data generated in K562 cells (FIG. 14A; N=36 data points after filtering), iPSCs (FIG. 14B; N=76 data points after filtering), and primary T cells (FIG. 14C; N=45 data points after filtering).

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein. Upper and lowercase single letters may be used within sequences to provide structural information such as complementary regions or the like (e.g., “acgtACGT”). All polypeptides are shown in the N→C-termini orientation and all nucleotide sequences are shown in the 5′→3′ orientation, respectively, unless otherwise noted.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjuctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

Described herein is the development and testing of large HDR data sets to confirm that HDR outcomes can be improved by the selection of gRNAs with a greater potential for HDR, namely gRNAs with a higher frequency of MMEJ-based edits (i.e., large deletions) in their indel profile and to identify additional key features of the indel profile that can be predictive of HDR outcomes. Also described is the development of an HDR prediction model that uses empirically determined gRNA indel profiles as an input to provide a ranking of HDR potential for a gRNA. This model is then demonstrated to apply across multiple cell types including iPSCs.

The process described herein can be used to provide a rank order classification of HDR potential based on empirical data generated by the user that is particularly useful for large scale HDR screening projects. HDR outcomes can be improved, and screening requirements greatly reduced through the appropriate selection of gRNAs that have a favorable indel profile for HDR. This invention is compatible with the use of the rhAmpSeq CRISPR Analysis System and provides a streamlined workflow for the initial characterization of gRNA activity and HDR potential and the downstream analysis of HDR experiments. In future iterations, this HDR prediction model could be implemented with an indel profile prediction tool to remove the requirement for pre-generated indel profile data. Additionally, future iterations could incorporate cell specific information (based on RNA-Seq data for example) with respect to expression of DNA repair pathways to provide a tunable cell line specific prediction.

The process described herein for a more reliable selection of top gRNAs for HDR than suggested solutions in prior art. The HDR prediction model incorporates more comprehensive indel profile attributes that improves performance beyond the “MMEJ-based deletion frequency” described in prior art. Furthermore, the single factor model in prior art does not allow for adjustments to remain cell line agnostic while the multi-factor approach described with this invention could allow for cell line specific predictions based on the larger indel profile.

One embodiment described herein is a computer implemented process for predicting the HDR potential of Cas9 guide RNAs (gRNAs) using an input of empirically generated editing data, the process comprising of: Cas9 editing components including the gRNA(s) of interest are delivered into the cell line of interest and genomic DNA is collected following CRISPR editing. Editing outcomes for the gRNA(s) of interest are analyzed and quantified using an NGS-based approach such as the rhAmpSeq CRISPR Analysis System. The HDR prediction tool uses this editing data as an input to characterize the indel profile for the Cas9 gRNA(s) by creating a set of features such as deletion frequencies, insertion frequencies, top alleles, top allele frequencies, inter alia. The HDR prediction tool feeds this set of features through a regression model built off of generalizable data (HAP1 HDR data+indel profiles) to output a predicted HDR rate. HDR rates are relative to individual cell lines, so the actual HDR may vary. For screening and selecting a target gRNA from multiple options, the prediction tool will take the predicted HDR rates for each gRNA as an input and provide a rank or score for HDR potential as an output.

Another embodiment described herein is a computer implemented process for predicting the HDR potential of Cas9 guide RNAs (gRNAs) using an input of software predicted editing data, the process comprising of: The sequence information of Cas9 gRNA(s) of interest are provided to a software tool, e.g., FORECasT, that provides predicted editing outcomes based on sequence context. See e.g., Allen et al, Nature Biotechnol. 37: 64-72 (2019), which is incorporated by reference herein for such teachings. The HDR prediction tool uses this in silico predicted editing data as an input to characterize the indel profile for the Cas9 gRNA(s) by creating a set of features such as deletion frequencies, insertion frequencies, top alleles, top allele frequencies, inter alia. The HDR prediction tool feeds this set of features through a regression model built off of generalizable data (HAP1 HDR data+indel profiles) to output a predicted HDR rate. HDR rates are relative to individual cell lines, so the actual HDR may vary. For screening and selecting a target gRNA from multiple options, the prediction tool will take the predicted HDR rates for each gRNA as an input and provide a rank or score for HDR potential as an output.

Another embodiment described herein is a method of using complete indel profile features (vs. deletion frequency alone) to predict HDR.

Another embodiment described herein is a method for using indel profiles to predict HDR potential for gRNAs

Another embodiment described herein is a method for using a cell line repair pathway expression to inform a cell line specific HDR prediction model.

One embodiment described herein is a method for predicting the homology-directed repair (HDR) potential of one or more Cas guide RNAs (gRNAs), the process comprising: (a) generating an empirical indel profile for one or more candidate gRNAs by: (i) performing one or more Cas enzyme editing experiments using one or more candidate gRNAs and obtaining edited genomic DNA; (ii) for each editing experiment, amplifying and sequencing the edited genomic DNA to generate sequenced edited genomic DNA; executing on a processor, for each editing experiment: (iii) receiving the sequenced edited genomic DNA; and (iv) analyzing the sequenced edited genomic DNA and outputting an empirical indel profile; (b) inputting the empirical indel profile from step (a) into an HDR predictive model and analyzing the indel profiles; and (c) outputting an HDR rate threshold, HDR score, or rank ordered listing of the candidate gRNAs indicating preferred candidate gRNAs for an HDR editing experiment and optimal editing sites.

Another embodiment described herein is a method for predicting the homology-directed repair (HDR) potential of one or more Cas guide RNAs (gRNAs), the process comprising: (a) generating an in silico indel profile for one or more candidate gRNAs by executing on a processor: (i) inputting a candidate gRNA sequence and editing locus; and (ii) receiving an in silico indel profile; (b) inputting the in silico indel profile from step (a) into an HDR predictive model and analyzing the indel profiles; and (c) outputting an HDR rate threshold, HDR score, or rank ordered listing of the candidate gRNAs indicating preferred candidate gRNAs for an HDR editing experiment and optimal editing sites.

Another embodiment described herein is a method for predicting the homology-directed repair (HDR) potential of one or more Cas guide RNAs (gRNAs), the process comprising: (a) generating an empirical indel profile for one or more candidate gRNAs by: (i) performing one or more Cas enzyme editing experiments using one or more candidate gRNAs and obtaining edited genomic DNA; (ii) for each editing experiment, amplifying and sequencing the edited genomic DNA to generate sequenced edited genomic DNA; executing on a processor, for each editing experiment: (iii) receiving the sequenced edited genomic DNA; and (iv) analyzing the sequenced edited genomic DNA and outputting an empirical indel profile; or (b) generating an in silico indel profile for one or more candidate gRNAs by executing on a processor: (i) inputting a candidate gRNA sequence and editing locus; and (ii) receiving an in silico indel profile; (c) inputting the empirical indel profile from step (a) or in silico indel profile from step (b) into an HDR predictive model and analyzing the indel profiles; and (d) outputting an HDR rate threshold, HDR score, or rank ordered listing of the candidate gRNAs indicating preferred candidate gRNAs for an HDR editing experiment and optimal editing sites.

In one aspect, step (a)(ii) comprises amplifying the genomic DNA using RNase H-dependent PCR (rhPCR) and performing next generation sequencing (NGS) to generate sequenced edited genomic DNA. In another aspect, the analyzing the sequenced edited genomic DNA in step (a)(iv) comprises merging the sequenced edited genomic DNA, binning the merged sequenced edited genomic DNA by alignment to the genome, and providing alignments of the edited genomic DNA and a characterization and quantitation of the empirical indel frequency. In another aspect, the analysis is performed using rhAmpSeq CRISPR Analysis System or CRISPAltRations. In another aspect, the empirical indel profile comprises one or more of allele frequency, templated insertion frequency, microhomology-mediated end joining (MMEJ) deletion frequency, entropy, insertion size frequency, GC insertion motif frequency, deletion size frequency, or combinations thereof. In another aspect, generating the in silico indel profile comprises predicting guide RNA efficacy and producing alignments and editing frequency, and mutational outcomes resulting from double stranded breaks. In another aspect, the input is a guide sequence, and the output is a set of alignments and predictions for on-target base editing efficacy. In another aspect, the generating the in silico indel profile is performed using FORECasT. In another aspect, the HDR predictive model in step comprises a gradient boosted regressor, ensemble method, lasso regression, Structural Equation Modeling (SEM), or traditional machine learning process that transforms the multi-dimensional indel profile into an HDR rate threshold, HDR score, or rank ordered output for the candidate gRNAs. In another aspect, the HDR predictive model is trained by executing on a processor: (i) creating a training set of data using the empirical indel profile or in silico indel profile; (ii) creating a test set of data using the empirical indel profile or in silico indel profile; and (iii) training and testing the HDR predictive model, wherein the HDR predictive model is trained using the training set of data, and wherein the HDR predictive model is tested using the testing set of data. In another aspect, the HDR predictive model is capable of accurately ranking candidate gRNAs for overall HDR potential with a Spearman correlation value of greater than 0.5. In another aspect, the HDR rates and preferred candidate gRNAs are specific for a particular cell type or cell line. In another aspect, the candidate gRNA sequences have a variable region from about 17 nucleotides to about 24 nucleotides in length. In another aspect, the candidate gRNA sequences have a variable region of about 20 nucleotides in length. In another aspect, the candidate gRNA sequences comprise one or more modifications on their 5′-termini, 3′-termini, or a combination thereof. In another aspect, the modification comprises a termini-blocking modification. In another aspect, the editing site or editing locus is Cas-enzyme specific and comprises from about 1 nucleotide to about 15 nucleotides. In another aspect, the Cas enzyme is Cas9 or Cas 12a. In another aspect, the genomic DNA is from a population of cells or subjects. In another aspect, the candidate gRNA sequences comprise sequences from one or more of SEQ ID NO: 1-255 or 1021-1068.

Another embodiment described herein is a research tool comprising a nucleotide sequence described herein.

Another embodiment described herein is a reagent comprising a nucleotide sequence described herein.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to (a) nucleotide sequences, or degenerate, homologous, or codon-optimized variants thereof, encoding polypeptides having the amino acid sequences in SEQ ID NOs: 1-1212; or (b) nucleotide sequences capable of hybridizing to the complement of any of the nucleotide sequences in (a).

By a polynucleotide having a nucleotide sequence at least, for example, 90-99% “identical” to a reference nucleotide sequence is intended that the nucleotide sequence of the polynucleotide be identical to the reference sequence except that the polynucleotide sequence can include up to about 10-to-1 point mutations, additions, or deletions per each 100 nucleotides of the reference nucleotide sequence.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′-or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. Alignment methods for polynucleotide or polypeptide sequences is provided by the local homology algorithm of Smith and Waterman, Advances in Applied Mathematics 2: 4 82-489 (1981) or Needleman and Wunsch, J. Mol. Biol. 48 (3): 443-453 (1970).

Another embodiment described herein is a polynucleotide vector comprising one or more nucleotide sequences described herein.

Another embodiment described herein is a cell comprising one or more nucleotide sequences described herein or a polynucleotide vector described herein.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A method for predicting the homology-directed repair (HDR) potential of one or more Cas guide RNAs (gRNAs), the process comprising:
(a) generating an empirical indel profile for one or more candidate gRNAs by:
   (i) performing one or more Cas enzyme editing experiments using one or more candidate gRNAs and obtaining edited genomic DNA;
   (ii) for each editing experiment, amplifying and sequencing the edited genomic DNA to generate sequenced edited genomic DNA;
   executing on a processor, for each editing experiment:
   (iii) receiving the sequenced edited genomic DNA; and
   (iv) analyzing the sequenced edited genomic DNA and outputting an empirical indel profile;
(b) inputting the empirical indel profile from step (a) into an HDR predictive model and analyzing the indel profiles; and
(c) outputting an HDR rate threshold, HDR score, or rank ordered listing of the candidate gRNAs indicating preferred candidate gRNAs for an HDR editing experiment and optimal editing sites.
   Clause 2. A method for predicting the homology-directed repair (HDR) potential of one or more Cas guide RNAs (gRNAs), the process comprising:
(a) generating an in silico indel profile for one or more candidate gRNAs by executing on a processor:
   (i) inputting a candidate gRNA sequence and editing locus; and
   (ii) receiving an in silico indel profile;
(b) inputting the in silico indel profile from step (a) into an HDR predictive model and analyzing the indel profiles; and
(c) outputting an HDR rate threshold, HDR score, or rank ordered listing of the candidate gRNAs indicating preferred candidate gRNAs for an HDR editing experiment and optimal editing sites.
   Clause 3. A method for predicting the homology-directed repair (HDR) potential of one or more Cas guide RNAs (gRNAs), the process comprising:
(a) generating an empirical indel profile for one or more candidate gRNAs by:
   (i) performing one or more Cas enzyme editing experiments using one or more candidate gRNAs and obtaining edited genomic DNA;
   (ii) for each editing experiment, amplifying and sequencing the edited genomic DNA to generate sequenced edited genomic DNA;
   executing on a processor, for each editing experiment:
   (iii) receiving the sequenced edited genomic DNA; and
   (iv) analyzing the sequenced edited genomic DNA and outputting an empirical indel profile;
or
(b) generating an in silico indel profile for one or more candidate gRNAs by executing on a processor:
   (i) inputting a candidate gRNA sequence and editing locus; and
   (ii) receiving an in silico indel profile;
(c) inputting the empirical indel profile from step (a) or in silico indel profile from step (b) into an HDR predictive model and analyzing the indel profiles; and
(d) outputting an HDR rate threshold, HDR score, or rank ordered listing of the candidate gRNAs indicating preferred candidate gRNAs for an HDR editing experiment and optimal editing sites.
   Clause 4. The method of clause 1 or 3, wherein step (a)(ii) comprises amplifying the genomic DNA using RNase H-dependent PCR (rhPCR) and performing next generation sequencing (NGS) to generate sequenced edited genomic DNA.
   Clause 5. The method of any one of clauses 1, 3, or 4, wherein the analyzing the sequenced edited genomic DNA in step (a)(iv) comprises merging the sequenced edited genomic DNA, binning the merged sequenced edited genomic DNA by alignment to the genome, and providing alignments of the edited genomic DNA and a characterization and quantitation of the empirical indel frequency.
   Clause 6. The method of clause 5, wherein the analysis is performed using rhAmpSeq CRISPR Analysis System or CRISPAltRations.
   Clause 7. The method of any one of clauses 1-6, wherein the empirical indel profile comprises one or more of allele frequency, templated insertion frequency, microhomology-mediated end joining (MMEJ) deletion frequency, entropy, insertion size frequency, GC insertion motif frequency, deletion size frequency, or combinations thereof.
   Clause 8. The method of clause 2 or 3, wherein generating the in silico indel profile comprises predicting guide RNA efficacy and producing alignments and editing frequency, and mutational outcomes resulting from double stranded breaks.
   Clause 9. The method of clause 8, wherein the input is a guide sequence, and the output is a set of alignments and predictions for on-target base editing efficacy.
   Clause 10. The method of clause 2 or 3, where the generating the in silico indel profile is performed using FORECasT.
   Clause 11. The method of any one of clauses 1-10, wherein the HDR predictive model in step comprises a gradient boosted regressor, ensemble method, lasso regression, Structural Equation Modeling (SEM), or traditional machine learning process that transforms the multi-dimensional indel profile into an HDR rate threshold, HDR score, or rank ordered output for the candidate gRNAs.
   Clause 12. The method of any one of clauses 1-11, wherein the HDR predictive model is trained by executing on a processor:
(i) creating a training set of data using the empirical indel profile or in silico indel profile;
(ii) creating a test set of data using the empirical indel profile or in silico indel profile; and
(iii) training and testing the HDR predictive model, wherein the HDR predictive model is trained using the training set of data, and wherein the HDR predictive model is tested using the testing set of data.
   Clause 13. The method of any one of clauses 1-12, wherein the HDR predictive model is capable of accurately ranking candidate gRNAs for overall HDR potential with a Spearman correlation value of greater than 0.5.
   Clause 14. The method of any one of clauses 1-13, wherein the HDR rates and preferred candidate gRNAs are specific for a particular cell type or cell line.
   Clause 15. The method of any one of clauses 1-14, wherein the candidate gRNA sequences have a variable region from about 17 nucleotides to about 24 nucleotides in length.
   Clause 16. The method of clause 15, wherein the candidate gRNA sequences have a variable region of about 20 nucleotides in length.
   Clause 17. The method of any one of clauses 1-16, wherein the candidate gRNA sequences comprise one or more modifications on their 5′-termini, 3′-termini, or a combination thereof.
   Clause 18. The method of clause 17, wherein the modification comprises a termini-blocking modification.
   Clause 19. The method of any one of clauses 1-18, wherein the editing site or editing locus is Cas-enzyme specific and comprises from about 1 nucleotide to about 15 nucleotides.
   Clause 20. The method of any one of clauses 1-19, wherein the Cas enzyme is Cas9 or Cas 5 Clause 20. 12a.
   Clause 21. The method of any one of clauses 1-20, wherein the genomic DNA is from a population of cells or subjects.
   Clause 22. The method of any one of clauses 1-21, wherein the candidate gRNA sequences comprise sequences from one or more of SEQ ID NO: 1-255 or 1021-1068.

### EXAMPLES

### Example 1

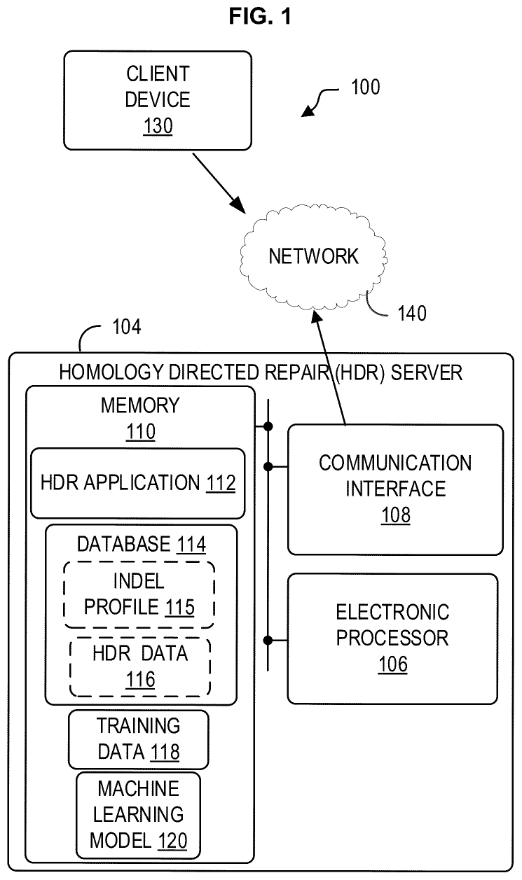

FIG. 1 shows a block diagram illustrating an example system for predicting the homology-directed repair (HDR) potential of one or more Cas9 guide RNAs (gRNAs), in accordance with various aspects of the present disclosure. In the example of FIG. 1, the system 100 includes a homology-directed repair (HDR) server 104 and a client device 130, and a network 140.

The HDR server 104 may be owned by, or operated by or on behalf of, an administrator. The HDR server 104 includes an electronic processor 106, a communication interface 108, and a memory 110. The electronic processor 106 is communicatively coupled to the communication interface 108 and the memory 110. The electronic processor 106 is a microprocessor or another suitable processing device. The communication interface 108 may be implemented as one or both of a wired network interface and a wireless network interface. The memory 110 is one or more of volatile memory (e.g., RAM) and non-volatile memory (e.g., ROM, FLASH, magnetic media, optical media, et cetera). In some examples, the memory 110 is also a non-transitory computer-readable medium. Although shown within the HDR server 104, memory 110 may be, at least in part, implemented as network storage that is external to the HDR server 104 and accessed via the communication interface 108. For example, all or part of memory 110 may be housed on the “cloud.”

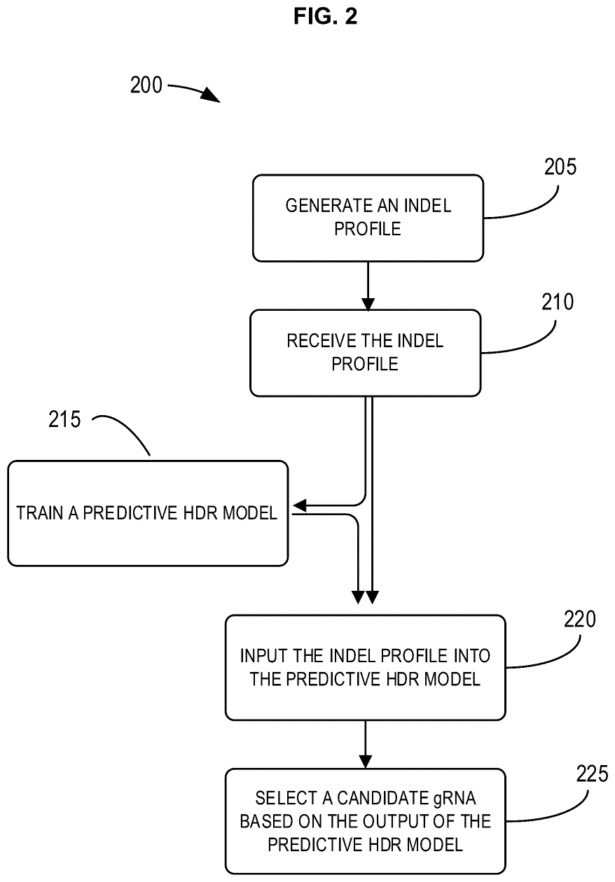

The HDR application 112 may be stored within a transitory or non-transitory portion of the memory 110. The HDR application 112 includes machine readable instructions that are executed by the electronic processor 106 to perform the functionality of the HDR server 104 as described below with respect to FIG. 2.

The memory 110 may include a database 114 for storing information about one or more Cas guide RNAs (gRNAs). The database 114 may be an RDF database, i.e., employ the Resource Description Framework. Alternatively, the database 114 may be another suitable database with features similar to the features of the Resource Description Framework, and various non-SQL databases, knowledge graphs, etc. The database 114 may include a plurality of data. The data may be associated with and contain information about one or more Cas9 editing experiments using the one or more candidate gRNAs. For example, in the illustrated embodiment, the database 114 includes indel profile 115 and HDR data 116. The indel profile 115 may include a plurality of sets of raw data associated with account users. In some instance, the raw data set 115 is generated based on transactions (e.g., requests) associated with the user device 150, the client device 140, and/or the data source 130. The HDR data 116 may include client data provided received from the client device 140 associated with account users. In some instances, the feedback data 116 includes fraud information associated with a user account. The memory 110 may also include a training data 118 and machine learning model 120. The training data 118 may include a set of historical requests (request history) associated with a user account. The labels 120 may include a set of labeled training examples for training a ML model for generating a score associated with a user.

The data source 130 may be on-premises, cloud, or edge-computing systems providing data and may include an electronic processor in communication with memory. The electronic processor is a microprocessor or another suitable processing device, the memory is one or more of volatile memory and non-volatile memory, and the communication interface may be a wireless or wired network interface. In some examples, the data source 130 may be accessed directly with the label server 104. In other examples, the data source 130 may be accessed indirectly over the network 160. For example, the data source 130 may be a source of transactions associated with a user account transmitted between the user device 150 and the data source 130. In some instances, the transactions include one or more requests of a user account. In some embodiments, the label creation application 112 retrieves data from the data source 130 via the network 160.

The client device 140 may be a web-compatible mobile computer, such as a laptop, a tablet, a smart phone, or other suitable computing device. Alternately, or in addition, the client device 140 may be a desktop computer. The client device 140 includes an electronic processor in communication with memory. The electronic processor is a microprocessor or another suitable processing device, the memory is one or more of volatile memory and non-volatile memory, and the communication interface may be a wireless or wired network interface.

An application, which contains software instructions implemented by the electronic processor of the client device 140 to perform the functions of the client device 140 as described herein, is stored within a transitory or a non-transitory portion of the memory. The application may have a graphical user interface that facilitates interaction between a user and the client device 140.

The client device 140 may communicate with the label server 104 over the network 160. The network 160 is preferably (but not necessarily) a wireless network, such as a wireless personal area network, local area network, or other suitable network. In some examples, the client device 140 may directly communicate with the label server 104. In other examples, the client device 140 may indirectly communicate with the label server 104 over network 160.

FIG. 2 is a flow chart illustrating an exemplary process 200 for predicting the homology-directed repair (HDR) potential of one or more Cas9 guide RNAs (gRNAs), in accordance with various aspects of the present disclosure. In the example of FIG. 2, the process 200 is described in a sequential flow, however, some of the process 200 may also be performed in parallel.

The process 200 generates an indel profile (at block 205). For example, the client device 130 generates the indel profile 115 (e.g., an empirical indel profile) for one or more candidate gRNAs. In this example, a user performs one or more Cas9 editing experiments using the one or more candidate gRNAs and obtains edited genomic DNA. When performing each experiment, the edited genomic DNA is amplified and sequenced to generate sequenced edited genomic DNA. In addition, the user inputs the sequenced edited genomic DNA into the client device 130, which analyzes the sequenced edited genomic DNA and outputs the empirical indel profile. In another example, the HDR server 104 generates the indel profile 115 (an in silico indel profile) for one or more candidate gRNAs. In this example, the HDR server 104 receives a candidate gRNA sequence and editing locus from the client device 130 and inputs the candidate gRNA sequence and the HDR application utilizes locally hosted software (e.g., FORECasT) to generate the in silico indel profile.

The process 200 receives the indel profile (at block 210). For example, the HDR server 104 receives the indel profile 115 (e.g., an in silico indel profile or an empirical indel profile) from the client device 130. In another example, the HDR server receives the indel profile 115 (e.g., an in silico indel profile) generated with the HDR application 112 and stores the indel profile 115 in the memory 110.

In the initial implementation, the process 200 trains a predictive HDR model (at block 215). For example, the HDR application 112 creates the training data 118 using the indel profile 115 and trains the machine learning algorithm 120. In some instances, the training data 118 includes a training set of data and testing set of data created with the empirical indel profile or in silico indel profile. In other instances, the machine learning model 120 is initially trained using a client generated empirical indel profile, which results increased accuracy of inferences determined by the machine learning model 120 in subsequent iterations of use. Subsequent runs of the process 200 may not need further training and thus block 215 becomes optional, although additional training could be beneficial for improving the accuracy of inferences determined by the machine learning model 120.

The process 200 inputs the indel profile into the predictive HDR model (at block 220). For example, the HDR application 112 inputs the indel profile 115 from block 210 into the machine learning model 120. The machine learning model 120 analyzes the indel profiles and generates an output. The outputs a value for each candidate gRNA that indicates a potential for HDR of each candidate gRNA.

The process 200 selects a candidate gRNA based on the output of the predictive HDR model (at block 225). For example, the HDR application 112 selects a candidate gRNA from a set of candidate gRNAs received. In some instances, the HDR application 112 determines an HDR rate threshold based on the values of each candidate gRNA. In other instances, the HDR application 112 orders a set of candidate gRNAs based on the values of each candidate gRNA.

### Example 2

### Important Attributes of Indel Profiles for Predicting HDR Potential

A large HDR dataset was generated by delivering CRISPR Cas9 HDR reagents targeting 263 sites into Jurkat and HAP1 cell lines. Cas9 ribonucleoprotein complex (RNP) was formed by mixing Alt-R™ S.p. Cas9 nuclease with either annealed Alt-R™ modified crRNA:tracrRNA (2-part gRNA) or Alt-R™ modified sgRNA (single-guide gRNA) at a 1:1.2 ratio of Cas9 protein to gRNA (Alt-R™ reagents from IDT, Coralville, IA). 4 μM Cas9 RNP complexes were delivered with 4 μM Alt-R™ Cas9 Electroporation Enhancer and 3 μM Alt-R™ HDR Donor Oligos using the Lonza 4D-Nucleofector 96-well system (Lonza, Basel, Switzerland). The Alt-R™ modifications comprise proprietary 5′-and 3′-termini blocking groups to prevent degradation of the nucleotide (IDT, Coralville, IA). HDR donors were designed to introduce a 6-bp “GAATTC” sequence at the DSB and corresponded to the non-targeting DNA strand relative to the gRNA. CRISPR reagents were delivered into 3E5 cells (HAP1) or 5E5 cells (Jurkat) using cell-line appropriate nucleofection conditions (DS-120 and CL-120 programs respectively). Conditions tested included RNP only (2-part gRNA), RNP only (sgRNA), RNP+HDR Donor (2-part gRNA), and untreated controls. DNA was extracted after 72 hours using QuickExtract™ DNA extraction solution (Lucigen, Madison, WI). Editing outcomes were quantified by NGS amplicon sequencing on the Illumina MiSeq platform using rhAmpSeq library preparation methods. Data analysis was conducted using IDT's in-house version of the rhAmpSeq CRISPR Analysis System. Sequences for gRNA protospacers, donor oligos, and sequencing primers are listed in Table 1.

TABLE 1
gRNAs, HDR Templates, and Sequencing Primers

| | | Target | SEQ ID |
|---|---|---|---|
| Purpose | Sequence | No. | NO. |
| gRNA protospacer | TAATCGGCAGTTGTCCACAC | 1 | 1 |
| gRNA protospacer | GCGCTGGCAAGACGTGTCGA | 2 | 2 |
| gRNA protospacer | GGCATCGTGTACTACCACGG | 3 | 3 |
| gRNA protospacer | CAGCTGGTGACTAACGCACA | 4 | 4 |
| gRNA protospacer | CCACGTTTTGCAACTAACGA | 5 | 5 |
| gRNA protospacer | GCACAAATTGTCGTCCTGAC | 6 | 6 |
| gRNA protospacer | CGCATGACCTCGACCATCTG | 7 | 7 |
| gRNA protospacer | ACCCTCGTGTGCCTCTTCGT | 8 | 8 |
| gRNA protospacer | TGCCAGATAGCACCGTCCAA | 9 | 9 |
| gRNA protospacer | GGCGGGCCACATACACCGAC | 10 | 10 |
| gRNA protospacer | ACTCGACTTCGAAGACCCAT | 11 | 11 |
| gRNA protospacer | CTGGTAAGTGTAGTAGACGA | 12 | 12 |
| gRNA protospacer | ACCTGGTCTCAACGCCATCC | 13 | 13 |
| gRNA protospacer | TCGTGTGGGAGCACGACATC | 14 | 14 |
| gRNA protospacer | CATGTGGCAGACCGACTGAT | 15 | 15 |
| gRNA protospacer | CGTGCAAAAAGACGACGGCC | 16 | 16 |
| gRNA protospacer | ATACATCCGCTTCCGACACC | 17 | 17 |
| gRNA protospacer | TTGGACGAAGTAGTAGACCC | 18 | 18 |
| gRNA protospacer | GATTGTCAGTTGAGTACTGC | 19 | 19 |
| gRNA protospacer | GCCTGGACGACATTGGCCAT | 20 | 20 |
| gRNA protospacer | AGGGACGTGTGTATCACTAC | 21 | 21 |
| gRNA protospacer | TCGACACGCCGGATGCCAGA | 22 | 22 |
| gRNA protospacer | AAGCTGCTCTACTCATCGAC | 23 | 23 |
| gRNA protospacer | TCAAGCTTTACCCCACCATA | 24 | 24 |
| gRNA protospacer | GCCGCCGAGACGATGACCAC | 25 | 25 |
| gRNA protospacer | GGATAGGTCGCGGTTGACAA | 26 | 26 |
| gRNA protospacer | GCATCTGACCCAAGAAACTA | 27 | 27 |
| gRNA protospacer | TTGCACGTGAGCTCGCCCAT | 28 | 28 |
| gRNA protospacer | GCAATAGGCACTCTCCACGG | 29 | 29 |
| gRNA protospacer | GAGCGTCCCGGCTGTACCAA | 30 | 30 |
| gRNA protospacer | GTCAGGATGACCGAATACGT | 31 | 31 |
| gRNA protospacer | TTTCCGGCTAGCACGTACCA | 32 | 32 |
| gRNA protospacer | ATGAAGCGCCCACACGAAAT | 33 | 33 |
| gRNA protospacer | AAGAAGCGTTCGTATTCGGT | 34 | 34 |
| gRNA protospacer | GGCTTGTTACACGTACTCTA | 35 | 35 |
| gRNA protospacer | AATACAATGGACTCCACCGC | 36 | 36 |
| gRNA protospacer | GTCTCTATGTGAACGGATCT | 37 | 37 |
| gRNA protospacer | TGGGACGTCCCACAATGGAT | 38 | 38 |
| gRNA protospacer | GTGCTTTGATCCACCGACAC | 39 | 39 |
| gRNA protospacer | GAGGGCTCGGTCATAAGTAC | 40 | 40 |
| gRNA protospacer | TGTAGGAGCACTGTCGACCC | 41 | 41 |
| gRNA protospacer | ACTGGTGTTGAACCGTGTTA | 42 | 42 |
| gRNA protospacer | CACCTCATATGGGTCGTCCG | 43 | 43 |
| gRNA protospacer | TACGAGTCAAACTCCCCTTC | 44 | 44 |
| gRNA protospacer | CCACGTAGTTGGCGACTTCC | 45 | 45 |
| gRNA protospacer | GCCAGTATCAGTACGTGTAA | 46 | 46 |
| gRNA protospacer | CTCGGACTGGACCCACCACG | 47 | 47 |
| gRNA protospacer | GACGCTAAGCACGATGGTGT | 48 | 48 |
| gRNA protospacer | TAACCGAACATGTGCTCCAC | 49 | 49 |
| gRNA protospacer | TCAAGGTTTTGAGTCGGTTC | 50 | 50 |
| gRNA protospacer | ACCGGATCAACGCCACGGTG | 51 | 51 |
| gRNA protospacer | CTACGGACGCGCATCAAGAG | 52 | 52 |
| gRNA protospacer | TATTAAAGTATCGGTACGAT | 53 | 53 |
| gRNA protospacer | TTTGAGTCCGACCACCAATC | 54 | 54 |
| gRNA protospacer | CTACGAGGAGCATTTGCACT | 55 | 55 |
| gRNA protospacer | CTTGCAGGACCTGAAGCAAC | 56 | 56 |
| gRNA protospacer | CCTGATAGCCTATACGTTCA | 57 | 57 |
| gRNA protospacer | AGCCCAAGGGAAGTCACCGC | 58 | 58 |
| gRNA protospacer | GCGGCCTCAACGACGAGACC | 59 | 59 |
| gRNA protospacer | CAACGTGTTCGTGACTTCGC | 60 | 60 |
| gRNA protospacer | GAACTCCTCGATCTCGTCGT | 61 | 61 |
| gRNA protospacer | ATAAGAGCTGCTCATCGCAT | 62 | 62 |
| gRNA protospacer | AAGGCGATGATGAGCACCGT | 63 | 63 |
| gRNA protospacer | TGGTGCACCGCTATCTGACG | 64 | 64 |
| gRNA protospacer | TGGAATATTGTGCTTGACTC | 65 | 65 |
| gRNA protospacer | TGGTGGTGCTGGAGATACCG | 66 | 66 |
| gRNA protospacer | ATTCCCATGTTGAACCCCGA | 67 | 67 |
| gRNA protospacer | GATCGACGTGTACCACTACG | 68 | 68 |
| gRNA protospacer | GTAGCACCACATCAACGGCA | 69 | 69 |
| gRNA protospacer | CATCGACCGGAAGCGCACGG | 70 | 70 |
| gRNA protospacer | GTACCAATGAGTGCAAAGCG | 71 | 71 |
| gRNA protospacer | AAGGATAACATCGTTACCAC | 72 | 72 |
| gRNA protospacer | CGGATCTTCTTAAACACGTT | 73 | 73 |
| gRNA protospacer | GGCCCCGCTGAACGACACCA | 74 | 74 |
| gRNA protospacer | TGCGGAAATGAGATCCTTAT | 75 | 75 |
| gRNA protospacer | CCAAGGTTGCCATCGGAACC | 76 | 76 |
| gRNA protospacer | TCCTGATTGATGGCTACCCG | 77 | 77 |
| gRNA protospacer | GAGTGGCCGTTCCTACCACG | 78 | 78 |
| gRNA protospacer | ATTCTGCACAATCTGTTTGC | 79 | 79 |
| gRNA protospacer | AGAAGCGGGACTATTTCTAC | 80 | 80 |
| gRNA protospacer | ACGCCAATGGCAACTACACT | 81 | 81 |
| gRNA protospacer | AAGAATATAGTCGTTATCAG | 82 | 82 |
| gRNA protospacer | GAACGTTGCTTTTCCACCGA | 83 | 83 |
| gRNA protospacer | GGACACCCCCATTGATTACT | 84 | 84 |
| gRNA protospacer | ACGGAGCTGACTTCGCCAAG | 85 | 85 |
| gRNA protospacer | TCGTTTATAACCACTACGAG | 86 | 86 |
| gRNA protospacer | TTCCATGGACGTTACGCCCC | 87 | 87 |
| gRNA protospacer | GTGGCACTCACTCTCTGTTC | 88 | 88 |
| gRNA protospacer | ACATCCAGGTCTGCATCCCC | 89 | 89 |
| gRNA protospacer | TGTCCCCGCACGGAGCCCAC | 90 | 90 |
| gRNA protospacer | ACGGAGACCCCGAAGTTTAC | 91 | 91 |
| gRNA protospacer | CCGCTACGAATACGATCACT | 92 | 92 |
| gRNA protospacer | GCAAATGAGTACGGCTTGTT | 93 | 93 |
| gRNA protospacer | GGATTCATACGACGTGACTG | 94 | 94 |
| gRNA protospacer | CGTCGAGCCCATACAGGAAC | 95 | 95 |
| gRNA protospacer | GATAACCCTAACCTACACCG | 96 | 96 |
| gRNA protospacer | ACAATGGTGTCGCGTACATG | 97 | 97 |
| gRNA protospacer | GAGTGGATATGGCCTCGACC | 98 | 98 |
| gRNA protospacer | GCACCACCAAATCATCCCCG | 99 | 99 |
| gRNA protospacer | ACGATTACACCTGTCGCCTG | 100 | 100 |
| gRNA protospacer | ATCTTTACCCAAGAGACTCG | 101 | 101 |
| gRNA protospacer | GATTAAGTGCTGGAACGGCG | 102 | 102 |
| gRNA protospacer | ACATTGTGAGCCGGGTCAAC | 103 | 103 |
| gRNA protospacer | ACAAGACGGACCGGAACCAC | 104 | 104 |
| gRNA protospacer | CCCATTCGGTCTTGCACATC | 105 | 105 |
| gRNA protospacer | GGTATTCTCACGGGATCCCG | 106 | 106 |
| gRNA protospacer | CTTCGACACAATGCCAACGT | 107 | 107 |
| gRNA protospacer | TAGACTGGATGCTGCTCGAC | 108 | 108 |
| gRNA protospacer | CCATTCGAGTCAAGCTTGGT | 109 | 109 |
| gRNA protospacer | CAAAGTTTCCAAACGACCCC | 110 | 110 |
| gRNA protospacer | GGCCACTCACGTGAACACTA | 111 | 111 |
| gRNA protospacer | TCGGAAGGCATATATCGTCA | 112 | 112 |
| gRNA protospacer | CCACGTTGAGGCTGCTCAAC | 113 | 113 |
| gRNA protospacer | AGAAGACTGCACTACGATCG | 114 | 114 |
| gRNA protospacer | GCTATACGGTTCGGGCCAAG | 115 | 115 |
| gRNA protospacer | GGACCGGTTTTTCAGATCAT | 116 | 116 |
| gRNA protospacer | GGGGGCCAACGTTTACACCC | 117 | 117 |
| gRNA protospacer | AGTGAGTACTCTCCTAGTAC | 118 | 118 |
| gRNA protospacer | AGAGATTGTGCATCGTTACG | 119 | 119 |
| gRNA protospacer | AAGCGCTTGCACGAATTAGT | 120 | 120 |
| gRNA protospacer | ACAACCGTTCGAAGGATGGT | 121 | 121 |
| gRNA protospacer | TTTCCATCTAGTCCTCAAGC | 122 | 122 |
| gRNA protospacer | TCTTTGCACACGGTTGGATC | 123 | 123 |
| gRNA protospacer | TTGGGACAACGTTGTCCAGC | 124 | 124 |
| gRNA protospacer | ACCAGGTGACATTGTACCGC | 125 | 125 |
| gRNA protospacer | CGGTCAAGATGGACCAGCAC | 126 | 126 |
| gRNA protospacer | TCCAAGGCACTGGAGACGTC | 127 | 127 |
| gRNA protospacer | GCAACAACAAGGAGTACCCG | 128 | 128 |
| gRNA protospacer | GAACCATTGCCACCCGTCTC | 129 | 129 |
| gRNA protospacer | ACGAGCCCAAGCCCGCAACT | 130 | 130 |
| gRNA protospacer | TGTAAAAGTGAACAGGTCGA | 131 | 131 |
| gRNA protospacer | GCGGAATTGACAAGTTCCGA | 132 | 132 |
| gRNA protospacer | CTGGGACGCAACCTCTCTCG | 133 | 133 |
| gRNA protospacer | GGACTATTTATGACTACGTG | 134 | 134 |
| gRNA protospacer | AGGACAAGATTCGACCCCTC | 135 | 135 |
| gRNA protospacer | CCCCCAACTTCAGTTTGTAC | 136 | 136 |
| gRNA protospacer | GTCCACCACGAGCGGAAGTA | 137 | 137 |
| gRNA protospacer | CATCGTGTACCCACCCGAGT | 138 | 138 |
| gRNA protospacer | TCAAGACTTGGCATACTCGC | 139 | 139 |
| gRNA protospacer | AAGGTGTTCGGACGGTCAAT | 140 | 140 |
| gRNA protospacer | TCGGATTTCCACACGACGTA | 141 | 141 |
| gRNA protospacer | GATGGCTTGGATCATCGACA | 142 | 142 |
| gRNA protospacer | TGCCAATTGGATACCGCTGT | 143 | 143 |
| gRNA protospacer | GTTCTCGTCAAGGACGGCGT | 144 | 144 |
| gRNA protospacer | CATGGCAACTAACTCTGATT | 145 | 145 |
| gRNA protospacer | TGGTCGACACGACGTTATCG | 146 | 146 |
| gRNA protospacer | TGCCACGATGTCAGTAAGAT | 147 | 147 |
| gRNA protospacer | AACTACTTTGAGAGCACCGA | 148 | 148 |
| gRNA protospacer | TGCCACTATTATCTAGCCCA | 149 | 149 |
| gRNA protospacer | CTACCCCACGACGTTCGTTA | 150 | 150 |
| gRNA protospacer | TTACCTGCTGGAGTCAACGG | 151 | 151 |
| gRNA protospacer | ACTACTGAGTAGCCCTGACC | 152 | 152 |
| gRNA protospacer | CACACGGATGTCGTCATCGA | 153 | 153 |
| gRNA protospacer | TTGAACTTGCCTCTCCGGAC | 154 | 154 |
| gRNA protospacer | CTCACGCGGCTGGAAACCAC | 155 | 155 |
| gRNA protospacer | AGTACAATTTGCACCACCGG | 156 | 156 |
| gRNA protospacer | CTAAGGATTCCACGGCCTCT | 157 | 157 |
| gRNA protospacer | TGATCACGGCGTATCGCAAC | 158 | 158 |
| gRNA protospacer | ATGGCTTTACCTCGTCTCAC | 159 | 159 |
| gRNA protospacer | GCGAGTCAAGTGCGTCAACG | 160 | 160 |
| gRNA protospacer | AGAACCGGAGCAAAATTCGC | 161 | 161 |
| gRNA protospacer | TGCCTACGACCGGCACCTTT | 162 | 162 |
| gRNA protospacer | CGTGTGGTACTGTTATCACG | 163 | 163 |
| gRNA protospacer | TGGGATGGCTCGTAGACTAT | 164 | 164 |
| gRNA protospacer | CGGCTTTTAACCACCCAACC | 165 | 165 |
| gRNA protospacer | GAGACCCACGCGTTCTTTGT | 166 | 166 |
| gRNA protospacer | GACGGTGGTGCCCAAATCGG | 167 | 167 |
| gRNA protospacer | TTCGGCGTCAACGAGAGTAC | 168 | 168 |
| gRNA protospacer | TTGCACAGATCTGGGAGTAT | 169 | 169 |
| gRNA protospacer | GCCAAGCTGGATCTTGATGC | 170 | 170 |
| gRNA protospacer | GTACTACACCACGGTTGAGC | 171 | 171 |
| gRNA protospacer | GAAACAGGCGATTACGGAGC | 172 | 172 |
| gRNA protospacer | TGTTTGGATAGGGGTACACG | 173 | 173 |
| gRNA protospacer | TTCAGTGCTGCAACTGCCAC | 174 | 174 |
| gRNA protospacer | GCCAACAACCGTGCCTACAA | 175 | 175 |
| gRNA protospacer | TAATCAGGTTGTCAAACCGC | 176 | 176 |
| gRNA protospacer | GTTCGGCAGCAACGTTGAGT | 177 | 177 |
| gRNA protospacer | TGCGAAGCCCATAACGCCAA | 178 | 178 |
| gRNA protospacer | ACTCTAACACGTTGGGGACG | 179 | 179 |
| gRNA protospacer | CCCACGGCGCAAGAGGTAGC | 180 | 180 |
| gRNA protospacer | ACCAATGGGCGCTTACGAAG | 181 | 181 |
| gRNA protospacer | GGAACTCTGAGTCATAGCGT | 182 | 182 |
| gRNA protospacer | CCAAAAGCACCGAGACTTCG | 183 | 183 |
| gRNA protospacer | GGTTTGGGGGACACACGGGT | 184 | 184 |
| gRNA protospacer | ACCGGAGCATCTGACAAACC | 185 | 185 |
| gRNA protospacer | GCCACCAATAATCGCAAGAG | 186 | 186 |
| gRNA protospacer | GGTGAATCACCAGTTCCCCC | 187 | 187 |
| gRNA protospacer | CTGCGGAACCCCACTTTCCA | 188 | 188 |
| gRNA protospacer | TACCGCCCAAGGAGCATTAA | 189 | 189 |
| gRNA protospacer | GTCAACTATGTGCGGTACAA | 190 | 190 |
| gRNA protospacer | TCACCGCCACGTTTGAGATC | 191 | 191 |
| gRNA protospacer | GAATGGCCTGCAACGTTGAC | 192 | 192 |
| gRNA protospacer | GTGTGCCTAGAGGAAATCGT | 193 | 193 |
| gRNA protospacer | ATGTTATCGACAAGCCTATT | 194 | 194 |
| gRNA protospacer | CGACCCCGGGGAACACCCTC | 195 | 195 |
| gRNA protospacer | CAACGAGGCAGCCGACACGT | 196 | 196 |
| gRNA protospacer | GATCCACCAAAGCTTCTGTC | 197 | 197 |
| gRNA protospacer | GTGTGTCTAACAATACAACT | 198 | 198 |
| gRNA protospacer | ACACGAAGCCAATCAGGTTC | 199 | 199 |
| gRNA protospacer | AGAGAGCAAGCTCCCGGGTT | 200 | 200 |
| gRNA protospacer | CTTCCTCAACGACGCGGACA | 201 | 201 |
| gRNA protospacer | TGGTGAAGAGCGTCCACCGG | 202 | 202 |
| gRNA protospacer | CGTCCACGAAGAACCCACTA | 203 | 203 |
| gRNA protospacer | GGTGTTCCGAATGGGACCAC | 204 | 204 |
| gRNA protospacer | GGTGGGACCGAAGTTACCTC | 205 | 205 |
| gRNA protospacer | GTACGATGACTTCCCCCACG | 206 | 206 |
| gRNA protospacer | GAACTTCTGTACTACAACGC | 207 | 207 |
| gRNA protospacer | CCGGAGGTTACCCACTGTGA | 208 | 208 |
| gRNA protospacer | TTCTCCCTTGAACGTGGTAC | 209 | 209 |
| gRNA protospacer | GCAACCAAGAGGAAACGGCG | 210 | 210 |
| gRNA protospacer | CAGGACGTCCGCCACATACT | 211 | 211 |
| gRNA protospacer | TCAACGCCAGATCTTGTCGT | 212 | 212 |
| gRNA protospacer | AAGCTTTTTGTCATCAGCTG | 213 | 213 |
| gRNA protospacer | GAGGGAACTTCTGATGGTAC | 214 | 214 |
| gRNA protospacer | TAATCTTCACGTCGAAGTGA | 215 | 215 |
| gRNA protospacer | GTAGTCTACCACCATGCCAC | 216 | 216 |
| gRNA protospacer | AACTGAAACCGGTACTGATT | 217 | 217 |
| gRNA protospacer | TCGGAGTCGCTGCAAAGTCG | 218 | 218 |
| gRNA protospacer | GGGGGACCTTTGGCACACGC | 219 | 219 |
| gRNA protospacer | ACCATCACGGCCAGACGCGT | 220 | 220 |
| gRNA protospacer | ATGCTTACCGGGGGACCAAA | 221 | 221 |
| gRNA protospacer | CTGGGCCACAAAAGGGATAC | 222 | 222 |
| gRNA protospacer | GTCCTGGGTGCTGATGTCAT | 223 | 223 |
| gRNA protospacer | AGACGCTGGACAGCATACGA | 224 | 224 |
| gRNA protospacer | TCGGTGCTGAAGTCCTCGTT | 225 | 225 |
| gRNA protospacer | GGGGTACGGCTGAAGACTCG | 226 | 226 |
| gRNA protospacer | CTGGTTTCATGGTTCCTCCG | 227 | 227 |
| gRNA protospacer | ACGGAATCCCACAGCTGGTA | 228 | 228 |
| gRNA protospacer | ATCCTCTGTCCAGAATGAGC | 229 | 229 |
| gRNA protospacer | CGCACCCCCCAACATCTACG | 230 | 230 |
| gRNA protospacer | AAGGATGCACGCTCCCCACC | 231 | 231 |
| gRNA protospacer | CCGAGTCCACATGTTAGCCC | 232 | 232 |
| gRNA protospacer | GCGGGCCAACTTCACTCTGC | 233 | 233 |
| gRNA protospacer | GCCCACCAAACCCCCGACGA | 234 | 234 |
| gRNA protospacer | GTCCCCACAAAGTTCAGGGC | 235 | 235 |
| gRNA protospacer | GCTACCGGAGCACAGTGCAC | 236 | 236 |
| gRNA protospacer | GGGGGCCTACACCTTCCAAC | 237 | 237 |
| gRNA protospacer | AACCCAAGGAGTTAATCCTA | 238 | 238 |
| gRNA protospacer | TTATTATGGACTGGTGCTTA | 239 | 239 |
| gRNA protospacer | CTCAGCAAGGACGAACGCCA | 240 | 240 |
| gRNA protospacer | TGTGCTGGTAGGATTTGTGC | 241 | 241 |
| gRNA protospacer | CAGGTCTTTGATCAACTCGA | 242 | 242 |
| gRNA protospacer | CACTAGAACGCCACCCAAAG | 243 | 243 |
| gRNA protospacer | TCTATCGTCCACACGGAGGA | 244 | 244 |
| gRNA protospacer | CACCCTGGACATAGCACGTC | 245 | 245 |
| gRNA protospacer | GTTCACCAGCTCCGTGTCGA | 246 | 246 |
| gRNA protospacer | GCCACCACACAGCCGACGAA | 247 | 247 |
| gRNA protospacer | ACCTCTGGGACCTTGGCGGT | 248 | 248 |
| gRNA protospacer | CGACGCACACTGCATTAATG | 249 | 249 |
| gRNA protospacer | TGGATGATCACTTAATACGA | 250 | 250 |
| gRNA protospacer | GGGTTGACCCCAAAGCTAAC | 251 | 251 |
| gRNA protospacer | CGGCATTGAACTCGGTCAGT | 252 | 252 |
| gRNA protospacer | CCTTGTACAGATGCAAACGG | 253 | 253 |
| gRNA protospacer | AAGAATGCCACGCTTGTCGC | 254 | 254 |
| gRNA protospacer | GTCAAAGGACGCCCTGAATC | 255 | 255 |
| HDR donor | +G*T*TTTTGGGATGCCCCCAACAAATAATCGGCAGTTGTCCA | 1 | 256 |
| | GAATTCCACTGGCACAAACCCCTAAGAGGCGCCCATTTCTGCA | | |
| | A*A*A+ | | |
| HDR donor | +G*T*GGAGCGCGTGGCGCGCGAGCGGCGCTGGCAAGACGTGT | 2 | 257 |
| | GAATTCCGATGGCGCGCATGCGCACGTTGCACGCGGCGCTGGG | | |
| | C*G*G+ | | |
| HDR donor | +C*A*TCCACCTGCACCCTGAAGCCTGGCATCGTGTACTACCA | 3 | 258 |
| | GAATTCCGGTGGCGGGGGCGTCATGGGGAGTTTGAGTAAGAAC | | |
| | C*A*T+ | | |
| HDR donor | +G*G*TCTCCTGAGAAGACGGAGGAGCAGCTGGTGACTAACGC | 4 | 259 |
| | GAATTCACAGGGTACGCCAGCAGCCATAGCTGCGCTCGCGAGC | | |
| | C*G*G+ | | |
| HDR donor | +T*G*CTGGTTGTGCAGAAGGTTTATCCACGTTTTGCAACTAA | 5 | 260 |
| | GAATTCCGAAGGTTTCAGAACCCTCCAGAAGTCTGTTAAACAT | | |
| | C*T*G+ | | |
| HDR donor | +T*A*ATGGCTCTTATTGTAAAATCTGCACAAATTGTCGTCCT | 6 | 261 |
| | GAATTCGACTGGTTTTGTGATGGTCTTCACCCTCTTTCTCCTC | | |
| | T*A*T+ | | |
| HDR donor | +A*C*CAAATGGCCCTTTTCATTCAGCGCATGACCTCGACCAT | 7 | 262 |
| | GAATTCCTGTGGTTTCCTGTTGGGATTTTTCAGGGGTTGGAAA | | |
| | C*T*G+ | | |
| HDR donor | +T*G*CGGGCCTCGTGGCTGGTCATCACCCTCGTGTGCCTCTT | 8 | 263 |
| | GAATTCCGTGGGCATCTATGCCATGGTGAAGCTGCTGCTCTTC | | |
| | T*C*A+ | | |
| HDR donor | +C*C*CACCAGCTTCAGCAGTCCAAGTGCCAGATAGCACCGTC | 9 | 264 |
| | GAATTCCAAAGGCCCCATACAAGGCCCTCCACAGCCGTGCCCC | | |
| | C*T*C+ | | |
| HDR donor | +G*C*AGCGGGCCAGCCTTCCGTCCAGGCGGGCCACATACACC | 10 | 265 |
| | GAATTCGACAGGAAGGTGCGGCTCACCAAGGCGGCCGAGTGCA | | |
| | G*G*G+ | | |
| HDR donor | +T*G*GTTGTTTCTGGGCTATTCCTAACTCGACTTCGAAGACC | 11 | 266 |
| | GAATTCCATTGGTAAGATGACAATAACTGAGCAAAAGTATGAA | | |
| | G*G*A+ | | |
| HDR donor | +G*G*GAGGCTTCACCTTTGGAAGCACTGGTAAGTGTAGTAGA | 12 | 267 |
| | GAATTCCGAGGGTGAACGGGGAGATGATGAGCTTGCTGGCCAT | | |
| | G*C*T+ | | |
| HDR donor | +T*G*TGTTTACAGTGGGATCATGAAACCTGGTCTCAACGCCA | 13 | 268 |
| | GAATTCTCCTGGGACCCACAGGTGGAGGCAAATCTTCGTGAGT | | |
| | A*T*A+ | | |
| HDR donor | +C*C*CGCCAGAGAGCAGGGCTGTCCTCGTGTGGGAGCACGAC | 14 | 269 |
| | GAATTCATCAGGCCCAGTGCCGTCAGGATCTCTGTCACCTGGC | | |
| | C*C*C+ | | |
| HDR donor | +T*C*ATGGCAGAGCTGAGTCTGAGCCATGTGGCAGACCGACT | 15 | 270 |
| | GAATTCGATTGGCAACTACAGCTTGGGGGGCATTTCCACGGGT | | |
| | G*A*G+ | | |
| HDR donor | +G*T*TAGTTTTGCTGCCGAAGAGCCCGTGCAAAAAGACGACG | 16 | 271 |
| | GAATTCGCCGGGAGGGCTGCCTCCCCGTCCAGAAGCCTGTAGG | | |
| | A*A*A+ | | |
| HDR donor | +T*C*CGACCTCAAGGAGGCGGTCACATACATCCGCTTCCGAC | 17 | 272 |
| | GAATTCACCCGGCGGCGCCGCTGTTCGCGGTGAGCGAAGGCTC | | |
| | G*G*G+ | | |
| HDR donor | +C*T*ATGCTTTTGACCTATTGGGTTTTGGACGAAGTAGTAGA | 18 | 273 |
| | GAATTCCCCAGGTTTGACAGTGATGCAGAAGAAGTGGAGAATC | | |
| | A*G*T+ | | |
| HDR donor | +T*T*CTTTGAGCCGTTGTACAAATTGATTGTCAGTTGAGTAC | 19 | 274 |
| | GAATTCTGCAGGCCTGTGGGATTCAAACCCAAATGGCTTGATG | | |
| | A*T*A+ | | |
| HDR donor | +C*A*GGAGACCCCTCAACTTTGGCTGCCTGGACGACATTGGC | 20 | 275 |
| | GAATTCCATGGGATCAAGGTAGAGAGAGGGGCCCTCCTCTTTC | | |
| | C*C*T+ | | |
| HDR donor | +G*C*TGTCCATCTCGCTCAGGTACGAGGGACGTGTGTATCAC | 21 | 276 |
| | GAATTCTACAGGATCAATACCACTGCAGATGGCAAGGTAAGAC | | |
| | T*C*C+ | | |
| HDR donor | +C*T*GTCTTCAGTTCTGGCTGCTTGTCGACACGCCGGATGCC | 22 | 277 |
| | GAATTCAGATGGAGGAACCTGTTGACAAATTCCCAAGAGGGAA | | |
| | A*T*G+ | | |
| HDR donor | +T*T*AACACCAAGTATAATAACAGAAAGCTGCTCTACTCATC | 23 | 278 |
| | GAATTCGACTGGAACATTCCTTATATAAACCTCAAAAAGGGTA | | |
| | A*A*T+ | | |
| HDR donor | +G*C*CCGTCTTTTACAATCAAATCTTCAAGCTTTACCCCACC | 24 | 279 |
| | GAATTCATATGGTGTTGATCCTCTGTTCATTACATATGGAACA | | |
| | T*T*G+ | | |
| HDR donor | +G*C*CACTCACCTATGATGGTCCGCGCCGCCGAGACGATGAC | 25 | 280 |
| | GAATTCCACAGGATCTGAGCCTGCATTCATCTTGCTTCTCCTG | | |
| | C*C*G+ | | |
| HDR donor | +A*C*AGAGATGTCAATCCCACGGATGGATAGGTCGCGGTTGA | 26 | 281 |
| | GAATTCCAAAGGCAGCTTCACAATCACTCAGTTGGGGAACTGG | | |
| | A*T*C+ | | |
| HDR donor | +C*C*AAAGGACTGCAAGTTCATCAAGCATCTGACCCAAGAAA | 27 | 282 |
| | GAATTCCTAAGGGTTGCATGGAAGTAGACGGATCCAATTCCTA | | |
| | G*A*A+ | | |
| HDR donor | +T*G*GAGAAGATGAGGTGTGTGACTTTGCACGTGAGCTCGCC | 28 | 283 |
| | GAATTCCATGGGCCACTGGTTGTGCTGCACGAGACTGACCACC | | |
| | C*A*G+ | | |
| HDR donor | +A*A*GTAGAAGAAGAGCATACCAATGCAATAGGCACTCTCCA | 29 | 284 |
| | GAATTCCGGCGGTTTGACAGCCACGTTAGTAGATAACATATCA | | |
| | A*C*A+ | | |
| HDR donor | +G*G*CCTCATCTCCTGCTGTCCTTCGAGCGTCCCGGCTGTAC | 30 | 285 |
| | GAATTCCAATGGAGCCTGAAGAGTTCGGCGCAGTTCCTGGGGT | | |
| | C*T*C+ | | |
| HDR donor | +A*C*CTGGAGCCCTGGCTTATGGGAGTCAGGATGACCGAATA | 31 | 286 |
| | GAATTCCGTCGGGGTGAGTCTGCCTTGAGACAGGGAAGTGGTT | | |
| | G*A*A+ | | |
| HDR donor | +A*G*ACATTGCCAAGGTGGTCATGGTTTCCGGCTAGCACGTA | 32 | 287 |
| | GAATTCCCAGGGCACTTTGCGAAGGGAGCGGTCAGAGAATACG | | |
| | T*C*C+ | | |
| HDR donor | +A*G*TGGGGCAGGCAGCCCGGCCCAATGAAGCGCCCACACGA | 33 | 288 |
| | GAATTCAATGGGAGGGGTGATCTCCACGAGGGCAATGTCATTT | | |
| | C*C*C+ | | |
| HDR donor | +T*C*CAGGTTGGAGTCAGCAGTGCGAAGAAGCGTTCGTATTC | 34 | 289 |
| | GAATTCGGTAGGAGAGAGAGGGCTGCCTGGAGTGGAGGCCTGA | | |
| | G*T*C+ | | |
| HDR donor | +G*T*ACAGTGGATCATCTCCGAATTGGCTTGTTACACGTACT | 35 | 290 |
| | GAATTCCTATGGTAGCTGTACCTCTGTATGACACCTTGGGACC | | |
| | A*G*A+ | | |
| HDR donor | +T*T*GTCTTAAGGTCAGGACACTCCAATACAATGGACTCCAC | 36 | 291 |
| | GAATTCCGCTGGGGCCACCTCCTCACTGGCCACAATGCACTTG | | |
| | G*C*C+ | | |
| HDR donor | +T*A*GTTTGCAGGCTTCTCTTCAGAGTCTCTATGTGAACGGA | 37 | 292 |
| | GAATTCTCTTGGATTCCAGCTGCAGTCTTCTCATCACTACAGG | | |
| | T*G*A+ | | |
| HDR donor | +C*A*TAATTTTCCTCACCTGATGTCTGGGACGTCCCACAATG | 38 | 293 |
| | GAATTCGATGGGAAAACAGCCCTGGGAGCATCGTCCCCAGCAA | | |
| | A*G*C+ | | |
| HDR donor | +C*T*GGAAGGATGGCCTCGGCTTCTGTGCTTTGATCCACCGA | 39 | 294 |
| | GAATTCCACCGGCCCGAGCTGATTGACTACGGGAAGCTGCGGA | | |
| | A*G*G+ | | |
| HDR donor | +C*T*CGAATTTCCCTAGGATTCAAAGAGGGCTCGGTCATAAG | 40 | 295 |
| | GAATTCTACAGGCTGTTGGCTGGGTTTTACTCCAAGCTCCCGC | | |
| | T*C*A+ | | |
| HDR donor | +C*A*CATTTGGTCTCTTTTTCTTCCTGTAGGAGCACTGTCGA | 41 | 296 |
| | GAATTCCCCTGGCATGCCCATACTGTCAGCATCGCAGCTATGG | | |
| | A*C*A+ | | |
| HDR donor | +G*G*GAAAAAGACAGAACCAATCAAACTGGTGTTGAACCGTG | 42 | 297 |
| | GAATTCTTATGGTGACAAAGATAAACGGCGGCATTGTTTTGCT | | |
| | A*C*C+ | | |
| HDR donor | +T*G*GTTCAGCTCTCGGGCTCTTGTCACCTCATATGGGTCGT | 43 | 298 |
| | GAATTCCCGGGGTGGGCCTGGAACTGTGGAGACGGGAATGGGG | | |
| | G*A*C+ | | |
| HDR donor | +G*C*TTCTCAGGAATGGTGACTTCATACGAGTCAAACTCCCC | 44 | 299 |
| | GAATTCTTCAGGGTGAAAAATTACATCTTCGCCAAGAGACTTA | | |
| | A*G*G+ | | |
| HDR donor | +C*T*CCCCGCCGCCCCCAACCTGGTCCACGTAGTTGGCGACT | 45 | 300 |
| | GAATTCTCCAGGAGACGCTGTTTGGTGTGGTTCAAGTTTCGGT | | |
| | G*C*C+ | | |
| HDR donor | +T*A*GTACACGAATATGATTCAGCTGCCAGTATCAGTACGTG | 46 | 301 |
| | GAATTCTAATGGTCTAAGGTAATGCAGAATATCTTTCTTTTTC | | |
| | C*T*T+ | | |
| HDR donor | +C*A*AAAAATGCCCACTGGCATCTACTCGGACTGGACCCACC | 47 | 302 |
| | GAATTCACGTGGTATTCTGGCAGGCCCTTGATAAAATGCTCTG | | |
| | A*A*A+ | | |
| HDR donor | +C*C*TCCCGGTGTTGCAGACAGGCAGACGCTAAGCACGATGG | 48 | 303 |
| | GAATTCTGTGGGAGGAGGAGGTGAGGACGCTGGCAATGATGGC | | |
| | G*T*C+ | | |
| HDR donor | +C*A*CCTGCGACCCTTGACCACAAGTAACCGAACATGTGCTC | 49 | 304 |
| | GAATTCCACTGGGACCACTCTTCCACACCAGATTCACCTGCGT | | |
| | G*C*A+ | | |
| HDR donor | +C*T*TAAAAAAGCATATGAAAGAATTCAAGGTTTTGAGTCGG | 50 | 305 |
| | GAATTCTTCAGGTCACCCAATTTCGGTAAGTAACACAATGGTC | | |
| | T*T*A+ | | |
| HDR donor | +G*G*ACATGGTGTCCTTCGAGGAGGACCGGATCAACGCCACG | 51 | 306 |
| | GAATTCGTGTGGAAGCTCCAGCCCACAGCCGGCCTCCAGGACC | | |
| | T*G*C+ | | |
| HDR donor | +C*G*CGCAACATCGTCAAGTATGACCTACGGACGCGCATCAA | 52 | 307 |
| | GAATTCGAGCGGGGAGACGGTCATCAATACCGCCAACTACCAT | | |
| | G*A*C+ | | |
| HDR donor | +A*A*ATCTTCTAAAGAAGCATATTCTATTAAAGTATCGGTAC | 53 | 308 |
| | GAATTCGATAGGGAGTCCAGGGCATGAAATAAATTTTATCTGC | | |
| | A*G*C+ | | |
| HDR donor | +G*A*GTATACCAGAGTGAACATTTGTTTGAGTCCGACCACCA | 54 | 309 |
| | GAATTCATCTGGGGCGTGGTGCAAAGACCCTCTGCAGGCATCT | | |
| | G*A*C+ | | |
| HDR donor | +C*T*TCTGCACTAGATTAAGATGTTCTACGAGGAGCATTTGC | 55 | 310 |
| | GAATTCACTTGGACGATGAGATCCGCTACATCCTGGATGGCAG | | |
| | T*G*G+ | | |
| HDR donor | +C*C*CGAAGCCATGGCAAGCAAGGGCTTGCAGGACCTGAAGC | 56 | 311 |
| | GAATTCAACAGGTGGAGGGGACCGCCCAGGAAGCCGGTGAGGA | | |
| | T*A*G+ | | |
| HDR donor | +T*G*TGTGCCCAATCATGGCAGTGACCTGATAGCCTATACGT | 57 | 312 |
| | GAATTCTCATGGTCTTCGATGACATGCTGTACCAAAGCTTCAT | | |
| | A*G*G+ | | |
| HDR donor | +G*A*AATAACAGCTCCTGTTCATTCAGCCCAAGGGAAGTCAC | 58 | 313 |
| | GAATTCCGCGGGAAAGACCTGCTAACAAAAACAACAACAGGTA | | |
| | T*C*T+ | | |
| HDR donor | +C*G*ACGGCGCCGCCTACCCGCAGTGCGGCCTCAACGACGAG | 59 | 314 |
| | GAATTCACCTGGTACATCCTGTCCTCCTGCATCGGCTCCTTCT | | |
| | T*C*G+ | | |
| HDR donor | +A*C*TCCGAGACTCCAGACCATGACCAACGTGTTCGTGACTT | 60 | 315 |
| | GAATTCCGCTGGCCGCAGCCGACCTGGTGATGGGACTCCTGGT | | |
| | G*G*T+ | | |
| HDR donor | +A*G*CTCTGACAGGAAGCCCTCGAGGAACTCCTCGATCTCGT | 61 | 316 |
| | GAATTCCGTCGGTCAGCACCGTCTGCGGGCGCCCTCCAGGGCA | | |
| | C*A*G+ | | |
| HDR donor | +T*C*CTCCTCTTCATCATACGACTCATAAGAGCTGCTCATCG | 62 | 317 |
| | GAATTCCATCGGAATCATAATCTGTAAAAAATAAACATACACT | | |
| | G*G*T+ | | |
| HDR donor | +C*T*CTTCTGATGGTGTAGAGCAAGAAGGCGATGATGAGCAC | 63 | 318 |
| | GAATTCCGTAGGCAGCATGCTCAGCAGAAAAGAGCTGGGGACA | | |
| | C*A*C+ | | |
| HDR donor | +C*C*TGTCTAGCGGGAAGTCAGCCCTGGTGCACCGCTATCTG | 64 | 319 |
| | GAATTCACGGGGACCTATGTCCAGGAGGAGTCCCCTGAAGGTG | | |
| | A*G*C+ | | |
| HDR donor | +C*A*CCTTCTGTTTGAGAGTTCCTCTGGAATATTGTGCTTGA | 65 | 320 |
| | GAATTCCTCTGGATTGGCAGAAAGGTTAAACTCCAAAGCTATA | | |
| | T*G*T+ | | |
| HDR donor | +G*G*GGGGCGGGTTGAGCTTGGGGCTGGTGGTGCTGGAGATA | 66 | 321 |
| | GAATTCCCGGGGCTGAAGCATATGGAGTCACCCAGCCCGGTGT | | |
| | C*C*A+ | | |
| HDR donor | +G*T*CGCCTCTTCGTCTTTAAGCTGATTCCCATGTTGAACCC | 67 | 322 |
| | GAATTCCGATGGTGTGGTCCGGGGACACTACCGGTAAGTGGCT | | |
| | T*C*C+ | | |
| HDR donor | +T*A*CTTTGAGGTGGACATCCCTAAGATCGACGTGTACCACT | 68 | 323 |
| | GAATTCACGAGGTGGACATCAAGCCGGATAAGTGTCCCCGTAG | | |
| | A*G*T+ | | |
| HDR donor | +C*C*ACTTCATGGAGGGCAGATGTCGTAGCACCACATCAACG | 69 | 324 |
| | GAATTCGCATGGACAGGGTTAGTGCTGATTGGCTTGTCTAATT | | |
| | C*C*A+ | | |
| HDR donor | +T*G*CTGGCTGGCAGGAGTCATCTTCATCGACCGGAAGCGCA | 70 | 325 |
| | GAATTCCGGGGGATGCCATCAGTGTCATGTCTGAGGTCGCCCA | | |
| | G*A*C+ | | |
| HDR donor | +G*G*GCCGGCCCCGGGAGGCTCTGAGTACCAATGAGTGCAAA | 71 | 326 |
| | GAATTCGCGCGGAGAGCCGCGTCGGCGGCCACGGCAGCGCCCA | | |
| | C*G*G+ | | |
| HDR donor | +T*T*AAAAAGCAAAACAGTGAGGTGAAGGATAACATCGTTAC | 72 | 327 |
| | GAATTCCACTGGACACTGTTGGCTTCATTGTCTGAATATATCT | | |
| | G*A*G+ | | |
| HDR donor | T*C*ACGCTCTCTTCCACAATGCCTCGGATCTTCTTAAACACG | 73 | 328 |
| | AATTCGTTTGGATACTTCTTATAAACCCAGTGGGTTAAGTCTC | | |
| | *C*C | | |
| HDR donor | +G*T*GTGCCAAGACTGCCCCCTGCTGGCCCCGCTGAACGACA | 74 | 329 |
| | GAATTCCCAGGGTGGTGCACGCCGCGAAAGCTGCCCTGGCCGC | | |
| | C*T*T+ | | |
| HDR donor | +A*G*AACGCTGAACTCCTTCAATCCTGCGGAAATGAGATCCT | 75 | 330 |
| | GAATTCTATTGGCCTTAAGAAGAGCCATCTGCCTGGGTAGAGA | | |
| | A*A*A+ | | |
| HDR donor | +C*T*CTGTTGCCAGGTACTTTATTACCAAGGTTGCCATCGGA | 76 | 331 |
| | GAATTCACCAGGAATGACATTACTCACTATCAGAATTGAGAAA | | |
| | A*T*T+ | | |
| HDR donor | +C*A*AAGTCAATACTTCCAAAGGCTTCCTGATTGATGGCTAC | 77 | 332 |
| | GAATTCCCGCGGGAGGTGCAGCAAGGAGAAGAGTTTGAGCGAC | | |
| | G*G*G+ | | |
| HDR donor | +T*C*TTTCAGGCTGATTCACCCCAAGAGTGGCCGTTCCTACC | 78 | 333 |
| | GAATTCACGAGGAGTTCAACCCTCCAAAAGAGCCCATGAAAGA | | |
| | T*G*A+ | | |
| HDR donor | +T*G*CTGCACAGCTTGCAGGATTGCATTCTGCACAATCTGTT | 79 | 334 |
| | GAATTCTGCTGGCATTCTGCAGCTTCACCTCTTCAGGCTCATT | | |
| | T*C*C+ | | |
| HDR donor | +T*T*TCCTGACAATTCTTTTAGTGGAGAAGCGGGACTATTTC | 80 | 335 |
| | GAATTCTACTGGCTACTGTAAGTGTAGAGGAAGATTCTTGGGT | | |
| | T*T*C+ | | |
| HDR donor | +G*C*GCATTCTGCAAGGTCTGCAGGACGCCAATGGCAACTAC | 81 | 336 |
| | GAATTCACTAGGCAGACGGACATTCTGCAAGCTTTCTCTGAAG | | |
| | A*G*A+ | | |
| HDR donor | +G*C*AAATTTTTCAGAACATAAAACAAGAATATAGTCGTTAT | 82 | 337 |
| | GAATTCCAGAGGTGGAGACATTTAGAAGTTGTTCTTAATCAGA | | |
| | G*T*G+ | | |
| HDR donor | +G*A*ACCTACCTCCCAGGCATCCAAGAACGTTGCTTTTCCAC | 83 | 338 |
| | GAATTCCGATGGCATTACCTTTATCATCTTTGGGGAAAAGGTC | | |
| | A*T*C+ | | |
| HDR donor | +C*T*TGAGCAGCTCCGTGGTCTCAGGGACACCCCCATTGATT | 84 | 339 |
| | GAATTCACTGGGTACAGATCCTTCCATGCAAGGAGAGAGGGGA | | |
| | G*G*C+ | | |
| HDR donor | +G*C*GCTGTGCCCAGTACAAGAAGGACGGAGCTGACTTCGCC | 85 | 340 |
| | GAATTCAAGTGGCGTTGTGTGCTGAAGATTGGGGAACACACCC | | |
| | C*C*T+ | | |
| HDR donor | +G*C*TGATGATTGTTCATCAACTTTTCGTTTATAACCACTAC | 86 | 341 |
| | GAATTCGAGTGGCTTTCCTTTTTCCAGAGTCTCCAAACAGCTT | | |
| | C*C*T+ | | |
| HDR donor | +C*T*GCGAATATGGGTAGTGCTTCGTTCCATGGACGTTACGC | 87 | 342 |
| | GAATTCCCCGGGAGTCTCTCAGTATCTTGGTAGTGGCTGGGTC | | |
| | C*G*G+ | | |
| HDR donor | +G*C*ACCGGCTCTTCATGAAGCTGGGTGGCACTCACTCTCTG | 88 | 343 |
| | GAATTCTTCAGGGCCTGGTAGGCCTCCCCTCCTCAGCTGCCTT | | |
| | C*T*C+ | | |
| HDR donor | +C*A*TCCACCAGAAGCATTTCCAGCACATCCAGGTCTGCATC | 89 | 344 |
| | GAATTCCCCTGGCTGGAGGGCCGAGGACTACCCCCGCTTCTAG | | |
| | G*T*G+ | | |
| HDR donor | +A*C*TCTTCCTGCTGGCGCTGGCTTTGTCCCCGCACGGAGCC | 90 | 345 |
| | GAATTCCACGGGAGGCCCCGGGGGCGCAGGGGAGCGCGCGTCA | | |
| | C*G*G+ | | |
| HDR donor | +T*G*CAGGTATAAAGAAGCGACTAAACGGAGACCCCGAAGTT | 91 | 346 |
| | GAATTCTACTGGAGAAACTGCGTTGGGTGACCGTAGGCTACCA | | |
| | T*T*A+ | | |
| HDR donor | +C*T*GGAGCCCGAGCTGCGCCGCCGCCGCTACGAATACGATC | 92 | 347 |
| | GAATTCACTGGGACGCGGTGAGACCGGCAGCGCCGGGGGCGAG | | |
| | G*G*A+ | | |
| HDR donor | +A*T*TCTTCTGTAGTTCTGTATCTTGCAAATGAGTACGGCTT | 93 | 348 |
| | GAATTCGTTAGGTGGCATTAAGAGAGCATCCACCAGTCCACAT | | |
| | T*T*C+ | | |
| HDR donor | +A*T*TGCCTGTTCTCCTTCCCAGGTGGATTCATACGACGTGA | 94 | 349 |
| | GAATTCCTGTGGACGAGGAACTGGGCGAGATCCAGCTGGTCAG | | |
| | A*A*T+ | | |
| HDR donor | +G*A*CCTGCAGAAGTTTTCCAGAGACGTCGAGCCCATACAGG | 95 | 350 |
| | GAATTCAACCGGTCCACCAAGAACACAATAGCCGCCGCAGCCG | | |
| | C*A*C+ | | |
| HDR donor | +A*G*TGACTATGAACTTTGCCCAGAGATAACCCTAACCTACA | 96 | 351 |
| | GAATTCCCGAGGAGTTTTCAGATGATGACCTGGAGTATCTGGA | | |
| | A*T*G+ | | |
| HDR donor | +C*T*TCCATAGTGGAGCAGTGACAGACAATGGTGTCGCGTAC | 97 | 352 |
| | GAATTCATGTGGGGAGAGAATTCTGCTGGCCAGTGTGCAGTAG | | |
| | C*C*A+ | | |
| HDR donor | +G*G*CACCACGCTGGTGGAGTGCAAGAGTGGATATGGCCTCG | 98 | 353 |
| | GAATTCACCTGGAGACCGAGCTCAAGATGCTGCGCGTGATTGA | | |
| | G*C*G+ | | |
| HDR donor | +T*A*CGAGCCCATGGGTGGATGGCTGCACCACCAAATCATCC | 99 | 354 |
| | GAATTCCCGTGGTGTCCCAACAGCACCCCCTGACTCACACCCT | | |
| | G*C*A+ | | |
| HDR donor | +T*A*GCTCAGTGATGGATTTTAAGAACGATTACACCTGTCGC | 100 | 355 |
| | GAATTCCTGTGGTCTGACTCCAGGCACTCGCGTCAGGTACTTC | | |
| | T*G*C+ | | |
| HDR donor | +T*G*TTGTGGGAATAACAATGATTGATCTTTACCCAAGAGAC | 101 | 356 |
| | GAATTCTCGTGGAATTTTGTCTTTGGACAGGCCTCTTTGACAG | | |
| | A*T*G+ | | |
| HDR donor | +G*G*CTCTGCTGCCATGAAGGTGAAGATTAAGTGCTGGAACG | 102 | 357 |
| | GAATTCGCGTGGCCACTTGGCTCTGGGTGGCCAACGATGAGAA | | |
| | C*T*G+ | | |
| HDR donor | +G*A*CACTCACGGTGCAGGCGGCTGACATTGTGAGCCGGGTC | 103 | 358 |
| | GAATTCAACTGGCTGGGCCATCTCGGGCAGCCTCTTTCTTCGG | | |
| | G*C*A+ | | |
| HDR donor | +T*A*CTGGTGTCACTGGTGCTACCCACAAGACGGACCGGAAC | 104 | 359 |
| | GAATTCCACAGGCACCAGTGGGGGTGGCGGCAGGACCTGGGGT | | |
| | G*A*C+ | | |
| HDR donor | +C*A*GCCCTCATTGTGGGAGTACAGCCCATTCGGTCTTGCAC | 105 | 360 |
| | GAATTCATCAGGGTGAGCACCAAGGTCAAGGAGAAAGCTGACC | | |
| | A*T*A+ | | |
| HDR donor | +G*C*CCTCAATCTGGAATCTCAAATGGTATTCTCACGGGATC | 106 | 361 |
| | GAATTCCCGAGGCTGAAGAAATAGAAGCTGAATATGCTGCATT | | |
| | A*G*A+ | | |
| HDR donor | +A*C*CTGGAGTCTGTGAGAGTGCTCCTTCGACACAATGCCAA | 107 | 362 |
| | GAATTCCGTGGGCAAAGAGAACCGCCAGGGCTGGGCAGGTACT | | |
| | G*C*A+ | | |
| HDR donor | +A*A*TGTAGGACTCAAACTGACAAGTAGACTGGATGCTGCTC | 108 | 363 |
| | GAATTCGACAGGCTTCCTTTGCAGGGTACTAAGACCTGGAAAA | | |
| | A*A*C+ | | |
| HDR donor | +G*A*GGACTGACTTACGGGGACTGGCCATTCGAGTCAAGCTT | 109 | 364 |
| | GAATTCGGTGGGTCGGGCAGATTTCCTGGAGGCCAGGGCAGCC | | |
| | A*C*G+ | | |
| HDR donor | +A*C*ATCCAGTTCTACCAGTGCTTTCAAAGTTTCCAAACGAC | 110 | 365 |
| | GAATTCCCCAGGCTGCAGCACAATGTAAGAGTGTGTACCCTAA | | |
| | C*A*A+ | | |
| HDR donor | +G*T*GCGGAGACTCCTTTCTGAAAAGGCCACTCACGTGAACA | 111 | 366 |
| | GAATTCCTAGGGATGAAGATGAGTATACCCCTCTTCATCGAGC | | |
| | A*G*C+ | | |
| HDR donor | +C*G*AAAAATAAGTTTAGATGACCTTCGGAAGGCATATATCG | 112 | 367 |
| | GAATTCTCAAGGATGTTCAGCAGTACATTCTTCATCGTTTAGA | | |
| | T*C*A+ | | |
| HDR donor | +A*G*CACTGCGCCCGCTCCTGTCAGCCACGTTGAGGCTGCTC | 113 | 368 |
| | GAATTCAACAGGGGTGCCAGAGCCTCAGCACACTTGGTGGCCC | | |
| | G*G*T+ | | |
| HDR donor | +A*A*ACTCTATGAAGCCATCATGAGAGAAGACTGCACTACGA | 114 | 369 |
| | GAATTCTCGAGGTACTCCTGAGAAATCACCCTGTCAACCAGCC | | |
| | C*A*T+ | | |
| HDR donor | +G*G*TGACACACAAAACTTTTCACCGCTATACGGTTCGGGCC | 115 | 370 |
| | GAATTCAAGCGGGGCACAGCCCAGGGGCTTCGGGATGCCCGAG | | |
| | G*T*G+ | | |
| HDR donor | +C*A*GTTGAGTGTACCAAAGGCTGAGGACCGGTTTTTCAGAT | 116 | 371 |
| | GAATTCCATTGGGTTTCAGAGGCAATTTGATGTGCATTATCTC | | |
| | A*G*C+ | | |
| HDR donor | +T*A*GGCTCAAAAGGCTCGGAAACTGGGGGCCAACGTTTACA | 117 | 372 |
| | GAATTCCCCTGGGTGTGGCTGATTATAATCTGGATCAGGTAAT | | |
| | T*C*C+ | | |
| HDR donor | +G*C*AGTCCCAAATCACAGCTGTCCAGTGAGTACTCTCCTAG | 118 | 373 |
| | GAATTCTACTGGATACAAAGGAAGAGGCCACGGCCCACGATCT | | |
| | T*C*T+ | | |
| HDR donor | +C*A*GGACAATGAGCTCTTGACGCTAGAGATTGTGCATCGTT | 119 | 374 |
| | GAATTCACGTGGAGCTGCTGGACAAATATTTTGGAAATGTAAG | | |
| | T*G*T+ | | |
| HDR donor | +T*C*TAATACTTGACAGAACTCTCAAAGCGCTTGCACGAATT | 120 | 375 |
| | GAATTCAGTTGGTTTGGGTCCTAAAAATAGTGCAAAAATATTC | | |
| | A*C*C+ | | |
| HDR donor | +C*T*GCCGTACCTGTGATGGTGTTGACAACCGTTCGAAGGAT | 121 | 376 |
| | GAATTCGGTAGGAGGCTTTATGAGTTCTTTAAGAATGTTCGAC | | |
| | T*C*G+ | | |
| HDR donor | +C*A*ACAGCAGATTGTTCGAGAGACTTTCCATCTAGTCCTCA | 122 | 377 |
| | GAATTCAGCGGGATGACAACATCTGTAACTTCTTGGAGGGTGG | | |
| | A*A*G+ | | |
| HDR donor | +G*G*GCTTCTGATGAAACTGCAGAATCTTTGCACACGGTTGG | 123 | 378 |
| | GAATTCATCAGGATGAGAGTTTTTCCCAGAGGCTTCCACTTAA | | |
| | T*A*T+ | | |
| HDR donor | +G*G*GGAGCTGGGTGAGGCACTGTCTTGGGACAACGTTGTCC | 124 | 379 |
| | GAATTCAGCTGGACTCCTCATCGCTCGGTGCCAGGTTCTGGAT | | |
| | C*C*G+ | | |
| HDR donor | +T*G*CACAGGGGCTGGAGGACACATACCAGGTGACATTGTAC | 125 | 380 |
| | GAATTCCGCAGGGCTGGGTCGAAGGCTGGCAGGATGGTGTTGA | | |
| | A*G*A+ | | |
| HDR donor | +G*G*AGCGCCTGGACAATGGCACCTCGGTCAAGATGGACCAG | 126 | 381 |
| | GAATTCCACAGGGGCTTTCTACACAACCAGGTGACCGACCCAG | | |
| | C*C*A+ | | |
| HDR donor | +T*G*TTTCCAAACTCCTTCAGCTTATCCAAGGCACTGGAGAC | 127 | 382 |
| | GAATTCGTCTGGGGTCCCCTGGGCTGGGGCTGGGCCTGCCAGG | | |
| | A*A*G+ | | |
| HDR donor | +C*A*GAGGCCAGGAGCGCCAGGAGGGCAACAACAAGGAGTAC | 128 | 383 |
| | GAATTCCCGGGGCTGCATGGCACCTCTGTTCCTGCAAGGAAGT | | |
| | G*T*C+ | | |
| HDR donor | +C*C*CAGCCCAGCACACCCTCACCAGAACCATTGCCACCCGT | 129 | 384 |
| | GAATTCCTCTGGTCCTGTTCACCACTGTCTCCAGCAGCTCCTT | | |
| | C*A*T+ | | |
| HDR donor | +G*A*ACTAGTAAACAAATTGCGGGCACGAGCCCAAGCCCGCA | 130 | 385 |
| | GAATTCACTTGGACCAAAGCGGCACCAATGTAGCAAAGGTGAT | | |
| | G*A*A+ | | |
| HDR donor | +C*A*ATGGAGATTCATTTTCAGGTATGTAAAAGTGAACAGGT | 131 | 386 |
| | GAATTCCGAAGGTTTGAATATTTATCTGGGGGTCCTATCCAAT | | |
| | C*A*T+ | | |
| HDR donor | +T*G*ACTACGGCATGTTGCTGCCCTGCGGAATTGACAAGTTC | 132 | 387 |
| | GAATTCCGAGGGGTAGAGTTTGTGTGTTGCCCACTGGCTGAAG | | |
| | A*A*A+ | | |
| HDR donor | +G*G*CGGCCACGGCGGCTCCAGGCTCTGGGACGCAACCTCTC | 133 | 388 |
| | GAATTCTCGGGGTGGCACTCCAGGGCCGACTGCGGCTGTGAAG | | |
| | G*T*T+ | | |
| HDR donor | +G*G*ACCGCGAGCGCCGAGGGGACCGGACTATTTATGACTAC | 134 | 389 |
| | GAATTCGTGCGGGGAAATTACCTGGATCCCCGGTCTTGCAAAG | | |
| | T*C*T+ | | |
| HDR donor | +T*A*CAGTGTGGGATGTGGGTGGCCAGGACAAGATTCGACCC | 135 | 390 |
| | GAATTCCTCTGGAGACACTACTTCCAGAACACCCAAGGTATGC | | |
| | T*C*A+ | | |
| HDR donor | +G*G*TTGGGATGGTGGTGACAATCTCCCCCAACTTCAGTTTG | 136 | 391 |
| | GAATTCTACAGGATTGTGGTCTTGCCAGCCGCATCCAAGCCAA | | |
| | C*T*G+ | | |
| HDR donor | +T*T*GGGCAGCGCCTAGAGGAAACAGTCCACCACGAGCGGAA | 137 | 392 |
| | GAATTCGTATGGCCCCCGCCTGGCGCCCCTGCTGGTGGAGCAG | | |
| | T*G*T+ | | |
| HDR donor | +T*T*TGGCTTCACTCTGCGCCACTTCATCGTGTACCCACCCG | 138 | 393 |
| | GAATTCAGTCGGCCGTGCACTGCAGCCTGAAGGTATGCCCGGC | | |
| | T*C*G+ | | |
| HDR donor | +G*C*TGAGGAAGCAAGGAGGCTTTGTCAAGACTTGGCATACT | 139 | 394 |
| | GAATTCCGCTGGTTTGTGCTCAAGGGGGATCAGCTCTATTATT | | |
| | T*C*A+ | | |
| HDR donor | +T*C*AAAATCAGAAACCACTTATCCAAGGTGTTCGGACGGTC | 140 | 395 |
| | GAATTCAATAGGCAGAGCTACAGAAAGAGAAAAAAGAAAAGAT | | |
| | A*C*A+ | | |
| HDR donor | +G*A*CGCGCAGCGCTGGTCCCTACCTCGGATTTCCACACGAC | 141 | 396 |
| | GAATTCGTAGGGGTGCCCGCGACTGTCGGGTGGGGACTGGAAC | | |
| | T*T*C+ | | |
| HDR donor | +T*G*CATTTCTTCTTGTTTGGAAGTGATGGCTTGGATCATCG | 142 | 397 |
| | GAATTCACATGGCATTGCTCAGCTCTTCCTAAAAAATAAGAAT | | |
| | T*G*C+ | | |
| HDR donor | +G*G*CCCCGGGAGTCCGACCCTGGATGCCAATTGGATACCGC | 143 | 398 |
| | GAATTCTGTGGGCCTGGAGGGCCATAGGAACCTCCTGGGTACG | | |
| | G*A*C+ | | |
| HDR donor | +G*C*TGCTGGGAGCAGCACTGCTCAGTTCTCGTCAAGGACGG | 144 | 399 |
| | GAATTCCGTGGGCGTGGGTGAGTCTGCCACAAAACTTATAAAA | | |
| | A*G*C+ | | |
| HDR donor | +T*G*GTTACGTTTTCTTACCTCCAACATGGCAACTAACTCTG | 145 | 400 |
| | GAATTCATTTGGAAATGCCAATTCGGTCTCGGTCACAACTGTC | | |
| | T*A*C+ | | |
| HDR donor | +A*A*CCCCAGCACAGTACCCACCTGTGGTCGACACGACGTTA | 146 | 401 |
| | GAATTCTCGGGGCTTTCACTGCAGAGTTTGGACAACAGACTGG | | |
| | T*T*T+ | | |
| HDR donor | +T*C*CCGGATGGTGATCTTTCTCTGTGCCACGATGTCAGTAA | 147 | 402 |
| | GAATTCGATTGGTGCCCACCTATAGGAAAAACCACGATGCAAA | | |
| | A*A*G+ | | |
| HDR donor | +A*G*TCCCTGCGGTCCTACTGGCGGAACTACTTTGAGAGCAC | 148 | 403 |
| | GAATTCCGATGGCCTCATCTGGGTAGTGGACAGCGCAGACCGC | | |
| | C*A*G+ | | |
| HDR donor | +A*A*GTTTGTTAATGATCGTCGTTTTGCCACTATTATCTAGC | 149 | 404 |
| | GAATTCCCAAGGCACAAAACATGAACCTCCTTCTTCTTCAGGC | | |
| | C*A*A+ | | |
| HDR donor | +G*T*CCTTCGCCGGATGAAGAAGCGCTACCCCACGACGTTCG | 150 | 405 |
| | GAATTCTTATGGTGGTCATGTTGGCGAGCTATTTCCTTATCTC | | |
| | C*A*T+ | | |
| HDR donor | +G*C*TGAACAACTTCAGAAACTCCTTTACCTGCTGGAGTCAA | 151 | 406 |
| | GAATTCCGGAGGATCCTGTAATTATTGAAAGAGCTTTGATTAC | | |
| | T*T*T+ | | |
| HDR donor | +G*G*GGGATTAGAGCCACTCATCAGACTACTGAGTAGCCCTG | 152 | 407 |
| | GAATTCACCCGGATGTAAAGAAGAACTCTATGGAATGCATTTA | | |
| | C*A*A+ | | |
| HDR donor | +T*G*GTTGTGGGCATGGCCAAAGGGCACACGGATGTCGTCAT | 153 | 408 |
| | GAATTCCGAAGGTCATGACACGCAGGGCCCAGCAGGCTTCCCT | | |
| | G*A*C+ | | |
| HDR donor | +T*A*TCATCAATTTTATAAGGAAAGTTGAACTTGCCTCTCCG | 154 | 409 |
| | GAATTCGACAGGCCATGTTGTAGCTGGAGCCCTGGTGCTCTTA | | |
| | C*T*T+ | | |
| HDR donor | +T*A*GCTGTTGGTCTTGTCCCTGGACTCACGCGGCTGGAAAC | 155 | 410 |
| | GAATTCCACAGGAGCAATGCACTGGTTCTCCTCTCTCAACACT | | |
| | T*T*A+ | | |
| HDR donor | +T*A*TGTGCGGCTGGTTGTCCGGAAAGTACAATTTGCACCAC | 156 | 411 |
| | GAATTCCGGAGGCAGGCCCTGGCCCCTCAGCCCAGACCATCCG | | |
| | C*C*G+ | | |
| HDR donor | +G*T*TCTGGGACAGGTGGCTGTTGCCTAAGGATTCCACGGCC | 157 | 412 |
| | GAATTCTCTCGGTACTGCTCTTCATTCTCCACATGGCCTGTGG | | |
| | A*G*G+ | | |
| HDR donor | +T*C*GGCAATAAGAGTGATTGCTTTTGATCACGGCGTATCGC | 158 | 413 |
| | GAATTCAACAGGAAACCATTCTGAATGTCGATGTTGGCATTGT | | |
| | G*G*T+ | | |
| HDR donor | +A*T*GGATCACTGATTGAATAATAGATGGCTTTACCTCGTCT | 159 | 414 |
| | GAATTCCACAGGAGCCTTGCGTTCCTTTTCAAATGTCACCAAG | | |
| | C*A*A+ | | |
| HDR donor | +A*A*GCGCAACGAGCGCGAGAGGCAGCGAGTCAAGTGCGTCA | 160 | 415 |
| | GAATTCACGAGGGCTACGCTCGCCTCCGCGGCCACCTCCCCGG | | |
| | C*G*C+ | | |
| HDR donor | +T*C*ATGGGATACACAATATTGTTCAGAACCGGAGCAAAATT | 161 | 416 |
| | GAATTCCGCAGGGTGCTCTGGTTGGTGGTGGTTCTGGGCTCAG | | |
| | T*C*T+ | | |
| HDR donor | +A*T*GGAGAAGTTCAACGCGTCCATTGCCTACGACCGGCACC | 162 | 417 |
| | GAATTCTTTGGGAGGTGGATGTTCAAGGCAGCAAAGCCTACAG | | |
| | C*A*G+ | | |
| HDR donor | +G*T*GGCCTGCCTCCTGGTCCTCTTCGTGTGGTACTGTTATC | 163 | 418 |
| | GAATTCACGTGGGCAGGGAGCAGCCCCGGCCCTACGTCTCCGT | | |
| | C*A*A+ | | |
| HDR donor | +G*G*CCCTGCTCCCAGCTAGTGGGCTGGGATGGCTCGTAGAC | 164 | 419 |
| | GAATTCTATGGGAAACTCCCCCCGGCCCCTGCCCCCCTGGCTC | | |
| | C*C*T+ | | |
| HDR donor | +T*G*GAGATCTTGCTAAAGCTGGTCCGGCTTTTAACCACCCA | 165 | 420 |
| | GAATTCACCTGGGGAGACGTAGGGGCAGCGCCCAGCGAGATGA | | |
| | A*G*C+ | | |
| HDR donor | +C*T*AAGCAACAAATTTTCTTTACTGAGACCCACGCGTTCTT | 166 | 421 |
| | GAATTCTGTTGGAATGTTCTCTATAAGAAAGACAGCATATAAA | | |
| | T*T*C+ | | |
| HDR donor | +G*A*AGCAGAAGAAGAGGCTCGGGTGACGGTGGTGCCCAAAT | 167 | 422 |
| | GAATTCCGGAGGAGGAGGAAGAAGAGGAGGAGAAAGAAGAGGA | | |
| | G*G*A+ | | |
| HDR donor | +C*G*GTGGAGGAGGTGCTGGGCCACTTCGGCGTCAACGAGAG | 168 | 423 |
| | GAATTCTACGGGGCTGAGCCTGGAACAGGTCAAGAAGCTTAAG | | |
| | G*A*G+ | | |
| HDR donor | +C*T*TCAGTGCATCTCTCACTGCTTTTGCACAGATCTGGGAG | 169 | 424 |
| | GAATTCTATCGGATGTAGCTGGGAGAAAATGAGAGAAGGTATA | | |
| | T*G*G+ | | |
| HDR donor | +C*T*GGAGGTCAAACTGGGGGAGCTGCCAAGCTGGATCTTGA | 170 | 425 |
| | GAATTCTGCGGGACTTCAGTCCTAGTGGCATTTTCGGAGCGTT | | |
| | T*C*A+ | | |
| HDR donor | +A*A*GCCTCGATTGGCCGCATTTTGGTACTACACCACGGTTG | 171 | 426 |
| | GAATTCAGCTGGTTCCTCCCACCCCTGCTGAGATCCCTAGAGC | | |
| | T*A*T+ | | |
| HDR donor | +T*G*AACTGAGAACAGAGTTTTCTTGAAACAGGCGATTACGG | 172 | 427 |
| | GAATTCAGCTGGCGCAAGGTGACTGAAAGGTCTTCAAACACTG | | |
| | A*G*T+ | | |
| HDR donor | +G*A*TCTCCCTCCGCTCCTCGGCTGTGTTTGGATAGGGGTAC | 173 | 428 |
| | GAATTCACGTGGCAGTGGTAGCTGGAAGACAAGAGTTGAACAC | | |
| | A*G*T+ | | |
| HDR donor | +T*T*CTCCTGGGTCTCCTTCTCCACTTCAGTGCTGCAACTGC | 174 | 429 |
| | GAATTCCACGGGATCCCAATGCCTGGAGATGGGGAGTGATGTC | | |
| | A*C*G+ | | |
| HDR donor | +C*A*GCATTCACCTGGAAGGTCCAGGCCAACAACCGTGCCTA | 175 | 430 |
| | GAATTCCAACGGGCAGTTCAAGGAGAAGGTGATCCTGTGCTGG | | |
| | C*A*A+ | | |
| HDR donor | +T*A*CTTGGTGGTGTGTAACGACTGTAATCAGGTTGTCAAAC | 176 | 431 |
| | GAATTCCGCAGGCATTTCAATCACATTATGGTAAGTGCTTAAC | | |
| | C*A*T+ | | |
| HDR donor | +C*T*TCGGGATTTTTACCTGGACCAGTTCGGCAGCAACGTTG | 177 | 432 |
| | GAATTCAGTCGGAGGCAGAGAGGCAGCTCTTGAAGGGCTCGAA | | |
| | C*C*A+ | | |
| HDR donor | +T*G*AACAAGACATCCTCTTTCTCCTGCGAAGCCCATAACGC | 178 | 433 |
| | GAATTCCAAGGGGGTCACCACATCCCGCACAGCCACCATCACA | | |
| | G*G*T+ | | |
| HDR donor | +G*G*CTGGGTCCCAGCCATCCAGGAACTCTAACACGTTGGGG | 179 | 434 |
| | GAATTCACGTGGACAAAGACATCGTCATCTCCCTTTAGCATGA | | |
| | A*A*T+ | | |
| HDR donor | +C*C*TGCGGGGGCTTTGCGGGGGCGCCCACGGCGCAAGAGGT | 180 | 435 |
| | GAATTCAGCCGGAGGCCGGGCGCGTCCCGGGTGCTCGCGTACA | | |
| | G*G*A+ | | |
| HDR donor | +T*G*CAGGCCTTGGGCTTCTCAGGAACCAATGGGCGCTTACG | 181 | 436 |
| | GAATTCAAGTGGGTAATTCTTGCGGCGCCCTGTGAGGTGACCT | | |
| | G*G*G+ | | |
| HDR donor | +C*T*GCAGGTGCTGCAACATGGTCTGGAACTCTGAGTCATAG | 182 | 437 |
| | GAATTCCGTCGGTTGATGTCGTCCCCGATGATGGCGAGCTGCC | | |
| | G*T*C+ | | |
| HDR donor | +A*G*CCTGATCAAGATGACAACCTCCCAAAAGCACCGAGACT | 183 | 438 |
| | GAATTCTCGTGGCAGAGCCCATGGGGGAGAAGCCAGTGGGGAG | | |
| | C*C*T+ | | |
| HDR donor | +C*T*CCTGTGGGCAAGAGAATAGGTGGTTTGGGGGACACACG | 184 | 439 |
| | GAATTCGGTTGGAGGCCCGTGCATATCCCAGGTGAGAAATGGC | | |
| | A*C*C+ | | |
| HDR donor | +A*G*AAAGTAGGCATAGTAAGACTCACCGGAGCATCTGACAA | 185 | 440 |
| | GAATTCACCAGGAGAAGTTTCAAAACTTGGGAAAAGGATGGGT | | |
| | T*T*C+ | | |
| HDR donor | +T*T*GAAACCTTGCAGAGCACAATTGCCACCAATAATCGCAA | 186 | 441 |
| | GAATTCGAGGGGAAGAAATGTCTCCCAATGTCCCCAGCACAAT | | |
| | T*G*C+ | | |
| HDR donor | +G*C*AAGCCTTCCCAGAAACATGCCGGTGAATCACCAGTTCC | 187 | 442 |
| | GAATTCCCCTGGCCTCATCCATGGACCTTCTGAGCAGCAGGTC | | |
| | C*C*C+ | | |
| HDR donor | +C*G*CTGGACTGGGGGATCCGGCGGCTGCGGAACCCCACTTT | 188 | 443 |
| | GAATTCCCACGGCACGCCGCTTAGACCTGGACGCCATGTTGCC | | |
| | G*C*T+ | | |
| HDR donor | +T*T*CCTAGCTTCATAAAGAGATGTTACCGCCCAAGGAGCAT | 189 | 444 |
| | GAATTCTAAAGGATCCACTTTGCAGAATGGCTCTGGTGAACAA | | |
| | T*G*A+ | | |
| HDR donor | +C*A*GCCATGACTGACAATACTAATGTCAACTATGTGCGGTA | 190 | 445 |
| | GAATTCCAAGGGTGATTACTACCTCTGCACTGAGACCAACTTT | | |
| | A*T*G+ | | |
| HDR donor | +C*C*GGCTGAAGGAAATCAACTGTTTCACCGCCACGTTTGAG | 191 | 446 |
| | GAATTCATCTGGGTGGAGGGCCCCTTGGGCGTCATCAATAACT | | |
| | T*C*A+ | | |
| HDR donor | +C*C*TCATGGTCTTGAGTTCTTGTAGAATGGCCTGCAACGTT | 192 | 447 |
| | GAATTCGACTGGCAGTTACAAGTACAGCAGTTTGATCCAGCTG | | |
| | A*T*G+ | | |
| HDR donor | +C*C*CTTTGGATGCTGGTGAATACTGTGTGCCTAGAGGAAAT | 193 | 448 |
| | GAATTCCGTAGGCGGTTCCGCGTTAGGCAGCCCATCCTGCAGT | | |
| | A*T*A+ | | |
| HDR donor | +C*T*CCATCTCCATCTATCATATCAATGTTATCGACAAGCCT | 194 | 449 |
| | GAATTCATTGGGCACATCCTCTCCAAAACCCGGGGCAGGTGGA | | |
| | G*C*C+ | | |
| HDR donor | +C*T*GGCGGCCGGGGAGCGGCCGTCCGACCCCGGGGAACACC | 195 | 450 |
| | GAATTCCTCAGGCCGAGCCTGGGTCTCTGGCCGAGGGGGCCGG | | |
| | A*C*C+ | | |
| HDR donor | +A*G*GCTGTGGGCCAGTTCTGACTGCAACGAGGCAGCCGACA | 196 | 451 |
| | GAATTCCGTCGGCGTCCTGGAGGCGTGACTCCTCCTCCAGCTC | | |
| | G*G*A+ | | |
| HDR donor | +A*G*GTCCATACCCCACATTGAGGTGATCCACCAAAGCTTCT | 197 | 452 |
| | GAATTCGTCAGGCAGGTCGCTGCAGTGATAGCCTCTCGCCTCC | | |
| | T*C*T+ | | |
| HDR donor | +A*A*ATTTTGAAATCTCTTGTTCCAGTGTGTCTAACAATACA | 198 | 453 |
| | GAATTCACTTGGTTCTGTCTGAAACAAAAACACACAGCCAAGA | | |
| | C*A*A+ | | |
| HDR donor | +C*A*AAGACATTGTGAGCCACCTCGACACGAAGCCAATCAGG | 199 | 454 |
| | GAATTCTTCAGGAACGCTGAGGAAGGGTGTGGATTCCGCAAGT | | |
| | C*C*C+ | | |
| HDR donor | +A*G*GCTCTGGGCAAACAGGTCAGCAGAGAGCAAGCTCCCGG | 200 | 455 |
| | GAATTCGTTGGGTCACCGGCTCCCCATCCTCTGGTTGGAACAC | | |
| | A*T*C+ | | |
| HDR donor | +C*T*GACCAGCGCGCAGGACAGCGCCTTCCTCAACGACGCGG | 201 | 456 |
| | GAATTCACATGGTCATGAGCTTTGTGAACCTGGGTAAGGATTT | | |
| | G*G*G+ | | |
| HDR donor | +G*G*AGCAGGAGAAGCTCTCCGGTGTGGTGAAGAGCGTCCAC | 202 | 457 |
| | GAATTCCGGCGGCTCCGCAAGAAGTACCGAGAAGGTAAACAGC | | |
| | G*C*G+ | | |
| HDR donor | +C*T*GCTGGCAACACAGGAACTTCCCGTCCACGAAGAACCCA | 203 | 458 |
| | GAATTCCTATGGTACTTGACCAGCAGGTGGGGGTTACCCCTTA | | |
| | T*C*T+ | | |
| HDR donor | +G*G*AGAAGATGGACATCACTGGAGGGTGTTCCGAATGGGAC | 204 | 459 |
| | GAATTCCACGGGAGCAGCGCGTAGACATGACTGTCATTGAGCC | | |
| | C*T*A+ | | |
| HDR donor | +T*G*AAATTACTTACAGGCCAAGGCGGTGGGACCGAAGTTAC | 205 | 460 |
| | GAATTCCTCTGGAGTATGAAAATAAGCAACACCATTATGGTAA | | |
| | G*T*A+ | | |
| HDR donor | +G*T*GGGCAACGTGCCCTTGGAGTGGTACGATGACTTCCCCC | 206 | 461 |
| | GAATTCACGTGGGCTACGACCTGGATGGCAGGCGCATCTACAA | | |
| | G*C*C+ | | |
| HDR donor | +G*T*CATAATTAACACACATCAGTGGAACTTCTGTACTACAA | 207 | 462 |
| | GAATTCCGCTGGTGAAATTTATAACCACATGTTTGACAGCGGA | | |
| | A*A*C+ | | |
| HDR donor | +C*G*TCAAGCAGTAAAAACACCCCCCCGGAGGTTACCCACTG | 208 | 463 |
| | GAATTCTGATGGTTCGCTCTCCTATAGATTCTGCCTCCCCAGG | | |
| | A*G*G+ | | |
| HDR donor | +T*C*TGTATTTCCTAAGACATCTATTTCTCCCTTGAACGTGG | 209 | 464 |
| | GAATTCTACAGGGAGCTTCAGTCAACTCCAGTTCACAAACTGC | | |
| | G*G*C+ | | |
| HDR donor | +G*C*GGGCGGCGAGGCAAGATGGCGGCAACCAAGAGGAAACG | 210 | 465 |
| | GAATTCGCGTGGAGGCTTTGCAGTTCAGGCGAAGAAGCCAAAA | | |
| | A*G*A+ | | |
| HDR donor | +A*C*TGCTCTTTGATTCTTCCCCGGCAGGACGTCCGCCACAT | 211 | 466 |
| | GAATTCACTTGGTGATGGCAGCACTCTGTAACTGCAGCCTCTC | | |
| | A*C*A+ | | |
| HDR donor | +A*A*CCTTGCGTTTATTTTCAATGCTCAACGCCAGATCTTGT | 212 | 467 |
| | GAATTCCGTTGGCATTCTACTTCAGCCCAGTTGAGAGCTGCCT | | |
| | C*A*A+ | | |
| HDR donor | +G*C*CAGTTTTTTTAGAGAACTCTGAAGCTTTTTGTCATCAG | 213 | 468 |
| | GAATTCCTGTGGCTGTTCTATGTACCACCTTCTTCTTTCTGCG | | |
| | A*G*C+ | | |
| HDR donor | +C*T*GGCTGTCTTTTCCAGGCCGAGGAGGGAACTTCTGATGG | 214 | 469 |
| | GAATTCTACAGGATACACCCTCCCCAGAGCTGCCTCTCATGAT | | |
| | C*A*C+ | | |
| HDR donor | +T*A*TCCTTCCACAGCACTGGGTTCTAATCTTCACGTCGAAG | 215 | 470 |
| | GAATTCTGAAGGGTTATGAGGATGGAGGGATCCATCTGGAGTG | | |
| | C*A*G+ | | |
| HDR donor | +T*T*CTTTGCTTTGGCACTGTTAGGGTAGTCTACCACCATGC | 216 | 471 |
| | GAATTCCACCGGAGAAGCCTGCCTTTGTGGCCTGGGTTGTGAT | | |
| | C*A*G+ | | |
| HDR donor | +C*A*AGACTGTATTATTCAAGGCTTAACTGAAACCGGTACTG | 217 | 472 |
| | GAATTCATTTGGAAGCAGTAGCTAAGTTTCTTGATGCTTCTGG | | |
| | A*G*C+ | | |
| HDR donor | +A*G*CAATGGAGACTCAGGCTACCATCGGAGTCGCTGCAAAG | 218 | 473 |
| | GAATTCTCGGGGACAAGTGATGAAGCGGCCGAATAACATTTTA | | |
| | A*A*G+ | | |
| HDR donor | +T*C*ACTGCTTTTCCTCCTCCTTGAGGGGGACCTTTGGCACA | 219 | 474 |
| | GAATTCCGCTGGGAGGCCATGAAAGCCAAGGCCACCGAGCTGC | | |
| | G*G*G+ | | |
| HDR donor | +C*C*CGCAGAGGGTTTGCATCCATCACCATCACGGCCAGACG | 220 | 475 |
| | GAATTCCGTGGGCCCCCCAGCCCGCGCCCTGGTGTGGGGGACT | | |
| | G*C*T+ | | |
| HDR donor | +A*G*AACTGGAAAAACAGATGGAAGATGCTTACCGGGGGACC | 221 | 476 |
| | GAATTCAAAAGGAAAATGCTACCCAGCAGTTCAAGGTGAAGTT | | |
| | G*C*A | | |
| HDR donor | +G*G*CTTCACTTCACAGGTAGGAGGCTGGGCCACAAAAGGGA | 222 | 477 |
| | GAATTCTACAGGAAGGAATGCTGGTGCTTACATCCTGCTCCAC | | |
| | T*T*C+ | | |
| HDR donor | +G*T*CAGAATAGGGACACTTACCCAGTCCTGGGTGCTGATGT | 223 | 478 |
| | GAATTCCATGGGTGTCATGGAGCAGTTGCTCATCCCTAAAGTG | | |
| | G*A*C+ | | |
| HDR donor | +T*G*GGAGCAAACAAGGGAACGTGCAGACGCTGGACAGCATA | 224 | 479 |
| | GAATTCCGATGGATGCCTGCAACTCCTGTACCTGCCCCCGAAT | | |
| | G*C*C+ | | |
| HDR donor | +C*A*GAGGAGTTAAACAGGGAGTACTCGGTGCTGAAGTCCTC | 225 | 480 |
| | GAATTCGTTGGGCGAGTCCATGAAAGCTCCCCCCATCATGGGC | | |
| | A*G*C+ | | |
| HDR donor | +T*G*ATGGAACTCGAGCTGGGGGCCGGGGTACGGCTGAAGAC | 226 | 481 |
| | GAATTCTCGGGGCGATGGTCCCTTCCTGGGTGAGCAATGCAAC | | |
| | C*C*T+ | | |
| HDR donor | +T*G*CCCTTAGCCTTGCTTCCTGGCCTGGTTTCATGGTTCCT | 227 | 482 |
| | GAATTCCCGGGGACTAGAGAGGGAAATGCCCAGGTCCTCTTGG | | |
| | G*C*C+ | | |
| HDR donor | +C*C*CAGACGACCGAGCTGACCTGCACGGAATCCCACAGCTG | 228 | 483 |
| | GAATTCGTAGGGCAAGTAGTCCGGGCTGACGCTATCAGGGAAG | | |
| | C*C*C+ | | |
| HDR donor | +T*T*TCTCGAGGGAGAAAAAGGGGAATCCTCTGTCCAGAATG | 229 | 484 |
| | GAATTCAGCAGGAAGGAGAGCCAAGCCTACAGTCACCCAGCTT | | |
| | A*G*A+ | | |
| HDR donor | +C*A*AGGACGCCCCGGCCACCCTGACGCACCCCCCAACATCT | 230 | 485 |
| | GAATTCACGAGGGGGGCCTGGGGTCCCCGCAGCCGCAGTGCCC | | |
| | C*A*G+ | | |
| HDR donor | +G*C*AGTCACTGATGTCCCTTTTCAAAGGATGCACGCTCCCC | 231 | 486 |
| | GAATTCACCGGGCGCCGGAGGTGTTTTGCAGCCGCTCTTCCAG | | |
| | A*G*G+ | | |
| HDR donor | +C*T*CACCCCCGACGGCTTCTTCTTCCGAGTCCACATGTTAG | 232 | 487 |
| | GAATTCCCCTGGACTCCTCCAGCTGCAATAAGCCGTGTCCAGA | | |
| | G*T*T+ | | |
| HDR donor | +C*T*GCCAGAGCCAGTGTCTGAGCTGCGGGCCAACTTCACTC | 233 | 488 |
| | GAATTCTGCAGGACAGAGGGGCAGGCCCCAGGGTGGAGATGAT | | |
| | C*T*G+ | | |
| HDR donor | +C*C*AACGGCGAGTCCCGGTGGGCCGCCCACCAAACCCCCGA | 234 | 489 |
| | GAATTCCGAAGGCCATGGCCCCTGTGACCAGGGCACCCTTCCC | | |
| | A*G*A+ | | |
| HDR donor | +C*C*ACGGGGGAGATCCCAAGCTCAGTCCCCACAAAGTTCAG | 235 | 490 |
| | GAATTCGGCCGGTCGGAGGCAGGGGCAGGTCCGGGTCCAAAGG | | |
| | T*A*A+ | | |
| HDR donor | +C*G*GCCCTGACTACGACTTCGCGCGCTACCGGAGCACAGTG | 236 | 491 |
| | GAATTCCACGGGGTGACCCAGGCCTTCGCCGCCGCCTCGCGGG | | |
| | A*G*G+ | | |
| HDR donor | +T*G*CCTCCTGCACAGCGCCCTGCTGGGGGCCTACACCTTCC | 237 | 492 |
| | GAATTCAACAGGCCTTGCCCTCTTGCCCCTGCTGCTCCCAGGC | | |
| | A*G*G+ | | |
| HDR donor | +T*T*TTCTCTTGTAGTTTATTTGGCAACCCAAGGAGTTAATC | 238 | 493 |
| | GAATTCCTAAGGAACATCCAGTAAAACAGGAATTGGTAAGATT | | |
| | T*T*A+ | | |
| HDR donor | +A*T*CTTTGTGTTTCAACTGGCTTTTTATTATGGACTGGTGC | 239 | 494 |
| | GAATTCTTAAGGCTCTGCCTAAATGAATAAAAAGAAAAGAATA | | |
| | C*A*A+ | | |
| HDR donor | +A*G*GAGGTCCAGAGGAGACCATCACTCAGCAAGGACGAACG | 240 | 495 |
| | GAATTCCCAAGGACAGTAACTGAGTCCAGCTCATCCCACCCTC | | |
| | C*T*G+ | | |
| HDR donor | +G*G*CACTGGGGCTCGGGCATGGCCTGTGCTGGTAGGATTTG | 241 | 496 |
| | GAATTCTGCTGGGGGCTGTGGTCCTCTCGCTCCTCATTGCACT | | |
| | T*G*C+ | | |
| HDR donor | +G*C*CTGGCGCACATCGTAACCGACCAGGTCTTTGATCAACT | 242 | 497 |
| | GAATTCCGATGGCTGCGGAAGGGCAGAGGTGCTGGAGAGAGGC | | |
| | C*C*C+ | | |
| HDR donor | +C*A*AAAGGATTATGTGATTCTTGCCACTAGAACGCCACCCA | 243 | 498 |
| | GAATTCAAGAGGAGCAAAGTGAGAACCTCAAACATCCCAAAGC | | |
| | T*A*A+ | | |
| HDR donor | +C*T*GTCCGTGGTGCTGGATGTGGCTCTATCGTCCACACGGA | 244 | 499 |
| | GAATTCGGAAGGAGGGTGGGAGGGTGGTGCAGAACCCACCCGC | | |
| | C*C*C+ | | |
| HDR donor | +A*T*AATAGCTCTGGCAAGGAGAAGCACCCTGGACATAGCAC | 245 | 500 |
| | GAATTCGTCTGGGTCTGGCAGGGAGCTGGACTTTTGATGTAGG | | |
| | T*C*A+ | | |
| HDR donor | +G*C*CAGGTCGAAGGCGCCGTCCAGGTTCACCAGCTCCGTGT | 246 | 501 |
| | GAATTCCGAAGGGCACCGCCTGGAAGTGGTCGGAGCTGTGCAG | | |
| | G*C*C+ | | |
| HDR donor | +T*G*AGACCCTCAACTGCTCCTCCTGCCACCACACAGCCGAC | 247 | 502 |
| | GAATTCGAATGGAACTGGCTTGATGCGTGCTCCAGGAAGACTA | | |
| | T*G*G+ | | |
| HDR donor | +A*G*TCTTGGCTGGACTCACTGCCCACCTCTGGGACCTTGGC | 248 | 503 |
| | GAATTCGGTGGGGCGGGAAGGAGGACCTCAAAGGCTCAGCGAG | | |
| | T*C*C+ | | |
| HDR donor | +C*T*GAACAGGATCGTTCAGCTGCACGACGCACACTGCATTA | 249 | 504 |
| | GAATTCATGTGGCCTGTAATTAAATAGAAGGGCATCGTGTTGG | | |
| | C*G*T+ | | |
| HDR donor | +T*G*CTATACATATGGATTCAAAAGTGGATGATCACTTAATA | 250 | 505 |
| | GAATTCCGAGGGACTGAAAAAAGCAGGTTGGAACCAGCGACTC | | |
| | A*G*T+ | | |
| HDR donor | +T*A*GATACTGTAGAGAAATCTGTGGGGTTGACCCCAAAGCT | 251 | 506 |
| | GAATTCAACAGGTAGAGCTAAGGAATCCTTAGGGATGCTGCTG | | |
| | C*A*G+ | | |
| HDR donor | +C*A*TGAGTCCAGGGGGCACGTAGGCGGCATTGAACTCGGTC | 252 | 507 |
| | GAATTCAGTAGGGTGCCCGCGCTGCGGGAGGCCATGGTGGGTG | | |
| | C*G*C+ | | |
| HDR donor | +A*A*GGTAAAGAGACAAAGAAAGTGCCTTGTACAGATGCAAA | 253 | 508 |
| | GAATTCCGGAGGTGTAGACTGTGCAGCTGCCAAAGTGGTGACA | | |
| | A*G*C+ | | |
| HDR donor | +T*G*GCTGTTGGGGTCTACTCAGCCAAGAATGCCACGCTTGT | 254 | 509 |
| | GAATTCCGCCGGCCGCTTCATCGAGGCTCGGCTGGGGAAGCCG | | |
| | T*C*C+ | | |
| HDR donor | +G*T*GTCCCTGCATCTGCAGGCCATGTCAAAGGACGCCCTGA | 255 | 510 |
| | GAATTCATCTGGCGCAGATGCAGGAGCAGACGCTGCAGTTGGA | | |
| | G*C*A+ | | |
| NGS F primer | acactctttccctacacgacgctcttccgatctCACCTTCAGT | 1 | 511 |
| | AACCTTTTTCATCT | | |
| NGS F primer | acactctttccctacacgacgctcttccgatctAAAAGTGCCG | 2 | 512 |
| | CTGAAGTG | | |
| NGS F primer | acactctttccctacacgacgctcttccgatctGCTCAGAATC | 3 | 513 |
| | TGTTCTATGCC | | |
| NGS F primer | acactctttccctacacgacgctcttccgatctCGCCTATCTA | 4 | 514 |
| | CTCACGTTG | | |
| NGS F primer | acactctttccctacacgacgctcttccgatctACAGCAGATG | 5 | 515 |
| | TTTAACAGACTT | | |
| NGS F primer | acactctttccctacacgacgctcttccgatctATTAACCAAC | 6 | 516 |
| | TCACCAAAGACAG | | |
| NGS F primer | acactctttccctacacgacgctcttccgatctAGAGGGCTGA | 7 | 517 |
| | CAGAAATAATAAC | | |
| NGS F primer | acactctttccctacacgacgctcttccgatctGCCGAGTGAA | 8 | 518 |
| | ATGTACGTC | | |
| NGS F primer | acactctttccctacacgacgctcttccgatctCACAGACTGC | 9 | 519 |
| | AGCCAAC | | |
| NGS F primer | acactctttccctacacgacgctcttccgatctCAAAGCTGGC | 10 | 520 |
| | ATGAACC | | |
| NGS F primer | acactctttccctacacgacgctcttccgatctCCCAGAACTT | 11 | 521 |
| | TGTGTATCTTTCT | | |
| NGS F primer | acactctttccctacacgacgctcttccgatctTAAGCTTCTC | 12 | 522 |
| | TTGGACCTTGA | | |
| NGS F primer | acactctttccctacacgacgctcttccgatctCACATTAAAA | 13 | 523 |
| | GTGCACAGAAAACG | | |
| NGS F primer | acactctttccctacacgacgctcttccgatctAGACTCCGAA | 14 | 524 |
| | GCTGACCT | | |
| NGS F primer | acactctttccctacacgacgctcttccgatctGCTAGTAACA | 15 | 525 |
| | GTTCTGGGTG | | |
| NGS F primer | acactctttccctacacgacgctcttccgatctCAAGATCTTG | 16 | 526 |
| | GCGATGGA | | |
| NGS F primer | acactctttccctacacgacgctcttccgatctACTCGCCCAG | 17 | 527 |
| | GTAGGA | | |
| NGS F primer | acactctttccctacacgacgctcttccgatctGGCACTGAAT | 18 | 528 |
| | TTTGGAGATCTTTG | | |
| NGS F primer | acactctttccctacacgacgctcttccgatctTATTAACTCT | 19 | 529 |
| | GGGCTGCTGT | | |
| NGS F primer | acactctttccctacacgacgctcttccgatctAAGGTCATCG | 20 | 530 |
| | CCCCAGA | | |
| NGS F primer | acactctttccctacacgacgctcttccgatctTGACCAGGGA | 21 | 531 |
| | GTCTTACCTT | | |
| NGS F primer | acactctttccctacacgacgctcttccgatctCCACCAACCT | 22 | 532 |
| | TGTTTCTGT | | |
| NGS F primer | acactctttccctacacgacgctcttccgatctCAGTGACTGG | 23 | 533 |
| | CCAACATTTA | | |
| NGS F primer | acactctttccctacacgacgctcttccgatctGGGTTTGCAA | 24 | 534 |
| | AATATTTGTATTAACATT | | |
| NGS F primer | acactctttccctacacgacgctcttccgatctCGGCAGGAGA | 25 | 535 |
| | AGCAAGAT | | |
| NGS F primer | acactctttccctacacgacgctcttccgatctAAGTGCCTCT | 26 | 536 |
| | TCCTTCTGAG | | |
| NGS F primer | acactctttccctacacgacgctcttccgatctGTTGTAATTG | 27 | 537 |
| | ATTCTAGGAATTGGAT | | |
| NGS F primer | acactctttccctacacgacgctcttccgatctCTGCTACATC | 28 | 538 |
| | TTGAACCTGG | | |
| NGS F primer | acactctttccctacacgacgctcttccgatctCTTAGATTAC | 29 | 539 |
| | TCTTGTCTCTGCTG | | |
| NGS F primer | acactctttccctacacgacgctcttccgatctCCCCATTCAG | 30 | 540 |
| | TTGTTCTCAG | | |
| NGS F primer | acactctttccctacacgacgctcttccgatctCATTCAACCA | 31 | 541 |
| | CTTCCCTGT | | |
| NGS F primer | acactctttccctacacgacgctcttccgatctTAGAGTATGC | 32 | 542 |
| | AATCTGGGCA | | |
| NGS F primer | acactctttccctacacgacgctcttccgatctACAGAGGGAA | 33 | 543 |
| | ATGACATTGC | | |
| NGS F primer | acactctttccctacacgacgctcttccgatctCAGGTAGTCT | 34 | 544 |
| | CTGCCTTC | | |
| NGS F primer | acactctttccctacacgacgctcttccgatctCCTCCAGTCC | 35 | 545 |
| | TTACTTGAACTT | | |
| NGS F primer | acactctttccctacacgacgctcttccgatctCTGACAGCAA | 36 | 546 |
| | AAGACATCCT | | |
| NGS F primer | acactctttccctacacgacgctcttccgatctCCTCAGACTT | 37 | 547 |
| | TCTTCCCTTC | | |
| NGS F primer | acactctttccctacacgacgctcttccgatctCAGAGGAACC | 38 | 548 |
| | TAATCTGTGT | | |
| NGS F primer | acactctttccctacacgacgctcttccgatctATACCTTCCG | 39 | 549 |
| | CAGCTTCC | | |
| NGS F primer | acactctttccctacacgacgctcttccgatctCACCGCATGA | 40 | 550 |
| | TTAGACAGGTA | | |
| NGS F primer | acactctttccctacacgacgctcttccgatctCAGCATTTAC | 41 | 551 |
| | CAGATTGCACT | | |
| NGS F primer | acactctttccctacacgacgctcttccgatctTAGGTGCTAT | 42 | 552 |
| | ACTTGGTAGATCAGAAA | | |
| NGS F primer | acactctttccctacacgacgctcttccgatctGCAGGTCAAG | 43 | 553 |
| | GACAAAGT | | |
| NGS F primer | acactctttccctacacgacgctcttccgatctCTTTTAACTG | 44 | 554 |
| | CAGTAGGTAGGA | | |
| NGS F primer | acactctttccctacacgacgctcttccgatctTCTTCCCCAA | 45 | 555 |
| | ACCCCAC | | |
| NGS F primer | acactctttccctacacgacgctcttccgatctTCATTGTTTT | 46 | 556 |
| | TACCAAGGATCCAT | | |
| NGS F primer | acactctttccctacacgacgctcttccgatctGAAATTCTGT | 47 | 557 |
| | AGTACACCCAGTC | | |
| NGS F primer | acactctttccctacacgacgctcttccgatctGCCCGTAGGT | 48 | 558 |
| | ATCGTTCTTC | | |
| NGS F primer | acactctttccctacacgacgctcttccgatctCATGTTTATT | 49 | 559 |
| | TGTTGTCTTGCACG | | |
| NGS F primer | acactctttccctacacgacgctcttccgatctGCCCTTCTAA | 50 | 560 |
| | GACCATTGTGTTA | | |
| NGS F primer | acactctttccctacacgacgctcttccgatctTGGAGTCGAA | 51 | 561 |
| | ACTGACCT | | |
| NGS F primer | acactctttccctacacgacgctcttccgatctGAGGTGTCAT | 52 | 562 |
| | GGTAGTTGG | | |
| NGS F primer | acactctttccctacacgacgctcttccgatctGACCTTGAAA | 53 | 563 |
| | GCAATTGTGGA | | |
| NGS F primer | acactctttccctacacgacgctcttccgatctCCTTCCAACA | 54 | 564 |
| | GTTTTTCTTTGTC | | |
| NGS F primer | acactctttccctacacgacgctcttccgatctGAAGTACCCA | 55 | 565 |
| | CTGCCATC | | |
| NGS F primer | acactctttccctacacgacgctcttccgatctAGCTGCTCTT | 56 | 566 |
| | GACGACT | | |
| NGS F primer | acactctttccctacacgacgctcttccgatctGCAATTAGCA | 57 | 567 |
| | TCAAGGGTTTG | | |
| NGS F primer | acactctttccctacacgacgctcttccgatctCTTTTACTCC | 58 | 568 |
| | TCCCATGTTCTTT | | |
| NGS F primer | acactctttccctacacgacgctcttccgatctTGTGGCTCAT | 59 | 569 |
| | CTCGGC | | |
| NGS F primer | acactctttccctacacgacgctcttccgatctACCACCAGGA | 60 | 570 |
| | GTCCCAT | | |
| NGS F primer | acactctttccctacacgacgctcttccgatctCTCAGCTGCC | 61 | 571 |
| | TCCTGG | | |
| NGS F primer | acactctttccctacacgacgctcttccgatctGTTTTCTTCC | 62 | 572 |
| | CCTTCCCATC | | |
| NGS F primer | acactctttccctacacgacgctcttccgatctCCTTGGCAGT | 63 | 573 |
| | GGTTTCTC | | |
| NGS F primer | acactctttccctacacgacgctcttccgatctTCTGCCCTCT | 64 | 574 |
| | GACCCA | | |
| NGS F primer | acactctttccctacacgacgctcttccgatctCTCCCTCTAT | 65 | 575 |
| | ACATATAGCTTTGGA | | |
| NGS F primer | acactctttccctacacgacgctcttccgatctTTCAAAGTGC | 66 | 576 |
| | CACGTTTG | | |
| NGS F primer | acactctttccctacacgacgctcttccgatctATGGCTTTCT | 67 | 577 |
| | GGACTTCATC | | |
| NGS F primer | acactctttccctacacgacgctcttccgatctGAAACCAATC | 68 | 578 |
| | AAGCTCCTGG | | |
| NGS F primer | acactctttccctacacgacgctcttccgatctGCCACTGGAA | 69 | 579 |
| | TTAGACAAGC | | |
| NGS F primer | acactctttccctacacgacgctcttccgatctCCATTTCCCC | 70 | 580 |
| | AGGATGA | | |
| NGS F primer | acactctttccctacacgacgctcttccgatctGTTCTCTCTG | 71 | 581 |
| | GCCATCTG | | |
| NGS F primer | acactctttccctacacgacgctcttccgatctGCAGAACCCA | 72 | 582 |
| | CTAATACAAAGGA | | |
| NGS F primer | acactctttccctacacgacgctcttccgatctGTCTTCTGGC | 73 | 583 |
| | TGTTCTATAGATC | | |
| NGS F primer | acactctttccctacacgacgctcttccgatctTGGTTTCCTC | 74 | 584 |
| | TCTCCGAG | | |
| NGS F primer | acactctttccctacacgacgctcttccgatctCTACCCTTTT | 75 | 585 |
| | CTCTACCCAGG | | |
| NGS F primer | acactctttccctacacgacgctcttccgatctCCAGCATCTT | 76 | 586 |
| | TCAAACCAATTTT | | |
| NGS F primer | acactctttccctacacgacgctcttccgatctTTGGGTCAGT | 77 | 587 |
| | GCCTTAC | | |
| NGS F primer | acactctttccctacacgacgctcttccgatctTCCTGAGTTT | 78 | 588 |
| | ACATACGTCATCTTT | | |
| NGS F primer | acactctttccctacacgacgctcttccgatctGTCACTGATT | 79 | 589 |
| | CTCTCTTCTCTG | | |
| NGS F primer | acactctttccctacacgacgctcttccgatctCACATATGTA | 80 | 590 |
| | CACACAAGAAAATCACATA | | |
| NGS F primer | acactctttccctacacgacgctcttccgatctCTTGAAACAG | 81 | 591 |
| | AGTTCTCCCTAAAG | | |
| NGS F primer | acactctttccctacacgacgctcttccgatctAAGATCTTTG | 82 | 592 |
| | TCTCTTCCCACTT | | |
| NGS F primer | acactctttccctacacgacgctcttccgatctAGATGTGGTA | 83 | 593 |
| | TTTAGCAAGAGTCA | | |
| NGS F primer | acactctttccctacacgacgctcttccgatctTCCAGGTCAC | 84 | 594 |
| | AGTTCTTGT | | |
| NGS F primer | acactctttccctacacgacgctcttccgatctTCTTCTCTTA | 85 | 595 |
| | GGGTTGGATGG | | |
| NGS F primer | acactctttccctacacgacgctcttccgatctTATTTGTAGG | 86 | 596 |
| | TGCAGGAAGCT | | |
| NGS F primer | acactctttccctacacgacgctcttccgatctTCGCCTCCTC | 87 | 597 |
| | GATACTTAC | | |
| NGS F primer | acactctttccctacacgacgctcttccgatctTGGGTTCCCA | 88 | 598 |
| | GGTCTG | | |
| NGS F primer | acactctttccctacacgacgctcttccgatctAGTATTACAG | 89 | 599 |
| | CCGCCTCAT | | |
| NGS F primer | acactctttccctacacgacgctcttccgatctTTGGGCTCCT | 90 | 600 |
| | TATCCGT | | |
| NGS F primer | acactctttccctacacgacgctcttccgatctCTGTCCCAGT | 91 | 601 |
| | TATAATGGTAGC | | |
| NGS F primer | acactctttccctacacgacgctcttccgatctAGACGCTGAG | 92 | 602 |
| | CCGAGAA | | |
| NGS F primer | acactctttccctacacgacgctcttccgatctCCACTACTTC | 93 | 603 |
| | TTTTCCATTGAGG | | |
| NGS F primer | acactctttccctacacgacgctcttccgatctAACACCTCCA | 94 | 604 |
| | GAACAAAGG | | |
| NGS F primer | acactctttccctacacgacgctcttccgatctGATGTTCCAC | 95 | 605 |
| | GCTGTTC | | |
| NGS F primer | acactctttccctacacgacgctcttccgatctCAGGTTCCTT | 96 | 606 |
| | TGCCAAACT | | |
| NGS F primer | acactctttccctacacgacgctcttccgatctCGCCAACTGT | 97 | 607 |
| | AAAATCCTGA | | |
| NGS F primer | acactctttccctacacgacgctcttccgatctGCTCCAGTGC | 98 | 608 |
| | ATGATGAG | | |
| NGS F primer | acactctttccctacacgacgctcttccgatctTGGTGATGAG | 99 | 609 |
| | ACTGCAGG | | |
| NGS F primer | acactctttccctacacgacgctcttccgatctAGGAACCATT | 100 | 610 |
| | GATGATGCTGT | | |
| NGS F primer | acactctttccctacacgacgctcttccgatctCATGCAGGTG | 101 | 611 |
| | AATTACACGA | | |
| NGS F primer | acactctttccctacacgacgctcttccgatctGTTGGTGCCT | 102 | 612 |
| | AAACGTTCTA | | |
| NGS F primer | acactctttccctacacgacgctcttccgatctGTCCCATCCT | 103 | 613 |
| | AGTTTGGC | | |
| NGS F primer | acactctttccctacacgacgctcttccgatctTAATGTCGAC | 104 | 614 |
| | TTACCCACAGG | | |
| NGS F primer | acactctttccctacacgacgctcttccgatctCTTTGCACTT | 105 | 615 |
| | AGCCTCAGTTT | | |
| NGS F primer | acactctttccctacacgacgctcttccgatctCAACACCACA | 106 | 616 |
| | AAGATTTGGC | | |
| NGS F primer | acactctttccctacacgacgctcttccgatctCTCTTCTCTC | 107 | 617 |
| | CTGCCCTTT | | |
| NGS F primer | acactctttccctacacgacgctcttccgatctGGTCTGAAAA | 108 | 618 |
| | TGCTCTTCCA | | |
| NGS F primer | acactctttccctacacgacgctcttccgatctCTTCAAAAGG | 109 | 619 |
| | GAGCCACAT | | |
| NGS F primer | acactctttccctacacgacgctcttccgatctCTGAGAATAT | 110 | 620 |
| | CTAGCAGCAACAT | | |
| NGS F primer | acactctttccctacacgacgctcttccgatctTTCTTCTCAG | 111 | 621 |
| | CTTACCACAGT | | |
| NGS F primer | acactctttccctacacgacgctcttccgatctCGCAAAGCTT | 112 | 622 |
| | CTTCTTGATCTAAAC | | |
| NGS F primer | acactctttccctacacgacgctcttccgatctCAAGATGCCC | 113 | 623 |
| | ACTATGCA | | |
| NGS F primer | acactctttccctacacgacgctcttccgatctCATCTGCAGC | 114 | 624 |
| | ACTTCACT | | |
| NGS F primer | acactctttccctacacgacgctcttccgatctTGGTCTCCAG | 115 | 625 |
| | TACTGAGTCT | | |
| NGS F primer | acactctttccctacacgacgctcttccgatctTGTACGTATG | 116 | 626 |
| | CTGAGATAATGCA | | |
| NGS F primer | acactctttccctacacgacgctcttccgatctCACATGCATT | 117 | 627 |
| | TCAGGACACT | | |
| NGS F primer | acactctttccctacacgacgctcttccgatctAGAGTCCGTT | 118 | 628 |
| | TTGCCAGTA | | |
| NGS F primer | acactctttccctacacgacgctcttccgatctGGGACTGTAG | 119 | 629 |
| | CTAATCCTAAC | | |
| NGS F primer | acactctttccctacacgacgctcttccgatctAGCATCATGA | 120 | 630 |
| | TAGGTACAATAATTGG | | |
| NGS F primer | acactctttccctacacgacgctcttccgatctTTTCCATTGG | 121 | 631 |
| | CTACCGAGT | | |
| NGS F primer | acactctttccctacacgacgctcttccgatctCTGACAGTTT | 122 | 632 |
| | ACCTTCCACC | | |
| NGS F primer | acactctttccctacacgacgctcttccgatctGGCCTTAAGT | 123 | 633 |
| | TCATTATTCTTTCC | | |
| NGS F primer | acactctttccctacacgacgctcttccgatctAGGCTCACGT | 124 | 634 |
| | TCCTCTCT | | |
| NGS F primer | acactctttccctacacgacgctcttccgatctTTTCTTCAAC | 125 | 635 |
| | ACCATCCTGC | | |
| NGS F primer | acactctttccctacacgacgctcttccgatctGGCATAAGAC | 126 | 636 |
| | CTACCTGTG | | |
| NGS F primer | acactctttccctacacgacgctcttccgatctATTTTGAACC | 127 | 637 |
| | CCTGCCCAT | | |
| NGS F primer | acactctttccctacacgacgctcttccgatctACAGGACACT | 128 | 638 |
| | TCCTTGCA | | |
| NGS F primer | acactctttccctacacgacgctcttccgatctTAAAGATGAG | 129 | 639 |
| | TCGCTGGAG | | |
| NGS F primer | acactctttccctacacgacgctcttccgatctACTCCTTCAT | 130 | 640 |
| | CACCTTTGCTA | | |
| NGS F primer | acactctttccctacacgacgctcttccgatctAAAGGTCTCA | 131 | 641 |
| | AGATTCTGCC | | |
| NGS F primer | acactctttccctacacgacgctcttccgatctCACATTGTCA | 132 | 642 |
| | CTTTCTTCAGC | | |
| NGS F primer | acactctttccctacacgacgctcttccgatctAACAGCAACC | 133 | 643 |
| | TTCACAGC | | |
| NGS F primer | acactctttccctacacgacgctcttccgatctTCCAATCCCA | 134 | 644 |
| | GGAGACTTTG | | |
| NGS F primer | acactctttccctacacgacgctcttccgatctACTCTCTGCT | 135 | 645 |
| | CATACCCAA | | |
| NGS F primer | acactctttccctacacgacgctcttccgatctATCCCTGTGC | 136 | 646 |
| | CCCTTTC | | |
| NGS F primer | acactctttccctacacgacgctcttccgatctATGAAGTCCA | 137 | 647 |
| | CACACTGCTC | | |
| NGS F primer | acactctttccctacacgacgctcttccgatctTGCTGCTGTA | 138 | 648 |
| | CAAAAGTCC | | |
| NGS F primer | acactctttccctacacgacgctcttccgatctGTTTGCTAAC | 139 | 649 |
| | TAGGAAAGTCCAT | | |
| NGS F primer | acactctttccctacacgacgctcttccgatctCAGATCAGGG | 140 | 650 |
| | CATTGGGAT | | |
| NGS F primer | acactctttccctacacgacgctcttccgatctCTTAGTGGGT | 141 | 651 |
| | GCCTTGCT | | |
| NGS F primer | acactctttccctacacgacgctcttccgatctCCCCATGTAC | 142 | 652 |
| | CACGTTAAAA | | |
| NGS F primer | acactctttccctacacgacgctcttccgatctGCAGTAACCC | 143 | 653 |
| | TCATTCTCA | | |
| NGS F primer | acactctttccctacacgacgctcttccgatctAAGGAAAACC | 144 | 654 |
| | TACTCTCTCTGG | | |
| NGS F primer | acactctttccctacacgacgctcttccgatctAATGACTGCC | 145 | 655 |
| | CCACATTTTA | | |
| NGS F primer | acactctttccctacacgacgctcttccgatctTGCACAGGAA | 146 | 656 |
| | ACTAGGACAT | | |
| NGS F primer | acactctttccctacacgacgctcttccgatctGCCCATATAG | 147 | 657 |
| | GATTACAACCC | | |
| NGS F primer | acactctttccctacacgacgctcttccgatctCTCCTTGCCC | 148 | 658 |
| | AGATTCAA | | |
| NGS F primer | acactctttccctacacgacgctcttccgatctCCAATATTTT | 149 | 659 |
| | CCATAACTTAAGGTGC | | |
| NGS F primer | acactctttccctacacgacgctcttccgatctGGCAGCCCAC | 150 | 660 |
| | AATAAAGAC | | |
| NGS F primer | acactctttccctacacgacgctcttccgatctTTCTTATGCA | 151 | 661 |
| | GAAGACTTAACTGATG | | |
| NGS F primer | acactctttccctacacgacgctcttccgatctCAAACATGTC | 152 | 662 |
| | TGCAGAGTACAC | | |
| NGS F primer | acactctttccctacacgacgctcttccgatctCGCTTGCCTG | 153 | 663 |
| | AAACATGAA | | |
| NGS F primer | acactctttccctacacgacgctcttccgatctGGGCAAGTGG | 154 | 664 |
| | AAAATCCAAG | | |
| NGS F primer | acactctttccctacacgacgctcttccgatctGCCCATAGGT | 155 | 665 |
| | AAAGTGTTGA | | |
| NGS F primer | acactctttccctacacgacgctcttccgatctCTGGCTAATC | 156 | 666 |
| | TCTTGGTCTCT | | |
| NGS F primer | acactctttccctacacgacgctcttccgatctTGAACACGGC | 157 | 667 |
| | CAAGTTTAG | | |
| NGS F primer | acactctttccctacacgacgctcttccgatctTCGCCATTAT | 158 | 668 |
| | CCGAGAGAG | | |
| NGS F primer | acactctttccctacacgacgctcttccgatctCAAATCTACC | 159 | 669 |
| | TTTAAGTCAGCCA | | |
| NGS F primer | acactctttccctacacgacgctcttccgatctTCTCCACCTT | 160 | 670 |
| | GCTGAGTC | | |
| NGS F primer | acactctttccctacacgacgctcttccgatctTAGATCTGCC | 161 | 671 |
| | ATGTCACAAGT | | |
| NGS F primer | acactctttccctacacgacgctcttccgatctAATTGTTCTT | 162 | 672 |
| | GCTCTCCTGG | | |
| NGS F primer | acactctttccctacacgacgctcttccgatctGCTGTCATCA | 163 | 673 |
| | CTGTGG | | |
| NGS F primer | acactctttccctacacgacgctcttccgatctTGTGCTACAG | 164 | 674 |
| | CCATGTCA | | |
| NGS F primer | acactctttccctacacgacgctcttccgatctGCTGTCATGC | 165 | 675 |
| | AAGTGCTT | | |
| NGS F primer | acactctttccctacacgacgctcttccgatctCAAGTCAAAA | 166 | 676 |
| | ACATTCAAGGGC | | |
| NGS F primer | acactctttccctacacgacgctcttccgatctCACTGTTCCA | 167 | 677 |
| | GGATGATCC | | |
| NGS F primer | acactctttccctacacgacgctcttccgatctAAGCCATGGA | 168 | 678 |
| | GAACGCG | | |
| NGS F primer | acactctttccctacacgacgctcttccgatctCCAGAAGTCT | 169 | 679 |
| | TCTCAGCATTT | | |
| NGS F primer | acactctttccctacacgacgctcttccgatctCTGACCTCTT | 170 | 680 |
| | TGAAACGCTC | | |
| NGS F primer | acactctttccctacacgacgctcttccgatctCTACCAGTCT | 171 | 681 |
| | GAGCACTACT | | |
| NGS F primer | acactctttccctacacgacgctcttccgatctTCTTCTTACA | 172 | 682 |
| | GAGAGTGTATATGGTA | | |
| NGS F primer | acactctttccctacacgacgctcttccgatctAGTTCTAGTG | 173 | 683 |
| | CTGACAGATGT | | |
| NGS F primer | acactctttccctacacgacgctcttccgatctGTTCTGATAA | 174 | 684 |
| | TCCCTCCGTGA | | |
| NGS F primer | acactctttccctacacgacgctcttccgatctCCGCCCACCT | 175 | 685 |
| | TGTATTT | | |
| NGS F primer | acactctttccctacacgacgctcttccgatctGTCCAGCCCA | 176 | 686 |
| | TGATGATTT | | |
| NGS F primer | acactctttccctacacgacgctcttccgatctTTTCTCCTCC | 177 | 687 |
| | TGCCCTAAT | | |
| NGS F primer | acactctttccctacacgacgctcttccgatctACCCTTCCCT | 178 | 688 |
| | CATATGACT | | |
| NGS F primer | acactctttccctacacgacgctcttccgatctAGGCCCATTT | 179 | 689 |
| | CATGCTAAA | | |
| NGS F primer | acactctttccctacacgacgctcttccgatctAGGAGCAAGA | 180 | 690 |
| | TCTGGCAG | | |
| NGS F primer | acactctttccctacacgacgctcttccgatctGAGAAAGTCC | 181 | 691 |
| | CTTCCCATG | | |
| NGS F primer | acactctttccctacacgacgctcttccgatctGTGGCAATCT | 182 | 692 |
| | TGGTGAAGTA | | |
| NGS F primer | acactctttccctacacgacgctcttccgatctTCTTACTATG | 183 | 693 |
| | AGATTGCTCGTGG | | |
| NGS F primer | acactctttccctacacgacgctcttccgatctGCCCTTCATA | 184 | 694 |
| | ACCACCTAC | | |
| NGS F primer | acactctttccctacacgacgctcttccgatctCAGCTACCCA | 185 | 695 |
| | CTTTGGATTTT | | |
| NGS F primer | acactctttccctacacgacgctcttccgatctATACCGTCCA | 186 | 696 |
| | AAAGAGATCACTT | | |
| NGS F primer | acactctttccctacacgacgctcttccgatctTGTATAGACA | 187 | 697 |
| | CATCTTGATAGGCAT | | |
| NGS F primer | acactctttccctacacgacgctcttccgatctGCTACTATGG | 188 | 698 |
| | GCTGGTC | | |
| NGS F primer | acactctttccctacacgacgctcttccgatctAGCTAAGTTC | 189 | 699 |
| | AACGTTCTGTTC | | |
| NGS F primer | acactctttccctacacgacgctcttccgatctAAATAACTCT | 190 | 700 |
| | AGGGTTTGGTTTCA | | |
| NGS F primer | acactctttccctacacgacgctcttccgatctAGAACCAGCT | 191 | 701 |
| | GCAGTATG | | |
| NGS F primer | acactctttccctacacgacgctcttccgatctGATCCTTGTA | 192 | 702 |
| | CCTGCTTGAATTT | | |
| NGS F primer | acactctttccctacacgacgctcttccgatctTCCAAGCCTA | 193 | 703 |
| | TGCATCATATC | | |
| NGS F primer | acactctttccctacacgacgctcttccgatctCCTTAGCTCT | 194 | 704 |
| | CTCATCTCCT | | |
| NGS F primer | acactctttccctacacgacgctcttccgatctGGAGCTAGAA | 195 | 705 |
| | CTGGCGTTA | | |
| NGS F primer | acactctttccctacacgacgctcttccgatctTGCAACCCTC | 196 | 706 |
| | TCGATGG | | |
| NGS F primer | acactctttccctacacgacgctcttccgatctCAACTAGCAG | 197 | 707 |
| | AATAGTAATGGATGG | | |
| NGS F primer | acactctttccctacacgacgctcttccgatctCACTTTAAAT | 198 | 708 |
| | ATGTAGAGTTTGTCTTGG | | |
| NGS F primer | acactctttccctacacgacgctcttccgatctCCTACAGTGT | 199 | 709 |
| | TTTCAGACTCCA | | |
| NGS F primer | acactctttccctacacgacgctcttccgatctAGAGTCTGGG | 200 | 710 |
| | TAGCTTTGT | | |
| NGS F primer | acactctttccctacacgacgctcttccgatctTCAACCGCAA | 201 | 711 |
| | GAGCCTT | | |
| NGS F primer | acactctttccctacacgacgctcttccgatctTTCCTCCCTC | 202 | 712 |
| | ACTCAGC | | |
| NGS F primer | acactctttccctacacgacgctcttccgatctCTTGTTTTCT | 203 | 713 |
| | TCCTGTCTGCT | | |
| NGS F primer | acactctttccctacacgacgctcttccgatctGTTGTATGTG | 204 | 714 |
| | GGATGTGACT | | |
| NGS F primer | acactctttccctacacgacgctcttccgatctGGTTGATGTG | 205 | 715 |
| | TGTTATTATTTGTAATTAT | | |
| NGS F primer | acactctttccctacacgacgctcttccgatctAACTGGTCCA | 206 | 716 |
| | GCTCATCC | | |
| NGS F primer | acactctttccctacacgacgctcttccgatctCCTCACAGAC | 207 | 717 |
| | TTTTAGACATCGTAG | | |
| NGS F primer | acactctttccctacacgacgctcttccgatctCTCTTCCATA | 208 | 718 |
| | GTGGTTGGAGT | | |
| NGS F primer | acactctttccctacacgacgctcttccgatctCGCAACAGAA | 209 | 719 |
| | AAAGTATTTAAGCAG | | |
| NGS F primer | acactctttccctacacgacgctcttccgatctGAGCCGCCGA | 210 | 720 |
| | ACCATA | | |
| NGS F primer | acactctttccctacacgacgctcttccgatctGAACAAGATT | 211 | 721 |
| | GTGGACCAGT | | |
| NGS F primer | acactctttccctacacgacgctcttccgatctGAAACTCTGA | 212 | 722 |
| | ATGCCAAAGAAATT | | |
| NGS F primer | acactctttccctacacgacgctcttccgatctTGTTTGGTTA | 213 | 723 |
| | TTTTTCAGGGTACA | | |
| NGS F primer | acactctttccctacacgacgctcttccgatctAGTAGCAGCA | 214 | 724 |
| | TCTGTGATCAT | | |
| NGS F primer | acactctttccctacacgacgctcttccgatctCAGGAGCTAT | 215 | 725 |
| | CCAGAATTTAGGC | | |
| NGS F primer | acactctttccctacacgacgctcttccgatctGCTGCCTTTC | 216 | 726 |
| | TTTCCTCA | | |
| NGS F primer | acactctttccctacacgacgctcttccgatctAGGTTTGACC | 217 | 727 |
| | CTACTCAGTTT | | |
| NGS F primer | acactctttccctacacgacgctcttccgatctGTCTTCTAAG | 218 | 728 |
| | TTCTGGCCAA | | |
| NGS F primer | acactctttccctacacgacgctcttccgatctTGAATTCCCG | 219 | 729 |
| | AGCTTCTCG | | |
| NGS F primer | acactctttccctacacgacgctcttccgatctAACAGTGTGT | 220 | 730 |
| | CCTCAGC | | |
| NGS F primer | acactctttccctacacgacgctcttccgatctAGGAATCAGA | 221 | 731 |
| | TATGTGGAAAATAAGAG | | |
| NGS F primer | acactctttccctacacgacgctcttccgatctTTCCAGGAGA | 222 | 732 |
| | AGTGGAGCA | | |
| NGS F primer | acactctttccctacacgacgctcttccgatctCAGAATGACT | 223 | 733 |
| | CTTCTCTGTGT | | |
| NGS F primer | acactctttccctacacgacgctcttccgatctTGTTTCAGTA | 224 | 734 |
| | GAGATGGCATATTT | | |
| NGS F primer | acactctttccctacacgacgctcttccgatctCATGGCCCTG | 225 | 735 |
| | GATAATTCT | | |
| NGS F primer | acactctttccctacacgacgctcttccgatctGGTTGCATTG | 226 | 736 |
| | CTCACC | | |
| NGS F primer | acactctttccctacacgacgctcttccgatctGGGACTTCAG | 227 | 737 |
| | TTAGTGACA | | |
| NGS F primer | acactctttccctacacgacgctcttccgatctGCACTGTCCT | 228 | 738 |
| | CTGCCC | | |
| NGS F primer | acactctttccctacacgacgctcttccgatctTCACAGCCAA | 229 | 739 |
| | CATTCAGAG | | |
| NGS F primer | acactctttccctacacgacgctcttccgatctAAGTTCTTGG | 230 | 740 |
| | GCTTGCTT | | |
| NGS F primer | acactctttccctacacgacgctcttccgatctTGATTCCCAG | 231 | 741 |
| | CCAGTG | | |
| NGS F primer | acactctttccctacacgacgctcttccgatctCAGGTTTAAA | 232 | 742 |
| | CTCTGGACACG | | |
| NGS F primer | acactctttccctacacgacgctcttccgatctCTACCTTCTC | 233 | 743 |
| | CCACCCTG | | |
| NGS F primer | acactctttccctacacgacgctcttccgatctTGTGAGACAC | 234 | 744 |
| | CTGCACTTA | | |
| NGS F primer | acactctttccctacacgacgctcttccgatctCAACCACCCA | 235 | 745 |
| | ACTTCTCTC | | |
| NGS F primer | acactctttccctacacgacgctcttccgatctTCAGGGTCCC | 236 | 746 |
| | CACATG | | |
| NGS F primer | acactctttccctacacgacgctcttccgatctTGTGCCTGAC | 237 | 747 |
| | TTCCCAG | | |
| NGS F primer | acactctttccctacacgacgctcttccgatctGCTCACTTTC | 238 | 748 |
| | ATAATTTCAACTCGAATT | | |
| NGS F primer | acactctttccctacacgacgctcttccgatctGAGGTCCTAA | 239 | 749 |
| | GTTACTTGATGTGTTA | | |
| NGS F primer | acactctttccctacacgacgctcttccgatctCTTCTGGCAA | 240 | 750 |
| | TGTGGATATTC | | |
| NGS F primer | acactctttccctacacgacgctcttccgatctTTTGGCAGCA | 241 | 751 |
| | AGTGCAAT | | |
| NGS F primer | acactctttccctacacgacgctcttccgatctCTGCACAGTG | 242 | 752 |
| | CTGATCAGTA | | |
| NGS F primer | acactctttccctacacgacgctcttccgatctGCTTTTTAAT | 243 | 753 |
| | TTGTTGTTGAAGTGTT | | |
| NGS F primer | acactctttccctacacgacgctcttccgatctCAAAGCTGAC | 244 | 754 |
| | TGCAAACAATT | | |
| NGS F primer | acactctttccctacacgacgctcttccgatctCAACCTATGT | 245 | 755 |
| | AAAATGCCCAA | | |
| NGS F primer | acactctttccctacacgacgctcttccgatctCAGGTGTGCA | 246 | 756 |
| | CGTTGAG | | |
| NGS F primer | acactctttccctacacgacgctcttccgatctGGTACCCCAT | 247 | 757 |
| | AGTCTTCCTG | | |
| NGS F primer | acactctttccctacacgacgctcttccgatctCCACCAACCC | 248 | 758 |
| | AAATCCTTTC | | |
| NGS F primer | acactctttccctacacgacgctcttccgatctAAGGTGAATT | 249 | 759 |
| | CCTCTTCCCA | | |
| NGS F primer | acactctttccctacacgacgctcttccgatctTTCACATTTG | 250 | 760 |
| | TTCAGCTATCCT | | |
| NGS F primer | acactctttccctacacgacgctcttccgatctCAGAATCTTC | 251 | 761 |
| | AGAAATGGCACAA | | |
| NGS F primer | acactctttccctacacgacgctcttccgatctAGGCTCGCTG | 252 | 762 |
| | TACTCG | | |
| NGS F primer | acactctttccctacacgacgctcttccgatctCACCTTAAAA | 253 | 763 |
| | TCAGGGCCATT | | |
| NGS F primer | acactctttccctacacgacgctcttccgatctCAGTGCGGTG | 254 | 764 |
| | TCTCTG | | |
| NGS F primer | acactctttccctacacgacgctcttccgatctTATCCTAACA | 255 | 765 |
| | CCTGCCCTC | | |
| NGS R primer | gtgactggagttcagacgtgtgctcttccgatctTCAACACTA | 1 | 766 |
| | TGAACCCAAACATC | | |
| NGS R primer | gtgactggagttcagacgtgtgctcttccgatctTGTACTACG | 2 | 767 |
| | TGTTCACCGAG | | |
| NGS R primer | gtgactggagttcagacgtgtgctcttccgatctGGTCTGAGA | 3 | 768 |
| | AAATGGTTCTTACT | | |
| NGS R primer | gtgactggagttcagacgtgtgctcttccgatctGGTCATTCC | 4 | 769 |
| | GAGAACGC | | |
| NGS R primer | gtgactggagttcagacgtgtgctcttccgatctCTGCAAACC | 5 | 770 |
| | TTGAAACAGAAT | | |
| NGS R primer | gtgactggagttcagacgtgtgctcttccgatctGTTTGATGT | 6 | 771 |
| | ATGCTGGCTTCAT | | |
| NGS R primer | gtgactggagttcagacgtgtgctcttccgatctAAAATCAAT | 7 | 772 |
| | GATGCCATAGCTGA | | |
| NGS R primer | gtgactggagttcagacgtgtgctcttccgatctCATTGGTGT | 8 | 773 |
| | GGCCAAGAA | | |
| NGS R primer | gtgactggagttcagacgtgtgctcttccgatctGAATCCCAA | 9 | 774 |
| | CATGGTCCC | | |
| NGS R primer | gtgactggagttcagacgtgtgctcttccgatctCGATGAGGA | 10 | 775 |
| | GCCACTG | | |
| NGS R primer | gtgactggagttcagacgtgtgctcttccgatctGTCTTTACC | 11 | 776 |
| | TGTTTGTGATGAGC | | |
| NGS R primer | gtgactggagttcagacgtgtgctcttccgatctTCCAGCTAC | 12 | 777 |
| | TCTGTGTCATT | | |
| NGS R primer | gtgactggagttcagacgtgtgctcttccgatctCAGTGCCTA | 13 | 778 |
| | CCCTAAATTAATAGAA | | |
| NGS R primer | gtgactggagttcagacgtgtgctcttccgatctTTGTTGACC | 14 | 779 |
| | AGCTCCAGG | | |
| NGS R primer | gtgactggagttcagacgtgtgctcttccgatctCCCATGAAT | 15 | 780 |
| | TGAAATAGCAGC | | |
| NGS R primer | gtgactggagttcagacgtgtgctcttccgatctTCAGACTCT | 16 | 781 |
| | GACCTCGATC | | |
| NGS R primer | gtgactggagttcagacgtgtgctcttccgatctGCTACTACC | 17 | 782 |
| | CGGTCATCTT | | |
| NGS R primer | gtgactggagttcagacgtgtgctcttccgatctCACATCTCC | 18 | 783 |
| | ACTCTTCAATGGATT | | |
| NGS R primer | gtgactggagttcagacgtgtgctcttccgatctGACATAGGA | 19 | 784 |
| | GCAGAGCTGAAG | | |
| NGS R primer | gtgactggagttcagacgtgtgctcttccgatctGGGAAAGAG | 20 | 785 |
| | GAGGGCC | | |
| NGS R primer | gtgactggagttcagacgtgtgctcttccgatctCAGCAGTCT | 21 | 786 |
| | AATCAATGGCAG | | |
| NGS R primer | gtgactggagttcagacgtgtgctcttccgatctCCCTGCAAC | 22 | 787 |
| | ATGGGAAATAA | | |
| NGS R primer | gtgactggagttcagacgtgtgctcttccgatctCTGCTTCAC | 23 | 788 |
| | AAAAACTTGCAG | | |
| NGS R primer | gtgactggagttcagacgtgtgctcttccgatctCAGTATTGG | 24 | 789 |
| | ACATTAGATAGCATTTAT | | |
| NGS R primer | gtgactggagttcagacgtgtgctcttccgatctCATACGCCC | 25 | 790 |
| | CTCTCCTACA | | |
| NGS R primer | gtgactggagttcagacgtgtgctcttccgatctTAGGCATTG | 26 | 791 |
| | TAGTCCTGGAAG | | |
| NGS R primer | gtgactggagttcagacgtgtgctcttccgatctCATTCCGAA | 27 | 792 |
| | AGATCTTTGGTAGATA | | |
| NGS R primer | gtgactggagttcagacgtgtgctcttccgatctTTGGTGAAG | 28 | 793 |
| | TAGGTGATGGA | | |
| NGS R primer | gtgactggagttcagacgtgtgctcttccgatctGCAGAGCCA | 29 | 794 |
| | TTGTTGATATGTTAT | | |
| NGS R primer | gtgactggagttcagacgtgtgctcttccgatctAACCCTAAT | 30 | 795 |
| | GATCTGACCAAC | | |
| NGS R primer | gtgactggagttcagacgtgtgctcttccgatctGTAATGAGA | 31 | 796 |
| | GATGGGCTCAC | | |
| NGS R primer | gtgactggagttcagacgtgtgctcttccgatctCTACAGGAG | 32 | 797 |
| | ACCTTTGAGGA | | |
| NGS R primer | gtgactggagttcagacgtgtgctcttccgatctTTCTATATA | 33 | 798 |
| | TCCCCAGCCGG | | |
| NGS R primer | gtgactggagttcagacgtgtgctcttccgatctCAGGATTCG | 34 | 799 |
| | ACTCAGGC | | |
| NGS R primer | gtgactggagttcagacgtgtgctcttccgatctTATGTACGA | 35 | 800 |
| | TGGCTTCTGGTC | | |
| NGS R primer | gtgactggagttcagacgtgtgctcttccgatctACCCATTCC | 36 | 801 |
| | AGCTTTGTG | | |
| NGS R primer | gtgactggagttcagacgtgtgctcttccgatctGTAGCCCCA | 37 | 802 |
| | AATACCAATGA | | |
| NGS R primer | gtgactggagttcagacgtgtgctcttccgatctCTCCAGCTA | 38 | 803 |
| | TGTGTGAAGAA | | |
| NGS R primer | gtgactggagttcagacgtgtgctcttccgatctAGCCTCATT | 39 | 804 |
| | TACCAGCCTG | | |
| NGS R primer | gtgactggagttcagacgtgtgctcttccgatctTATGAGGCC | 40 | 805 |
| | CTACATTTGCA | | |
| NGS R primer | gtgactggagttcagacgtgtgctcttccgatctGCTGGCCAT | 41 | 806 |
| | CATTATTACTGTG | | |
| NGS R primer | gtgactggagttcagacgtgtgctcttccgatctCAGAAATAT | 42 | 807 |
| | TCTTCCAGGTAGCAAAA | | |
| NGS R primer | gtgactggagttcagacgtgtgctcttccgatctCCCCTTAAA | 43 | 808 |
| | CCACTGAAGA | | |
| NGS R primer | gtgactggagttcagacgtgtgctcttccgatctGACAATGCT | 44 | 809 |
| | CCTTAAGTCTCTT | | |
| NGS R primer | gtgactggagttcagacgtgtgctcttccgatctTGCCTTCCC | 45 | 810 |
| | AGTTCTTGA | | |
| NGS R primer | gtgactggagttcagacgtgtgctcttccgatctTAAATCATA | 46 | 811 |
| | GGCTACAGCTGAAA | | |
| NGS R primer | gtgactggagttcagacgtgtgctcttccgatctGGCTTTGCT | 47 | 812 |
| | TTACTACCAATCTC | | |
| NGS R primer | gtgactggagttcagacgtgtgctcttccgatctTGGGATGTG | 48 | 813 |
| | GAAAGTCATTCT | | |
| NGS R primer | gtgactggagttcagacgtgtgctcttccgatctCGTAAGGTG | 49 | 814 |
| | ATACACAAGTTCTG | | |
| NGS R primer | gtgactggagttcagacgtgtgctcttccgatctGCTCAAATG | 50 | 815 |
| | TCTTTCTGAAGTACG | | |
| NGS R primer | gtgactggagttcagacgtgtgctcttccgatctGAGTGGATG | 51 | 816 |
| | TGCAGGTC | | |
| NGS R primer | gtgactggagttcagacgtgtgctcttccgatctTGCCGTCTT | 52 | 817 |
| | CTACAACAA | | |
| NGS R primer | gtgactggagttcagacgtgtgctcttccgatctCCATCATAC | 53 | 818 |
| | ACCACAAATCCA | | |
| NGS R primer | gtgactggagttcagacgtgtgctcttccgatctGATGAATAC | 54 | 819 |
| | TCAGTCAGGGTATCA | | |
| NGS R primer | gtgactggagttcagacgtgtgctcttccgatctGTGACAGGT | 55 | 820 |
| | CTCTGGTAGA | | |
| NGS R primer | gtgactggagttcagacgtgtgctcttccgatctTAGGTCGCG | 56 | 821 |
| | CGCTATC | | |
| NGS R primer | gtgactggagttcagacgtgtgctcttccgatctCCTTTTCAT | 57 | 822 |
| | GCCAAAGTCCTAT | | |
| NGS R primer | gtgactggagttcagacgtgtgctcttccgatctGTCATAAGA | 58 | 823 |
| | CATCTTAGGAGCCA | | |
| NGS R primer | gtgactggagttcagacgtgtgctcttccgatctATGATGAGG | 59 | 824 |
| | CAGGGCG | | |
| NGS R primer | gtgactggagttcagacgtgtgctcttccgatctAACCTGCTG | 60 | 825 |
| | GTCATCGTG | | |
| NGS R primer | gtgactggagttcagacgtgtgctcttccgatctGGGCTCAGG | 61 | 826 |
| | TTCTAGCT | | |
| NGS R primer | gtgactggagttcagacgtgtgctcttccgatctGGTCCCTTT | 62 | 827 |
| | CTCATTCAGTTA | | |
| NGS R primer | gtgactggagttcagacgtgtgctcttccgatctAGTCCTAAC | 63 | 828 |
| | CCGTGTTGC | | |
| NGS R primer | gtgactggagttcagacgtgtgctcttccgatctGGCCTGTGA | 64 | 829 |
| | CGCTCAC | | |
| NGS R primer | gtgactggagttcagacgtgtgctcttccgatctCTCAAAGAA | 65 | 830 |
| | GCCTAAACAAAGTAC | | |
| NGS R primer | gtgactggagttcagacgtgtgctcttccgatctCTAGCCACA | 66 | 831 |
| | TCAGCCT | | |
| NGS R primer | gtgactggagttcagacgtgtgctcttccgatctTATTAGGGT | 67 | 832 |
| | GCAAGAGGACTG | | |
| NGS R primer | gtgactggagttcagacgtgtgctcttccgatctTGTGCATCA | 68 | 833 |
| | CTTACCGGTT | | |
| NGS R primer | gtgactggagttcagacgtgtgctcttccgatctAAAGGTTTA | 69 | 834 |
| | ACCAAGGGAAGC | | |
| NGS R primer | gtgactggagttcagacgtgtgctcttccgatctGAGCTACTG | 70 | 835 |
| | TGGGCTG | | |
| NGS R primer | gtgactggagttcagacgtgtgctcttccgatctGCTACTCAC | 71 | 836 |
| | CGCTTCTTTG | | |
| NGS R primer | gtgactggagttcagacgtgtgctcttccgatctCACAGACAA | 72 | 837 |
| | GCCCTCAGATATATT | | |
| NGS R primer | gtgactggagttcagacgtgtgctcttccgatctTCCAGAATC | 73 | 838 |
| | TGTTACCTGTGAA | | |
| NGS R primer | gtgactggagttcagacgtgtgctcttccgatctTCCAGCTGA | 74 | 839 |
| | AAATTGGAGC | | |
| NGS R primer | gtgactggagttcagacgtgtgctcttccgatctAAATGGAAA | 75 | 840 |
| | GCTTGGACTCAC | | |
| NGS R primer | gtgactggagttcagacgtgtgctcttccgatctACTGTGAAA | 76 | 841 |
| | TGATATGGAGCTTTT | | |
| NGS R primer | gtgactggagttcagacgtgtgctcttccgatctGAGACAGTG | 77 | 842 |
| | TTGGACATG | | |
| NGS R primer | gtgactggagttcagacgtgtgctcttccgatctCGTACTGTC | 78 | 843 |
| | AGTTAACCTAACTCAT | | |
| NGS R primer | gtgactggagttcagacgtgtgctcttccgatctTAGGTGAGA | 79 | 844 |
| | AACCTGGAAATGA | | |
| NGS R primer | gtgactggagttcagacgtgtgctcttccgatctAATAATAGA | 80 | 845 |
| | ACTAACAGCACTCAGAATCA | | |
| NGS R primer | gtgactggagttcagacgtgtgctcttccgatctGCTTAACTT | 81 | 846 |
| | CAAGGAAAACACTCA | | |
| NGS R primer | gtgactggagttcagacgtgtgctcttccgatctCACAAGCTT | 82 | 847 |
| | CACTCTGATTAAGAA | | |
| NGS R primer | gtgactggagttcagacgtgtgctcttccgatctCATGGATAA | 83 | 848 |
| | CTGAAGATTTCTCTCC | | |
| NGS R primer | gtgactggagttcagacgtgtgctcttccgatctTGGTCTTTG | 84 | 849 |
| | CTGAGCCTC | | |
| NGS R primer | gtgactggagttcagacgtgtgctcttccgatctAACATTGGC | 85 | 850 |
| | ATTTTCCATGATG | | |
| NGS R primer | gtgactggagttcagacgtgtgctcttccgatctTGACCCTCT | 86 | 85 |
| | TTGTGTAGCTG | | |
| NGS R primer | gtgactggagttcagacgtgtgctcttccgatctTGTGCGTTC | 87 | 852 |
| | TCGTTCTAG | | |
| NGS R primer | gtgactggagttcagacgtgtgctcttccgatctACCTCTGGA | 88 | 853 |
| | CCTGCTG | | |
| NGS R primer | gtgactggagttcagacgtgtgctcttccgatctTCAGAAAAG | 89 | 854 |
| | GTACACCCCG | | |
| NGS R primer | gtgactggagttcagacgtgtgctcttccgatctCTTGCGAAC | 90 | 855 |
| | ATGCGGC | | |
| NGS R primer | gtgactggagttcagacgtgtgctcttccgatctCCTCTTTTG | 91 | 856 |
| | TCACCAATCTTTG | | |
| NGS R primer | gtgactggagttcagacgtgtgctcttccgatctCGCCCCATC | 92 | 857 |
| | TGATGCTC | | |
| NGS R primer | gtgactggagttcagacgtgtgctcttccgatctTCCCGGTTT | 93 | 858 |
| | TAGAGAAATGTG | | |
| NGS R primer | gtgactggagttcagacgtgtgctcttccgatctAGGTACCAG | 94 | 859 |
| | TCGTCATTCAG | | |
| NGS R primer | gtgactggagttcagacgtgtgctcttccgatctTTGTGGAGA | 95 | 860 |
| | CCTTTGGC | | |
| NGS R primer | gtgactggagttcagacgtgtgctcttccgatctAGCTTTCCA | 96 | 861 |
| | GTGAAGACTCT | | |
| NGS R primer | gtgactggagttcagacgtgtgctcttccgatctTAGCCCCAT | 97 | 862 |
| | TCTAGAAAATGC | | |
| NGS R primer | gtgactggagttcagacgtgtgctcttccgatctGCAGTAGGT | 98 | 863 |
| | AGCCGAGAT | | |
| NGS R primer | gtgactggagttcagacgtgtgctcttccgatctAGCCAATGG | 99 | 864 |
| | TAAACCTGCAT | | |
| NGS R primer | gtgactggagttcagacgtgtgctcttccgatctGCTGGTCTT | 100 | 865 |
| | TTGGTATCGTAG | | |
| NGS R primer | gtgactggagttcagacgtgtgctcttccgatctAATACCATC | 101 | 866 |
| | TGTCAAAGAGGC | | |
| NGS R primer | gtgactggagttcagacgtgtgctcttccgatctCAGATGCCA | 102 | 867 |
| | CAGTTCTCAT | | |
| NGS R primer | gtgactggagttcagacgtgtgctcttccgatctTGGACCTGA | 103 | 868 |
| | CAAGGAGAG | | |
| NGS R primer | gtgactggagttcagacgtgtgctcttccgatctTGCAATTAC | 104 | 869 |
| | ACCTGACTTTCTCC | | |
| NGS R primer | gtgactggagttcagacgtgtgctcttccgatctTCCATTGAC | 105 | 870 |
| | AATTCATGGCC | | |
| NGS R primer | gtgactggagttcagacgtgtgctcttccgatctAAACTGACC | 106 | 871 |
| | TCTCGTTTGTCT | | |
| NGS R primer | gtgactggagttcagacgtgtgctcttccgatctTTGTCCTCT | 107 | 872 |
| | GCAGTACCTG | | |
| NGS R primer | gtgactggagttcagacgtgtgctcttccgatctTTCTGCTGA | 108 | 873 |
| | GGTGGTAAATGG | | |
| NGS R primer | gtgactggagttcagacgtgtgctcttccgatctAACCGCCAT | 109 | 874 |
| | GATCAGAAG | | |
| NGS R primer | gtgactggagttcagacgtgtgctcttccgatctGTCTGTTTG | 110 | 875 |
| | ATCTCACCATCTT | | |
| NGS R primer | gtgactggagttcagacgtgtgctcttccgatctGCTCCTGAA | 111 | 876 |
| | CAATATCTAAGTGT | | |
| NGS R primer | gtgactggagttcagacgtgtgctcttccgatctGTTGACATT | 112 | 877 |
| | CTCTTTGAAGATATGGT | | |
| NGS R primer | gtgactggagttcagacgtgtgctcttccgatctTTCAGCAGA | 113 | 878 |
| | TGTGAATGCC | | |
| NGS R primer | gtgactggagttcagacgtgtgctcttccgatctCAGAATGGT | 114 | 879 |
| | GATGGGCTG | | |
| NGS R primer | gtgactggagttcagacgtgtgctcttccgatctCTTCATTGT | 115 | 880 |
| | AGCGCCTCAG | | |
| NGS R primer | gtgactggagttcagacgtgtgctcttccgatctCTACACTGA | 116 | 881 |
| | GGACTTTGGTAAAC | | |
| NGS R primer | gtgactggagttcagacgtgtgctcttccgatctGGTTGGCTC | 117 | 882 |
| | GAAAGTGAC | | |
| NGS R primer | gtgactggagttcagacgtgtgctcttccgatctAGCCTCCAC | 118 | 883 |
| | CTATTGTGA | | |
| NGS R primer | gtgactggagttcagacgtgtgctcttccgatctTTCGTGGGA | 119 | 884 |
| | AAAACTGTCTC | | |
| NGS R primer | gtgactggagttcagacgtgtgctcttccgatctTGCTATTGT | 120 | 885 |
| | CATATTACACCCTTTAAG | | |
| NGS R primer | gtgactggagttcagacgtgtgctcttccgatctCTGTAGGAT | 121 | 886 |
| | GGCCACTATCT | | |
| NGS R primer | gtgactggagttcagacgtgtgctcttccgatctGGATGTAGT | 122 | 887 |
| | TTTTCAGGCTTG | | |
| NGS R primer | gtgactggagttcagacgtgtgctcttccgatctTAGCCTCTT | 123 | 888 |
| | CAATATTAAGTGGAA | | |
| NGS R primer | gtgactggagttcagacgtgtgctcttccgatctGGCCAAAAC | 124 | 889 |
| | CGACTGTG | | |
| NGS R primer | gtgactggagttcagacgtgtgctcttccgatctCAGGTTCTT | 125 | 890 |
| | GGTCTTGCTAAG | | |
| NGS R primer | gtgactggagttcagacgtgtgctcttccgatctTGACAAACA | 126 | 891 |
| | CTGCAGGAAA | | |
| NGS R primer | gtgactggagttcagacgtgtgctcttccgatctAGCCTTGTC | 127 | 892 |
| | CTCCAGTGT | | |
| NGS R primer | gtgactggagttcagacgtgtgctcttccgatctCCACCAAGT | 128 | 893 |
| | GCTTACGG | | |
| NGS R primer | gtgactggagttcagacgtgtgctcttccgatctCTTTCCTCC | 129 | 894 |
| | TCCCTGAGA | | |
| NGS R primer | gtgactggagttcagacgtgtgctcttccgatctGATTACCTG | 130 | 895 |
| | CAGTGTGGTAGAA | | |
| NGS R primer | gtgactggagttcagacgtgtgctcttccgatctGTTTGCCCA | 131 | 896 |
| | GAACTGTTGATT | | |
| NGS R primer | gtgactggagttcagacgtgtgctcttccgatctCCTTACACT | 132 | 897 |
| | TTGTAGACATGCA | | |
| NGS R primer | gtgactggagttcagacgtgtgctcttccgatctAGCTGAGTC | 133 | 898 |
| | ATCCTCGTC | | |
| NGS R primer | gtgactggagttcagacgtgtgctcttccgatctACGTGTAGT | 134 | 899 |
| | CAGCTTCCTC | | |
| NGS R primer | gtgactggagttcagacgtgtgctcttccgatctCAAAGGGTT | 135 | 900 |
| | CAATGTGGAGA | | |
| NGS R primer | gtgactggagttcagacgtgtgctcttccgatctCGGGCTCAC | 136 | 901 |
| | CTATGGTT | | |
| NGS R primer | gtgactggagttcagacgtgtgctcttccgatctAGGGTGTGA | 137 | 902 |
| | CTAGCTCCT | | |
| NGS R primer | gtgactggagttcagacgtgtgctcttccgatctCCCTCATGA | 138 | 903 |
| | AAGCTTCCC | | |
| NGS R primer | gtgactggagttcagacgtgtgctcttccgatctGGCCCTTAA | 139 | 904 |
| | TGCTTTACATTTTCT | | |
| NGS R primer | gtgactggagttcagacgtgtgctcttccgatctCCTCTGCTG | 140 | 905 |
| | TCCTCTCAA | | |
| NGS R primer | gtgactggagttcagacgtgtgctcttccgatctAAGTTCCAG | 141 | 906 |
| | TCCCCACC | | |
| NGS R primer | gtgactggagttcagacgtgtgctcttccgatctCTGTTGAAG | 142 | 907 |
| | CTGCTCGATTTT | | |
| NGS R primer | gtgactggagttcagacgtgtgctcttccgatctGAGGGTACT | 143 | 908 |
| | GCATTCCG | | |
| NGS R primer | gtgactggagttcagacgtgtgctcttccgatctGAGTCAAAG | 144 | 909 |
| | ATAAACACTTCATGC | | |
| NGS R primer | gtgactggagttcagacgtgtgctcttccgatctCTCTAGGCC | 145 | 910 |
| | ATACTGGAGAT | | |
| NGS R primer | gtgactggagttcagacgtgtgctcttccgatctGTTTGCATA | 146 | 911 |
| | GGCCTCACAG | | |
| NGS R primer | gtgactggagttcagacgtgtgctcttccgatctCTCTTCAGG | 147 | 912 |
| | AGTTCACAACG | | |
| NGS R primer | gtgactggagttcagacgtgtgctcttccgatctCATCTGCTG | 148 | 913 |
| | AGAAGGCAG | | |
| NGS R primer | gtgactggagttcagacgtgtgctcttccgatctGCATCTAAC | 149 | 914 |
| | AAAGATACTTACATTTGAA | | |
| NGS R primer | gtgactggagttcagacgtgtgctcttccgatctCACAAAGAC | 150 | 915 |
| | CATGACTCCTC | | |
| NGS R primer | gtgactggagttcagacgtgtgctcttccgatctTGCATTGTT | 151 | 916 |
| | ACCCAAAGTAATCAAAG | | |
| NGS R primer | gtgactggagttcagacgtgtgctcttccgatctCTTACCTGC | 152 | 917 |
| | ACCAAGTTGTAAAT | | |
| NGS R primer | gtgactggagttcagacgtgtgctcttccgatctCCTTTGTTC | 153 | 918 |
| | TCCTGCACAAT | | |
| NGS R primer | gtgactggagttcagacgtgtgctcttccgatctTTGTTACCC | 154 | 919 |
| | AGAGTGACCAA | | |
| NGS R primer | gtgactggagttcagacgtgtgctcttccgatctGTGGAAACT | 155 | 920 |
| | CTGTCATGTGT | | |
| NGS R primer | gtgactggagttcagacgtgtgctcttccgatctATCAGTGAA | 156 | 921 |
| | GAAAGGGCATCA | | |
| NGS R primer | gtgactggagttcagacgtgtgctcttccgatctAGCTAACTC | 157 | 922 |
| | CAAGCTCCC | | |
| NGS R primer | gtgactggagttcagacgtgtgctcttccgatctCCTCTCTGA | 158 | 923 |
| | AGGAACATTCG | | |
| NGS R primer | gtgactggagttcagacgtgtgctcttccgatctGTTTCAGGT | 159 | 924 |
| | TTCTTGCTGTATG | | |
| NGS R primer | gtgactggagttcagacgtgtgctcttccgatctAATACCCCT | 160 | 925 |
| | TCGAGCCAG | | |
| NGS R primer | gtgactggagttcagacgtgtgctcttccgatctAGCGAAAGA | 161 | 926 |
| | AGTTTGACCATGA | | |
| NGS R primer | gtgactggagttcagacgtgtgctcttccgatctTCTAGGCCA | 162 | 927 |
| | TGGAGTATCTG | | |
| NGS R primer | gtgactggagttcagacgtgtgctcttccgatctCGTAGCCAT | 163 | 928 |
| | TCTGCAG | | |
| NGS R primer | gtgactggagttcagacgtgtgctcttccgatctAAGGACCTC | 164 | 929 |
| | ATAGGGAGC | | |
| NGS R primer | gtgactggagttcagacgtgtgctcttccgatctCACAGAGGT | 165 | 930 |
| | GGATGCTG | | |
| NGS R primer | gtgactggagttcagacgtgtgctcttccgatctGTGTTTCTA | 166 | 931 |
| | ATGGTGCATCCT | | |
| NGS R primer | gtgactggagttcagacgtgtgctcttccgatctCTCTGGATC | 167 | 932 |
| | CCACAGGTATT | | |
| NGS R primer | gtgactggagttcagacgtgtgctcttccgatctCACCTACCG | 168 | 933 |
| | TTGGAGCC | | |
| NGS R primer | gtgactggagttcagacgtgtgctcttccgatctCACAGTAAC | 169 | 934 |
| | AGCTGTCTGG | | |
| NGS R primer | gtgactggagttcagacgtgtgctcttccgatctCCGTTTCAG | 170 | 935 |
| | TACCAGTGAAG | | |
| NGS R primer | gtgactggagttcagacgtgtgctcttccgatctTGCTGTGAC | 171 | 936 |
| | TTACTTGAAGC | | |
| NGS R primer | gtgactggagttcagacgtgtgctcttccgatctCTTACTTCA | 172 | 937 |
| | TTGTTCCTACTCAGAG | | |
| NGS R primer | gtgactggagttcagacgtgtgctcttccgatctCACTTCCAG | 173 | 938 |
| | CTTACTCACAGT | | |
| NGS R primer | gtgactggagttcagacgtgtgctcttccgatctGGTTGTCCA | 174 | 939 |
| | AGACTTCATCC | | |
| NGS R primer | gtgactggagttcagacgtgtgctcttccgatctGGTGTTTGT | 175 | 940 |
| | CCTGGGC | | |
| NGS R primer | gtgactggagttcagacgtgtgctcttccgatctACATTATGT | 176 | 941 |
| | CCCATGCATGC | | |
| NGS R primer | gtgactggagttcagacgtgtgctcttccgatctGTGAGTTAT | 177 | 942 |
| | TGGTTCGAGCC | | |
| NGS R primer | gtgactggagttcagacgtgtgctcttccgatctCTCCCAACT | 178 | 943 |
| | CTGTCACCT | | |
| NGS R primer | gtgactggagttcagacgtgtgctcttccgatctTGGCTCTGG | 179 | 944 |
| | ACATGACATAT | | |
| NGS R primer | gtgactggagttcagacgtgtgctcttccgatctGTGCGCTCT | 180 | 945 |
| | GGTCCTT | | |
| NGS R primer | gtgactggagttcagacgtgtgctcttccgatctATTCTCCAG | 181 | 946 |
| | GCGAGTCAG | | |
| NGS R primer | gtgactggagttcagacgtgtgctcttccgatctTGGTTACTG | 182 | 947 |
| | GCTCACCTG | | |
| NGS R primer | gtgactggagttcagacgtgtgctcttccgatctACACCTTGT | 183 | 948 |
| | CAAAACCCCTTTC | | |
| NGS R primer | gtgactggagttcagacgtgtgctcttccgatctATGACATGG | 184 | 949 |
| | TTGCTGAATGG | | |
| NGS R primer | gtgactggagttcagacgtgtgctcttccgatctGTCCTCAGT | 185 | 950 |
| | TGGGAACTATTT | | |
| NGS R primer | gtgactggagttcagacgtgtgctcttccgatctCTTTTCTTA | 186 | 951 |
| | GGTAGCAGATGGG | | |
| NGS R primer | gtgactggagttcagacgtgtgctcttccgatctGAATTATGG | 187 | 952 |
| | CTGCAGGAAAATTTG | | |
| NGS R primer | gtgactggagttcagacgtgtgctcttccgatctTTTCCTCTT | 188 | 953 |
| | CCTCGTCG | | |
| NGS R primer | gtgactggagttcagacgtgtgctcttccgatctTAGCCTGCA | 189 | 954 |
| | TTTGCTTTCTC | | |
| NGS R primer | gtgactggagttcagacgtgtgctcttccgatctCTTTATTCA | 190 | 955 |
| | TAAAGTTGGTCTCAGT | | |
| NGS R primer | gtgactggagttcagacgtgtgctcttccgatctGGCCCAACC | 191 | 956 |
| | TGAAGTTATT | | |
| NGS R primer | gtgactggagttcagacgtgtgctcttccgatctGTGAATTCA | 192 | 957 |
| | TCAGCTGGATCAAA | | |
| NGS R primer | gtgactggagttcagacgtgtgctcttccgatctGCAGTAAAC | 193 | 958 |
| | AGTCTCAGCAT | | |
| NGS R primer | gtgactggagttcagacgtgtgctcttccgatctCAAAGTTGT | 194 | 959 |
| | GGAGAAGGCC | | |
| NGS R primer | gtgactggagttcagacgtgtgctcttccgatctGCTCATGTC | 195 | 960 |
| | CTGGTTCCT | | |
| NGS R primer | gtgactggagttcagacgtgtgctcttccgatctTCCGAGCTG | 196 | 961 |
| | GAGGAGG | | |
| NGS R primer | gtgactggagttcagacgtgtgctcttccgatctGAGGAAACT | 197 | 962 |
| | GATGTTGATAAGAGGT | | |
| NGS R primer | gtgactggagttcagacgtgtgctcttccgatctCCAAAGCAT | 198 | 963 |
| | TAATATCCAACATAGAATGA | | |
| NGS R primer | gtgactggagttcagacgtgtgctcttccgatctCTGGGCTTT | 199 | 964 |
| | CCATGAATTATGAA | | |
| NGS R primer | gtgactggagttcagacgtgtgctcttccgatctAGGATGATG | 200 | 965 |
| | TGTTCCAACC | | |
| NGS R primer | gtgactggagttcagacgtgtgctcttccgatctCGTTACCCC | 201 | 966 |
| | AAATCCTTACC | | |
| NGS R primer | gtgactggagttcagacgtgtgctcttccgatctGCCGAAATA | 202 | 967 |
| | CTGCTCGT | | |
| NGS R primer | gtgactggagttcagacgtgtgctcttccgatctCACATTACA | 203 | 968 |
| | TGCTTCCCAGAG | | |
| NGS R primer | gtgactggagttcagacgtgtgctcttccgatctATGCTAGCC | 204 | 969 |
| | ATTACCTCCAT | | |
| NGS R primer | gtgactggagttcagacgtgtgctcttccgatctCTAACAACT | 205 | 970 |
| | TCAACTGGATATCCTTATA | | |
| NGS R primer | gtgactggagttcagacgtgtgctcttccgatctTAGGATCAG | 206 | 971 |
| | ATGCCGACAT | | |
| NGS R primer | gtgactggagttcagacgtgtgctcttccgatctTCACCTTAG | 207 | 972 |
| | CATTTTGTGACTTTT | | |
| NGS R primer | gtgactggagttcagacgtgtgctcttccgatctCTGCTTTAG | 208 | 973 |
| | TAATGCAACATACCT | | |
| NGS R primer | gtgactggagttcagacgtgtgctcttccgatctTTGCTAGAC | 209 | 974 |
| | GCTGAAGACTAATTTT | | |
| NGS R primer | gtgactggagttcagacgtgtgctcttccgatctCGGCTCCGC | 210 | 975 |
| | ATCTATTTC | | |
| NGS R primer | gtgactggagttcagacgtgtgctcttccgatctTCCCTAAGT | 211 | 976 |
| | CTAAGGCCTTAT | | |
| NGS R primer | gtgactggagttcagacgtgtgctcttccgatctCAAGTAAAG | 212 | 977 |
| | TGCCTTTCCTAGAA | | |
| NGS R primer | gtgactggagttcagacgtgtgctcttccgatctGCAAAGTGA | 213 | 978 |
| | TCATACCTCTTCAATA | | |
| NGS R primer | gtgactggagttcagacgtgtgctcttccgatctAGGTTGACT | 214 | 979 |
| | GCAGACACTAA | | |
| NGS R primer | gtgactggagttcagacgtgtgctcttccgatctAGATGCTGC | 215 | 980 |
| | TACTTCATATAGGC | | |
| NGS R primer | gtgactggagttcagacgtgtgctcttccgatctGCTATGCCA | 216 | 981 |
| | CTACCCTCC | | |
| NGS R primer | gtgactggagttcagacgtgtgctcttccgatctCGACGGTAA | 217 | 982 |
| | TCAAGTTTTGCT | | |
| NGS R primer | gtgactggagttcagacgtgtgctcttccgatctGAATATCCC | 218 | 983 |
| | GTCCAGTGTC | | |
| NGS R primer | gtgactggagttcagacgtgtgctcttccgatctGATCGCAGG | 219 | 984 |
| | GCTCATTATGT | | |
| NGS R primer | gtgactggagttcagacgtgtgctcttccgatctGATTAGGAA | 220 | 985 |
| | TCCCGGCAC | | |
| NGS R primer | gtgactggagttcagacgtgtgctcttccgatctGAATTATTT | 221 | 986 |
| | TAGTTCTCAGAGCTGCAA | | |
| NGS R primer | gtgactggagttcagacgtgtgctcttccgatctCTGTGGAGT | 222 | 987 |
| | ACCTCTTCCGT | | |
| NGS R primer | gtgactggagttcagacgtgtgctcttccgatctCCATCTCCC | 223 | 988 |
| | TTGAACATTGT | | |
| NGS R primer | gtgactggagttcagacgtgtgctcttccgatctCCTGACAGT | 224 | 989 |
| | TCTAATAAGGTACC | | |
| NGS R primer | gtgactggagttcagacgtgtgctcttccgatctAATCCATAG | 225 | 990 |
| | CAAGACGGC | | |
| NGS R primer | gtgactggagttcagacgtgtgctcttccgatctACACAGTGG | 226 | 991 |
| | CGTTCTG | | |
| NGS R primer | gtgactggagttcagacgtgtgctcttccgatctGAAACTGAG | 227 | 992 |
| | AACCCTGCTA | | |
| NGS R primer | gtgactggagttcagacgtgtgctcttccgatctCGTGTTCCT | 228 | 993 |
| | GCCTCAGG | | |
| NGS R primer | gtgactggagttcagacgtgtgctcttccgatctGCTCTAAGC | 229 | 994 |
| | TGGGTGACT | | |
| NGS R primer | gtgactggagttcagacgtgtgctcttccgatctCTGTCTCCT | 230 | 995 |
| | CTGCAGATG | | |
| NGS R primer | gtgactggagttcagacgtgtgctcttccgatctCGTTTCTCC | 231 | 996 |
| | AGGGTAGA | | |
| NGS R primer | gtgactggagttcagacgtgtgctcttccgatctGGGTAATGC | 232 | 997 |
| | TCTTCTCCAAA | | |
| NGS R primer | gtgactggagttcagacgtgtgctcttccgatctCTTCCCGAT | 233 | 998 |
| | CAGGCTGTT | | |
| NGS R primer | gtgactggagttcagacgtgtgctcttccgatctATCATGAAG | 234 | 999 |
| | CTGCTGTGCT | | |
| NGS R primer | gtgactggagttcagacgtgtgctcttccgatctGACTTACCT | 235 | 1000 |
| | TTGGACCCG | | |
| NGS R primer | gtgactggagttcagacgtgtgctcttccgatctAGCTCTGCT | 236 | 1001 |
| | TCCACCG | | |
| NGS R primer | gtgactggagttcagacgtgtgctcttccgatctATACGCCTG | 237 | 1002 |
| | GACACCCT | | |
| NGS R primer | gtgactggagttcagacgtgtgctcttccgatctCATTCACCA | 238 | 1003 |
| | TTTTATTCCATGAAATTTTT | | |
| NGS R primer | gtgactggagttcagacgtgtgctcttccgatctAGGAGAAAA | 239 | 1004 |
| | GAATGTCTTCACACAA | | |
| NGS R primer | gtgactggagttcagacgtgtgctcttccgatctTCAGGAAGT | 240 | 1005 |
| | CATTGCTTTCC | | |
| NGS R primer | gtgactggagttcagacgtgtgctcttccgatctGTCCCTTTC | 241 | 1006 |
| | TCTGCAGCA | | |
| NGS R primer | gtgactggagttcagacgtgtgctcttccgatctGAGATCTCC | 242 | 1007 |
| | TTGACCGACG | | |
| NGS R primer | gtgactggagttcagacgtgtgctcttccgatctCATTCTTCA | 243 | 1008 |
| | TCCAAGTTATCCAACTTA | | |
| NGS R primer | gtgactggagttcagacgtgtgctcttccgatctTCTCTTCCC | 244 | 1009 |
| | TTTAGCTTCTCAC | | |
| NGS R primer | gtgactggagttcagacgtgtgctcttccgatctCTTGCAGGA | 245 | 1010 |
| | GCTTGAACATA | | |
| NGS R primer | gtgactggagttcagacgtgtgctcttccgatctCTACGCCGC | 246 | 1011 |
| | CTTCTCC | | |
| NGS R primer | gtgactggagttcagacgtgtgctcttccgatctTGTACTTGG | 247 | 1012 |
| | TACCACAGCATT | | |
| NGS R primer | gtgactggagttcagacgtgtgctcttccgatctCTTGCGAGT | 248 | 1013 |
| | CTCAGGTACTAA | | |
| NGS R primer | gtgactggagttcagacgtgtgctcttccgatctAAAATAAAC | 249 | 1014 |
| | GCCAACACGATG | | |
| NGS R primer | gtgactggagttcagacgtgtgctcttccgatctGAAATAACT | 250 | 1015 |
| | GAGTCGCTGGTT | | |
| NGS R primer | gtgactggagttcagacgtgtgctcttccgatctATTTTGGTA | 251 | 1016 |
| | CCTGAAGATCTGG | | |
| NGS R primer | gtgactggagttcagacgtgtgctcttccgatctGAGGAGGGC | 252 | 1017 |
| | GCTAGTG | | |
| NGS R primer | gtgactggagttcagacgtgtgctcttccgatctATTGCTTGT | 253 | 1018 |
| | CACCACTTTGG | | |
| NGS R primer | gtgactggagttcagacgtgtgctcttccgatctCGCTGCTAC | 254 | 1019 |
| | CTGGATG | | |
| NGS R primer | gtgactggagttcagacgtgtgctcttccgatctAATCTCCAC | 255 | 1020 |
| | GCCCGAAC | | |

All gRNAs, HDR templates, and primers were synthesized by IDT (Coralville, IA). SEQ ID NO: 1-255 represent the 20 nt protospacer sequence corresponding to gRNAs used in Example 2. The generated gRNAs have 5′- and 3′-Alt-R ™ termini modifications and were produced in both Cas9 crRNA and sgRNA formats. SEQ ID NO: 256-510 represent the HDR templates tested in Example 2. HDR templates have 5′- and 3′-Alt-R™ termini modifications (+) and phosphorothioate (*) linkages between nucleotides 1-2, 2-3, 84-85, and 85-86. The 5′- and 3′-Alt-R ™ termini modifications are a proprietary termini-blocking technology available from IDT (Coralville, IA). SEQ ID NO: 511-1020 represent the NGS primers used in Example 2. Uppercase nucleotides indicate the gene specific portion of the primers, lowercase nucleotides indicates constant regions for subsequent NGS barcoding steps.

To create features for predictive modeling, software was developed to describe the resulting NHEJ/MMEJ profile of CRISPR Cas9 editing and connected this to the output of the rhAmpSeq CRISPR Analysis System. This includes additional indel profile features such as top allele frequency, templated insertion frequency, MMEJ deletion frequency, entropy, insertion size frequency, GC insertion motif frequency, and deletion size frequency. Definitions for these features are described (Table 2). Indel profiles were characterized in “RNP only control” conditions (i.e., no HDR template added). To remove sites that could introduce confounding factors for modeling (e.g., insufficient editing, insufficient data, etc.), sites were filtered that had <90% Cas9 editing in RNP only controls, >10% background editing called in unedited controls or <500 sequencing reads in either the RNP only controls or the HDR conditions. After applying filters, 150 sites in HAP1 were used as an input for further correlative analyses and modeling efforts.

TABLE 2
Metric Definitions of Different NHEJ/MMEJ Repair Features
Used as Inputs into the HDR Predictive Model

| Indel Profile Feature | Definition |
|---|---|
| percentEdited | % reads with SNP and/or indel events |
| percentUnedited | % reads with no mutations relative to the reference |
| percentIndels | % reads with indels or indels + SNP(s) |
| percentNHEJ | % reads with indels or indels + SNP(s) |
| percentOther | % reads with only SNP variant(s) |
| percentPerfectHDR | % reads with no mutations relative to the reference with donor |
| | mutations incorporated (post-HDR) |
| percentImperfectHDR | % reads with 1 or more mutations relative to the reference with donor |
| | mutations incorporated |
| percentHDR | % ImperfectHDR + % PerfectHDR |
| percentInFrame | % reads with a total indel size of 0, or a multiple of 3 |
| percentFrameshift | % reads with a total indel size that is not 0, or a multiple of 3 |
| percentInsertions | % reads with an insertion |
| percentDeletions | % reads with a deletion |
| percentSNPLines | % reads with a SNP variant call |
| percentMMEJ | % deletion containing reads with microhomology characteristic (≥2 bp) |
| | of the microhomology-mediated end joining (MMEJ) pathway |
| percentTemplatedInsertion | % insertion containing reads with 100% homology to the adjacent |
| | genomic region (5′ or 3′) |
| percentGC-Insertion | % insertion containing reads with ≥2 bp of only GC content with no |
| | template homology |
| TopNAF | defined as the sum of the editing frequencies for the top “N” most |
| | common editing outcomes within an indel profile |
| N + DelFreq | defined as the sum of the editing frequencies for all indels |
| | corresponding to a deletion event of “N” bp or greater in length |
| N + InsFreq | defined as the sum of the editing frequencies for all indels |
| | corresponding to a insertion event of “N” bp or greater in length |
| | defined as the sum of the editing frequencies for all indels |
| MMEJN+ | corresponding to a MMEJ deletion event with “N” bp or greater in |
| | microhomology length |
| NDelFreq | defined as the sum of the editing frequencies for all indels |
| | corresponding to a deletion event of “N” bp |
| NInsFreq | defined as the sum of the editing frequencies for all indels |
| | corresponding to a insertion event of “N” bp |
| | defined as the sum of the editing frequencies for all indels |
| InsHomologyN+ | corresponding to a insertion event of “N” bp or greater homology to the |
| | region adjacent to insertion |
| Entropy | A measure of disorder of the indel profile using the unique indels and |
| | their frequencies through the SciPy computation for Entropy |
| Predicted -- KLDivergence | KL Divergence compared to an in silico predicted indel profile at the |
| | same location using FORECasT |
| Predicted -- FSFrequency | Frameshift frequency predicted by an in silico predicted indel profile at |
| | the same location using FORECasT |

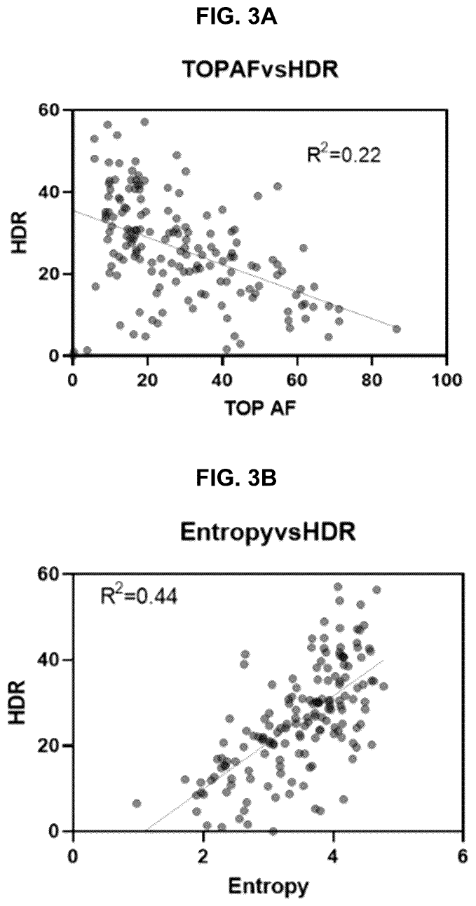

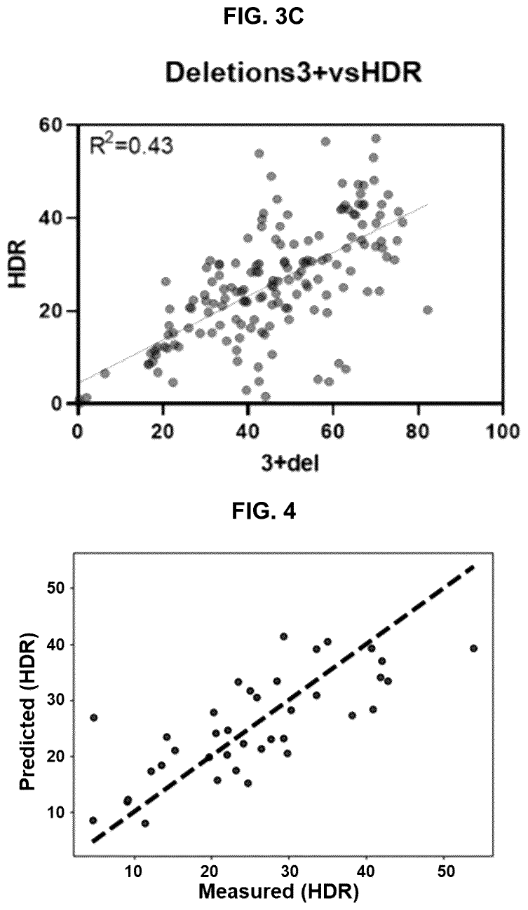

Pearson correlations (R) between individual indel profile attributes and HDR outcomes were calculated to first determine key predictive features for HDR (Table 3). Several indel profile features were identified as candidates for HDR prediction (FIG. 3). A negative correlation between HDR rates and the TopAF was observed (R²=0.22). Positive correlations were observed between HDR rates and the indel profile Entropy (R²=0.44) and the Deletions 3+ (R²=0.43). These findings confirm the observation made by Tatiossian et al. that MMEJ-based large deletions are predictive for HDR. See Tatiossian et al., Mol. Ther. 29(3): 1057-1069 (2021). However, indel profile complexity is another key feature as evidenced by the TopAF and Entropy results. While the concept of targeting top alleles with recursive editing or double-tap methods to improve HDR was introduced by Möller et al. and Bodai et al., the predictive nature of the top allele feature for selecting good HDR gRNAs was not proposed. See Möller et al., Nature Commun. 13(1): 4550 (2022); Bodai et al., Nature Commun. 13(1): 2351 (2022). The negative correlation between HDR frequency and TopAF additionally suggests that the top candidates for recursive editing/double-tap methods may be the worst initial candidates for HDR, or that recursive editing methods may need to be applied in a manner to reduce the prevalence of these high frequency repair outcomes before HDR can maximally enhanced.

TABLE 3
Pearson Correlation Values between HDR Editing Frequencies and
Indel Profile Attributes of the RNP Only Control Samples in
HAP1 cells. HDR editing frequency included as a control.

| Indel Profile Attribute | Pearson Correlation |
|---|---|
| HDR | 1 |
| Entropy | 0.661 |
| 3 + DelFreq | 0.610 |
| 6 + DelFreq | 0.567 |
| percentDeletions | 0.548 |
| 10 + DelFreq | 0.514 |
| 3 + InsFreq | 0.509 |
| InsHomology5+ | 0.411 |
| MMEJ3+ | 0.309 |
| 20 + DelFreq | 0.245 |
| MMEJ5+ | 0.220 |
| percentGCInsertion | 0.180 |
| MMEJ6+ | 0.174 |
| MMEJ10+ | 0.093 |
| InsHomology10+ | 0.016 |
| percentMMEJ | 0.011 |
| InsHomology20+ | −0.018 |
| Predicted--FSFrequency | −0.027 |
| 1DelFreq | −0.047 |
| Predicted--KL_Divergence | −0.426 |
| percentFrameshift | −0.429 |
| percentTemplatedInsertion | −0.527 |
| percentInsertions | −0.556 |
| TopAFFreq | −0.585 |
| 1InsFreq | −0.592 |
| Top3AFFreq | −0.674 |

### Example 3

### Development of HDR Predictive Model

While single features within NHEJ/MMEJ indel profiles were shown to be correlative to HDR outcomes, it is likely that the correlations could be enhanced by collectively evaluating the features in the context of the dependent variable within a constructed model. For the 150 HAP1 sites evaluated in Example 2, features were used to first construct a Multiple Linear Regression in GraphPad Prism (Dotmatics) with the sites paired HDR value as the dependent variable to identify and remove features contributing to multi-collinearity issues as according to the program. The dataset was then split into training and test datasets (75/25 split; 100 bootstraps) and features were then used to construct a Gradient Boosting Regressor using SciKit-Learn and evaluated using the bootstrapped test datasets. Analysis of the model in HAP1 showed that the model a good Pearson correlation of determination (R²=0.45±0.13) and strong Spearman correlation for rank-order determination (Spearman correlation=0.67±0.09) across 100 bootstraps (FIG. 4; sample test result).

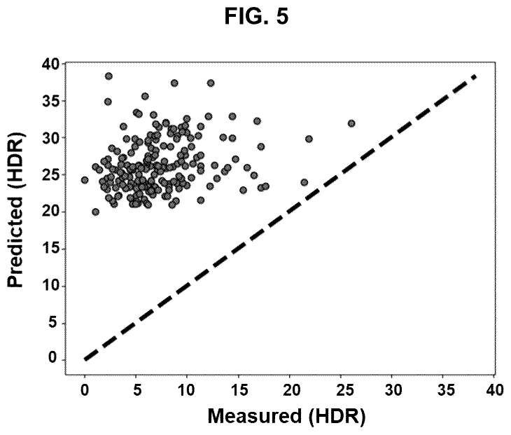

To test if the model was directly translatable to a cell line with known NHEJ/MMEJ repair differences, the HDR prediction model built on HAP1 data was further tested using the Jurkat HDR and indel profile data generated for the same sites as described in Example 2. It can be seen that the HAP1 model predicted HDR rates do not generalize well to the measured Jurkat HDR rates (FIG. 5). However, it has been previously observed by Kurgan et al. that Jurkat Cas9 indel profiles are very different to what has been reported as general NHEJ/MMEJ repair profiles for other cell lines. See Kurgan et al., Mol. Ther.—Methods Clin. Dev. 21: 478-491 (2021), which is incorporated by reference herein for such teachings.

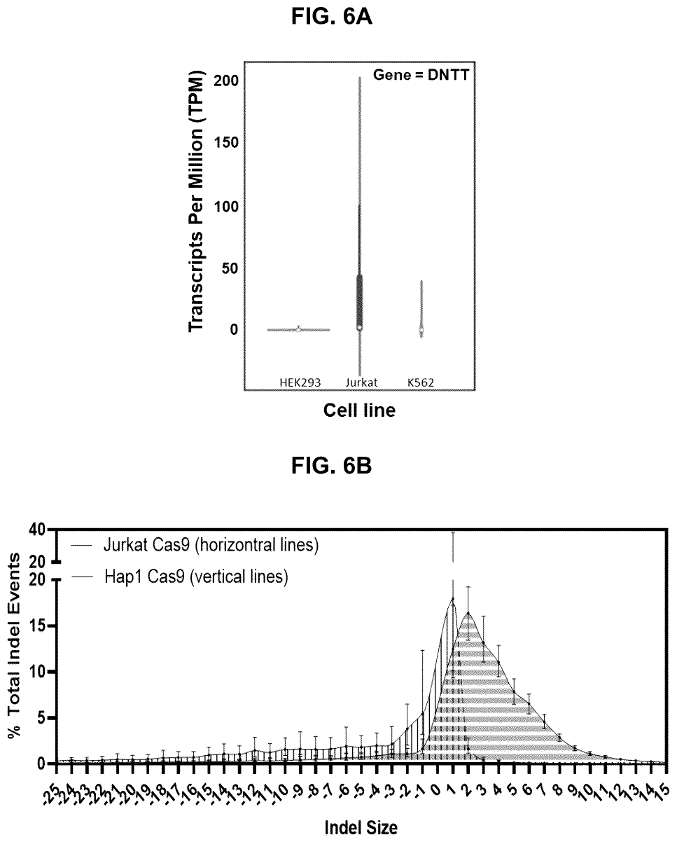

Expression profiles of DNA repair factors may contribute to unique sets of HDR prediction factors and thus impact this model's ability to accurately predict HDR outcomes in specific cell types. In the case of Jurkat cells, higher expression of the immune cell-specific terminal deoxynucleotidyl transferase (TdT) relative to other commonly used laboratory cell lines (FIG. 6A) contributes to a unique set of HDR predicting factors for Jurkat cells, and thus a lack of generalization for the HAP1 model. The TdT protein is a template-independent DNA polymerase that contributes to V(D)J recombination in lymphocytes through the random addition of nucleotides to an available 3′-terminus at a DSB. As such, a higher frequency of insertions was observed in the indel profiles in Jurkat cells (FIG. 6B) and altered Pearson correlations between HDR and indel profile attributes when compared to HAP1 cells (Table 4).

TABLE 4
Pearson Correlation Values between HDR Editing Frequencies and
Indel Profile Attributes of the RNP-only Control Samples in
Jurkat Cells. HDR editing frequency included as a control.

| Indel Profile Attribute | Pearson Correlation |
|---|---|
| HDR | 1 |
| InsHomology10+ | 0.384 |
| percentDeletions | 0.227 |
| Entropy | 0.215 |
| 3 + DelFreq | 0.183 |
| 6 + DelFreq | 0.123 |
| InsHomology5+ | 0.121 |
| 1DelFreq | 0.115 |
| Predicted--FORECasT_FrameshiftFrequency | 0.101 |
| percentTemplatedInsertion | 0.089 |
| InsHomology20+ | 0.072 |
| MMEJ3+ | 0.030 |
| percentMMEJ | 0.026 |
| 10 + DelFreq | −0.009 |
| MMEJ5+ | −0.015 |
| MMEJ6+ | −0.034 |
| TopAFFreq | −0.037 |
| percentFrameshift | −0.040 |
| 1InsFreq | −0.077 |
| Top3AFFreq | −0.085 |
| MMEJ10+ | −0.089 |
| 20 + DelFreq | −0.109 |
| 3 + InsFreq | −0.154 |
| percentGCInsertion | −0.160 |
| percentInsertions | −0.200 |
| Predicted--FORECasT_KL_Divergence | −0.398 |

### Example 4

### Application of Key Attributes and HDR Prediction Model Across Cell Types

To explore the performance of the HAP1 based HDR prediction model across additional cell types, a subset of 48 sites was selected from the initial 263 sites described in Example 2. Sites selected had >90% editing in RNP only controls, <10% background editing in unedited controls, and HDR rates that ranged from 2-50% in HAP1. CRISPR Cas9 HDR reagents for these 48 sites were delivered into K562, iPSC, and primary T cell lines to evaluate editing outcomes.

Cas9 RNP (consisting of Alt-R™ S.p. Cas9 nuclease and Alt-R™ sgRNA) was formed at a 1:1.2 ratio of Cas9 protein to gRNA. For K562 cells, 2 μM Cas9 RNP complexes were delivered with 2 μM Alt-R Cas9 Electroporation Enhancer and 2 μM Alt-R HDR Donor Oligos using the Lonza 4D-Nucleofector 96-well system (Lonza, Basel, Switzerland) and cell line appropriate conditions (FF-120). For iPSCs, 4 μM Cas9 RNP complexes were delivered with 4 μM Alt-R Cas9 Electroporation Enhancer (RNP only controls) and 4 μM Alt-R HDR Donor Oligos (HDR conditions) using the Lonza 4D-Nucleofector 96-well system (Lonza, Basel, Switzerland) and cell line appropriate conditions (CA-137). For primary T cells, 4 μM Cas9 RNP complexes were delivered with 3 μM Alt-R Cas9 Electroporation Enhancer and 2 μM Alt-R HDR Donor Oligos using the Lonza 4D-Nucleofector 96-well system (Lonza, Basel, Switzerland) and cell line appropriate conditions (ER-115). HDR donors were designed to introduce a 6 bp “GAATTC” sequence at the DSB and corresponded to the non-targeting DNA strand relative to the gRNA. Conditions tested included RNP only, RNP+HDR Donor, and untreated controls. DNA was extracted after 48 hours (K62, primary T cells) or 96 hours (iPSCs) using QuickExtract™ DNA extraction solution (Lucigen, Madison, WI). Editing outcomes were quantified by NGS amplicon sequencing on the Illumina MiSeq platform using rhAmpSeq library preparation methods. Data analysis was conducted using IDT's in-house version of the rhAmpSeq CRISPR Analysis System. Sequences for gRNA protospacers, Donor Oligos, and sequencing primers are listed in Table 5.

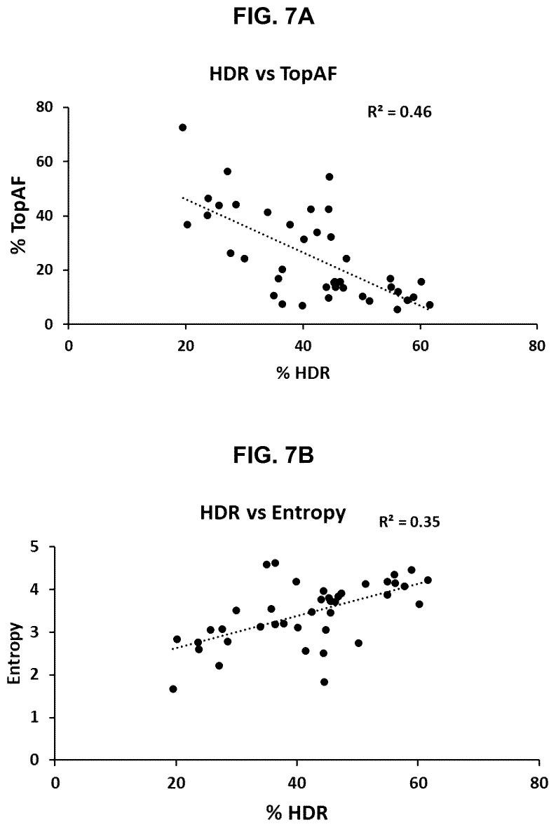

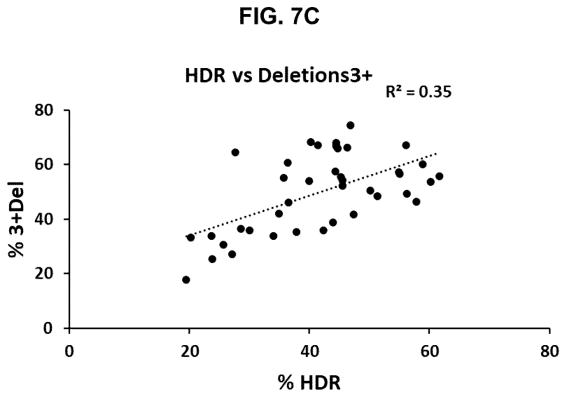

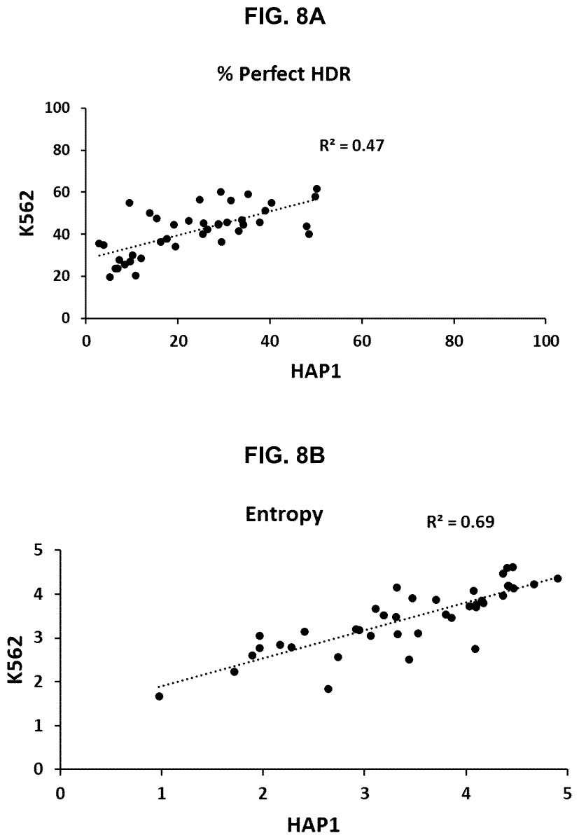

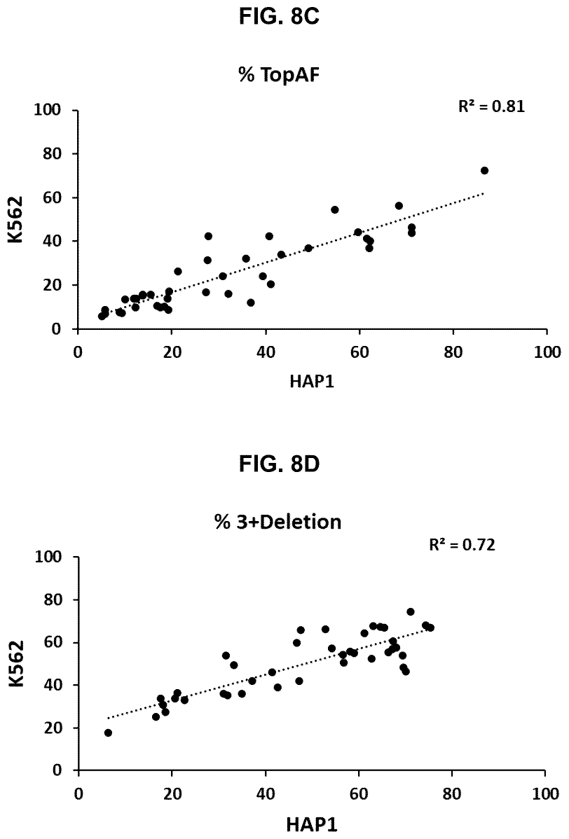

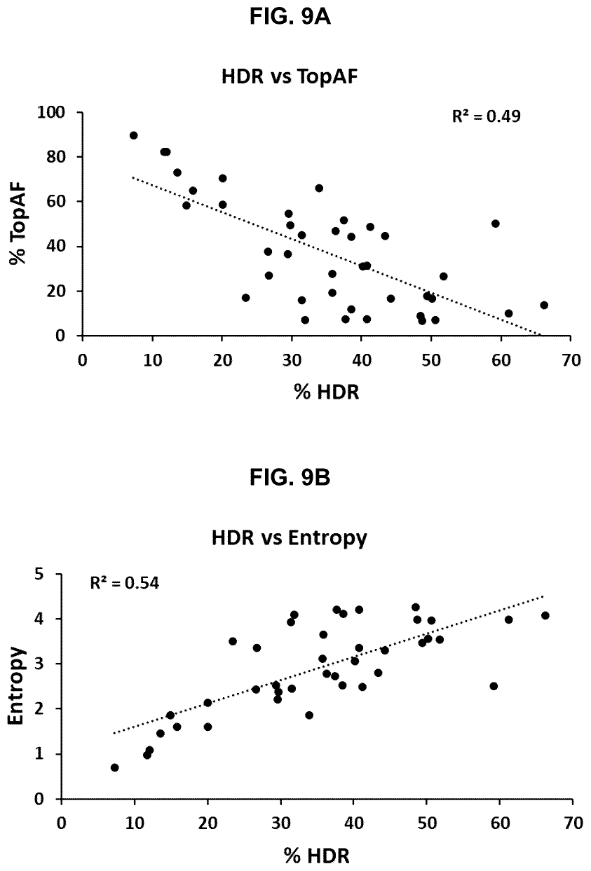

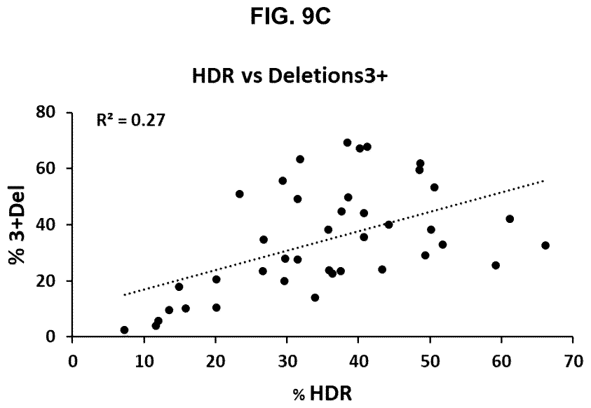

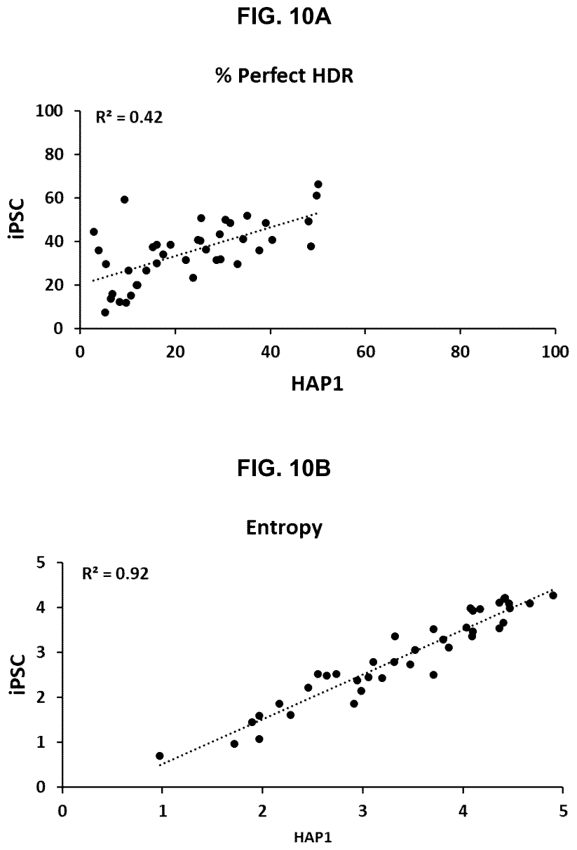

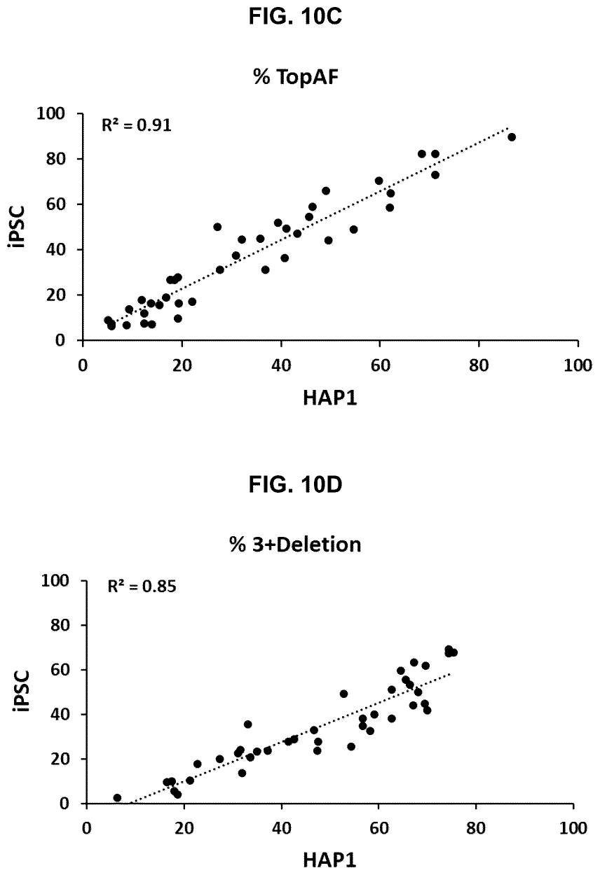

Similar correlations between HDR and key indel profile attributes were observed in K562 cells, iPSCs, and primary T cells, with some notable exceptions (FIG. 7, 9, 11). A negative correlation between HDR rates and the TopAF was observed (R²=0.46 for K562, R²=0.49 for iPSCs). Positive correlations were observed between HDR rates and the indel profile Entropy (R²=0.35 for K562, R²=0.54 for iPSCs, R²=0.35 for primary T cells) and the Deletions 3+ (R²=0.35 for K562, R²=0.27 for iPSCs, R²=0.27 for primary T cells). Furthermore, the HDR and indel profile attributes were well correlated (R²=0.47-0.81 for K562, R²=0.42-0.92 for iPSCs, R²=0.19-0.63 for primary T cells) when results were compared to the original HAP1 data set (FIG. 8, 10, 12).

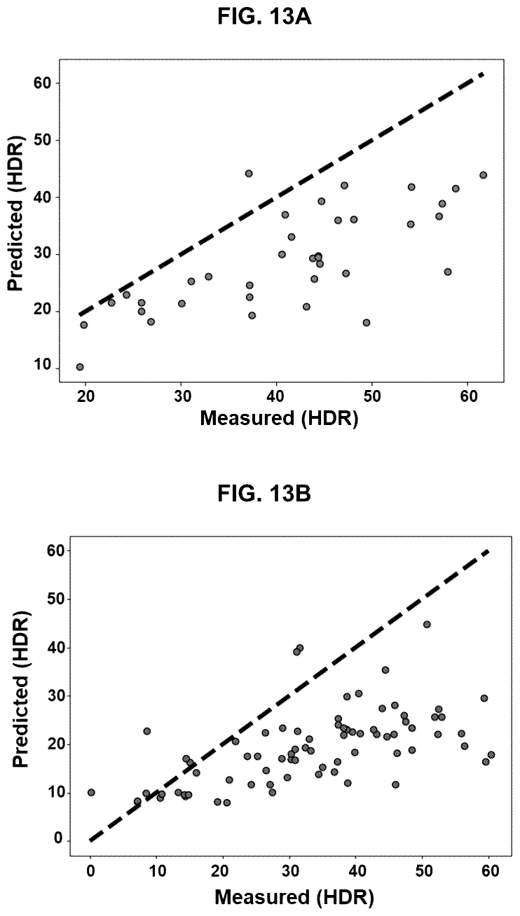

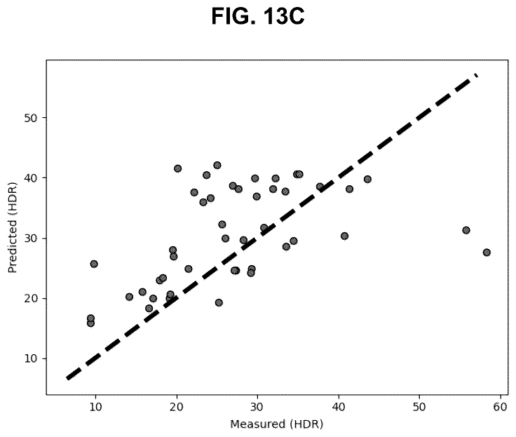

The K562, iPSC, and primary T cell indel profile data was then processed through the 100 bootstrapped iterations of the HAP1 based HDR prediction model and compared against the measured HDR rate in each cell type (sample results depicted in FIG. 13). While the model was not able to accurately predict the absolute % HDR (Pearson correlation=−0.80±0.28 for K562, −0.63±0.17 for iPSCs, 0.03±0.19 for primary T cells), the model was able to accurately rank gRNAs for overall HDR potential (Spearman correlation=0.66±0.06 for K562, 0.66±0.03 for iPSCs, 0.53±0.10 for primary T cells). The inability to predict absolute HDR values was not surprising due to the variability in HDR rates observed between cell lines. However, the ability of the model to provide a ranking of gRNAs independent of the cell line is a valuable feature for CRISPR HDR applications.

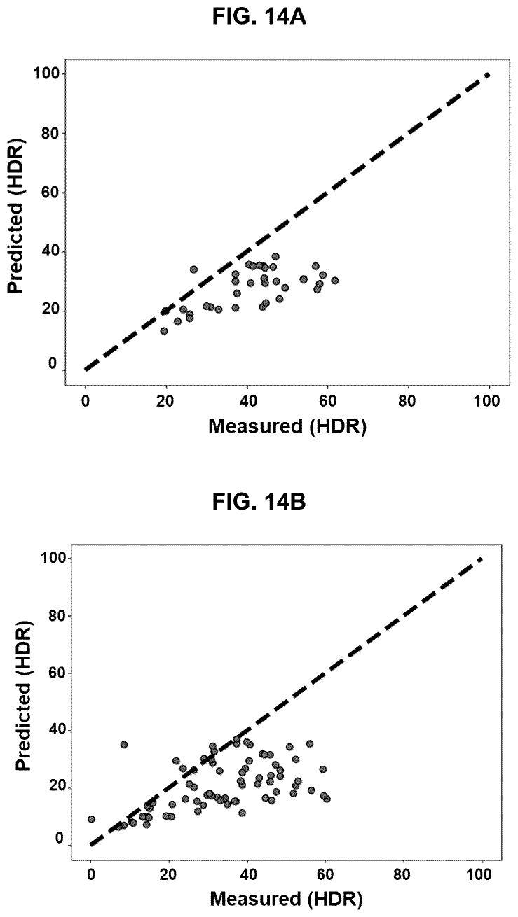

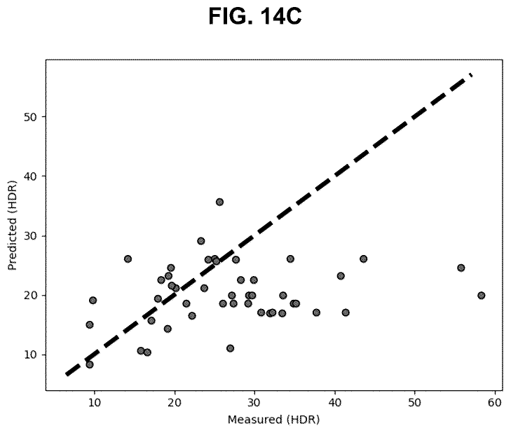

To investigate the performance of the HDR prediction model described here relative to prior art, a comparison to the predictive value of large deletion frequencies in isolation was conducted. A secondary prediction model was created using the HAP1 3+Del frequency as the sole predictive feature. Using this model, predicted HDR rates were compared against measured HDR rates from the K562, iPSC, and primary T cell data sets (FIG. 14). The deletion-based model was successful in ranking the HDR potential of gRNAs for some cell lines (Spearman correlation=0.52 for K562 cells and 0.53 for iPSCs) but did not reach the same degree of HDR ranking accuracy as the full tool (Spearman correlation=0.66±0.06 for K562 cells and 0.66±0.03 for iPSCs). In the case of primary T cells, the deletion-based model was unsuccessful in ranking the

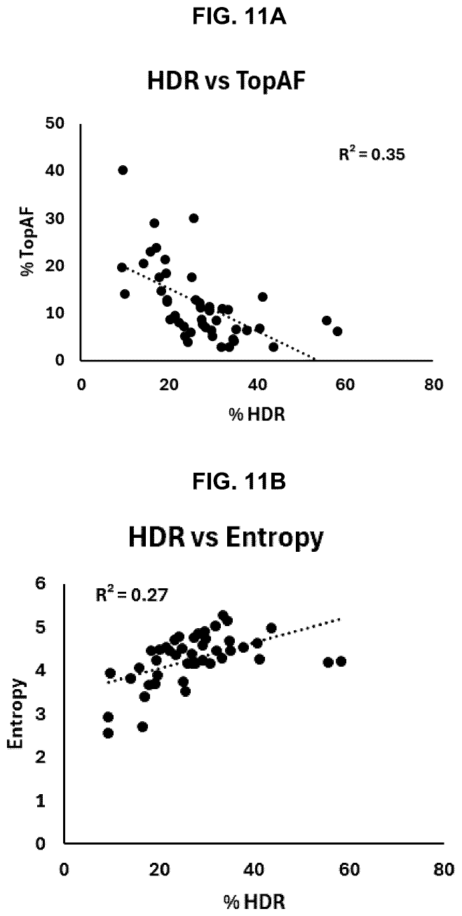

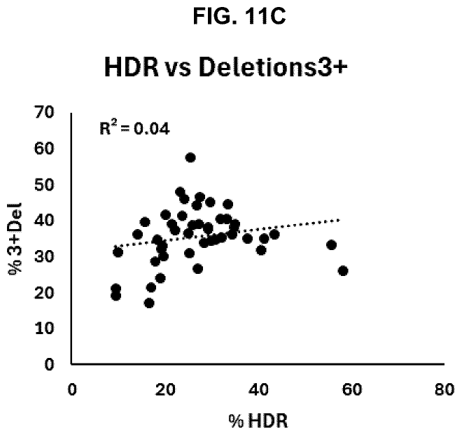

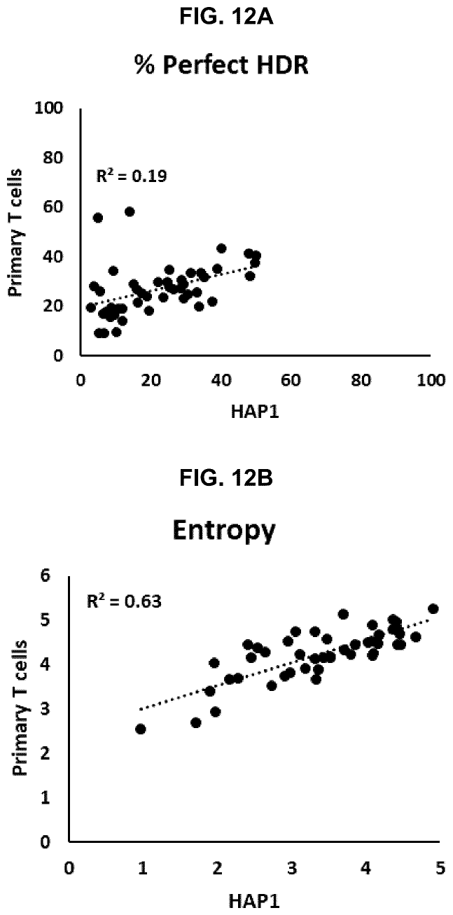

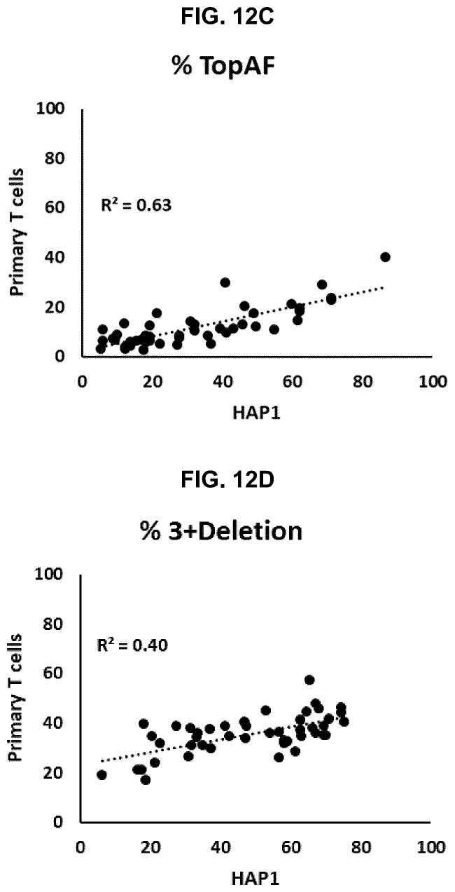

HDR potential of gRNAs (Spearman correlation=0.16±0.07) when compared to the comprehensive full prediction tool (Spearman correlation=0.53±0.10). This discrepancy is largely due to the poor correlation between HDR and large deletions observed in primary T cells (FIG. 11). This further demonstrates the benefit of the comprehensive model over prior art, where the full profile of indel features can compensate for poor correlations of an individual feature that may be cell-line specific.

Taken together, these data establish the ability of an HDR prediction model to provide rank HDR potential for Cas9 gRNAs based on indel profile features including large deletion frequencies, entropy, and top allele frequencies among other factors. These data further demonstrate the benefit of a model based on comprehensive indel profile features over the published prior art utilizing deletion frequency alone. This model is applicable across multiple cell types, including clinically relevant cell types such as iPSCs and primary T cells. It may be possible to develop cell type specific HDR models based on the expression profiles of key DNA repair genes that contribute to unique indel profile features.

TABLE 5
gRNAs, HDR Templates, and Sequencing Primers

| | | Target | SEQ ID |
|---|---|---|---|
| Purpose | Sequence (5′→3′) | No. | NO. |
| gRNA protospacer | CGCATGACCTCGACCATCTG | 1 | 1021 |
| gRNA protospacer | TGCCAGATAGCACCGTCCAA | 2 | 1022 |
| gRNA protospacer | TCGTGTGGGAGCACGACATC | 3 | 1023 |
| gRNA protospacer | GCCTGGACGACATTGGCCAT | 4 | 1024 |
| gRNA protospacer | GTCAGGATGACCGAATACGT | 5 | 1025 |
| gRNA protospacer | TTTCCGGCTAGCACGTACCA | 6 | 1026 |
| gRNA protospacer | ATGAAGCGCCCACACGAAAT | 7 | 1027 |
| gRNA protospacer | AAGAAGCGTTCGTATTCGGT | 8 | 1028 |
| gRNA protospacer | GGCTTGTTACACGTACTCTA | 9 | 1029 |
| gRNA protospacer | ATAAGAGCTGCTCATCGCAT | 10 | 1030 |
| gRNA protospacer | GATCGACGTGTACCACTACG | 11 | 1031 |
| gRNA protospacer | GGCCCCGCTGAACGACACCA | 12 | 1032 |
| gRNA protospacer | ACGGAGCTGACTTCGCCAAG | 13 | 1033 |
| gRNA protospacer | GCAAATGAGTACGGCTTGTT | 14 | 1034 |
| gRNA protospacer | GAGTGGATATGGCCTCGACC | 15 | 1035 |
| gRNA protospacer | ACATTGTGAGCCGGGTCAAC | 16 | 1036 |
| gRNA protospacer | CTTCGACACAATGCCAACGT | 17 | 1037 |
| gRNA protospacer | CCATTCGAGTCAAGCTTGGT | 18 | 1038 |
| gRNA protospacer | GGCCACTCACGTGAACACTA | 19 | 1039 |
| gRNA protospacer | AGAGATTGTGCATCGTTACG | 20 | 1040 |
| gRNA protospacer | GCAACAACAAGGAGTACCCG | 21 | 1041 |
| gRNA protospacer | GAACCATTGCCACCCGTCTC | 22 | 1042 |
| gRNA protospacer | TGTAAAAGTGAACAGGTCGA | 23 | 1043 |
| gRNA protospacer | GTTCTCGTCAAGGACGGCGT | 24 | 1044 |
| gRNA protospacer | CATGGCAACTAACTCTGATT | 25 | 1045 |
| gRNA protospacer | CTCACGCGGCTGGAAACCAC | 26 | 1046 |
| gRNA protospacer | TTGCACAGATCTGGGAGTAT | 27 | 1047 |
| gRNA protospacer | GCCAACAACCGTGCCTACAA | 28 | 1048 |
| gRNA protospacer | GTTCGGCAGCAACGTTGAGT | 29 | 1049 |
| gRNA protospacer | ACTCTAACACGTTGGGGACG | 30 | 1050 |
| gRNA protospacer | GCCACCAATAATCGCAAGAG | 31 | 1051 |
| gRNA protospacer | CAACGAGGCAGCCGACACGT | 32 | 1052 |
| gRNA protospacer | GATCCACCAAAGCTTCTGTC | 33 | 1053 |
| gRNA protospacer | GTGTGTCTAACAATACAACT | 34 | 1054 |
| gRNA protospacer | ACACGAAGCCAATCAGGTTC | 35 | 1055 |
| gRNA protospacer | TGGTGAAGAGCGTCCACCGG | 36 | 1056 |
| gRNA protospacer | GGTGTTCCGAATGGGACCAC | 37 | 1057 |
| gRNA protospacer | GTACGATGACTTCCCCCACG | 38 | 1058 |
| gRNA protospacer | TCAACGCCAGATCTTGTCGT | 39 | 1059 |
| gRNA protospacer | GTAGTCTACCACCATGCCAC | 40 | 1060 |
| gRNA protospacer | CTGGGCCACAAAAGGGATAC | 41 | 1061 |
| gRNA protospacer | CCGAGTCCACATGTTAGCCC | 42 | 1062 |
| gRNA protospacer | GCCCACCAAACCCCCGACGA | 43 | 1063 |
| gRNA protospacer | GTCCCCACAAAGTTCAGGGC | 44 | 1064 |
| gRNA protospacer | CTCAGCAAGGACGAACGCCA | 45 | 1065 |
| gRNA protospacer | CACTAGAACGCCACCCAAAG | 46 | 1066 |
| gRNA protospacer | GTTCACCAGCTCCGTGTCGA | 47 | 1067 |
| gRNA protospacer | GGGTTGACCCCAAAGCTAAC | 48 | 1068 |
| HDR donor | +A*C*CAAATGGCCCTTTTCATTCAGCGCATGACCTCGACCAT | 1 | 1069 |
| | GAATTCCTGTGGTTTCCTGTTGGGATTTTTCAGGGGTTGGAAA | | |
| | C*T*G+ | | |
| HDR donor | +C*C*CACCAGCTTCAGCAGTCCAAGTGCCAGATAGCACCGTC | 2 | 1070 |
| | GAATTCCAAAGGCCCCATACAAGGCCCTCCACAGCCGTGCCCC | | |
| | C*T*C+ | | |
| HDR donor | +C*C*CGCCAGAGAGCAGGGCTGTCCTCGTGTGGGAGCACGAC | 3 | 1071 |
| | GAATTCATCAGGCCCAGTGCCGTCAGGATCTCTGTCACCTGGC | | |
| | C*C*C+ | | |
| HDR donor | +C*A*GGAGACCCCTCAACTTTGGCTGCCTGGACGACATTGGC | 4 | 1072 |
| | GAATTCCATGGGATCAAGGTAGAGAGAGGGGCCCTCCTCTTTC | | |
| | C*C*T+ | | |
| HDR donor | +A*C*CTGGAGCCCTGGCTTATGGGAGTCAGGATGACCGAATA | 5 | 1073 |
| | GAATTCCGTCGGGGTGAGTCTGCCTTGAGACAGGGAAGTGGTT | | |
| | G*A*A+ | | |
| HDR donor | +A*G*ACATTGCCAAGGTGGTCATGGTTTCCGGCTAGCACGTA | 6 | 1074 |
| | GAATTCCCAGGGCACTTTGCGAAGGGAGCGGTCAGAGAATACG | | |
| | T*C*C+ | | |
| HDR donor | +T*C*CAGGTTGGAGTCAGCAGTGCGAAGAAGCGTTCGTATTC | 7 | 1075 |
| | GAATTCGGTAGGAGAGAGAGGGCTGCCTGGAGTGGAGGCCTGA | | |
| | G*T*C+ | | |
| HDR donor | +A*G*TGGGGCAGGCAGCCCGGCCCAATGAAGCGCCCACACGA | 8 | 1076 |
| | GAATTCAATGGGAGGGGTGATCTCCACGAGGGCAATGTCATTT | | |
| | C*C*C+ | | |
| HDR donor | +G*T*ACAGTGGATCATCTCCGAATTGGCTTGTTACACGTACT | 9 | 1077 |
| | GAATTCCTATGGTAGCTGTACCTCTGTATGACACCTTGGGACC | | |
| | A*G*A+ | | |
| HDR donor | +T*C*CTCCTCTTCATCATACGACTCATAAGAGCTGCTCATCG | 10 | 1078 |
| | GAATTCCATCGGAATCATAATCTGTAAAAAATAAACATACACT | | |
| | G*G*T+ | | |
| HDR donor | +T*A*CTTTGAGGTGGACATCCCTAAGATCGACGTGTACCACT | 11 | 1079 |
| | GAATTCACGAGGTGGACATCAAGCCGGATAAGTGTCCCCGTAG | | |
| | A*G*T+ | | |
| HDR donor | +G*T*GTGCCAAGACTGCCCCCTGCTGGCCCCGCTGAACGACA | 12 | 1080 |
| | GAATTCCCAGGGTGGTGCACGCCGCGAAAGCTGCCCTGGCCGC | | |
| | C*T*T+ | | |
| HDR donor | +G*C*GCTGTGCCCAGTACAAGAAGGACGGAGCTGACTTCGCC | 13 | 1081 |
| | GAATTCAAGTGGCGTTGTGTGCTGAAGATTGGGGAACACACCC | | |
| | C*C*T+ | | |
| HDR donor | +A*T*TCTTCTGTAGTTCTGTATCTTGCAAATGAGTACGGCTT | 14 | 1082 |
| | GAATTCGTTAGGTGGCATTAAGAGAGCATCCACCAGTCCACAT | | |
| | T*T*C+ | | |
| HDR donor | +G*G*CACCACGCTGGTGGAGTGCAAGAGTGGATATGGCCTCG | 15 | 1083 |
| | GAATTCACCTGGAGACCGAGCTCAAGATGCTGCGCGTGATTGA | | |
| | G*C*G+ | | |
| HDR donor | +G*A*CACTCACGGTGCAGGCGGCTGACATTGTGAGCCGGGTC | 16 | 1084 |
| | GAATTCAACTGGCTGGGCCATCTCGGGCAGCCTCTTTCTTCGG | | |
| | G*C*A+ | | |
| HDR donor | +A*C*CTGGAGTCTGTGAGAGTGCTCCTTCGACACAATGCCAA | 17 | 1085 |
| | GAATTCCGTGGGCAAAGAGAACCGCCAGGGCTGGGCAGGTACT | | |
| | G*C*A+ | | |
| HDR donor | +G*A*GGACTGACTTACGGGGACTGGCCATTCGAGTCAAGCTT | 18 | 1086 |
| | GAATTCGGTGGGTCGGGCAGATTTCCTGGAGGCCAGGGCAGCC | | |
| | A*C*G+ | | |
| HDR donor | +G*T*GCGGAGACTCCTTTCTGAAAAGGCCACTCACGTGAACA | 19 | 1087 |
| | GAATTCCTAGGGATGAAGATGAGTATACCCCTCTTCATCGAGC | | |
| | A*G*C+ | | |
| HDR donor | +C*A*GGACAATGAGCTCTTGACGCTAGAGATTGTGCATCGTT | 20 | 1088 |
| | GAATTCACGTGGAGCTGCTGGACAAATATTTTGGAAATGTAAG | | |
| | T*G*T+ | | |
| HDR donor | +C*A*GAGGCCAGGAGCGCCAGGAGGGCAACAACAAGGAGTAC | 21 | 1089 |
| | GAATTCCCGGGGCTGCATGGCACCTCTGTTCCTGCAAGGAAGT | | |
| | G*T*C+ | | |
| HDR donor | +C*C*CAGCCCAGCACACCCTCACCAGAACCATTGCCACCCGT | 22 | 1090 |
| | GAATTCCTCTGGTCCTGTTCACCACTGTCTCCAGCAGCTCCTT | | |
| | C*A*T+ | | |
| HDR donor | +C*A*ATGGAGATTCATTTTCAGGTATGTAAAAGTGAACAGGT | 23 | 1091 |
| | GAATTCCGAAGGTTTGAATATTTATCTGGGGGTCCTATCCAAT | | |
| | C*A*T+ | | |
| HDR donor | +G*C*TGCTGGGAGCAGCACTGCTCAGTTCTCGTCAAGGACGG | 24 | 1092 |
| | GAATTCCGTGGGCGTGGGTGAGTCTGCCACAAAACTTATAAAA | | |
| | A*G*C+ | | |
| HDR donor | +T*G*GTTACGTTTTCTTACCTCCAACATGGCAACTAACTCTG | 25 | 1093 |
| | GAATTCATTTGGAAATGCCAATTCGGTCTCGGTCACAACTGTC | | |
| | T*A*C+ | | |
| HDR donor | +T*A*GCTGTTGGTCTTGTCCCTGGACTCACGCGGCTGGAAAC | 26 | 1094 |
| | GAATTCCACAGGAGCAATGCACTGGTTCTCCTCTCTCAACACT | | |
| | T*T*A+ | | |
| HDR donor | +C*T*TCAGTGCATCTCTCACTGCTTTTGCACAGATCTGGGAG | 27 | 1095 |
| | GAATTCTATCGGATGTAGCTGGGAGAAAATGAGAGAAGGTATA | | |
| | T*G*G+ | | |
| HDR donor | +C*A*GCATTCACCTGGAAGGTCCAGGCCAACAACCGTGCCTA | 28 | 1096 |
| | GAATTCCAACGGGCAGTTCAAGGAGAAGGTGATCCTGTGCTGG | | |
| | C*A*A+ | | |
| HDR donor | +C*T*TCGGGATTTTTACCTGGACCAGTTCGGCAGCAACGTTG | 29 | 1097 |
| | GAATTCAGTCGGAGGCAGAGAGGCAGCTCTTGAAGGGCTCGAA | | |
| | C*C*A+ | | |
| HDR donor | +G*G*CTGGGTCCCAGCCATCCAGGAACTCTAACACGTTGGGG | 30 | 1098 |
| | GAATTCACGTGGACAAAGACATCGTCATCTCCCTTTAGCATGA | | |
| | A*A*T+ | | |
| HDR donor | +T*T*GAAACCTTGCAGAGCACAATTGCCACCAATAATCGCAA | 31 | 1099 |
| | GAATTCGAGGGGAAGAAATGTCTCCCAATGTCCCCAGCACAAT | | |
| | T*G*C+ | | |
| HDR donor | +A*G*GCTGTGGGCCAGTTCTGACTGCAACGAGGCAGCCGACA | 32 | 1100 |
| | GAATTCCGTCGGCGTCCTGGAGGCGTGACTCCTCCTCCAGCTC | | |
| | G*G*A+ | | |
| HDR donor | +A*G*GTCCATACCCCACATTGAGGTGATCCACCAAAGCTTCT | 33 | 1101 |
| | GAATTCGTCAGGCAGGTCGCTGCAGTGATAGCCTCTCGCCTCC | | |
| | T*C*T+ | | |
| HDR donor | +A*A*ATTTTGAAATCTCTTGTTCCAGTGTGTCTAACAATACA | 34 | 1102 |
| | GAATTCACTTGGTTCTGTCTGAAACAAAAACACACAGCCAAGA | | |
| | C*A*A+ | | |
| HDR donor | +C*A*AAGACATTGTGAGCCACCTCGACACGAAGCCAATCAGG | 35 | 1103 |
| | GAATTCTTCAGGAACGCTGAGGAAGGGTGTGGATTCCGCAAGT | | |
| | C*C*C+ | | |
| HDR donor | +G*G*AGCAGGAGAAGCTCTCCGGTGTGGTGAAGAGCGTCCAC | 36 | 1104 |
| | GAATTCCGGCGGCTCCGCAAGAAGTACCGAGAAGGTAAACAGC | | |
| | G*C*G+ | | |
| HDR donor | +G*G*AGAAGATGGACATCACTGGAGGGTGTTCCGAATGGGAC | 37 | 1105 |
| | GAATTCCACGGGAGCAGCGCGTAGACATGACTGTCATTGAGCC | | |
| | C*T*A+ | | |
| HDR donor | +G*T*GGGCAACGTGCCCTTGGAGTGGTACGATGACTTCCCCC | 38 | 1106 |
| | GAATTCACGTGGGCTACGACCTGGATGGCAGGCGCATCTACAA | | |
| | G*C*C+ | | |
| HDR donor | +A*A*CCTTGCGTTTATTTTCAATGCTCAACGCCAGATCTTGT | 39 | 1107 |
| | GAATTCCGTTGGCATTCTACTTCAGCCCAGTTGAGAGCTGCCT | | |
| | C*A*A | | |
| HDR donor | +T*T*CTTTGCTTTGGCACTGTTAGGGTAGTCTACCACCATGC | 40 | 1108 |
| | GAATTCCACCGGAGAAGCCTGCCTTTGTGGCCTGGGTTGTGAT | | |
| | C*A*G+ | | |
| HDR donor | +G*G*CTTCACTTCACAGGTAGGAGGCTGGGCCACAAAAGGGA | 41 | 1109 |
| | GAATTCTACAGGAAGGAATGCTGGTGCTTACATCCTGCTCCAC | | |
| | T*T*C+ | | |
| HDR donor | +C*T*CACCCCCGACGGCTTCTTCTTCCGAGTCCACATGTTAG | 42 | 1110 |
| | GAATTCCCCTGGACTCCTCCAGCTGCAATAAGCCGTGTCCAGA | | |
| | G*T*T+ | | |
| HDR donor | +C*C*AACGGCGAGTCCCGGTGGGCCGCCCACCAAACCCCCGA | 43 | 1111 |
| | GAATTCCGAAGGCCATGGCCCCTGTGACCAGGGCACCCTTCCC | | |
| | A*G*A+ | | |
| HDR donor | +C*C*ACGGGGGAGATCCCAAGCTCAGTCCCCACAAAGTTCAG | 44 | 1112 |
| | GAATTCGGCCGGTCGGAGGCAGGGGCAGGTCCGGGTCCAAAGG | | |
| | T*A*A+ | | |
| HDR donor | +A*G*GAGGTCCAGAGGAGACCATCACTCAGCAAGGACGAACG | 45 | 1113 |
| | GAATTCCCAAGGACAGTAACTGAGTCCAGCTCATCCCACCCTC | | |
| | C*T*G+ | | |
| HDR donor | +C*A*AAAGGATTATGTGATTCTTGCCACTAGAACGCCACCCA | 46 | 1114 |
| | GAATTCAAGAGGAGCAAAGTGAGAACCTCAAACATCCCAAAGC | | |
| | T*A*A+ | | |
| HDR donor | +G*C*CAGGTCGAAGGCGCCGTCCAGGTTCACCAGCTCCGTGT | 47 | 1115 |
| | GAATTCCGAAGGGCACCGCCTGGAAGTGGTCGGAGCTGTGCAG | | |
| | G*C*C+ | | |
| HDR donor | +T*A*GATACTGTAGAGAAATCTGTGGGGTTGACCCCAAAGCT | 48 | 1116 |
| | GAATTCAACAGGTAGAGCTAAGGAATCCTTAGGGATGCTGCTG | | |
| | C*A*G+ | | |
| NGS F primer | acactctttccctacacgacgctcttccgatctAGAGGGCTGA | 1 | 1117 |
| | CAGAAATAATAAC | | |
| NGS F primer | acactctttccctacacgacgctcttccgatctCACAGACTGC | 2 | 1118 |
| | AGCCAAC | | |
| NGS F primer | acactctttccctacacgacgctcttccgatctAGACTCCGAA | 3 | 1119 |
| | GCTGACCT | | |
| NGS F primer | acactctttccctacacgacgctcttccgatctAAGGTCATCG | 4 | 1120 |
| | CCCCAGA | | |
| NGS F primer | acactctttccctacacgacgctcttccgatctCATTCAACCA | 5 | 1121 |
| | CTTCCCTGT | | |
| NGS F primer | acactctttccctacacgacgctcttccgatctTAGAGTATGC | 6 | 1122 |
| | AATCTGGGCA | | |
| NGS F primer | acactctttccctacacgacgctcttccgatctCAGGTAGTCT | 7 | 1123 |
| | CTGCCTTC | | |
| NGS F primer | acactctttccctacacgacgctcttccgatctACAGAGGGAA | 8 | 1124 |
| | ATGACATTGC | | |
| NGS F primer | acactctttccctacacgacgctcttccgatctCCTCCAGTCC | 9 | 1125 |
| | TTACTTGAACTT | | |
| NGS F primer | acactctttccctacacgacgctcttccgatctGTTTTCTTCC | 10 | 1126 |
| | CCTTCCCATC | | |
| NGS F primer | acactctttccctacacgacgctcttccgatctGAAACCAATC | 11 | 1127 |
| | AAGCTCCTGG | | |
| NGS F primer | acactctttccctacacgacgctcttccgatctTGGTTTCCTC | 12 | 1128 |
| | TCTCCGAG | | |
| NGS F primer | acactctttccctacacgacgctcttccgatctTCTTCTCTTA | 13 | 1129 |
| | GGGTTGGATGG | | |
| NGS F primer | acactctttccctacacgacgctcttccgatctCCACTACTTC | 14 | 1130 |
| | TTTTCCATTGAGG | | |
| NGS F primer | acactctttccctacacgacgctcttccgatctGCTCCAGTGC | 15 | 1131 |
| | ATGATGAG | | |
| NGS F primer | acactctttccctacacgacgctcttccgatctGTCCCATCCT | 16 | 1132 |
| | AGTTTGGC | | |
| NGS F primer | acactctttccctacacgacgctcttccgatctCTCTTCTCTC | 17 | 1133 |
| | CTGCCCTTT | | |
| NGS F primer | acactctttccctacacgacgctcttccgatctCTTCAAAAGG | 18 | 1134 |
| | GAGCCACAT | | |
| NGS F primer | acactctttccctacacgacgctcttccgatctTTCTTCTCAG | 19 | 1135 |
| | CTTACCACAGT | | |
| NGS F primer | acactctttccctacacgacgctcttccgatctGGGACTGTAG | 20 | 1136 |
| | CTAATCCTAAC | | |
| NGS F primer | acactctttccctacacgacgctcttccgatctACAGGACACT | 21 | 1137 |
| | TCCTTGCA | | |
| NGS F primer | acactctttccctacacgacgctcttccgatctTAAAGATGAG | 22 | 1138 |
| | TCGCTGGAG | | |
| NGS F primer | acactctttccctacacgacgctcttccgatctAAAGGTCTCA | 23 | 1139 |
| | AGATTCTGCC | | |
| NGS F primer | acactctttccctacacgacgctcttccgatctAAGGAAAACC | 24 | 1140 |
| | TACTCTCTCTGG | | |
| NGS F primer | acactctttccctacacgacgctcttccgatctAATGACTGCC | 25 | 1141 |
| | CCACATTTTA | | |
| NGS F primer | acactctttccctacacgacgctcttccgatctGCCCATAGGT | 26 | 1142 |
| | AAAGTGTTGA | | |
| NGS F primer | acactctttccctacacgacgctcttccgatctCCAGAAGTCT | 27 | 1143 |
| | TCTCAGCATTT | | |
| NGS F primer | acactctttccctacacgacgctcttccgatctCCGCCCACCT | 28 | 1144 |
| | TGTATTT | | |
| NGS F primer | acactctttccctacacgacgctcttccgatctTTTCTCCTCC | 29 | 1145 |
| | TGCCCTAAT | | |
| NGS F primer | acactctttccctacacgacgctcttccgatctAGGCCCATTT | 30 | 1146 |
| | CATGCTAAA | | |
| NGS F primer | acactctttccctacacgacgctcttccgatctATACCGTCCA | 31 | 1147 |
| | AAAGAGATCACTT | | |
| NGS F primer | acactctttccctacacgacgctcttccgatctTGCAACCCTC | 32 | 1148 |
| | TCGATGG | | |
| NGS F primer | acactctttccctacacgacgctcttccgatctCAACTAGCAG | 33 | 1149 |
| | AATAGTAATGGATGG | | |
| NGS F primer | acactctttccctacacgacgctcttccgatctCACTTTAAAT | 34 | 1150 |
| | ATGTAGAGTTTGTCTTGG | | |
| NGS F primer | acactctttccctacacgacgctcttccgatctCCTACAGTGT | 35 | 1151 |
| | TTTCAGACTCCA | | |
| NGS F primer | acactctttccctacacgacgctcttccgatctTTCCTCCCTC | 36 | 1152 |
| | ACTCAGC | | |
| NGS F primer | acactctttccctacacgacgctcttccgatctGTTGTATGTG | 37 | 1153 |
| | GGATGTGACT | | |
| NGS F primer | acactctttccctacacgacgctcttccgatctAACTGGTCCA | 38 | 1154 |
| | GCTCATCC | | |
| NGS F primer | acactctttccctacacgacgctcttccgatctGAAACTCTGA | 39 | 1155 |
| | ATGCCAAAGAAATT | | |
| NGS F primer | acactctttccctacacgacgctcttccgatctGCTGCCTTTC | 40 | 1156 |
| | TTTCCTCA | | |
| NGS F primer | acactctttccctacacgacgctcttccgatctTTCCAGGAGA | 41 | 1157 |
| | AGTGGAGCA | | |
| NGS F primer | acactctttccctacacgacgctcttccgatctCAGGTTTAAA | 42 | 1158 |
| | CTCTGGACACG | | |
| NGS F primer | acactctttccctacacgacgctcttccgatctTGTGAGACAC | 43 | 1159 |
| | CTGCACTTA | | |
| NGS F primer | acactctttccctacacgacgctcttccgatctCAACCACCCA | 44 | 1160 |
| | ACTTCTCTC | | |
| NGS F primer | acactctttccctacacgacgctcttccgatctCTTCTGGCAA | 45 | 1161 |
| | TGTGGATATTC | | |
| NGS F primer | acactctttccctacacgacgctcttccgatctGCTTTTTAAT | 46 | 1162 |
| | TTGTTGTTGAAGTGTT | | |
| NGS F primer | acactctttccctacacgacgctcttccgatctCAGGTGTGCA | 47 | 1163 |
| | CGTTGAG | | |
| NGS F primer | acactctttccctacacgacgctcttccgatctCAGAATCTTC | 48 | 1164 |
| | AGAAATGGCACAA | | |
| NGS R primer | gtgactggagttcagacgtgtgctcttccgatctAAAATCAAT | 1 | 1165 |
| | GATGCCATAGCTGA | | |
| NGS R primer | gtgactggagttcagacgtgtgctcttccgatctGAATCCCAA | 2 | 1166 |
| | CATGGTCCC | | |
| NGS R primer | gtgactggagttcagacgtgtgctcttccgatctTTGTTGACC | 3 | 1167 |
| | AGCTCCAGG | | |
| NGS R primer | gtgactggagttcagacgtgtgctcttccgatctGGGAAAGAG | 4 | 1168 |
| | GAGGGCC | | |
| NGS R primer | gtgactggagttcagacgtgtgctcttccgatctGTAATGAGA | 5 | 1169 |
| | GATGGGCTCAC | | |
| NGS R primer | gtgactggagttcagacgtgtgctcttccgatctCTACAGGAG | 6 | 1170 |
| | ACCTTTGAGGA | | |
| NGS R primer | gtgactggagttcagacgtgtgctcttccgatctCAGGATTCG | 7 | 1171 |
| | ACTCAGGC | | |
| NGS R primer | gtgactggagttcagacgtgtgctcttccgatctTTCTATATA | 8 | 1172 |
| | TCCCCAGCCGG | | |
| NGS R primer | gtgactggagttcagacgtgtgctcttccgatctTATGTACGA | 9 | 1173 |
| | TGGCTTCTGGTC | | |
| NGS R primer | gtgactggagttcagacgtgtgctcttccgatctGGTCCCTTT | 10 | 1174 |
| | CTCATTCAGTTA | | |
| NGS R primer | gtgactggagttcagacgtgtgctcttccgatctTGTGCATCA | 11 | 1175 |
| | CTTACCGGTT | | |
| NGS R primer | gtgactggagttcagacgtgtgctcttccgatctTCCAGCTGA | 12 | 1176 |
| | AAATTGGAGC | | |
| NGS R primer | gtgactggagttcagacgtgtgctcttccgatctAACATTGGC | 13 | 1177 |
| | ATTTTCCATGATG | | |
| NGS R primer | gtgactggagttcagacgtgtgctcttccgatctTCCCGGTTT | 14 | 1178 |
| | TAGAGAAATGTG | | |
| NGS R primer | gtgactggagttcagacgtgtgctcttccgatctGCAGTAGGT | 15 | 1179 |
| | AGCCGAGAT | | |
| NGS R primer | gtgactggagttcagacgtgtgctcttccgatctTGGACCTGA | 16 | 1180 |
| | CAAGGAGAG | | |
| NGS R primer | gtgactggagttcagacgtgtgctcttccgatctTTGTCCTCT | 17 | 1181 |
| | GCAGTACCTG | | |
| NGS R primer | gtgactggagttcagacgtgtgctcttccgatctAACCGCCAT | 18 | 1182 |
| | GATCAGAAG | | |
| NGS R primer | gtgactggagttcagacgtgtgctcttccgatctGCTCCTGAA | 19 | 1183 |
| | CAATATCTAAGTGT | | |
| NGS R primer | gtgactggagttcagacgtgtgctcttccgatctTTCGTGGGA | 20 | 1184 |
| | AAAACTGTCTC | | |
| NGS R primer | gtgactggagttcagacgtgtgctcttccgatctCCACCAAGT | 21 | 1185 |
| | GCTTACGG | | |
| NGS R primer | gtgactggagttcagacgtgtgctcttccgatctCTTTCCTCC | 22 | 1186 |
| | TCCCTGAGA | | |
| NGS R primer | gtgactggagttcagacgtgtgctcttccgatctGTTTGCCCA | 23 | 1187 |
| | GAACTGTTGATT | | |
| NGS R primer | gtgactggagttcagacgtgtgctcttccgatctGAGTCAAAG | 24 | 1188 |
| | ATAAACACTTCATGC | | |
| NGS R primer | gtgactggagttcagacgtgtgctcttccgatctCTCTAGGCC | 25 | 1189 |
| | ATACTGGAGAT | | |
| NGS R primer | gtgactggagttcagacgtgtgctcttccgatctGTGGAAACT | 26 | 1190 |
| | CTGTCATGTGT | | |
| NGS R primer | gtgactggagttcagacgtgtgctcttccgatctCACAGTAAC | 27 | 1191 |
| | AGCTGTCTGG | | |
| NGS R primer | gtgactggagttcagacgtgtgctcttccgatctGGTGTTTGT | 28 | 1192 |
| | CCTGGGC | | |
| NGS R primer | gtgactggagttcagacgtgtgctcttccgatctGTGAGTTAT | 29 | 1193 |
| | TGGTTCGAGCC | | |
| NGS R primer | gtgactggagttcagacgtgtgctcttccgatctTGGCTCTGG | 30 | 1194 |
| | ACATGACATAT | | |
| NGS R primer | gtgactggagttcagacgtgtgctcttccgatctCTTTTCTTA | 31 | 1195 |
| | GGTAGCAGATGGG | | |
| NGS R primer | gtgactggagttcagacgtgtgctcttccgatctTCCGAGCTG | 32 | 1196 |
| | GAGGAGG | | |
| NGS R primer | gtgactggagttcagacgtgtgctcttccgatctGAGGAAACT | 33 | 1197 |
| | GATGTTGATAAGAGGT | | |
| NGS R primer | gtgactggagttcagacgtgtgctcttccgatctCCAAAGCAT | 34 | 1198 |
| | TAATATCCAACATAGAATGA | | |
| NGS R primer | gtgactggagttcagacgtgtgctcttccgatctCTGGGCTTT | 35 | 1199 |
| | CCATGAATTATGAA | | |
| NGS R primer | gtgactggagttcagacgtgtgctcttccgatctGCCGAAATA | 36 | 1200 |
| | CTGCTCGT | | |
| NGS R primer | gtgactggagttcagacgtgtgctcttccgatctATGCTAGCC | 37 | 1201 |
| | ATTACCTCCAT | | |
| NGS R primer | gtgactggagttcagacgtgtgctcttccgatctTAGGATCAG | 38 | 1202 |
| | ATGCCGACAT | | |
| NGS R primer | gtgactggagttcagacgtgtgctcttccgatctCAAGTAAAG | 39 | 1203 |
| | TGCCTTTCCTAGAA | | |
| NGS R primer | gtgactggagttcagacgtgtgctcttccgatctGCTATGCCA | 40 | 1204 |
| | CTACCCTCC | | |
| NGS R primer | gtgactggagttcagacgtgtgctcttccgatctCTGTGGAGT | 41 | 1205 |
| | ACCTCTTCCGT | | |
| NGS R primer | gtgactggagttcagacgtgtgctcttccgatctGGGTAATGC | 42 | 1206 |
| | TCTTCTCCAAA | | |
| NGS R primer | gtgactggagttcagacgtgtgctcttccgatctATCATGAAG | 43 | 1207 |
| | CTGCTGTGCT | | |
| NGS R primer | gtgactggagttcagacgtgtgctcttccgatctGACTTACCT | 44 | 1208 |
| | TTGGACCCG | | |
| NGS R primer | gtgactggagttcagacgtgtgctcttccgatctTCAGGAAGT | 45 | 1209 |
| | CATTGCTTTCC | | |
| NGS R primer | gtgactggagttcagacgtgtgctcttccgatctCATTCTTCA | 46 | 1210 |
| | TCCAAGTTATCCAACTTA | | |
| NGS R primer | gtgactggagttcagacgtgtgctcttccgatctCTACGCCGC | 47 | 1211 |
| | CTTCTCC | | |
| NGS R primer | gtgactggagttcagacgtgtgctcttccgatctATTTTGGTA | 48 | 1212 |
| | CCTGAAGATCTGG | | |

All gRNAs, HDR templates, and primers were synthesized by IDT (Coralville, IA). SEQ ID NO: 1021-1068 represent the 20 nt protospacer sequence corresponding to gRNAs used in Example 4. The generated gRNAs have 5′- and 3′-Alt-R ™ termini modifications and were in Cas9 crRNA format. SEQ ID NO: 1069-1116 represent the HDR templates tested in Example 4. HDR templates have 5′- and 3′-Alt-R ™ termini modifications (+) and phosphorothioate (*) linkages between nucleotides 1-2, 2-3, 84-85, and 85-86. The 5′- and 3′-Alt-R ™ termini modifications are a proprietary termini-blocking technology available from IDT (Coralville, IA). SEQ ID NO: 1117-1212 represent the NGS primers used in Example 4. Uppercase nucleotides indicate the gene specific portion of the primers, lowercase nucleotides indicates constant regions for subsequent NGS barcoding steps.

1. A method for predicting the homology-directed repair (HDR) potential of one or more Cas guide RNAs (gRNAs), the process comprising:
(a) generating an empirical indel profile for one or more candidate gRNAs by:
(i) performing one or more Cas enzyme editing experiments using one or more candidate gRNAs and obtaining edited genomic DNA;
(ii) for each editing experiment, amplifying and sequencing the edited genomic DNA to generate sequenced edited genomic DNA;
executing on a processor, for each editing experiment:
(iii) receiving the sequenced edited genomic DNA; and
(iv) analyzing the sequenced edited genomic DNA and outputting an empirical indel profile;
(b) inputting the empirical indel profile from step (a) into an HDR predictive model and analyzing the indel profiles; and
(c) outputting an HDR rate threshold, HDR score, or rank ordered listing of the candidate gRNAs indicating preferred candidate gRNAs for an HDR editing experiment and optimal editing sites.

2. A method for predicting the homology-directed repair (HDR) potential of one or more Cas guide RNAs (gRNAs), the process comprising:
(a) generating an in silico indel profile for one or more candidate gRNAs by executing on a processor:
(i) inputting a candidate gRNA sequence and editing locus; and
(ii) receiving an in silico indel profile;
(b) inputting the in silico indel profile from step (a) into an HDR predictive model and analyzing the indel profiles; and
(c) outputting an HDR rate threshold, HDR score, or rank ordered listing of the candidate gRNAs indicating preferred candidate gRNAs for an HDR editing experiment and optimal editing sites.

3. A method for predicting the homology-directed repair (HDR) potential of one or more Cas guide RNAs (gRNAs), the process comprising:
(a) generating an empirical indel profile for one or more candidate gRNAs by:
(i) performing one or more Cas enzyme editing experiments using one or more candidate gRNAs and obtaining edited genomic DNA;
(ii) for each editing experiment, amplifying and sequencing the edited genomic DNA to generate sequenced edited genomic DNA;
executing on a processor, for each editing experiment:
(iii) receiving the sequenced edited genomic DNA; and
(iv) analyzing the sequenced edited genomic DNA and outputting an empirical indel profile;
or
(b) generating an in silico indel profile for one or more candidate gRNAs by executing on a processor:
(i) inputting a candidate gRNA sequence and editing locus; and
(ii) receiving an in silico indel profile;
(c) inputting the empirical indel profile from step (a) or in silico indel profile from step (b) into an HDR predictive model and analyzing the indel profiles; and
(d) outputting an HDR rate threshold, HDR score, or rank ordered listing of the candidate gRNAs indicating preferred candidate gRNAs for an HDR editing experiment and optimal editing sites.

4. The method of claim 3, wherein step (a)(ii) comprises amplifying the genomic DNA using RNase H-dependent PCR (rhPCR) and performing next generation sequencing (NGS) to generate sequenced edited genomic DNA.

5. The method of claim 3, wherein the analyzing the sequenced edited genomic DNA in step (a)(iv) comprises merging the sequenced edited genomic DNA, binning the merged sequenced edited genomic DNA by alignment to the genome, and providing alignments of the edited genomic DNA and a characterization and quantitation of the empirical indel frequency.

6. The method of claim 5, wherein the analysis is performed using rhAmpSeq CRISPR Analysis System or CRISPAltRations.

7. The method of claim 3, wherein the empirical indel profile comprises one or more of allele frequency, templated insertion frequency, microhomology-mediated end joining (MMEJ) deletion frequency, entropy, insertion size frequency, GC insertion motif frequency, deletion size frequency, or combinations thereof.

8. The method of claim 3, wherein generating the in silico indel profile comprises predicting guide RNA efficacy and producing alignments and editing frequency, and mutational outcomes resulting from double stranded breaks.

9. The method of claim 8, wherein the input is a guide sequence, and the output is a set of alignments and predictions for on-target base editing efficacy.

10. The method of claim 3, where the generating the in silico indel profile is performed using FORECasT.

11. The method of claim 3, wherein the HDR predictive model in step (c) comprises a gradient boosted regressor, ensemble method, lasso regression, Structural Equation Modeling (SEM), or traditional machine learning process that transforms the multi-dimensional indel profile into an HDR rate threshold, HDR score, or rank ordered output for the candidate gRNAs.

12. The method of claim 3, wherein the HDR predictive model is trained by executing on a processor:
(i) creating a training set of data using the empirical indel profile or in silico indel profile;
(ii) creating a test set of data using the empirical indel profile or in silico indel profile; and
(iii) training and testing the HDR predictive model, wherein the HDR predictive model is trained using the training set of data, and wherein the HDR predictive model is tested using the testing set of data.

13. The method of claim 3, wherein the HDR predictive model is capable of accurately ranking candidate gRNAs for overall HDR potential with a Spearman correlation value of greater than 0.5.

14. The method of claim 3, wherein the HDR rates and preferred candidate gRNAs are specific for a particular cell type or cell line.

15. The method of claim 3, wherein the candidate gRNA sequences have a variable region from about 17 nucleotides to about 24 nucleotides in length.

16. (canceled)

17. The method of claim 3, wherein the candidate gRNA sequences comprise one or more termini-blocking modifications on their 5′-termini, 3′-termini, or a combination thereof.

18. (canceled)

19. The method of claim 3, wherein the editing site or editing locus is Cas-enzyme specific and comprises from about 1 nucleotide to about 15 nucleotides.

20. The method of claim 3, wherein the Cas enzyme is Cas9 or Cas 12a.

21. The method of claim 3, wherein the genomic DNA is from a population of cells or subjects.

22. The method of claim 3, wherein the candidate gRNA sequences comprise sequences from one or more of SEQ ID NO: 1-255 or 1021-1068.
